(19) United States
(12) Patent Application Publication
(10) Pub. No.: US 2026/0357209 A1
(43) Pub. Date: Nov. 12, 2026

(54) ENGINEERED INDUCED STEM CELL DERIVED MYELOID CELLS AND METHODS OF DIFFERENTIATING AND USING SAME

(71) Applicant: Umoja Biopharma, Inc., Seattle, WA (US)

(72) Inventors: Dillon JARRELL, Seattle, WA (US); Ryan KONING, Denver, CO (US)

(73) Assignee: Umoja Biopharma, Inc., Seattle, WA (US)

(21) Appl. No.: 19/704,338

(22) Filed: Jun. 10, 2026

Related U.S. Application Data
(63) Continuation of application No. PCT/US2024/060149, filed on Dec. 13, 2024.
(60) Provisional application No. 63/609,821, filed on Dec. 13, 2023.

(51) Int. Cl. C12N 5/0786; A61K 31/439; A61K 40/17; A61K 40/31; A61K 40/42; A61P 35/00; C07K 14/715; C07K 16/28; C12N 5/074; C12N 9/90; C12N 15/90

(52) U.S. Cl. CPC C12N 5/0645; A61K 31/439; A61K 40/17; A61K 40/31; A61K 40/4211; A61P 35/00; C07K 14/7155; C07K 16/2803; C12N 5/0696; C12N 9/90; C12N 15/907; C12Y 502/01008; C12N 2501/115; C12N 2501/125; C12N 2501/155; C12N 2501/165; C12N 2501/22; C12N 2501/2303; C12N 2501/727; C12N 2501/999; C12N 2506/45; C12N 2510/00

(57) ABSTRACT

Provided are compositions and methods for a cell population comprising macrophage cells comprising a synthetic cytokine receptor for a non-physiological ligand. The non-physiological ligand activates the synthetic cytokine receptor in the engineered stem cells to induce differentiation of the stem cells and, expansion and/or activation of resulting cytotoxic macrophage cells.

### CROSS-REFERENCE TO RELATED APPLICATIONS

This application is a continuation of International Application No. PCT/US2024/060149, filed internationally on Dec. 13, 2024, which claims priority from U.S. Provisional Application No. 63/609,821 filed Dec. 13, 2023, entitled “ENGINEERED INDUCED STEM CELL DERIVED MYELOID CELLS AND METHODS OF DIFFERENTIATING AND USING SAME”, the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 260132001401SeqList.XML, created Jun. 9, 2026, which is 97,096 bytes. The information in the electronic format of the Sequence Listing is herein incorporated by reference in its entirety.

### FIELD

The present disclosure provides compositions and methods related to a cell population comprising engineered myeloid cells derived from stem cells comprising a synthetic cytokine receptor for a non-physiological ligand. The non-physiological ligand activates the synthetic cytokine receptor in the engineered stem cells to induce differentiation and, expansion and/or activation of resulting myeloid cells (e.g., macrophages).

### BACKGROUND

Macrophages are a class of immune cells that may be used in immunotherapy including cancer immunotherapy. Macrophages are type of cell generally identified as positive for the cell surface protein CD14 (CD14+) and other markers and as having phagocytic and cytotoxic activity.

Macrophage cells for use in immunotherapy can be derived from obtained from primary sources such as peripheral blood or umbilical cord blood. Artificial sources for macrophages include pluripotent stem cells, including induced pluripotent stem cells (iPSCs), which are cells derived from somatic cells (generally fibroblasts or peripheral blood mononuclear cells [PBMCs]), and human embryonic stem cells (hESCs), either induced to become capable of unlimited proliferation and of differentiation into other cell types when subjected to appropriate differentiation conditions.

Methods for differentiating iPSCs into CD34+ HPCs using either embryoid embodies (EBs) or culture of single-cell iPSCs on feeder cells are known. CD34+ HPCs may then be differentiated into myeloid progenitor cells. There is a need for improved methods, including methods for differentiating stem cells into macrophages, and methods of using them in immunotherapy.

### SUMMARY

In some aspects, provided herein is an engineered cell that is a myeloid progenitor cell comprising a synthetic cytokine receptor for a non-physiological ligand, wherein the synthetic cytokine receptor comprises: a synthetic gamma chain polypeptide comprising a first dimerization domain, a first transmembrane domain, and an interleukin-2 receptor subunit gamma (IL-2RG) intracellular domain, and a synthetic beta chain polypeptide comprising a second dimerization domain, a second transmembrane domain, and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain. In some of any of the provided embodiments, the myeloid progenitor cell is a Granulocyte/Monocyte Progenitor Cell (“GMP”). In some of any of the provided embodiments, the myeloid progenitor cell is characterized with a surface phenotype CD34+, CD90−, and CD45RA+. In some of any of the provided embodiments, the myeloid progenitor cells are characterized with a surface phenotype CD34+, CD90−, CD123+, and CD45RA+.

In some aspects, provided herein is an engineered cell that is a myeloid cell comprising a synthetic cytokine receptor for a non-physiological ligand, wherein the synthetic cytokine receptor comprises: a synthetic gamma chain polypeptide comprising a first dimerization domain, a first transmembrane domain, and an interleukin-2 receptor subunit gamma (IL-2RG) intracellular domain, and a synthetic beta chain polypeptide comprising a second dimerization domain, a second transmembrane domain, and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain. In some of any of the provided embodiments, the myeloid cell is a macrophage, a neutrophil, a megakaryocyte, a monocyte, a basophil, an eosinophil, and/or an erythrocyte cell.

In some aspects, provided herein is an engineered cell that is a macrophage comprising a synthetic cytokine receptor for a non-physiological ligand, wherein the synthetic cytokine receptor comprises: a synthetic gamma chain polypeptide comprising a first dimerization domain, a first transmembrane domain, and an interleukin-2 receptor subunit gamma (IL-2RG) intracellular domain, and a synthetic beta chain polypeptide comprising a second dimerization domain, a second transmembrane domain, and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain. In some of any of the provided embodiments, the macrophage is a mature macrophage that expresses CD14.

In some aspects, provided herein is an engineered cell that is a neutrophil comprising a synthetic cytokine receptor for a non-physiological ligand, wherein the synthetic cytokine receptor comprises: a synthetic gamma chain polypeptide comprising a first dimerization domain, a first transmembrane domain, and an interleukin-2 receptor subunit gamma (IL-2RG) intracellular domain, and a synthetic beta chain polypeptide comprising a second dimerization domain, a second transmembrane domain, and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain. In some of any of the provided embodiments, the engineered cell has been differentiated from a stem cell. In some of any of the provided embodiments, the stem cell is a pluripotent stem cell. In some of any of the provided embodiments, the stemcell is an induced pluripotent stem cell. In some of any of the provided embodiments, the myeloid cell is an induced myeloid cell (iMC) differentiated from a stem cell engineered with the synthetic cytokine receptor. In some of any of the provided embodiments, wherein the macrophage is an induced macrophage (iMAC) differentiated from a stem cell engineered with the synthetic cytokine receptor. In some of any of the provided embodiments, the neutrophil is an induced neutrophil (iNEU) differentiated from a stem cell engineered with the synthetic cytokine receptor. In some of any of the provided embodiments, the synthetic gamma chain polypeptide comprises, in N- to C-terminal order, the first dimerization domain, the first transmembrane domain, and the IL-2RG intracellular domain, and the synthetic beta chain polypeptide comprises, in N- to C-terminal order, the second dimerization domain, the second transmembrane domain, and the beta chain intracellular domain. In some of any of the provided embodiments, the first dimerization domain and the second dimerization domain are extracellular domains.

In some of any of the provided embodiments, the IL-2RG intracellular domain comprises a polypeptide sequence at least 95% identical to SEQ ID NO: 1, or a polypeptide sequence as set forth in SEQ ID NO: 1. In some of any of the provided embodiments, the first transmembrane domain comprises the IL-2RG transmembrane domain. In some of any of the provided embodiments, the IL-2RG transmembrane domain comprises a polypeptide sequence at least 95% identical to SEQ ID NO: 8 or 31, or a polypeptide sequence as set forth in SEQ ID NO: 8 or 31. In some of any of the provided embodiments, the beta chain intracellular domain comprises the IL-2RB intracellular domain. In some of any of the provided embodiments, the IL-2RB intracellular domain comprises a polypeptide sequence at least 95% identical to SEQ ID NO: 2, or a polypeptide sequence as set forth in SEQ ID NO: 2. In some of any of the provided embodiments, the beta chain intracellular domain is an IL-7RB intracellular domain. In some of any of the provided embodiments, the IL-7RB intracellular domain comprises a polypeptide sequence at least 95% identical to SEQ ID NO: 3, or a polypeptide sequence as set forth in SEQ ID NO: 3. In some of any of the provided embodiments, the beta chain intracellular domain comprises the IL-21RB intracellular domain. In some of any of the provided embodiments, the IL-21RB intracellular domain comprises a polypeptide sequence at least 95% identical to SEQ ID NO: 4, or a polypeptide sequence as set forth in SEQ ID NO: 4. In some of any of the provided embodiments, the second transmembrane domain comprises a transmembrane domain from the same polypeptide as the beta chain intracellular domain. In some of any of the provided embodiments, the second transmembrane domain is a transmembrane domain of IL-2RB comprising a polypeptide sequence at least 95% identical to SEQ ID NO: 35 or 36, or a polypeptide sequence as set forth in SEQ ID NO: 35 or 36.

In some of any of the provided embodiments, the first transmembrane domain of the synthetic gamma chain polypeptide is an IL-2RG transmembrane domain comprising the sequence set forth in SEQ ID NO: 8 or 31 and the IL-2RG intracellular domain comprises the sequence set forth in SEQ ID NO:1; and the second transmembrane domain of the synthetic beta chain polypeptide is an IL-2RB transmembrane domain comprising the sequence set forth in SEQ ID NO: 35 or 36, and the beta chain intracellular domain is an IL-2RB intracellular domain comprising the sequence set forth in SEQ ID NO:2. In some of any of the provided embodiments, the first transmembrane domain and the IL-2RG intracellular domain of the synthetic gamma chain polypeptide comprise the sequences set forth in SEQ ID NOs: 31 and SEQ ID NO: 1; and the second transmembrane domain and the beta chain intracellular domain of the synthetic beta chain polypeptide comprise the sequences set forth in SEQ ID NOs: 35 and SEQ ID NO: 2.

In some of any of the provided embodiments, the first dimerization domain and the second dimerization domain are heterodimerization domains selected from FKBP12-rapamycin binding (FRB) domain and a FK506-Binding Protein of size 12 kD (FKBP); and/or wherein the non-physiological ligand is rapamycin or a rapalog. In some of any of the provided embodiments, the first dimerization domain is FKBP and the second dimerization domain is FRB. In some of any of the provided embodiments, the first dimerization domain is FRB and the second dimerization domain is FKBP. In some of any of the provided embodiments, the FRB domain comprises a polypeptide sequence at least 95% identical to SEQ ID NO: 6 or SEQ ID NO: 7. In some of any of the provided embodiments, the FRB domain comprises the polypeptide sequence set forth in SEQ ID NO: 6 or SEQ ID NO: 7. In some of any of the provided embodiments, the FKBP domain comprises a polypeptide sequence at least 95% identical to SEQ ID NO: 5 or SEQ ID NO:30. In some of any of the provided embodiments, the FKBP domain comprises the polypeptide sequence set forth in SEQ ID NO: 5 or SEQ ID NO:30.

In some of any of the provided embodiments, the synthetic gamma chain polypeptide has the sequence of amino acids set forth in SEQ ID NO: 28 or a sequence of amino acids that is at least 90%, at least 95%, at least 96%, at least 97%, at least 98%, at least 99%, or 100% identical to SEQ ID NO: 28, and the synthetic beta chain polypeptide has the sequence of amino acids set forth in SEQ ID NO: 55 or a sequence of amino acids that is at least 90%, at least 95%, at least 96%, at least 97%, at least 98%, at least 99%, or 100% identical to SEQ ID NO: 55. In some of any of the provided embodiments, the synthetic gamma chain polypeptide has the sequence of amino acids set forth in SEQ ID NO: 28 and the synthetic beta chain polypeptide has the sequence of amino acids set forth in SEQ ID NO: 55. In some of any of the provided embodiments, the synthetic gamma chain polypeptide has the sequence of amino acids set forth in SEQ ID NO: 28 or a sequence of amino acids that is at least 90%, at least 95%, at least 96%, at least 97%, at least 98%, at least 99%, or 100% identical to SEQ ID NO: 28, and the synthetic beta chain polypeptide has the sequence of amino acids set forth in SEQ ID NO: 55 or a sequence of amino acids that is at least 90%, at least 95%, at least 96%, at least 97%, at least 98%, at least 99%, or 100% identical to SEQ ID NO: 55. In some of any of the provided embodiments, the synthetic gamma chain polypeptide has the sequence of amino acids set forth in SEQ ID NO: 28 and the synthetic beta chain polypeptide has the sequence of amino acids set forth in SEQ ID NO: 55.

In some of any of the provided embodiments, the synthetic gamma chain polypeptide has the sequence of amino acids set forth in SEQ ID NO: 56 or a sequence of amino acids that is at least 90%, at least 95%, at least 96%, at least 97%, at least 98%, at least 99%, or 100% identical to SEQ ID NO: 56, and the synthetic beta chain polypeptide has the sequence of amino acids set forth in SEQ ID NO: 57 or a sequence of amino acids that is at least 90%, at least 95%, at least 96%, at least 97%, at least 98%, at least 99%, or 100% identical to SEQ ID NO: 57. In some of any of the provided embodiments, the synthetic gamma chain polypeptide has the sequence of amino acids set forth in SEQ ID NO: 56 and the synthetic beta chain polypeptide has the sequence of amino acids set forth in SEQ ID NO: 57.

In some of any of the provided embodiments, the first dimerization domain and the second dimerization domain are homodimerization domains selected from: i) FK506-Binding Protein of size 12 kD (FKBP); ii) cyclophiliA (CypA); or iii) gyrase B (CyrB); and the non-physiological ligand is, respectively: i) FK1012, AP1510, AP1903, or AP20187 or an analog thereof; ii) cyclosporin-A (CsA) or an analog thereof, or iii) coumermycin or an analog thereof. In some of any of the provided embodiments, the engineered cell is resistant to rapamycin-mediated mTOR inhibition. In some of any of the provided embodiments, the engineered cell expresses a cytosolic polypeptide that binds to the non-physiological ligand, optionally wherein the cytosolic polypeptide is a cytosolic FRB domain. In some of any of the provided embodiments, the non-physiological ligand is rapamycin or a rapalog, and the engineered cell expresses a cytosolic FRB domain or variant thereof.

In some of any of the provided embodiments, the cytosolic FRB domain comprises a polypeptide sequence at least 95% identical to SEQ ID NO: 6 or SEQ ID NO: 7. In some of any of the provided embodiments, the cytosolic FRB domain comprises a polypeptide sequence at least 98% identical to SEQ ID NO: 6 or SEQ ID NO: 7. In some of any of the provided embodiments, the engineered cell comprises a disrupted FKBP12 gene that reduces expression of FKBP12 in the cell. In some of any of the provided embodiments, the engineered cell comprises knock out of the FKBP12 gene. in some of any of the provided embodiments, the engineered cell comprises a nucleotide sequence encoding the synthetic cytokine receptor inserted into the genome of the cell. In some of any of the provided embodiments, the nucleotide sequence encoding the synthetic cytokine receptor is inserted into a non-target locus in the genome of the cell. In some of any of the provided embodiments, the nucleotide sequence encoding the synthetic cytokine receptor is inserted into an endogenous gene of the cell. In some of any of the provided embodiments, the insertion reduces expression of the endogenous gene in the locus. In some of any of the provided embodiments, the insertion knocks out the endogenous gene in the locus. In some of any of the provided embodiments, the insertion is by homology-directed repair. In some of any of the provided embodiments, the endogenous gene is a housekeeping gene, a blood-lineage specific loci or an immune-related gene. In some of any of the provided embodiments, the housekeeping gene is selected from eukaryotic translation elongation factor 1 alpha (EEF1A), glylceraldehyde-3-phosphate dehydrogenase (GAPDH), ubiquitin C (UBC), and actin beta (ACTB). In some of any of the provided embodiments, the immune-related gene is selected from a beta-2-microglobulin (B2M) gene, and a signal regulatory protein alpha (SIRPA) gene. In some of any of the provided embodiments, the engineered cell comprises a B2M knockout. In some of any of the provided embodiments, the engineered cell comprises a B2M knockout and a FKBP12 knockout.

In some of any of the provided embodiments, the engineered cell comprises a chimeric antigen receptor (CAR). In some of any of the provided embodiments, binding of the non-physiological ligand to the synthetic cytokine receptor activates the synthetic cytokine receptor in the engineered cell to induce expansion and/or activation of the engineered cell in a cell population.

In some aspects, provided herein is a population comprising any engineered myeloid progenitor cells provided herein. In some aspects, provided herein is a population comprising any engineered myeloid cells provided herein. In some aspects, provided herein is a population comprising any engineered macrophages provided herein. In some aspects, provided herein is a population comprising any engineered neutrophils provided herein.

In some aspects, provided herein is a method of generating genetically engineered myeloid cells differentiated from stem cells, the method comprising: a) culturing a population of stem cells engineered with a synthetic cytokine receptor under conditions to differentiate cells into a population of hematopoietic progenitors (HP), wherein the synthetic cytokine receptor comprises: a synthetic gamma chain polypeptide comprising a first dimerization domain, a first transmembrane domain, and an interleukin-2 receptor subunit gamma (IL-2RG) intracellular domain, and a synthetic beta chain polypeptide comprising a second dimerization domain, a second transmembrane domain, and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain; and b) culturing the cells produced in a) by incubation under conditions to generate myeloid cells, wherein at least a portion of one or both of steps a) andb) are carried out in the presence of a non-physiological ligand of the synthetic cytokine receptor. In some of any of the provided embodiments, the myeloid cell is a macrophage or a neutrophil.

In some aspects, provided herein is a method of generating genetically engineered macrophages differentiated from stem cells, the method comprising: a) culturing a population of stem cells engineered with a synthetic cytokine receptor under conditions to differentiate cells into a population of hematopoietic progenitors (HP), wherein the synthetic cytokine receptor comprises: a synthetic gamma chain polypeptide comprising a first dimerization domain, a first transmembrane domain and an interleukin-2 receptor subunit gamma (IL-2RG) intracellular domain, and a synthetic beta chain polypeptide comprising a second dimerization domain, a second transmembrane domain and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain; and b) culturing the cells produced in a) by incubation under conditions to generate macrophages, wherein at least a portion of one or both of steps a) and b) are carried out in the presence of a non-physiological ligand of the synthetic cytokine receptor.

In some of any of the provided embodiments, the culturing in a) is carried out by a first incubation under conditions to produce an Embryoid Body (EB) followed by one or more further incubations in the presence of the non-physiological ligand and optionally one or more myeloid cell differentiation factors selected from one or more of IL-3, M-CSF and GM-CSF. In some of any of the provided embodiments, the one or more myeloid cell differentiation factors is IL-3, M-CSF and GM-CSF. In some of any of the provided embodiments, the one or more further incubations comprises a second incubation in a second media comprising one or more of IL-3, GM-CSF, and M-CSF, and a third incubation in a third media comprising one or more of IL-3, GM-CSF, and M-CSF, wherein one or both of the second media and the third media comprises the non-physiological ligand.

In some of any of the provided embodiments, step a) comprises: (i) performing a first incubation comprising culturing the population of stem cells engineered with the synthetic cytokine receptor under conditions to form a first aggregate in a first media, (ii) contacting the aggregate with a dissociating agent to form a population of dissociated cells; (iii) performing a second incubation comprising culturing the population of dissociated cells under conditions to form the second aggregate in a second media; and (iv) performing a third incubation comprising culturing the population of cells in (iii) in a third media. In some embodiments, the second media comprises one or more of IL-3, GM-CSF, and M-CSF. In some embodiments, the third media comprises one or more of IL-3, GM-CSF, and M-CSF. In some of any of the provided embodiments, the first incubation is in a first media comprising one or more of BMP4, FGF2, VEGF-165 and a Rock Inhibitor. In some of any of the provided embodiments, the first incubation is in a first media comprising BMP4, FGF2, VEGF-165 and a Rock Inhibitor. In some of any of the provided embodiments, the Rock Inhibitor is Y27632. In some of any of the provided embodiments, the second media further comprises the non-physiological ligand of the synthetic cytokine receptor. In some of any of the provided embodiments, the second media does not comprise the non-physiological ligand of the synthetic cytokine receptor. In some of any of the provided embodiments, the culturing in the first media is for 1 to 3 days. In some of any of the provided embodiments, the second media comprises one or more of BMP4, FGF2, VEGF, LY294002, IL-3, GM-CSF, and M-CSF. In some of any of the provided embodiments, the second media comprises BMP4, FGF2, VEGF, LY294002, IL-3, and M-CSF. In some of any of the provided embodiments, the second media further comprises a non-physiological ligand of the synthetic cytokine receptor. In some of any of the provided embodiments, the second media does not comprise a non-physiological ligand of the synthetic cytokine receptor. In some of any of the provided embodiments, the culturing in the second media is for 3 to 6 days. In some of any of the provided embodiments, the third media comprises one or more of UM729, StemRegenin-1, BMP4, FGF2, VEGF, LY294002, IL-3, GM-CSF, and M-CSF. In some of any of the provided embodiments, the third media comprises UM729, StemRegenin-1, BMP4, FGF2, VEGF, LY294002, IL-3, GM-CSF, and M-CSF. In some of any of the provided embodiments, the third media further comprises a non-physiological ligand of the synthetic cytokine receptor. In some of any of the provided embodiments, the third media does not comprise a non-physiological ligand of the synthetic cytokine receptor. In some of any of the provided embodiments, the culturing in the third media is for 6 to 12 days.

In some of any of the provided embodiments, the culturing in a) produces myeloid progenitor cells. In some of any of the provided embodiments, the myeloid progenitor cell is a Granulocyte/Monocyte Progenitor Cells (“GMPs”). In some of any of the provided embodiments, the myeloid progenitor cells are characterized with a surface phenotype CD34+, CD90−, and CD45RA+. In some of any of the provided embodiments, the myeloid progenitor cells are characterized with a surface phenotype CD34+, CD90−, CD123+, and CD45RA+. In some of any of the provided embodiments, the culturing in b) is in a media comprising one or more of UM729, SCF, StemRegenin1, IL-3, GM-CSF, and M-CSF. In some of any of the provided embodiments, the culturing in b) is in a media comprising UM729, SCF, StemRegenin1, IL-3, GM-CSF, and M-CSF. In some of any of the provided embodiments, the culturing in b) is in a media further comprising a non-physiological ligand of the synthetic cytokine receptor. In some of any of the provided embodiments, the culturing in b) is in a media that does not comprise a non-physiological ligand of the synthetic cytokine receptor. In some of any of the provided embodiments, the culturing in b) is for 12 to 24 days.

In some aspects, provided herein is a method of generating genetically engineered myeloid cells to express a synthetic cytokine receptor, comprising: culturing a population of hematopoietic progenitors (HP) cells derived from stem cells engineered with a synthetic cytokine receptor under conditions to differentiate cells into a population of myeloid cells, wherein at least a portion of the culturing is carried out in the presence of a non-physiological ligand of the synthetic cytokine receptor, and wherein the synthetic cytokine receptor comprises: a synthetic gamma chain polypeptide comprising a first dimerization domain, a first transmembrane domain, and an interleukin-2 receptor subunit gamma (IL-2RG) intracellular domain, and a synthetic beta chain polypeptide comprising a second dimerization domain, a second transmembrane domain and beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

In some aspects, provided herein is a method of generating genetically engineered myeloid cells to express a synthetic cytokine receptor, comprising: culturing a population of myeloid progenitors cells derived from stem cells engineered with a synthetic cytokine receptor under conditions to differentiate cells into a population of myeloid cells, wherein at least a portion of the culturing is carried out in the presence of a non-physiological ligand of the synthetic cytokine receptor, and wherein the synthetic cytokine receptor comprises: a synthetic gamma chain polypeptide comprising a first dimerization domain, a first transmembrane domain, and an interleukin-2 receptor subunit gamma (IL-2RG) intracellular domain, and a synthetic beta chain polypeptide comprising a second dimerization domain, a second transmembrane domain and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain. In some of any of the provided embodiments, the myeloid cells are macrophages or neutrophils.

In some aspects, provided herein is a method of generating genetically engineered macrophages to express a synthetic cytokine receptor, comprising: culturing a population of hematopoietic progenitors (HP) cells derived from stem cells engineered with a synthetic cytokine receptor under conditions to differentiate cells into a population of macrophages, wherein at least a portion of the culturing is carried out in the presence of a non-physiological ligand of the synthetic cytokine receptor, and wherein the synthetic cytokine receptor comprises: a synthetic gamma chain polypeptide comprising a first dimerization domain, a first transmembrane domain and an interleukin-2 receptor subunit gamma (IL-2RG) intracellular domain, and a synthetic beta chain polypeptide comprising a second dimerization domain, a second transmembrane domain and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

In some aspects, provided herein is a method of generating genetically engineered macrophages to express a synthetic cytokine receptor, comprising: culturing a population of myeloid progenitors cells derived from stem cells engineered with a synthetic cytokine receptor under conditions to differentiate cells into a population of macrophages, wherein at least a portion of the culturing is carried out in the presence of a non-physiological ligand of the synthetic cytokine receptor, and wherein the synthetic cytokine receptor comprises: a synthetic gamma chain polypeptide comprising a first dimerization domain, a first transmembrane domain and an interleukin-2 receptor subunit gamma (IL-2RG) intracellular domain, and a synthetic beta chain polypeptide comprising a second dimerization domain, a second transmembrane domain and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

In some of any of the provided embodiments, the culturing is in a media comprising one or more of UM729, SCF, StemRegenin1, IL-3, GM-CSF, and M-CSF. In some of any of the provided embodiments, the culturing is in a media comprising UM729, SCF, StemRegenin1, IL-3, GM-CSF, and M-CSF. In some of any of the provided embodiments, the culturing is for 12 to 24 days. In some of any of the provided embodiments, the culturing of one or both steps a) andb) is carried out in a bioreactor. In some of any of the provided embodiments, the culturing is carried out in a bioreactor. In some of any of the provided embodiments, the bioreactor is a vertical wheel bioreactor. In some of any of the provided embodiments, the bioreactor is a vertical wheel bioreactor with a volume of about 10 mL to about 1000 mL. In some of any of the provided embodiments, the culturing in a) is carried out in a bioreactor and wherein the bioreactor is a vertical wheel bioreactor with a volume of about 100 mL. In some of any of the provided embodiments, the culturing in b) is carried out in a bioreactor and the bioreactor is a vertical wheel bioreactor with a volume of about 500 mL. In some of any of the provided embodiments, the bioreactor i is a vertical wheel bioreactor with a volume of about 500 mL. In some of any of the provided embodiments, the stem cells are pluripotent stem cells. In some of any of the provided embodiments, the pluripotent stem cells are induced pluripotent stem cells.

In some of any of the provided embodiments, the synthetic gamma chain polypeptide comprises, in N- to C-terminal order, the first dimerization domain, the first transmembrane domain, and the IL-2RG intracellular domain, and the synthetic beta chain polypeptide comprises, in N- to C-terminal order, the second dimerization domain, the second transmembrane domain, and the intracellular domain. In some of any of the provided embodiments, the first dimerization domain and the second dimerization domain are extracellular domains. In some of any of the provided embodiments, the IL-2RG intracellular domain comprises a polypeptide sequence at least 95% identical to SEQ ID NO: 1, or a polypeptide sequence as set forth in SEQ ID NO: 1. In some of any of the provided embodiments, the first transmembrane domain comprises the IL-2RG transmembrane domain.

In some of any of the provided embodiments, the IL-2RG transmembrane domain comprises a polypeptide sequence at least 95% identical to SEQ ID NO: 8 or 31, or a polypeptide sequence as set forth in SEQ ID NO: 8 or 31. In some of any of the provided embodiments, the beta chain intracellular domain comprises the IL-2RB intracellular domain. In some of any of the provided embodiments, the IL-2RB intracellular domain comprises a polypeptide sequence at least 95% identical to SEQ ID NO: 2, or a polypeptide sequence as set forth in SEQ ID NO: 2. In some of any of the provided embodiments, the beta chain intracellular domain comprises the IL-7RB intracellular domain. In some of any of the provided embodiments, the IL-7RB intracellular domain comprises a polypeptide sequence at least 95% identical to SEQ ID NO: 3, or a polypeptide sequence as set forth in SEQ ID NO: 3. In some of any of the provided embodiments, the beta chain intracellular domain comprises the IL-21RB intracellular domain.

In some of any of the provided embodiments, the IL-21RB intracellular domain comprises a polypeptide sequence at least 95% identical to SEQ ID NO: 4, or a polypeptide sequence as set forth in SEQ ID NO: 4. In some of any of the provided embodiments, the second transmembrane domain comprises a transmembrane domain from the same polypeptide as the intracellular domain.

In some of any of the provided embodiments, the second transmembrane domain is a transmembrane domain of IL-2RB comprising a polypeptide sequence at least 95% identical to SEQ ID NO: 35 or 36, or a polypeptide sequence as set forth in SEQ ID NO: 35 or 36. In some of any of the provided embodiments, the first transmembrane domain of the synthetic gamma chain polypeptide is an IL-2RG transmembrane domain comprising the sequence set forth in SEQ ID NO: 8 or 31 and the IL-2RG intracellular domain comprises the sequence set forth in SEQ ID NO:1; and the second transmembrane domain of the synthetic beta chain polypeptide is an IL-2RB transmembrane domain comprising the sequences set forth in SEQ ID NO: 35 or 36 and the beta chainintracellular domain comprising the sequence set forth in SEQ ID NO:2.

In some of any of the provided embodiments, the first transmembrane domain and the IL-2RG intracellular domain of the synthetic gamma chain polypeptide comprises the sequences set forth in SEQ ID NOs:31 and SEQ ID NO: 1; and the second transmembrane domain and the beta chain intracellular domain of the synthetic beta chain polypeptide comprises the sequences set forth in SEQ ID NOs: 35 and SEQ ID NO: 2. In some of any of the provided embodiments, the first dimerization domain and the second dimerization domain are heterodimerization domains selected from selected from FKBP12-rapamycin binding (FRB) domain and a FK506-Binding Protein of size 12 kD (FKBP); and/or wherein the non-physiological ligand is rapamycin or a rapalog. In some of any of the provided embodiments, the first dimerization domain is FKBP and the second dimerization domain is FRB. In some of any of the provided embodiments, the first dimerization domain is FRB and the second dimerization domain is FKBP. In some of any of the provided embodiments, the FRB domain comprises a polypeptide sequence at least 95% identical to SEQ ID NO: 6 or SEQ ID NO: 7. In some of any of the provided embodiments, the FRB domain comprises the polypeptide sequence set forth in SEQ ID NO: 6 or SEQ ID NO: 7. In some of any of the provided embodiments, the FKBP domain comprises a polypeptide sequence at least 95% identical to SEQ ID NO: 5 or SEQ ID NO:30. In some of any of the provided embodiments, the FKBP domain comprises the polypeptide sequence set forth in SEQ ID NO: 5 or SEQ ID NO:30.

In some of any of the provided embodiments, the synthetic gamma chain polypeptide has the sequence of amino acids set forth in SEQ ID NO: 28 or a sequence of amino acids that is at least 90%, at least 95%, at least 96%, at least 97%, at least 98%, at least 99%, or 100% identical to SEQ ID NO: 28, and the synthetic beta chain polypeptide has the sequence of amino acids set forth in SEQ ID NO: 55 or a sequence of amino acids that is at least 90%, at least 95%, at least 96%, at least 97%, at least 98%, at least 99%, or 100% identical to SEQ ID NO: 55. In some of any of the provided embodiments, the synthetic gamma chain polypeptide has the sequence of amino acids set forth in SEQ ID NO: 56 or a sequence of amino acids that is at least 90%, at least 95%, at least 96%, at least 97%, at least 98%, at least 99%, or 100% identical to SEQ ID NO: 56, and the synthetic beta chain polypeptide has the sequence of amino acids set forth in SEQ ID NO: 57 or a sequence of amino acids that is at least 90%, at least 95%, at least 96%, at least 97%, at least 98%, at least 99%, or 100% identical to SEQ ID NO: 57.

In some of any of the provided embodiments, the first dimerization domain and the second dimerization domain are homodimerization domains selected from: i) FK506-Binding Protein of size 12 kD (FKBP); ii) cyclophiliA (CypA); or iii) gyrase B (CyrB); and the non-physiological ligand is, respectively: i) FK1012, AP1510, AP1903, or AP20187 or an analog thereof; ii) cyclosporin-A (CsA) or an analog thereof, or iii) coumermycin or an analog thereof.

In some of any of the provided embodiments, cells of the population of cells (the population of stem cells, the population of hematopoietic progenitor cells or the population of myeloid progenitor cells) are resistant to rapamycin-mediated mTOR inhibition. In some of any of the provided embodiments, cells of the population of cells (the population of stem cells, the population of hematopoietic progenitor cells or the population of myeloid progenitor cells) express a cytosolic polypeptide that binds to the non-physiological ligand, optionally wherein the cytosolic polypeptide is a cytosolic FRB domain. In some of any of the provided embodiments, the non-physiological ligand is rapamycin or a rapalog, and the cells of the population express a cytosolic FRB domain or variant thereof. In some of any of the provided embodiments, the cytosolic FRB domain comprises a polypeptide sequence at least 95% identical to SEQ ID NO: 6 or SEQ ID NO: 7. In some of any of the provided embodiments, the cytosolic FRB domain comprises a polypeptide sequence at least 98% identical to SEQ ID NO: 6 or SEQ ID NO: 7. In some of any of the provided embodiments, cells of the population of cells (the population of stem cells, the population of hematopoietic progenitor cells or the population of myeloid progenitor cells) comprise a disrupted FKBP12 gene that reduces expression of FKBP12 in the cell. In some of any of the provided embodiments, cells of the population of cells (the population of stem cells, the population of hematopoietic progenitor cells or the population of myeloid progenitor cells) comprise knock out of the FKBP12 gene.

In some of any of the provided embodiments, the synthetic cytokine receptor is integrated into an endogenous gene of cells of the population of cells (the population of stem cells, the population of hematopoietic progenitor cells or the population of myeloid progenitor cells) by targeted integration of the nucleotide sequence encoding the synthetic cytokine receptor into the endogenous gene. In some of any of the provided embodiments, the targeted integration is by non-homologous end joining (NHEJ). In some of any of the provided embodiments, the targeted integration is by homology directed repair. In some of any of the provided embodiments, the insertion reduces expression of the endogenous gene in the locus. In some of any of the provided embodiments, the insertion knocks out the endogenous gene in the locus. In some of any of the provided embodiments, the insertion is by homology-directed repair.

In some of any of the provided embodiments, the endogenous gene is a housekeeping gene, a blood-lineage specific loci or an immune-related gene. In some of any of the provided embodiments, the housekeeping gene is selected from eukaryotic translation elongation factor 1 alpha (EEF1A), glylceraldehyde-3-phosphate dehydrogenase (GAPDH), ubiquitin C (UBC), and actin beta (ACTB). In some of any of the provided embodiments, the immune-related gene is selected from a beta-2-microglobulin (B2M) gene, and a signal regulatory protein alpha (SIRPA) gene. In some of any of the provided embodiments, cells of the population of cells (the population of stem cells, the population of hematopoietic progenitor cells or the population of myeloid progenitor cells) comprises a B2M knockout. In some of any of the provided embodiments, cells of the population of cells (the population of stem cells, the population of hematopoietic progenitor cells or the population of myeloid progenitor cells) comprise a B2M knockout and a FKBP12 knockout. In some of any of the provided embodiments, cells of the population of cells (the population of stem cells, the population of hematopoietic progenitor cells or the population of myeloid progenitor cells) comprising a chimeric antigen receptor (CAR). In some of any of the provided embodiments, macrophages are mature macrophages that express CD14.

In some of any of the provided embodiments, the non-physiological ligand is rapamycin or a rapamycin analog. In some of any of the provided embodiments, the rapamycin analog is rapalog. In some of any of the provided embodiments, the non-physiological ligand is added to the media at a concentration of between 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM, 10 nM and 50 nM, 10 nM and 20 nM, 20 nM and 200 nM, 20 nM and 150 nM 20 nM and 100 nM, 20 nM and 50 nM, 50 nM and 200 nM, 50 nM and 150 nM, 50 nM and 100 nM, 100 nM and 200 nM, 100 nM and 150 nM and 150 nM and 200 nM. In some of any of the provided embodiments, the non-physiological ligand is added to the media at a concentration of at or about 10 nM. In some of any of the provided embodiments, the non-physiological ligand is added to the media at a concentration of at or about 100 nM.

In some aspects, provided herein is a population of myeloid cells produced by any method provided herein. In some aspects, provided herein is a population of macrophages produced by any method provided herein. In some of any of the provided embodiments, the population of macrophages express CD14. In some aspects, provided herein is a pharmaceutical composition comprising any population of engineered myeloid cells provided herein. In some aspects, provided herein is a pharmaceutical composition comprising any population of engineered macrophages provided herein. In some of any of the provided embodiments, the pharmaceutical composition comprises a pharmaceutically acceptable excipient. In some of any of the provided embodiments, the pharmaceutical composition comprises a cryoprotectant.

In some aspects, provided herein is a method of expanding myeloid cells, the method comprising contacting any population of myeloid cells provided herein, or any pharmaceutical composition provided herein with the non-physiological ligand of the synthetic cytokine receptor. In some aspects, provided herein is a method of expanding macrophages, the method comprising contacting any population of macrophages provided herein, or any pharmaceutical composition provided herein with the non-physiological ligand of the synthetic cytokine receptor. In some of any of the provided embodiments, the method is performed in vitro or ex vivo.

In some of any of the provided embodiments, the non-physiological ligand is rapamycin or a rapamycin analog. In some of any of the provided embodiments, the rapamycin analog is rapalog. In some of any of the provided embodiments, the non-physiological ligand is contacted at a concentration of between 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM, 10 nM and 50 nM, 10 nM and 20 nM, 20 nM and 200 nM, 20 nM and 150 nM 20 nM and 100 nM, 20 nM and 50 nM, 50 nM and 200 nM, 50 nM and 150 nM, 50 nM and 100 nM, 100 nM and 200 nM, 100 nM and 150 nM and 150 nM and 200 nM. In some of any of the provided embodiments, the non-physiological ligand is contacted at a concentration of at or about 10 nM. In some of any of the provided embodiments, the non-physiological ligand is contacted at a concentration of at or about 100 nM. In some of any of the provided embodiments, the method is performed in vivo in a subject and the non-physiological ligand is administered to the subject.

In some aspects, provided herein is a method of treating a disease or condition in a subject, comprising administering to the subject an effective amount of any population of myeloid cells provided herein, or any pharmaceutical composition provided herein. In some aspects, provided herein is a method of treating a disease or condition in a subject, comprising administering to the subject an effective amount of any population of macrophages provided herein, or any pharmaceutical composition provided herein with the non-physiological ligand of the synthetic cytokine receptor. In some of any of the provided embodiments, the disease or condition is a cancer. In some of any of the provided embodiments, the cells express a CAR directed against an antigen expressed by cells of the disease or condition. In some of any of the provided embodiments, the CAR targets a tumor antigen.

In some of any of the provided embodiments, the method comprises administering to the subject a non-physiological ligand of the synthetic cytokine receptor. In some of any of the provided embodiments, the non-physiological ligand is rapamycin or a rapamycin analog. In some of any of the provided embodiments, the rapamycin analog is rapalog. In some of any of the provided embodiments, the non-physiological ligand is administered at a dose of 1 mg to 100 mg, optionally between 10-100 mg, optionally at or about 10 mg, 20 mg, 25 mg, 30 mg, 40 mg, 50 mg or any value between any of the foregoing. In some of any of the provided embodiments, multiple doses of the non-physiological ligand are administered to the subject. In some of any of the provided embodiments, the multiple doses are administered intermittently or at regular intervals after administration of the macrophage population or composition thereof to the subject, optionally for a predetermined period of time. In some of any of the provided embodiments, 2 to 8 doses of the non-physiological ligand are administered to the subject. In some of any of the provided embodiments, a single dose of the non-physiological ligand is administered to the subject.

In some aspects, provided herein is a kit comprising any engineered cell provided herein or the population of engineered myeloid cells provided herein, the population of macrophages provided herein or any pharmaceutical composition provided herein and instructions for administering to a subject in need thereof. In some of any of the provided embodiments, the kit further comprises a container comprising the non-physiological ligand and instructions for administering the non-physiological ligand to the subject after administration of the cell population. In some of any of the provided embodiments, the subject has a cancer.

### BRIEF DESCRIPTION OF THE DRAWINGS

FIG. 1A shows a schematic for CRISPR-Cas mediated site-specific knock-in of constructs encoding RACR.

FIG. 1B shows a panel of histograms that depicts RACR protein detection after knock-in at various promoters.

FIG. 1C is a graph that shows RACR protein detection after knock-in at various promoters including locus one (EEF1A1) and locus 2 (ACTB).

FIG. 2A shows a schematic depicting the role of FKBP12 in the inhibition of proliferation by rapamycin via mTOR.

FIG. 2B is a graph showing protection from rapamycin-mediated inhibition of iPSC proliferation in polyclonal FKBP12 knock-out (KO) lines (left panel) and phase-contrast images of morphology in wild type and FKBP12 KO cells (right panel).

FIG. 2C is a graph showing confluency of wildtype iPSCs after four days of treatment with varying doses of rapamycin.

FIG. 2D is a graph showing confluency of FKBP12 KO iPSCs growth during four days of treatment with varying doses of rapamycin.

FIG. 2E is a graph showing ratio of hematopoietic progenitors (HPs) to iPSCs of clonal FKBP12 KO iPSCs compared to control iPSCs.

FIG. 2F is a graph showing confluency of FKBP12 KO iPSCs growth during four days of treatment with 25 nM of rapamycin.

FIG. 3 is a schematic shows the timeline of the experiment.

FIG. 4 are graphs showing the CD14 expression on the cells from days 19-24 of differentiation via flow cytometry.

FIG. 5 are graphs showing the CD19 CAR expression on the cells from days 19-24 of differentiation via flow cytometry.

FIG. 6A are graphs showing the ratio of tumor cells phagocytosed by the engineered macrophage cells over 29 hours.

FIG. 6B are images showing the progression of phagocytosed tumor cells by the engineered macrophage cells at 0, 24, and 48 hours.

### DETAILED DESCRIPTION

Provided herein are genetically engineered myeloid cells, such as macrophages and neutrophils, that have been differentiated from progenitor cells engineered with a synthetic cytokine receptor, and the resulting methods of differentiation. In some embodiments, genetically engineered myeloid cells are derived from stem cells or myeloid progenitor cells containing a synthetic cytokine receptor. In some embodiments, the synthetic cytokine receptor contains a common gamma chain intracellular signaling domain (e.g. interleukin-2 receptor subunit gamma, IL-2RG) and a intracellular domain from interleukin-2 receptor subunit beta (IL-2RB). In some embodiments, the synthetic cytokine receptor also contains an extracellular domain that is able to be bound by a non-physiological ligand (e.g. rapamycin or an analog). In this way, binding of the non-physiological ligand to the extracellular domain of the synthetic cytokine receptor activates cytokine receptor-mediated signaling to include JAK/STAT signaling, which is an important pathway for differentiation of stem cells, such as iPSCs or other pluripotent stem cells, to downstream cell lineages, such as myeloid cells (e.g., macrophages or neutrophils). Thus, in the presence of a non-physiological ligand (e.g. rapamycin) the synthetic cytokine receptor can be engaged during cell differentiation removing the need for endogenous receptors or exogenous growth factors. In some embodiments, this increases the control and decreases the variability of JAK/STAT signaling during cell differentiation to thereby permit efficient generation of induced myeloid cells.

Myeloid cells for use in immunotherapy can be derived from obtained from primary sources such as peripheral blood or umbilical cord blood. Artificial sources for myeloid cells include pluripotent stem cells, including induced pluripotent stem cells (iPSCs), which are cells derived from somatic cells (generally fibroblasts or peripheral blood mononuclear cells [PBMCs]), and human embryonic stem cells (hESCs), either induced to become capable of unlimited proliferation and of differentiation into other cell types when subjected to appropriate differentiation conditions. Induced pluripotent stem cells (iPSCs) are a renewable, modifiable, and scalable source of material for cell therapy manufacturing. iPSCs can be made by reprogramming adult cells into a cellular state akin to embryonic stem cells. iPSCs are thought to be capable of differentiating into all cell types found in the human body and possess an unlimited expansion capacity, meaning they can reproduce and proliferate indefinitely, generating a nearly endless supply of starting material. Additionally, iPSCs are amenable to precision multiplex genome editing, allowing safe introduction of multiple genetic modifications. Because of these properties, iPSCs provide a consistent starting material, originating from a single cell (clone), which enables consistent genome integrity in process intermediates and the final cell product.

From iPSCs, myeloid cells may be derived by sequentially differentiating the iPSCs into hematopoietic progenitor cells (HPCs), also termed hematopoietic stem cells (HSCs); the HPCs into myeloid progenitor cells; and then the myeloid progenitor cells into myeloid cells, such as macrophages—termed iPSC-derived macrophages (iPSC-macrophages). Generally, iPSC-macrophage cells express CD14 and have phagocytic and cytotoxic activity, like macrophages; but iPSC-macrophage cells may differ from macrophage cells phenotypically and in other respects.

However, major challenges exist in current iPSC-based approaches to cell therapy. Specifically, current approaches for differentiating iPSCs into therapeutic immune cell types require exogenous growth factors, and in some cases, the presence of feeder cells. Overall, iPSC-based cell therapy is generally inefficient in generating the necessary intermediate progenitor cells, resulting in a low initial yield of the therapeutic cell type (e.g., macrophages), which then requires feeder cell-driven expansion. This feeder cell-driven expansion can dramatically reduce the proliferative capacity of the final cell therapy product. Thus, in order to achieve the necessary engraftment to have any therapeutic effect, high cell numbers (~1 billion cells) and repeat dosing are required in addition to repeated cycles of lymphodepleting chemotherapy.

Further, current approaches to cell therapy manufacturing include either autologous or allogeneic cellular starting material from which a disease-targeted therapeutic cell product is engineered. In some embodiments, an allogeneic or “off-the-shelf” cell therapy has the potential to transform cell therapy from personalized medicine into a routine treatment. However, current “off-the-shelf” cell therapies are struggling to show the same engraftment and persistence of cells in vivo that has been reached by approved autologous cell therapy products. In some aspects, this is due to the foreign nature of allogeneic or even engineered elements of autologous cells, which can be recognized and rejected by the host immune system. To date, cell therapies achieve cell engraftment by treating patients with highly toxic chemotherapy regimens, termed lymphodepletion (LD), given prior to administration of the cell therapy product. LD essentially removes the host immune system and provides many benefits to the cell therapy product, firstly providing free “homeostatic cytokines” for an ex vivo cell therapy product as well as reducing anti-graft responses against the foreign graft by the host immune system. However, LD is a transient solution, and the host immune system rapidly reconstitutes. Thus, to sustain allogeneic cell exposure, multiple rounds of LD and cell infusion are required. Additionally, exogenous cytokines such as IL-2 are administered, and these cytokine treatments have low exposure times with high toxicities associated with their use. Finding better ways to increase cell persistence is key to achieving durable tumor remission and has proven to be a challenge in the allogeneic cell therapy space.

Allogeneic cells can be further broken down into donor- or iPSC-derived cells.

Donor-derived cells are generally sourced from the circulation or cord blood of a healthy donor and the therapeutic cell type (e.g., myeloid cells, such as macrophages and neutrophils) is selected, subsequently harvested, and expanded in a complex cell culture process that generally includes multiple cytokines, growth factors, gene engineering, and feeder cells to generate many doses. Alternatively, iPSC-derived cells, which also require multiple complex cell culture conditions, must have these conditions implemented in a stepwise fashion to drive cells through the necessary progenitor stages to ultimately obtain the intended final cell product (e.g., immune effector cell). For example, macrophages are classically activated by certain molecular patterns commonly present in pathogenic organisms, such as lipopolysaccharide (LPS) or the nucleic acid CpG. Further, effective methods for macrophage activation and expansion for clinical-scale purposes are known to require exogenous cytokines, including IL-4 and/or IL-13, as well as antigen molecules, co-stimulatory molecules, and/or cell adhesion molecules. Macrophages are phagocytic cells characterized by their ability to discriminate between self and non-self by monitoring the expression of MHC class I molecules, release of cytokines, and directly kill and/or engulf non-self or infected target cells. It is known in the art that macrophages do not represent a uniform population. Rather, there are many distinguishable subsets of macrophages. Effectively expanding macrophages that rely on a large quantity of diverse exogenous factors using currently known methods often requires complex and expensive manufacturing processes.

Provided herein are methods of generating and expanding genetically engineered myeloid cells, such as macrophages and neutrophils, utilizing a synthetic cytokine receptor, such that the exogenous factors detailed above are supplemented during ex vivo expansion of macrophages and/or after infusion of the macrophages into a subject.

Furthermore, the gold standard for cell therapy is autologous chimeric antigen receptor (CAR) T cell therapy. Decades of CAR T cell therapy efforts, starting with “first generation” CAR T cell therapies in the 1990s and leading to the first CAR T cell therapies receiving FDA approval in 2017, have resulted in successes in treating B cell malignancies, with long-term remission achieved in 30-40% of certain patient populations. Importantly, CAR T cell efficacy requires lymphodepleting chemotherapy to eliminate sinks for survival factors such as IL-15. While CAR T cell therapies have revolutionized the treatment of malignancies (e.g., hematologic), major limitations hinder its widespread application. The allogeneic CAR T therapy field has shown promising early clinical results; however, the durable response profile has been generally poor in comparison to autologous CAR T cell therapies, despite the use of ever increasing intensity LD regimens. This is likely due to limitations of the drug product cell type, manufacturing processes, as well as anti-allograft responses against the therapeutic cells. Thus, despite the promising clinical efficacy of CAR T cells in hematologic malignancies, significant challenges remain, including patient access, complex manufacturing, and high cost.

Compared with T cells, the myeloid cells, such as macrophages, have the advantage of being easier to enter the solid tumor and less likely to be inhibited by other types of cells, and therefore can play a better role in tumor immunotherapy. Since the expressed chimeric antigen receptor is located on the surface of the macrophage, the macrophage can accurately target tumor cells. The provided engineered macrophages and methods related to the same provide for “off-the-shelf” cancer therapies to overcome these challenges.

iPSCs can also be modified via CRISPR to express a CAR to overcome challenges associated with targeting, for example, the heterogeneous solid tumor microenvironment.

Provided herein is a Synthetic Receptor Enabled Differentiation (ShRED), a directed differentiation and expansion process controlled by the synthetic cytokine receptor, e.g., Rapamycin-Activated Cytokine Receptor (RACR). RACR is activated via the addition of its synthetic ligand rapamycin, which induces a JAK/STAT signal that drives differentiation and expansion of cells into hematopoietic progenitors (HPs) and then into immune effector cells, termed RACR-induced myeloid cells, such as macrophages (RACR-iMACs). Furthermore, because rapamycin is a safe, effective, and approved therapeutic for immune suppression, RACR can also be engaged in vivo through rapamycin dosing to increase the persistence of RACR expressing myeloid cells, while simultaneously protecting these cells from allogeneic rejection

Provided herein is a platform for producing immune effector cells in the absence of exogenous cytokines and feeder cells by genetically modifying iPSCs and iPSC-derived progenitor cells to express a synthetic cytokine receptor. A synthetic small molecule ligand (e.g., rapamycin) activates the receptor to drive the differentiation and expansion of immune effector cells. The compositions and methods provided herein comprise myeloid cells (e.g., macrophages and neutrophils) engineered to express a synthetic cytokine receptor. Non-limiting advantages of the engineered myeloid cells include superior and controllable expansion when administered to a subject, similar cytotoxic activity and phagocytosis as compared to native myeloid cells, improved iPSC-derived cell manufacturing and enhanced anti-tumor activity.

In provided aspects, the RACR engineering platform provided herein improves iPSC-derived cell manufacturing by controlling cell production. Through rapamycin dosing and activation of RACR, a more reproducible differentiation process and homogeneous cell product results. The RACR engineering platform also reduces manufacturing costs as RACR activation eliminates the need to add expensive growth factors, cytokines and other raw materials. In certain embodiments, the methods disclosed herein may further enhance activation through the ability of the macrophages described herein to be expanded without or with fewer exogenous factors, such as without TGF-β and/or IFN-γ. In some embodiments, the methods disclosed herein may further enhance activation and tumor killing through the ability of the macrophages described herein to be generated with the removal of one or more exogenous factors as compared to a conventional process. The RACR engineering platform increases yields of highly pure intermediate and final cell products. The RACR engineering platform provided herein generated highly pure hematopoietic progenitors (HPs), an intermediated progenitor population, and resultant myeloid cells that are highly pure and phenotypically mature, removing the need for cell sorting after differentiation of the macrophages. The RACR engineering platform increases patient-compatibility of the cells as the manufacturing process is completely feeder cell and xenogeneic cell free. The RACR engineering platform is also compatible with cells in suspension, promoting scalability of cell production.

In other provided aspects, the RACR engineering platform removes the need for additional physical processing of differentiated progenitor cells. In conventional processes of differentiating progenitor cells, residual cell aggregates must be removed prior to blood cell differentiation. Physical processing includes enzymatic digestion (e.g., collagenase or TrypLE™ enzymes) and filtration (e.g., cells are strained to remove undesired cell aggregates). In contrast, the RACR engineering platform results in embryoid bodies that completely dissociate into pure HPs with no cell filtration required.

In provided aspects, the RACR engineering platform provided herein improves anti-tumor activity of iPSC-derived cells by increasing cell engraftment, persistence and effector function. The RACR engineering platform provided herein also improves anti-tumor activity of iPSC-derived cells by inhibiting host immune response via rapamycin dosing, which further enables engraftment of the cells (e.g., macrophages). The RACR engineering platform provided herein also improves anti-tumor activity of iPSC-derived cells by removing the need for toxic LD due by activating the RACR system to selectively support RACR cell expansion and survival.

In some aspects, among advantages of the RACR system on macrophages, including the engineered synthetic cytokine receptor and activation thereof with rapamycin or rapalog, includes: the ability to engineer unlimited starting material that is highly efficient at generating immediate progenitors and that is characterized by minimized expansion requirements on the final cell type; the ability to efficiently edit cells; no requirement for feeder cells, thereby minimizing complex, raw materials; no requirement for lymphodepletion in subjects receiving RACR-engineered cells; low to no cytokine release syndrome (CRS) or Immune effector cell-associated neurotoxicity syndrome (ICAN); and promotion of engraftment, expansion, and persistence with administration of rapamycin or rapalogs.

Provided here are synthetic cytokine receptors that can be applied to support the derivation of macrophages. Macrophages may be derived from stem or progenitor cells, and such cells are termed herein “induced macrophage” (iMAC) cells. iMAC cells share distinguishing cell surface markers and functional attributes as described herein. As used herein, the terms “induced macrophage” or “iMAC” refers to a macrophage made by inducing differentiation of progenitor cells. As disclosed herein, iMAC may be made and/or expanded by expressing a synthetic cytokine receptor in a stem or progenitor cell and acting the synthetic cytokine receptor by the non-physiological ligand. Such a process may involve differentiation of a progenitor cell engineered to express a synthetic cytokine receptor by activation of the synthetic cytokine receptor. The process may also or alternatively involve expansion of the progenitor cell or the macrophage by activation of the synthetic cytokine receptor.

In some embodiments, the present disclosure provides stem cells (e.g., iPSCs) and macrophages engineered to express a rapamycin activated cytokine receptor (RACR), a synthetic cytokine receptor activated by the small molecule rapamycin or rapalogs. Macrophages comprising a RACR and activated with rapamycin or a rapalog are termed herein “RACR-iMAC” cells. Stem cells comprising a RACR and activated with rapamycin or a rapalog are termed herein “RACR-SCs”. RACR is demonstrated to support differentiation and/or expansion of RACR-SCs and RACR-iMAC cells in a feeder-free manufacturing process. RACR-iMAC cells express multiple innate tumor targeting receptors and when engineered to express a chimeric antigen receptor (CAR), are able to exert CAR-directed cytolytic activity. Accordingly, RACR-iMAC cells provide an “off-the-shelf” allogeneic cell therapy.

The disclosure relates, in part, to the surprising discovery that stem cells engineered to express a synthetic cytokine receptor differentiate to hematopoietic progenitors or myeloid progenitor cells in response to the receptor's cognate non-physiological ligand. Macrophages differentiated from the engineered stem cells retain the synthetic cytokine receptor and expand in response to the receptor's cognate non-physiological ligand. The engineered macrophages may be generated in high quantities and with functional activity equal to or greater than macrophages from other sources.

The macrophages may be derived from iPSCs, myeloid progenitor cells, or other stem or progenitor cells. Further provided herein are stem or progenitor cells engineered to express synthetic cytokine receptors, and methods of differentiating engineered stem or progenitor cells into macrophages by contacting the stem or progenitor cells with the cognate non-physiological ligand for the cytokine receptor.

In certain embodiments, provided herein are ex vivo generated macrophages. The engineered cells described herein, and related compositions, may be used for immunotherapy with ligand-controlled ex vivo expansion. Further provided herein are methods of expanding macrophages by contacting the cells with the cognate non-physiological ligand for the synthetic cytokine receptor. Moreover, the engineered macrophages disclosed herein may be further engineered to express a chimeric antigen receptor (CAR), enabling targeting of the engineered macrophages to cells expressing or labelled with the antigen recognized by the CAR.

In some embodiments, the provided engineered macrophages and methods provided for an improved immunotherapy compared to existing strategies. While chimeric antigen receptor (CAR) T cell therapies have revolutionized the treatment of hematologic malignancies, major limitations hinder their widespread application.

In some embodiments, engagement of the synthetic cytokine receptor not only is able to promote differentiation but also is able to increase cell growth and promote expansion through engagement of the synthetic cytokine receptor on provided engineered macrophages. In some aspects, the provided engineered macrophages and related methods can be used to increase cell growth and expansion in vivo of the engineered cell therapy through rapamycin dosing of patients after the cell therapy product. In some embodiments, rapamycin simultaneously expands and protects the cells. Expansion is achieved through the JAK/STAT signal activation and protection is achieved through rapamycin suppression of host anti-graft responses. In some embodiments, the need for lymphodepletion as well as exogenous cytokine dosing is not necessary. In some embodiments, provided methods of administration and treatment with the engineered macrophages can be carried out without lymphodepletion (e.g. without the need to administer a lymphodepleting therapy such as cyclophosphamide and/or fludarabine). In some embodiments, provided methods of administration and treatment with the engineered macrophages can be carried out without exogenous cytokine administration.

In some embodiments, the provided methods also can include administering the non-physiological ligand of the synthetic cytokine receptor (e.g. rapamycin or rapalog) to the subject to expand or reinvigorate the engineered macrophages in the subject. Thus, in some embodiments, it is not necessary to further re-dose the subject with macrophages, since it is possible to expand the cells in vivo with the non-physiological ligand. However, re-dosing of macrophages also is possible due to the hypoimmune engineering as described herein making allogeneic cell therapy possible. Furthermore, since the provided methods can be carried out without lymphodepletion this further provides advantages to promote expansion of the transferred cells as well as promote a host anti-tumor response. This is because without lymphodepletion the host immune system remains and is not heavily depleted. The immune response generated by the macrophages (e.g. release of cytokines and other pro-inflammatory factors) therefore could stimulate the existing immune system of the host against the tumor. Moreover, the exemplary non-physiological ligand rapamycin not only promotes expansion of the transferred cells via engagement of the synthetic cytokine receptor, but transient mTOR suppression like achieved via rapamycin can reinvigorate T cells as well as promote apoptosis of suppressive macrophages. Also, while the non-physiological ligand rapamycin or an analog can suppress an anti-graft response by the host, this is expected to be only transient and such that a more normal host anti-tumor response would resume once administration of the non-physiological ligand is discontinued.

In some embodiments, engineered cells herein are further modified to be resistant to the effects of rapamycin on inhibiting or reducing cell growth and expansion. In some embodiments, the cells can be made “rapamycin resistant” by providing free cytosolic FRB to the cell in order to complex with rapamycin and thereby eliminate or reduce rapamycin-mediated growth inhibition of a source cell or iMAC. In other embodiments, the cells can be made “rapamycin resistant” by disrupting, such as inactivating or knocking out, FKBP12 in the engineered cell. In some cases, overexpression of FRB may not result in free-FRB that is able to completely quench rapamycin. Thus, in cases, editing endogenous genes in the cell, such as by FKBP12 knockout, can provide for full rapamycin resistance of cells.

Accordingly, provided embodiments employing a synthetic cytokine receptor system, such as a rapamycin activated cytokine receptor (RACR) that can be engaged by rapamycin or an analog, e.g. rapalog, both protects and expands cells in a single technology. In addition, the additional inclusion of genetic disruption, such as knockout of certain immune genes such as beta-2-microgloublin (B2M), also can produce “stealth” cells that have additional advantages for allogeneic cell therapy.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The Section headings used herein are for organizational purposes only and are not to be construed as limiting the subject matter described. Those skilled in the art will recognize that several embodiments are possible within the scope and spirit of the present disclosure. The following description illustrates the disclosure and, of course, should not be construed in any way as limiting the scope of the inventions described herein.

### I. DEFINITIONS

All publications, patent applications, patents and other references mentioned herein are incorporated by reference in their entirety.

Unless the context indicates otherwise, the various features described herein can be used in any combination with any feature or combination of features set forth herein, and each feature can be excluded or omitted from the combination.

As used herein, the singular forms “a”, “an”, and “the” are include the plural forms as well, unless the context indicates otherwise. The conjugation “and/or” denotes all possible combinations of one or more of listed items.

“Subject” as used herein refers to the recipient of an engineered macrophage or other agent. The term includes mammal, such as primate, mouse, rat, dog, cat, cow, horse, goat, camel, sheep or a pig, preferably a human.

“Treat,” “treating” or “treatment” as used herein refers to any type of action or administration that imparts a benefit to a subject that has a disease or disorder, including improvement in the condition of the patient (i.e., improvement, reduction, or amelioration of one or more symptoms, and partial or complete response to treatment).

The term “effective amount” refers to an amount effective to generate a desired biochemical, cellular, or physiological response. The term “therapeutically effective amount” refer to the amount, dosage, or dosage regime of a therapy effective to cause a desire treatment effect.

“Polynucleotide” as used herein refers to a biopolymer composed of two or more nucleotide monomers covalently bonded through ester linkages between the phosphoryl group of one nucleotide and the hydroxyl group of the sugar component of the next nucleotide in a chain. DNA and RNA are non-limiting examples of polynucleotides.

“Polypeptide” as used herein refers to a polymer consisting of amino acid residues chained together by peptide bonds, forming part of (or the whole of) a protein.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described herein to reflect the codon usage of any particular host organism in which the polypeptides are to be expressed.

Nucleic acids may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The term “variant” means a polynucleotide or polypeptide having at least one substitution, insertion, or deletion in its sequence compared to a reference polynucleotide or polypeptide. A “functional variant” is a variant that retains one or functions of the reference polynucleotide or polypeptide.

As used herein the term “sequence identity”, or “identity” in relation to polynucleotides or polypeptide sequences, refers to the extent to which two optimally aligned polynucleotides or polypeptide sequences match at each position in the alignment across the full length of the reference sequence. The “percent identity” is the number of matched positions in the optimal alignment, divided by length of the reference sequence plus the sum of the lengths of any gaps in the reference sequence in the alignment. The optimal alignment is the alignment that results in the maximum percent identity. Alignment of sequences to determine percent identity can be accomplished by a number of well-known methods, including for example by using mathematical algorithms, such as, for example, those in the BLAST suite or Clustal Omega sequence analysis programs. Unless noted otherwise, the term “sequence identity” in the claims refers to sequence identity as calculated by BLAST version 2.12.0 using default parameters. And, unless noted otherwise, the alignment is an alignment of all or a portion of the polynucleotide or polypeptide sequences of interest across the full length of the reference sequence.

As used herein, “small molecule” refers to a low molecular weight (<1000 Daltons), organic compound. Small molecules may bind specific biological macromolecules and can have a variety of biological functions or applications including, but not limited to, serving as cell signaling molecules, drugs, secondary metabolites, or various other modes of action.

The term “analog” in relation to a small molecule refers to a compound having a structure and/or function similar to that of another compound but differing from it in respect to a certain component. The analog may differ in one or more atoms, functional groups, or substructures, which are replaced with other atoms, groups, or substructures. Despite a high structural and/or functional similarity, analogs can have different physical, chemical, physiochemical, biochemical, or pharmacological properties.

The term “rapalog” is an art-recognized group of analogs of rapamycin analog that share structural and functional similarity to rapamycin. Certain rapalogs are known to share some but not all functional attributes of rapamycin. For example, some rapalogs are suitable for uses as a non-physiological ligand because they promote dimerization but have substantially no immunosuppressive activity (e.g., AP21967, AP23102, or iRAP).

An illustrative rapalog of the disclosure is AP21967

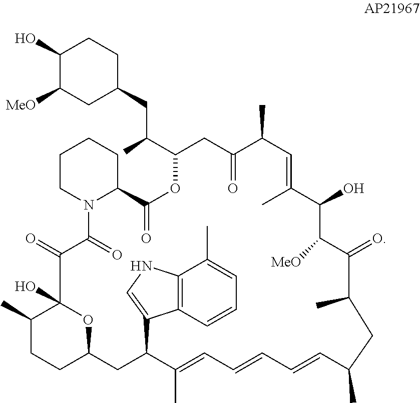 [SMILES: CO[C@@H]1C[C@H](C[C@H](C)[C@@H]2CC(=O)[C@@H](C)/C=C(\C)[C@@H](O)[C@@H](OC)C(=O)[C@H](C)C[C@@H](C)/C=C/C=C/C=C(\C)[C@H](c3c[nH]c4c(C)cccc34)C[C@H]3CC[C@@H](C)[C@@](O)(O3)C(=O)C(=O)N3CCCC[C@H]3C(=O)O2)CC[C@@H]1O]

An illustrative rapalog of the disclosure is AP23102

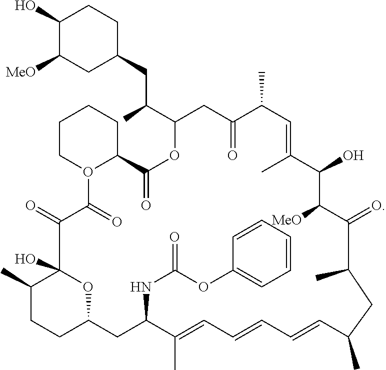

An illustrative rapalog of the disclosure is iRAP

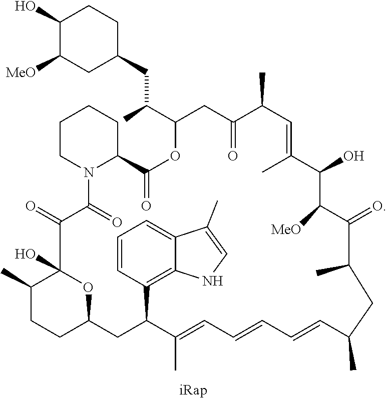

The term “cell population” refers to mixture of cells suspended in solution, attached to a substrate, or stored in a container. The characteristics of a cell population as a whole can be studied with bulk measurements of sample volumes having a plurality of cells. Flow cytometry methods may be employed to reduce problems with background fluorescence which are encountered in bulk cell population measurements.

As used herein, the term “macrophage” or “iMac” is used to refer to a class of phagocytic white blood cell that constitute a major component of the innate immune system. Macrophages recognize foreign pathogens for uptake through several mechanisms, including both non-specific bulk endocytosis and through engagement of specific receptors on the cell surface that either bind to epitopes on the bacterial surface itself or bind mammalian proteins that have bound to the bacterial surface (antibodies, complement proteins, or other opsonins). Following internalization of a pathogen by the macrophage, the pathogen becomes encapsulated in a membrane bound compartment called the phagosome. The phagosome is fused with a lysosome to form a phagolysosome. The phagolysosome contains enzymes, reactive oxygen species, and other toxic molecules that break-down the pathogen. Macrophages also internalize and breakdown infected cells and cell debris from the site of an active infection, helping prevent further spread of the infection and limiting the area of tissue damage. Macrophages also play a role in innate immunity and adaptive immunity by recruiting other immune cells to the site of an infection. In humans, mature macrophages may express one or more of CD14, CD11b, CD68, CD163, F4/80, CD16, CD54, CD49e, CD38, Egr2, CD71, TLR2, TLR4.

As used herein, the term “engineered” refers to a cell that has been stably transduced with a heterologous polynucleotide or subjected to gene editing to introduce, delete, or modify polynucleotides in the cell, or cells transiently transduced with a polynucleotide in a manner that causes a stable phenotypic change in the cell.

As used herein, the term “stem cell” is used to describe a cell with an undifferentiated phenotype, capable, for example, of differentiating into hematopoietic progenitors, myeloid progenitors, macrophage progenitors, myeloid cells, monocytes and/or macrophage cells.

As used herein, the term “pluripotent” means the stem cell is capable of forming substantially all of the differentiated cell types of an organism, at least in culture. For example, embryonic stem cells are a type of pluripotent stem cells that are able to form cells from each of the three germs layers, the ectoderm, the mesoderm, and the endoderm.

As used herein, the terms “induced pluripotent stem cell” and “iPSC” are used to refer to cells, derived from somatic cells, that have been reprogrammed back to a pluripotent state and are capable of proliferation, selectable differentiation, and maturation. iPSCs are stem cells produced from differentiated adult, neonatal, or fetal cells that have been induced or changed, i.e., reprogrammed, into cells capable of differentiating into tissues of all three germ or dermal layers: mesoderm, endoderm, and ectoderm. The iPSCs produced do not refer to cells as they are found in nature.

As used herein, the term “hematopoietic stem cell” refers to stem cells capable of giving rise to both mature myeloid and lymphoid cell types including macrophages, natural killer cells, T cells, and B cells. Hematopoietic stem cells are typically characterized as CD34+.

The term “progenitor” refers to a cell partially differentiated into a desired cell type. Progenitor cells retain a degree of pluripotency and may differentiate to multiple cell types.

As used herein, the term “hematopoietic progenitor cell” refers to cells of an intermediate cell type capable of differentiating down blood cell lineages, wherein the hematopoietic progenitor cell may differentiate into either common myeloid progenitor cells or common lymphoid progenitor cells. Hematopoietic progenitor cells are typically characterized as CD34+ and CD45+. CD38 is also considered a marker for hematopoietic progenitor cells. CD45 is considered a hematopoietic lineage marker.

As used herein, the terms “myeloid progenitor cell” or “macrophage progenitor cell” refer to cells that are precursors to myeloid cells, e.g., monocyte and macrophage cells. Myeloid progenitor cells are the first stage of differentiation of hematopoietic stem cells that follow the myeloid lineage of differentiation. As used herein, the term “myeloid progenitor” refers to cells capable of hematopoietic transition to hematopoietic cell-types. Myeloid progenitor cells may be characterized by being CD45+CD34+CD150+ and FcR+.

As used herein, “differentiate” or “differentiated” are used to refer to the process and conditions by which undifferentiated, or immature (e.g., unspecialized), cells acquire characteristics becoming mature (specialized) cells thereby acquiring particular form and function. Stem cells (unspecialized) are often exposed to varying conditions (e.g., growth factors and morphogenic factors) to induce specified lineage commitment, or differentiation, of said stem cells.

As used herein, “expand” or “expansion” refer to an increase in the number and/or purity of a cell type within a cell population through mitotic division of cells having limited proliferative capacity, e.g., macrophage cells.

As used herein, “activity”, “activate”, or “activation” refer to stimulation of activating receptors on a macrophage leading to cell division, cytokine secretion, and/or release of cytolytic granules to regulate or assist in an immune response.

### II. CELLS ENGINEERED WITH A SYNTHETIC CYTOKINE RECEPTOR AND METHODS OF DIFFERENTIATING SAME TO MYELOID CELLS

Provided herein are myeloid cells derived from stem or progenitor cells containing a synthetic cytokine receptor. In some embodiments, the synthetic cytokine receptor is any as described in Section II. B. In some embodiments, the synthetic cytokine receptor contains a common gamma chain intracellular signaling domains (e.g. interleukin-2 receptor subunit gamma, IL-2RG) and a intracellular domain from interleukin-2 receptor subunit beta (IL-2RB). In some embodiments, the synthetic cytokine receptor also contains an extracellular domain that is able to be bound by a non-physiological ligand (e.g. rapamycin or an analog). In this way, binding of the non-physiological ligand to the extracellular domain of the synthetic cytokine receptor activates cytokine receptor-mediated signaling to include JAK/STAT signaling, which is an important pathway for differentiation of stem cells, such as iPSCs or other pluripotent stem cells, to downstream cell lineages, such as macrophages. Thus, in the presence of a non-physiological ligand (e.g. rapamycin) the synthetic cytokine receptor can be engaged during cell differentiation removing the need for endogenous receptors or exogenous growth factors. In some embodiments, this increases the control and decreases the variability of JAK/STAT signaling during cell differentiation to thereby permit efficient generation of induced myeloid cells (iMCs), e.g., induced macrophages (iMACs).

As described above, provided herein are stem or progenitor cells that may be differentiated into myeloid cells using a synthetic cytokine receptor complex activated by a non-physiological ligand, and differentiated cells produced from those stem or progenitor cells for use in medical treatment. The differentiated cells may be, but are not limited to, iMCs. As a non-limiting illustration of the compositions and methods described herein, macrophages may be produced from pluripotent stem cells, such as induced pluripotent stem cells, engineered to express synthetic cytokine receptor able to be activated by a non-physiological ligand (e.g. rapamycin) as described to induce differentiation, in addition to or instead of an exogenous cytokine. In some embodiments, the synthetic cytokine receptor is a rapamycin activated cytokine receptor (RACR) using rapamycin or a rapalog to induce differentiation, in addition to or instead of an exogenous cytokine. Advantages of embodiments may include the ability to generate from a plentiful cell source (e.g., induced pluripotent stem cells) effector cells expressing synthetic cytokine receptor complex activated by a non-physiological ligand, so that proliferation of the effector cells in patients may be controlled by administering or ceasing administration of the non-physiological ligand. Other advantages of embodiments include, but are not limited to, the ability to generate a homogenous population of effector cells from source cells, removing the need for cell sorting after differentiation. Further, additional advantages of effector cells (e.g., macrophages) expressing a synthetic cytokine receptor complex activated by a non-physiological ligand include the activation of the effector cells (e.g., macrophages) against tumor cells without the use of exogenous stimulation (e.g., TGF-β and/or IFN-γ).

### A. Stem Cells and Engineered Stem Cells

In some embodiments, provided herein are cells that are engineered with a synthetic cytokine receptor, such as a synthetic cytokine receptor described in Section II.B. In some embodiments, the cells are stem cells. In some embodiments, the stem cells are pluripotent stem cells. In some embodiments, the pluripotent stem cells are induced pluripotent stem cells (iPSCs).

The methods provided herein for producing myeloid cells from the genetically engineered iPSCs may comprise an ex vivo culturing process, wherein the myeloid cells are differentiated from a non-terminally differentiated cell. In some embodiments, the non-terminally differentiated cell is a stem cell. In some embodiments, the non-terminally differentiated cell is an iPSC cell. In some embodiments, the non-terminally differentiated cell is a progenitor cell. In embodiments, the non-terminally differentiated cells (e.g. stem cells, such as iPSC) expresses a synthetic cytokine receptor. In an aspect, the disclosure provides a method of producing macrophages comprising providing stem or progenitor cells and differentiating the cells into macrophages by controlled activation of the synthetic cytokine receptor, or without activation of the synthetic cytokine receptor. In some embodiments, the differentiation is carried out by activation of the synthetic cytokine receptor with one or more additional cytokines. In some embodiments, differentiation also may be carried out with cytokines without activation of the synthetic cytokine receptor.

In some embodiments, the stem cells are pluripotent stem cells. Various sources of pluripotent stem cells can be used in the method, including embryonic stem (ES) cells and induced pluripotent stem cells (iPSCs). Various sources of pluripotent stem cells can be used in the method, including embryonic stem (ES) cells and induced pluripotent stem cells (iPSCs). In some embodiments, pluripotent stem cells are induced pluripotent stem cells (iPSCs), artificially derived from a non-pluripotent cell. In some aspects, a non-pluripotent cell is a cell of lesser potency to self-renew and differentiate than a pluripotent stem cell. iPSCs may be generated by a process known as reprogramming, wherein non-pluripotent cells are effectively “dedifferentiated” to an embryonic stem cell-like state by engineering them to express genes such as OCT4, SOX2, and KLF4. Takahashi and Yamanaka Cell (2006) 126: 663-76.

In some embodiments, source cells may be human embryonic stem cell (hESC) or induced pluripotent stem cell (iPSC). In immunotherapy, source cells be allogeneic or autologous, meaning from a donor or from the subject, respectively. For example, when the subject being treated using the compositions of the present disclosure has received high-dose chemotherapy or radiation treatment to destroy the subject's immune system, allogenic cells may be used.

In some embodiments, myeloid cells may be generated from induced pluripotent stem cells (iPSCs). iPSCs are a type of pluripotent stem cell derived from adult somatic cells that have been genetically reprogrammed to an embryonic stem cell-like state through the forced expression of genes and factors important for maintaining the defining properties of embryonic stem cells. iPSCs may be generated from tissues with somatic cells, including, but not limited to, the skin, dental tissue, peripheral blood, and urine. To generate iPSCs, somatic cells may be reprogrammed through methods including, but not limited to, the transient expression of reprogramming factors, virus-free methods, adenoviruses, plasmids, minicircle vectors, episomal vectors, Sendai viruses, synthetic mRNAs, self-replicating RNAs, retroviruses, lentiviruses, PhiC31 integrases, excisable transposons, CRISPR-based gene editing, or recombinant proteins.

While the cell therapy industry has demonstrated the transformative potential of using gene-engineered, patient-derived cells for treating specific disease indications, many challenges remain with using patient-derived materials, including limited expansion capacity and scalability, manufacturing complexity, high cost, variability from patient to patient, and patient access. In contrast, iPSCs are pluripotent stem cells, a type of cell theoretically capable of differentiating into any other cell type—including macrophages that are applicable to the treatment of cancer. Using iPSCs, it is possible to provide a scalable and simplified manufacturing of a target cell-fighting (e.g. cancer fighting) cell therapy like macrophages, thus reducing costs and improving patient access to the cell therapies. iPSCs possess an unlimited expansion capacity, meaning they can reproduce and proliferate indefinitely, potentially generating a nearly endless supply of differentiated immune cells for therapy, such as for cancer therapies. iPSCs are also amenable to precision multiplex genome editing, allowing introduction of multiple genetic modifications to enhance their disease targeting capabilities and safety of the immune cells they eventually become. iPSCs can similarly be engineered with the goal of protecting them against allogeneic rejection by the patient's own immune system, improving both their initial expansion and duration of engraftment. Furthermore, while either patient-derived or donor-derived blood materials are inconsistent, iPSCs provide a consistent starting material originating from a single cellular clone, which can permit genomic consistency and integrity in the final cellular product.

In some embodiments, the PSCs (e.g. iPSCs) are autologous to the subject to be treated, i.e. the PSCs are derived from the same subject to whom the differentiated cells are administered. In some embodiments, non-pluripotent cells (e.g., fibroblasts) derived from patients to be treated are reprogrammed to become iPSCs before differentiation into macrophages as described herein. In some embodiments, fibroblasts may be reprogrammed to iPSCs by transforming fibroblasts with genes (OCT4, SOX2, NANOG, LIN28, and KLF4) cloned into a plasmid (for example, see, Yu, et al., Science DOI: 10.1126/science.1172482). In some embodiments, non-pluripotent fibroblasts derived from patients are reprogrammed to become iPSCs before differentiation into macrophages, such as by use of the non-integrating Sendai virus to reprogram the cells (e.g., use of CTS™ CytoTune™-iPS 2.1 Sendai Reprogramming Kit). In some embodiments, the resulting differentiated cells are then administered to the patient from whom they are derived in an autologous cell therapy.

In some embodiments, the PSCs (e.g., iPSCs) are allogeneic to the subject to be treated, i.e. the PSCs are derived from a different individual than the subject to whom the differentiated cells will be administered. In some embodiments, non-pluripotent cells (e.g., fibroblasts) derived from another individual (e.g. an individual not having a disease or condition to be treated, such as a healthy subject) are reprogrammed to become iPSCs before differentiation into macrophages. In some embodiments, reprogramming is accomplished, at least in part, by use of the non-integrating Sendai virus to reprogram the cells (e.g., use of CTS™ CytoTune™-iPS 2.1 Sendai Reprogramming Kit). In some embodiments, the resulting differentiated cells are then administered to an individual who is not the same individual from whom the differentiated cells are derived (e.g. allogeneic cell therapy or allogeneic cell transplantation). In such embodiments, the PSCs described herein (e.g. allogeneic cells) may be genetically engineered to be hypoimmunogenic. Methods for reducing the immunogenicity are known, and include ablating polymorphic HLA-A/-B/-C and HLA class II molecule expression. Exemplary methods for reducing one or more HLA molecules include disrupting the beta-2-microglobulin (B2M) gene, such as described herein.

In some embodiments, the disclosure provides engineered stem cells transiently or stably expressing a synthetic cytokine receptor complex. In some embodiments, the disclosure provides engineered stem cells stably expressing a synthetic cytokine receptor complex. In some embodiments, the disclosure provides engineered stem cells stably expressing a synthetic cytokine receptor complex and a chimeric antigen receptor (CAR).

In some embodiments, the engineered iPSC further comprises a disrupted B2M, and/or SIRPA locus. In some embodiments, the genome further comprises a disrupted FKBP12 locus. In some embodiments, the genome further comprises a disrupted AAVS1 locus. In some embodiments, the engineered synthetic cytokine receptor is integrated into the disrupted B2M locus, such as by HDR or other methods. In some embodiments, the CAR is integrated into the disrupted AAVS1 locus, such as by HDR or other methods. In some embodiments, the cells are further disrupted in a gene encoding FKBP12 such as to reduce expression or knockout the gene encoding FKBP12. In some embodiments, a locus of a gene is disrupted by gene editing technologies, such as CRISPR-Cas systems. In some embodiments, a disrupted locus inactivates the gene in the cell. In some embodiments, a disrupted locus involves knockout of the gene in the cell. In some embodiments, the disrupted locus comprises an indel in the endogenous gene or a deletion of a contiguous stretch of genomic DNA of the endogenous gene. In some embodiments, the indel is a frameshift mutation or a deletion of a contiguous stretch of genomic DNA of the gene. In some embodiments, the indel is in both alleles of the gene (indel/indel). Methods of gene editing and engineering are known, including methods described in Section III. Any of such methods can be used to generate engineered stem cells that further comprise a synthetic cytokine receptor complex as described herein. In some cases, the engineered stem cells further comprise a polynucleotide encoding a chimeric antigen receptor (CAR), thereby generating engineered stem cells expressing the CAR. Exemplary CARs and methods for engineering cells with a CAR are described in Sections III and IV.

In some cases, the engineered stem cells further comprise a polynucleotide encoding FRB, thereby generating engineered stem cells expressing cytosolic FRB. The FRB can have a sequence as described in Section II.C. Methods of engineering cells, such as with an exogenous FRB, are known, including any as described in Section III.

In some embodiments, engineered stem cells are iPSCs. In some embodiments, the engineered iPSCs are sequentially differentiated into hematopoietic progenitor cells (HPCs) and then into a myeloid cell type (e.g, iMC), such as a macrophage (e.g., iMACs) or a neutrophil (e.g., iNEU) by provided methods involving engagement of the engineered synthetic cytokine receptor with the non-physiological ligand. In some embodiments, the differentiation pathway may include an intermediate differentiation into myeloid progenitor cells. In some embodiments, the engineered iPSCs are sequentially differentiated into hematopoietic progenitor cells (HPCs); the HPCs into myeloid progenitors; and then the myeloid progenitors into myeloid cells, such as macrophages—termed “iMAC” cells. In a variation, macrophages may be derived from HPCs by sequentially differentiating the HPCs into myeloid progenitor cells; and then the myeloid progenitor cells into iMAC cells. In a further variation, macrophages may be derived by differentiating myeloid progenitor cells into iMAC cells.

In some embodiments, hematopoietic stem cells may be engineered to express a synthetic cytokine receptor. In some embodiments, myeloid progenitor cells may be engineered to express a synthetic cytokine receptor. In some embodiments, macrophages may be engineered to express a synthetic cytokine receptor.

In any of the provided embodiments, the synthetic cytokine receptor may be any as described herein that is able to be activated by a non-physiological ligand (e.g. rapamycin). In some embodiments, the synthetic cytokine receptor is a rapamycin activated cytokine receptor (RACR) that is able to be activated by rapamycin or a rapalog. In provided embodiments, activation of the synthetic cytokine receptor induces differentiation, in addition to or instead of an exogenous cytokine.

In some embodiments, the non-physiological ligand may induce differentiation, in addition to or instead of an exogenous cytokine. In some embodiments, the non-physiological ligand may induce differentiation during one or more of mesoderm formation, hematopoietic specification, myeloid progenitor cell differentiation, myeloid cell differentiation, and macrophage cell differentiation.

In some embodiments, engineering cells to express a synthetic cytokine receptor and activating the receptor with a non-physiological ligand allows for the generation of a pure population of myeloid cells (e.g., macrophages) that may be differentiated and/or expanded without the use of cell sorting.

### B. Synthetic Cytokine Receptor Complex

The synthetic cytokine receptors of the present disclosure comprise a synthetic gamma chain and a synthetic beta chain, each comprising a dimerization domain. The dimerization domains controllable dimerize in the present of a non-physiological ligand, thereby activating signaling the synthetic cytokine receptor.

In some embodiments, the synthetic gamma chain polypeptide comprises a first dimerization domain, a first transmembrane domain, and an interleukin-2 receptor subunit gamma (IL-2RG) intracellular domain. In some embodiments, the synthetic beta chain polypeptide comprises a second dimerization domain, a second transmembrane domain, and a beta chain intracellular domain selected from an interleukin-2 receptor subunit beta (IL-2RB) intracellular domain, an interleukin-7 receptor subunit beta (IL-7RB) intracellular domain, or an interleukin-21 receptor subunit beta (IL-21RB) intracellular domain. In particular embodiments, the beta chain intracellular domain is a IL-2RB intracellular domain. In some of any such embodiments, the first and/or second transmembrane domain are independently a transmembrane domain sequence heterologous to the sequence from which the gamma chain or beta chain intracellular domains are derived. In some embodiments, the first and second transmembrane domains are different. In some of any such embodiments, the first and/or second transmembrane domain are independently a transmembrane sequence the sequence from the same protein from which the gamma chain or beta chain intracellular domains are derived. In some embodiments, the first transmembrane domain is a IL-2RG transmembrane domain. In some embodiments, the second transmembrane domain is a beta chain transmembrane domain selected from an interleukin-2 receptor subunit beta (IL-2RB) transmembrane domain, an interleukin-7 receptor subunit beta (IL-7RB) transmembrane domain, or an interleukin-21 receptor subunit beta (IL-21RB) transmembrane domain. In some embodiments, the synthetic gamma chain polypeptide comprises a first dimerization domain, an interleukin-2 receptor subunit gamma (IL-2RG) transmembrane domain, and an interleukin-2 receptor subunit gamma (IL-2RG) intracellular domain. The dimerization domain may be extracellular (N-terminal to the transmembrane domain) or intracellular (C-terminal to the transmembrane domain and N- or C-terminal to the IL-2G intracellular domain.

The synthetic beta chain polypeptide comprises a second dimerization domain, an interleukin-2 receptor subunit beta (IL-2RB) transmembrane domain, and an interleukin-2 receptor subunit beta (IL-2RB) intracellular domain. The dimerization domain may be extracellular (N-terminal to the transmembrane domain) or intracellular (C-terminal to the transmembrane domain and N- or C-terminal to the IL-2RB intracellular domain).

In some embodiments, the synthetic gamma chain polypeptide is encoded by a nucleic acid sequence that encodes a signal peptide. In some embodiments, the synthetic beta chain polypeptide is encoded by a nucleic acid sequence that encodes a signal peptide. A skilled artisan is readily familiar with signal peptides that can provide a signal to transport a nascent protein in the cells. Any of a variety of signal peptides can be employed. It is understood that the signal peptide is cleaved and a mature synthetic cytokine receptor (without the signal peptide) is generated for expression on the cell surface.

In some embodiments, the signal peptide is a CD8a signal sequence shown as SEQ ID NO: 12: MALPVTALLLPLALLLHAARP.

In some embodiments, the signal peptide is a signal sequence shown as SEQ ID NO: 29: MPLGLLWLGLALLGALHAQA

In some embodiments, the non-physiological ligand activates the synthetic cytokine receptor in the macrophages to induce expansion and/or activation of the engineered macrophages. In some embodiments, the non-physiological ligand is rapamycin or a rapalog, such synthetic cytokine receptor termed a rapamycin-activated cytokine receptor (RACR).

In some embodiments, the non-physiological ligand activates the synthetic cytokine receptor in the macrophages to induce expansion of the macrophages. In some embodiments, the activation of the synthetic cytokine receptor results in at least about 10-fold, at least about 50-fold, at least about 100-fold, at least about 200-fold, at least about 300-fold, at least about 400-fold, at least about 500-fold, at least about 1000-fold, at least about 1500-fold, at least about 2000-fold, at least about 2500-fold, at least about 3000-fold, at least about 3500-fold, or at least about 4000-fold increased number of macrophages compared to uninduced cells. In some embodiments, the activation of the synthetic cytokine receptor results in at least about 5000-fold, at least about 6000-fold, at least about 7000-fold, at least about 8000-fold, at least about 9000-fold, at least about 10,000-fold, at least about 50,000-fold, at least about 100,000-fold, at least about 250,000-fold, at least about 500,000-fold, at least about 750,000-fold, or at least about 1,000,000-fold increased number of macrophages compared to uninduced cells.

In some embodiments, the macrophages increase by about 10-fold to about 100-fold, about 50-fold to about 200-fold, about 100-fold to about 300-fold, about 200-fold to about 400-fold, about 300-fold to about 500-fold, about 400-fold to about 1000-fold, about 500-fold to about 1500-fold, about 1000-fold to about 2000-fold, about 1500-fold to about 2500-fold, about 2000-fold to about 3000-fold, about 2500-fold to about 3500-fold, about 3000-fold to about 4000-fold, or any value in between these ranges. In some embodiments, the macrophages increase by about 4000-fold to about 6000-fold, about 5000-fold to about 7000-fold, about 6000-fold to about 8000-fold, about 7000-fold to about 9000-fold, about 8000-fold to about 10000-fold, about 9000-fold to about 50,000-fold, about 10000-fold to about 100,000-fold, about 50,000-fold to about 250,000-fold, about 100,000-fold to about 500,000-fold, about 250,000-fold to about 750,000-fold, about 500,000-fold to about 1,000,000-fold, or any value in between these ranges.

In some embodiments, the non-physiological ligand activates the synthetic cytokine receptor in the stem cells to induce differentiation. In some embodiments, the non-physiological ligand is rapamycin or a rapalog, such synthetic cytokine receptor termed a rapamycin-activated cytokine receptor (RACR).

In some embodiments, the non-physiological ligand activates the synthetic cytokine receptor in the stem cells to induce expansion of the hematopoietic progenitors differentiated from the stem cells. In some embodiments, the activation of the synthetic cytokine receptor results in at least about 10-fold, at least about 50-fold, at least about 100-fold, at least about 200-fold, at least about 300-fold, at least about 400-fold, at least about 500-fold, at least about 1000-fold, at least about 1500-fold, at least about 2000-fold, at least about 2500-fold, at least about 3000-fold, at least about 3500-fold, or at least about 4000-fold increased number of hematopoietic progenitors compared to non-engineered cells.

In some embodiments, the hematopoietic progenitors increase by about 10-fold to about 100-fold, about 50-fold to about 200-fold, about 100-fold to about 300-fold, about 200-fold to about 400-fold, about 300-fold to about 500-fold, about 400-fold to about 1000-fold, about 500-fold to about 1500-fold, about 1000-fold to about 2000-fold, about 1500-fold to about 2500-fold, about 2000-fold to about 3000-fold, about 2500-fold to about 3500-fold, about 3000-fold to about 4000-fold, or any value in between these ranges.

### 1. Intracellular Domain

In some embodiments, the intracellular signaling domain of the first transmembrane receptor protein comprises an interleukin-2 receptor subunit gamma (IL2Rg) domain. In some embodiments, the IL2Rg domain comprises the sequence set forth in SEQ ID NO: 1. In some embodiments, the IL2Rg Common Gamma Chain Intracellular domain has at least 80% amino acid identity, at least 85% amino acid identity, at least 90% amino acid identity, at least 95% amino acid identity, or 100% amino acid identity to SEQ ID NO: 1.

The sequence of a IL2RG Common Gamma Chain Intracellular domain is set forth in SEQ ID NO: 1:

ERTMPRIPTLKNLEDLVTEYHGNFSAWSGVSKGLAESLQPDYSER
LCLVSEIPPKGGALGEGPGASPCNQHSPYWAPPCYTLKPET.

In some embodiments, the synthetic cytokine receptor comprises a first transmembrane receptor protein comprising an IL-2RG intracellular domain, a first dimerization domain, a second transmembrane receptor protein comprising an IL-2RB intracellular domain, and a second dimerization domain.

In some embodiments, the synthetic beta chain comprises an interleukin-2 receptor subunit beta (IL2RB) intracellular domain. IL2RB is also known as IL15RB or CD122. Thus, when referred to herein, IL2RB can also mean IL15RB. That is, the terms are used interchangeably in the present disclosure. In some embodiments, the IL2RB intracellular domain comprises the sequence set forth in SEQ ID NO: 2. In some embodiments, the IL2RB intracellular domain has at least 80% amino acid identity, at least 85% amino acid identity, at least 90% amino acid identity, at least 95% amino acid identity, or 100% amino acid identity to SEQ ID NO: 2.

The sequence of a IL2RB intracellular domain is set forth in SEQ ID NO: 2:

NCRNTGPWLKKVLKCNTPDPSKFFSQLSSEHGGDVQKWLSSPFPS
SSFSPGGLAPEISPLEVLERDKVTQLLLQQDKVPEPASLSSNHSL
TSCFTNQGYFFFHLPDALEIEACQVYFTYDPYSEEDPDEGVAGAP
TGSSPQPLQPLSGEDDAYCTFPSRDDLLLFSPSLLGGPSPPSTAP
GGSGAGEERMPPSLQERVPRDWDPQPLGPPTPGVPDLVDFQPPPE
LVLREAGEEVPDAGPREGVSFPWSRPPGQGEFRALNARLPLNTDA
YLSLQELQGQDPTHLV

In some embodiments, the synthetic cytokine receptor comprises a first transmembrane receptor protein comprising an IL-2RG intracellular domain, a first dimerization domain, a second transmembrane receptor protein comprising an IL-7RB intracellular domain, and a second dimerization domain.

In some embodiments, the synthetic beta chain comprises an interleukin-7 receptor subunit beta (IL7RB) intracellular domain. In some embodiments, the IL7RB intracellular domain comprises the sequence set forth in SEQ ID NO: 3. In some embodiments, the IL7RB intracellular domain has at least 80% amino acid identity, at least 85% amino acid identity, at least 90% amino acid identity, at least 95% amino acid identity, or 100% amino acid identity to SEQ ID NO: 3.

The sequence of a IL7RB intracellular domain is set forth in SEQ ID NO: 3:

KKRIKPIVWPSLPDHKKTLEHLCKKPRKNLNVSFNPESFLDCQIH
RVDDIQARDEVEGFLQDTFPQQLEESEKQRLGGDVQSPNCPSEDV
VITPESFGRDSSLTCLAGNVSACDAPILSSSRSLDCRESGKNGPH
VYQDLLLSLGTTNSTLPPPFSLQSGILTLNPVAQGQPILTSLGSN
QEEAYVTMSSFYQNQ

In some embodiments, the synthetic cytokine receptor comprises a first transmembrane receptor protein comprising an IL-2RG intracellular domain, a first dimerization domain, a second transmembrane receptor protein comprising an IL-21RB intracellular domain, and a second dimerization domain.

In some embodiments, the synthetic beta chain comprises an interleukin-21 receptor subunit beta (IL21RB) intracellular domain. In some embodiments, the IL21RB intracellular domain comprises the sequence set forth in SEQ ID NO: 4. In some embodiments, the IL21RB intracellular domain has at least 80% amino acid identity, at least 85% amino acid identity, at least 90% amino acid identity, at least 95% amino acid identity, or 100% amino acid identity to SEQ ID NO: 4.

The sequence of a IL21RB intracellular domain is set forth in SEQ ID NO: 4:

SLKTHPLWRLWKKIWAVPSPERFFMPLYKGCSGDFKKWVGAPFTG
SSLELGPWSPEVPSTLEVYSCHPPRSPAKRLQLTELQEPAELVES
DGVPKPSFWPTAQNSGGSAYSEERDRPYGLVSIDTVTVLDAEGPC
TWPCSCEDDGYPALDLDAGLEPSPGLEDPLLDAGTTVLSCGCVSA
GSPGLGGPLGSLLDRLKPPLADGEDWAGGLPWGGRSPGGVSESEA
GSPLAGLDMDTFDSGFVGSDCSSPVECDFTSPGDEGPPRSYLRQW
VVIPPPLSSPGPQAS

### 2. Transmembrane Domains

The transmembrane (TM) domain is the sequence of the synthetic cytokine receptor that spans the membrane. The transmembrane domain may comprise a hydrophobic alpha helix.

In some embodiments, the transmembrane domain is derived from a human protein.

The sequence of a transmembrane domain is shown as SEQ ID NO: 8:

VVISVGSMGLIISLLCVYFWL

The sequence of a TM domain is shown as SEQ ID NO: 9:

VAVAGCVFLLISVLLLSGL

The sequence of TM domain is shown as SEQ ID NO: 10:

PILLTISILSFFSVALLVILACVLW

The sequence of a TM domain is shown as SEQ ID NO: 11:

GWNPHLLLLLLLVIVFIPAFW

The sequence of a TM domain is shown as SEQ ID NO: 36:

IPWLGHLLVGLSGAFGFIILVYLLI.

In some embodiments, the TM domain and the intracellular signaling domain are from the same cytokine receptor. In some embodiments, the synthetic gamma chain polypeptide contains an IL-2RG TM domain and a IL-2RG intracellular domain. In some embodiments, the synthetic beta chain polypeptide contains an IL-2RB TM domain and a IL-2RB intracellular domain. In some embodiments, the synthetic beta chain polypeptide contains an IL-7RB TM domain and a IL-7RB intracellular domain. In some embodiments, the synthetic beta chain polypeptide contains an IL-21RB TM domain and a IL-21RB intracellular domain.

In some embodiments, one or more additional contiguous amino acids of the ectodomain directly adjacent to the TM domain of the cytokine receptor also can be included as part of the polypeptide sequence of a chain of the synthetic cytokine receptor. In some embodiments, 1-20 contiguous amino acids of the ectodomain adjacent to the TM domain of the cytokine receptor is included as part of the polypeptide sequence of a chain of the synthetic cytokine receptor. The portion of the ectodomain may be a contiguous sequence of 1, 2, 3, 4, 5, 6, 7, 8, 9, 10, 11, 12, 13, 14, 15, 16, 17, 18, 19 or 20 amino acids directly adjacent (e.g. N-terminal to) the TM sequence.

In some embodiments, the synthetic gamma chain polypeptide contains an IL-2RG TM domain comprising the sequence set forth in SEQ ID NO: 8 and a IL-2RG intracellular domain comprising the sequence set forth in SEQ ID NO:1. In some embodiments, the synthetic gamma chain polypeptide contains an IL-2RG TM domain comprising the sequence set forth in SEQ ID NO: 31 and a IL-2RG intracellular domain comprising the sequence set forth in SEQ ID NO:1.

In some embodiments, the synthetic beta chain polypeptide contains an IL-2RB TM domain comprising the sequence set forth in SEQ ID NO: 36 and a IL-2RB intracellular domain comprising the sequence set forth in SEQ ID NO:2. In some embodiments, the synthetic beta chain polypeptide contains an IL-2RB TM domain comprising the sequence set forth in SEQ ID NO: 35 and a IL-2RB intracellular domain comprising the sequence set forth in SEQ ID NO:2.

### 3. Dimerization Domain

The dimerization domains may be heterodimerization domains, including but not limited to FK506-Binding Protein of size 12 kD (FKBP) and a FKBP12-rapamycin binding (FRB) domain, which are known in the art to dimerize in the presence of rapamycin or a rapalog. In some embodiments, the FRB domain may comprise a polypeptide sequence at least 80%, at least 90%, at least 95%, at least 96%, at least 97%, at least 98%, at least 99%, or 100% identical to SEQ ID NO: 6 or SEQ ID NO:7. In some embodiments, The FKBP domain may comprise a polypeptide sequence at least 80%, at least 90%, at least 95%, at least 96%, at least 97%, at least 98%, at least 99%, or 100% identical to SEQ ID NO: 5. In some embodiments, The FKBP domain may comprise a polypeptide sequence at least 80%, at least 90%, at least 95%, at least 96%, at least 97%, at least 98%, at least 99%, or 100% identical to SEQ ID NO: 49. In some embodiments, The FKBP domain may comprise a polypeptide sequence at least 80%, at least 90%, at least 95%, at least 96%, at least 97%, at least 98%, at least 99%, or 100% identical to SEQ ID NO: 30.

In some embodiments, the sequence of an illustrative FKBP domain is set forth in SEQ ID NO: 5:

GVQVETISPGDGRTFPKRGQTCVVHYTGMLEDGKKFDSSRDRNKP
FKFMLGKQEVIRGWEEGVAQMSVGQRAKLTISPDYAYGATGHPGI
IPPHATLVFDVELLKLE

In some embodiments, the sequence of an illustrative FKBP domain is set forth in SEQ ID NO: 49:

GVQVETISPGDGRTFPKRGQTCVVHYTGMLEDGKKFDSSRDRNKP
FKFMLGKQEVIRGWEEGVAQMSVGQRAKLTISPDYAYGATGHPGI
IPPHATLVFDVELLKL

In some embodiments, the sequence of an illustrative FKBP domain is set forth in SEQ ID NO: 30:

GVQVETISPGDGRTFPKRGQTCVVHYTGMLEDGKKFDSSRDRNKP
FKFMLGKQEVIRGWEEGVAQMSVGQRAKLTISPDYAYGATGHPGI
IPPHATLVFDVELLKLGE

In some embodiments, the sequence of an illustrative FRB domain is set forth in SEQ ID NO: 6:

ILWHEMWHEGLEEASRLYFGERNVKGMFEVLEPLHAMMERGPQTL
KETSFNQAYGRDLMEAQEWCRKYMKSGNVKDLTQAWDLYYHVFRR
ISK

In some embodiments, the sequence of variant FRB domain (FRB mutant domain) is set forth in SEQ ID NO: 7:

ILWHEMWHEGLEEASRLYFGERNVKGMFEVLEPLHAMMERGPQTL
KETSFNQAYGRDLMEAQEWCRKYMKSGNVKDLLQAWDLYYHVFRR
ISK

In some embodiments, the first dimerization domain is set forth in SEQ ID NO:5 and the second dimerization domain is set forth in SEQ ID NO:6.

In some embodiments, the first dimerization domain is set forth in SEQ ID NO:49 and the second dimerization domain is set forth in SEQ ID NO:6.

In some embodiments, the first dimerization domain is set forth in SEQ ID NO:30 and the second dimerization domain is set forth in SEQ ID NO:6.

In some embodiments, the first dimerization domain is set forth in SEQ ID NO:5 and the second dimerization domain is set forth in SEQ ID NO:7.

In some embodiments, the first dimerization domain is set forth in SEQ ID NO:49 and the second dimerization domain is set forth in SEQ ID NO:7.

In some embodiments, the first dimerization domain is set forth in SEQ ID NO:30 and the second dimerization domain is set forth in SEQ ID NO:7.

Alternatively, the first dimerization domain and the second dimerization domain may be a FK506-Binding Protein of size 12 kD (FKBP) and a calcineurin domain, which are known in the art to dimerize in the presence of FK506 or an analogue thereof.

In some embodiments the dimerization domains are homodimerization domains selected from:
i) FK506-Binding Protein of size 12 kD (FKBP);
ii) ii) cyclophiliA (CypA); or
iii) iii) gyrase B (CyrB);
with the corresponding non-physiological ligands being, respectively
i) FK1012, AP1510, AP1903, or AP20187;
ii) ii) cyclosporin-A (CsA); or
iii) iii) coumermycin or analogs thereof.

In some embodiments, the first and second dimerization domains of the transmembrane receptor proteins are a FKBP domain and a cyclophilin domain.

In some embodiments, the first and second dimerization domains of the transmembrane receptor proteins are a FKBP domain and a bacterial dihydrofolate reductase (DHFR) domain.

In some embodiments, the first and second dimerization domains of the transmembrane receptor proteins are a calcineurin domain and a cyclophilin domain.

In some embodiments, the first and second dimerization domains of the transmembrane receptor proteins are PYR1-like 1 (PYL1) and abscisic acid insensitive 1 (ABI1).

In some embodiments, the synthetic cytokine receptor is composed of a synthetic gamma chain polypeptide that contains a FKBP12 dimerization domain and an IL-2RG intracellular domain, and a synthetic beta chain polypeptide that contains a FRB dimerization domain and an IL-2RB intracellular domain. In some embodiments, the synthetic gamma chain polypeptide has the sequence of amino acids set forth in SEQ ID NO:28 or a sequence of amino acids that is at least 90%, at least 95%, at least 96%, at least 97%, at least 98%, at least 99%, or 100% identical to SEQ ID NO: 28. In some embodiments, the synthetic beta chain polypeptide has the sequence of amino acids set forth in SEQ ID NO:33 or a sequence of amino acids that is at least 90%, at least 95%, at least 96%, at least 97%, at least 98%, at least 99%, or 100% identical to SEQ ID NO: 33.

In some embodiments, the synthetic cytokine receptor is composed of a synthetic gamma chain polypeptide set forth in SEQ ID NO:28 and a synthetic beta chain polypeptide set forth in SEQ ID NO:33.

In some embodiments, the synthetic cytokine receptor is composed of a synthetic gamma chain polypeptide that contains a FKBP12 dimerization domain and an IL-2RG intracellular domain, and a synthetic beta chain polypeptide that contains a FRB dimerization domain and an IL-2RB intracellular domain. In some embodiments, the synthetic gamma chain polypeptide has the sequence of amino acids set forth in SEQ ID NO:28 or a sequence of amino acids that is at least 90%, at least 95%, at least 96%, at least 97%, at least 98%, at least 99%, or 100% identical to SEQ ID NO: 28. In some embodiments, the synthetic beta chain polypeptide has the sequence of amino acids set forth in SEQ ID NO:55 or a sequence of amino acids that is at least 90%, at least 95%, at least 96%, at least 97%, at least 98%, at least 99%, or 100% identical to SEQ ID NO: 55.

In some embodiments, the synthetic cytokine receptor is composed of a synthetic gamma chain polypeptide set forth in SEQ ID NO:28 and a synthetic beta chain polypeptide set forth in SEQ ID NO:55.

In some embodiments, the synthetic cytokine receptor is composed of a synthetic gamma chain polypeptide that contains a FKBP12 dimerization domain and an IL-2RG intracellular domain, and a synthetic beta chain polypeptide that contains a FRB dimerization domain and an IL-2RB intracellular domain. In some embodiments, the synthetic gamma chain polypeptide has the sequence of amino acids set forth in SEQ ID NO:56 or a sequence of amino acids that is at least 90%, at least 95%, at least 96%, at least 97%, at least 98%, at least 99%, or 100% identical to SEQ ID NO: 56. In some embodiments, the synthetic beta chain polypeptide has the sequence of amino acids set forth in SEQ ID NO:57 or a sequence of amino acids that is at least 90%, at least 95%, at least 96%, at least 97%, at least 98%, at least 99%, or 100% identical to SEQ ID NO: 57.

In some embodiments, the synthetic cytokine receptor is composed of a synthetic gamma chain polypeptide set forth in SEQ ID NO:56 and a synthetic beta chain polypeptide set forth in SEQ ID NO:57.

In some embodiments, the synthetic cytokine receptor is able to be bound by the non-physiological ligand rapamycin or a rapamycin analog. In some embodiments, the synthetic cytokine receptor is responsive to the non-physiological ligand rapamycin or a rapamycin analog, in which binding of the non-physiological ligand to the dimerization domains of the synthetic cytokine receptor induces cytokine receptor-mediated signaling in the cell, such as via the JAK/STAT pathway.

### 4. Non-Physiological Ligand

In various embodiments of the compositions and methods of the disclosure, the system comprises a non-physiological ligand. Illustrative small molecules useful as ligands include, without limitation: rapamycin, fluorescein, fluorescein isothiocyanate (FITC), 4-[(6-methylpyrazin-2-yl) oxy]benzoic acid (aMPOB), folate, rhodamine, acetazolamide, and a CA9 ligand.

In some embodiments, the synthetic cytokine receptor is activated by a ligand. In some embodiments, the ligand is a non-physiological ligand.

In some embodiments, the non-physiological ligand is a rapalog.

In some embodiments, the non-physiological ligand is rapamycin.

In some embodiments, the non-physiological ligand is AP21967.

In some embodiments, the non-physiological ligand is FK506.

In some embodiments, the non-physiological ligand is FK1012. In some embodiments, the non-physiological ligand is AP1510. In some embodiments, the non-physiological ligand is AP1903. In some embodiments, the non-physiological ligand is AP20187. In some embodiments, the non-physiological ligand is cyclosporin-A (CsA). In some embodiments, the non-physiological ligand is coumermycin.

In some embodiments, the synthetic cytokine receptor complex activated by folate, fluorescein, aMPOB, acetazolamide, a CA9 ligand, tacrolimus, rapamycin, a rapalog (a rapamycin analog), CD28 ligand, poly(his) tag, Strep-tag, FLAG-tag, VS-tag, Myc-tag, HA-tag, NE-tag, biotin, digoxigenin, dinitrophenol, or a derivative thereof.

In some embodiments, the non-physiological ligand may be an inorganic or organic compound that is less than 1000 Daltons.

In some embodiments, the ligand may be rapamycin or a rapamycin analog (rapalog). In some embodiments, the rapalog comprises variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring.

Thus, in some embodiments, the rapalog is everolimus, novolimus, pimecrolimus, ridaforolimus, tacrolimus, temsirolimus, umirolimus, zotarolimus, Temsirolimus (CCI-779), C20-methallylrapamycin, C16-(S)-3-methylindolerapamycin, C16-(S)-3-methylindolerapamycin (C16-iRap), AP21967 (A/C Heterodimerizer, Takara Bio®), sodium mycophenolic acid, benidipine hydrochloride, rapamine, AP23573 (Ridaforolimus), AP1903 (Rimiducid), or metabolites, derivatives, and/or combinations thereof.

In some embodiments, the ligand comprises FK1012 (a semisynthetic dimer of FK506), tacrolimus (FK506), FKCsA (a composite of FK506 and cyclosporine), rapamycin, coumermycin, gibberellin, HaXS dimerizer (chemical dimerizers of HaloTag and SNAP-tag), TmP-HTag (trimethoprim haloenzyme protein dimerizer), or ABT-737 or functional derivatives thereof.

In some embodiments, the non-physiological ligand is present or provided in an amount from 0 nM to 1000 nM such as, e.g., 0.05 nM, 0.1 nM, 0.5. nM, 1.0 nM, 5.0 nM, 10.0 nM, 15.0 nM, 20.0 nM, 25.0 nM, 30.0 nM, 35.0 nM, 40.0 nM, 45.0 nM, 50.0 nM, 55.0 nM, 60.0 nM, 65.0 nM, 70.0 nM, 75.0 nM, 80.0 nM, 90.0 nM, 95.0 nM, 100 nM, 200 nM, 300 nM, 400 nM, 500 nM, 600 nM, 700 nM, 800 nM, 900 nM, or 1000 nM, or an amount that is within a range defined by any two of the aforementioned amounts.

In some embodiments, the non-physiological ligand is AP21967 and is present or provided at 10 nM. In some embodiments, the non-physiological ligand is AP21967 and is present or provided at 20 nM. In some embodiments, the non-physiological ligand is AP21967 and is present or provided at 50 nM. In some embodiments, the non-physiological ligand is AP21967 and is present or provided at 100 nM.

In some embodiments, the non-physiological ligand is rapamycin and is present or provided at 1 nM. In some embodiments, the non-physiological ligand is rapamycin and is present or provided at 10 nM. In some embodiments, the non-physiological ligand is rapamycin and is present or provided at 20 nM. In some embodiments, the non-physiological ligand is rapamycin and is present or provided at 50 nM.

In some embodiments, the non-physiological ligand is a rapalog and is present or provided at 1 nM. In some embodiments, the non-physiological ligand is a rapalog and is present or provided at 10 nM. In some embodiments, the non-physiological ligand is a rapalog and is present or provided at 20 nM. In some embodiments, the non-physiological ligand is a rapalog and is present or provided at 50 nM. In some embodiments, the non-physiological ligand is a rapalog and is present or provided at 100 nM.

In some embodiments, the non-physiological ligand is present or provided at 1 nM.

In some embodiments, the non-physiological ligand is present or provided at 10 nM.

In some embodiments, the non-physiological ligand is present or provided at 100 nM.

In some embodiments, the non-physiological ligand is present or provided at 1000 nM.

### C. Cytosolic FRB

In some embodiments, the engineered cells, such as stem cells or macrophages, can be contacted with free cytosolic FRB. As described in more detail elsewhere herein, rapamycin normally binds to FBP12, and the FKBP12-rapamycin complex then binds to the FRB subunit of mTOR and blocks mTOR signaling. Thus, contacting a cell with rapamycin could, in some cases, inhibit or reduce cell growth and expansion. In some embodiments, the cells can be made “rapamycin resistance” by providing free cytosolic FRB to the cell in order to complex with rapamycin and thereby eliminate or reduce rapamycin-mediated growth inhibition of a source cell or iMAC.

In some embodiments, soluble FRB can be microinjected into a stem cell or macrophage to eliminate or reduce rapamycin-mediated growth inhibition. In some embodiments, a stem cell or macrophage can be transduced with a vector containing soluble FRB to eliminate or reduce rapamycin-mediated growth inhibition. In some embodiments, soluble FRB can be added to cell culture media to eliminate or reduce rapamycin mediated growth inhibition.

In an embodiment where soluble FRB is microinjected into a stem cell or macrophage, the soluble FRB is injected at a concentration of 4 mg/mL, 4.5 mg/mL, 5 mg/mL, 5.5 mg/mL, or 6 mg/mL. In an embodiment where soluble FRB is microinjected into a stem cell or macrophage, the soluble FRB is injected at a concentration of 1 μM.

In some embodiments, a nucleic acid molecule encoding FRB, such as by introduction of a vector construct encoding FRB, is introduced into the cell. In some embodiments, the construct is designed for insertion of the nucleic acid encoding FRB into an endogenous locus in the cell. Methods of gene insertion or knock-in are known, including any of the methods described in Section III. In some embodiments, insertion of an FRB-encoding construct is by homology directed repair, such as by using a CRISPR-Cas system. In some embodiments, the engineered cell that expresses FRB at an endogenous loci is able to express free cytosolic FRB in the cell.

The FRB domain is an approximately 100 amino acid domain derived from the mTOR protein kinase. It may be expressed in the cytosol as a freely diffusible soluble protein. Advantageously, the FRB domain reduces the inhibitory effects of rapamycin on mTOR in the engineered cells and promote consistent activation of engineered cells giving the cells a proliferative advantage over native cells.

In some embodiments, synthetic cytokine receptor complex comprises a cytosolic polypeptide that binds to the ligand or a complex comprising the ligand.

In some embodiments, the cytosolic polypeptide comprises an FRB domain. In some embodiments, the cytosolic polypeptide comprises an FRB domain and the ligand is rapamycin. The cytosolic FRB domain may comprise a polypeptide sequence at least 80%, at least 90%, at least 95%, at least 96%, at least 97%, at least 98%, at least 99%, or 100% identical to SEQ ID NO: 6 or SEQ ID NO: 7. FRB domain may be a naked FRB domain consisting essentially of a polypeptide having a polypeptide sequence at least 80%, at least 90%, at least 95%, at least 96%, at least 97%, at least 98%, at least 99%, or 100% identical to SEQ ID NO: 6 or SEQ ID NO: 7.

In some embodiments, the cells are contacted with an FRB domain protein that has the sequence set forth in SEQ ID NO:6.

In some embodiments, the cells are contacted with an FRB domain protein that has the sequence set forth in SEQ ID NO:7.

Advantageously, the cytosolic FRB confers resistance to the immunosuppressive effect of the non-physiological ligand (e.g., rapamycin or rapalog).

### D. Differentiation of Stem Cells

Provided herein are methods of differentiating pluripotent stem cells, such as iPSCs, engineered with a synthetic cytokine receptor into a myeloid cell. Provided herein are methods of generating myeloid cells derived from pluripotent stem cells, such as iPSCs, engineered with a synthetic cytokine receptor. In some embodiments, the iPSC differentiation is by a pathway that includes differentiation into hematopoietic progenitors (HP) and myeloid progenitor. In some embodiments, the iPSC differentiation is by a pathway that includes differentiation into hematopoietic progenitors (HP) cells.

Although iPSC-derived cell therapies are promising, the current processes of deriving an immune cell from an iPSC is complex, variable, and costly. Current protocols for deriving downstream cell types from iPSCs use on sequential steps of “coaxing” of iPSCs down a differentiation pathway by feeding in external factors to engage endogenous receptors. In some embodiments, this process can require long culture times, expensive protein material, and can be highly variable due to dependency on constantly changing expression patterns of endogenous genes and receptors.

In some embodiments provided herein, iPSC differentiation is directed, at least in part, by the signaling induced from the synthetic cytokine receptor. In embodiments of the provided methods, engagement of the synthetic cytokine receptor (e.g. RACR) by its cognate non-physiological ligand (e.g. rapamycin) is able to deliver a cytokine signal into the cell inducing the JAK/STAT pathway and driving differentiation. The provided embodiments in which iPSCs are engineered with a synthetic cytokine receptor promotes differentiation through highly controlled synthetic receptors, which has the potential to reduce the variability of cell differentiation as well as decrease the cost of manufacturing of these cells by replacing expensive growth factors and cytokines with small molecule engagers (e.g. rapamycin). In some aspects, the requirement for further growth factors or cytokines to drive differentiation at one or more different steps of the process is reduced or eliminated, thereby providing for a directed and consistent differentiation.

In some embodiments, the provided methods include culturing the pluripotent stem cells (e.g. iPSCs) engineered with a synthetic cytokine receptor under conditions to differentiate cells into a population of myeloid cells. In some embodiments, the methods can include one more incubations in which different molecules are added to the culture media. In some embodiments, the methods include incubations in the presence of the non-physiological ligand (e.g., rapamycin or a rapalog). In some embodiments, the methods can include replacement of media to supplement or add any one or more molecules to the culture media.

In some embodiments, the provided methods include culturing the pluripotent stem cells (e.g. iPSCs) engineered with a synthetic cytokine receptor under conditions to differentiate cells into a population of myeloid cells or a progenitor thereof (e.g., hematopoietic progenitors (HP) or myeloid progenitor cells).

In some embodiments, the provided methods include culturing the pluripotent stem cells (e.g. iPSCs) engineered with a synthetic cytokine receptor under conditions to differentiate cells into a population of progenitor cells. In some embodiments, the provided methods include a) culturing the pluripotent stem cells (e.g. iPSCs) engineered with a synthetic cytokine receptor under conditions to differentiate cells into a population of progenitor cells and b) culturing the cells produced in a) by incubation under conditions to generate myeloid cells.

Examples of methods for differentiating stem cells (e.g., iPSCs) into multipotent hematopoietic progenitor cells are provided in U.S. Pat. No. 9,624,470, U.S. Patent Appl. No. 2020/0080059, as well as Mesquitta et al., Sci. Rep. 9:6622 (2019), the disclosures of which are incorporated by reference herein in their entireties. In some embodiments, culturing in a) is carried out by a first incubation under conditions to produce an Embryoid Body (EB) followed by one or more further incubations in the presence of the non-physiological ligand. In some embodiments, the culturing during the further incubations comprise optionally one or more myeloid cell differentiation factors selected from one or more of IL-3, M-CSF and GM-CSF.

In some embodiments, the first incubation is carried out in a first media. In some embodiments, the one or more further incubations comprises a second incubation in a second media. In some embodiments, the one or more further incubations comprises a second incubation in a second media and a third incubation in a third media.

In some embodiments, culturing in b) is carried out by a one or more further incubations under conditions to produce a myeloid cell. In some embodiments, culturing in b) is carried out by a fourth incubation in a fourth media to produce a myeloid cell. In some embodiments, the fourth incubation is in the presence of the non-physiological ligand.

In some embodiments, the culturing of the cell populations may be carried out under serum-free conditions. Examples of commercially available serum-free media suitable for cell attachment and/or induction include mTeSR™1, STEMdiff APEL 2 Medium, or TeSR™2 from Stem Cell Technologies (Vancouver, Canada), Primate ES/iPS cell medium from ReproCELL (Boston, Mass.), StemPro®-34 from Invitrogen (Carlsbad, Calif), StemPro® hESC SFM from Invitrogen, and X-VIVO™ from Lonza (Basel, Switzerland).

In some embodiments, the media comprises one or more of the following: nutrients, extracts, growth factors, hormones, cytokines and medium additives. Illustrative nutrients and extracts may include, for example, DMEM/F-12 (Dulbecco's Modified Eagle Medium/Nutrient Mixture F-12), which is a widely used basal medium for supporting the growth of many different mammalian cells; KOSR (knockout serum replacement); L-glut; NEAA (Non-Essential Amino Acids). Medium additives may include, but are not limited to, MTG, ITS, (ME, anti-oxidants (for example, ascorbic acid). In some embodiments, a media of the present invention comprises one or more of the following cytokines or growth factors: basic fibroblast growth factor (bFGF, also known as FGF2), transforming growth factor beta (TGF-β), bone morphogenetic protein (BMP4), vascular endothelial cell growth factor (VEGF or VEGF-165), various interleukins (e.g., IL-3), various colony-stimulating factors (such as granulocyte/macrophage colony-stimulating factor (GM-CSF) and/or macrophage colony-stimulating factor (M-CSF)), various interferons (such as IFNγ) and other cytokines such as stem cell factor (SCF).

These cytokines may be obtained commercially and may be either natural or recombinant. In some embodiments, the growth factors, mitogens, and cytokines are stage and/or cell type specific in concentrations that are determined empirically or as guided by the established cytokine art. Examples of exogenous cell culture media additives and supplements and cell selection kit components are provided in WO 2020/124256, the disclosure of which is incorporated by reference herein in its entirety.

In some embodiments, a media of the present invention comprises one or more of the following: a PI3K inhibitor, an AHR antagonist, a pyrimido-[4,5-b]-indole derivative, and a ROCK inhibitor. In some embodiments the PI3K inhibitor is LY294002. In some embodiments, the AHR antagonist is StemRegenin-1. In some embodiments, the pyrimido-[4,5-b]-indole derivative is UM729. In some embodiments the ROCK inhibitor is Y27632.

In some embodiments, the first media comprises one or more of BMP4, FGF2, VEGF-165 and a Rock Inhibitor, optionally wherein the Rock Inhibitor is Y27632. In some embodiments, the second media comprises one or more of BMP4, FGF2, VEGF, LY294002, IL-3, GM-CSF, and M-CSF. In some embodiments, the third media comprises one or more of UM729, StemRegenin-1, BMP4, FGF2, VEGF, LY294002, IL-3, GM-CSF, and M-CSF. In some embodiments, the fourth media comprises one or more of least SCF, GM-CSF, M-CSF, IL-3, an AHR antagonist, a pyrimido-[4,5-b]-indole derivative (e.g. UM729) and StemRegenin-1. In some embodiments, the first, second, third, and fourth media further comprises the non-physiological ligand.

In some embodiments, the culturing in the first media is for 1 to 4 days. In some days, the culturing is for at or about 1 day, 2 days, at or about 3 days or at or about 4 days.

In some embodiments, the culturing in the second media is for 3 to 6 days. In some days, the culturing is for at or about 3 days, at or about 4 days, at or about 5 days or at or about 6 days.

In some embodiments, the culturing in the third media is for 3 to 6 days. In some days, the culturing is for at or about 3 days, at or about 4 days, at or about 5 days or at or about 6 days.

In some embodiments, the culturing in the fourth media is for 9 to 15 days. In some days, the culturing is for at or about 9 days, at or about 10 days, at or about 11 days, at or about 12 days, at or about 13 days, at or about 14 days, or at or about 15 days.

In some embodiments, the concentration of the BMP4 in the media is from about 0.5 ng/mL-2.5 ng/mL, 0.5 ng/mL-5 ng/mL, 0.5 ng/mL-10 ng/mL, 0.5 ng/mL-15 ng/mL, 0.5 ng/mL-20 ng/mL, 0.5 ng/mL-30 ng/mL, 0.5 ng/mL-50 ng/mL, 2.5 ng/mL-5 ng/mL, 2.5 ng/mL-10 ng/mL, 2.5 ng/mL-15 ng/mL, 2.5 ng/mL-20 ng/mL, 2.5 ng/mL-30 ng/mL, 2.5 ng/mL-50 ng/mL, 5 ng/mL-10 ng/mL, 5 ng/mL-15 ng/mL, 5 ng/mL-20 ng/mL, 5 ng/mL-30 ng/mL, 5 ng/mL-50 ng/mL, 10 ng/mL-15 ng/mL, 10 ng/mL-20 ng/mL, 10 ng/mL-30 ng/mL, 10 ng/mL-50 ng/mL, each inclusive. In some embodiments, the concentration of BMP4 in the media is at least about 0.5 ng/mL, 2.5 ng/mL, 5 ng/mL, 10 ng/mL, 15 ng/mL, 20 ng/mL, 30 ng/mL, or 50 ng/mL, In some embodiments, the concentration of BMP4 in the media is about 10 ng/mL.

In some embodiments, the concentration of the FGF2 in the media is from about 0.5 ng/mL-2.5 ng/mL, 0.5 ng/mL-5 ng/mL, 0.5 ng/mL-10 ng/mL, 0.5 ng/mL-15 ng/mL, 0.5 ng/mL-20 ng/mL, 0.5 ng/mL-30 ng/mL, 0.5 ng/mL-50 ng/mL, 2.5 ng/mL-5 ng/mL, 2.5 ng/mL-10 ng/mL, 2.5 ng/mL-15 ng/mL, 2.5 ng/mL-20 ng/mL, 2.5 ng/mL-30 ng/mL, 2.5 ng/mL-50 ng/mL, 5 ng/mL-10 ng/mL, 5 ng/mL-15 ng/mL, 5 ng/mL-20 ng/mL, 5 ng/mL-30 ng/mL, 5 ng/mL-50 ng/mL, 10 ng/mL-15 ng/mL, 10 ng/mL-20 ng/mL, 10 ng/mL-30 ng/mL, 10 ng/mL-50 ng/mL, each inclusive. In some embodiments, the concentration of FGF2 in the media is at least about 0.5 ng/mL, 2.5 ng/mL, 5 ng/mL, 10 ng/mL, 15 ng/mL, 20 ng/mL, 30 ng/mL, or 50 ng/mL. In some embodiments, the concentration of FGF2 in the media is about 10 ng/mL. In some embodiments, the concentration of FGF2 in the media is about 50 ng/mL.

In some embodiments, the concentration of the VEGF in the media is from about 0.5 ng/mL-2.5 ng/mL, 0.5 ng/mL-5 ng/mL, 0.5 ng/mL-10 ng/mL, 0.5 ng/mL-15 ng/mL, 0.5 ng/mL-20 ng/mL, 0.5 ng/mL-30 ng/mL, 0.5 ng/mL-50 ng/mL, 0.5 ng/mL-100 ng/mL, 2.5 ng/mL-5 ng/mL, 2.5 ng/mL-10 ng/mL, 2.5 ng/mL-15 ng/mL, 2.5 ng/mL-20 ng/mL, 2.5 ng/mL-30 ng/mL, 2.5 ng/mL-50 ng/mL, 2.5 ng/mL-100 ng/mL, 5 ng/mL-10 ng/mL, 5 ng/mL-15 ng/mL, 5 ng/mL-20 ng/mL, 5 ng/mL-30 ng/mL, 5 ng/mL-50 ng/mL, 5 ng/mL-100 ng/mL, 10 ng/mL-15 ng/mL, 10 ng/mL-20 ng/mL, 10 ng/mL-30 ng/mL, 10 ng/mL-50 ng/mL, 10 ng/mL-100 ng/mL, each inclusive. In some embodiments, the concentration of VEGF in the media is at least about 0.5 ng/mL, 2.5 ng/mL, 5 ng/mL, 10 ng/mL, 15 ng/mL, 20 ng/mL, 30 ng/mL, 50 ng/mL, or 100 ng/mL, In some embodiments, the concentration of VEGF in the media is about 50 ng/mL.

In some embodiments, the concentration of the Y27632 in the media is from about 0.5 μM-2.5 μM, 0.5 μM-5 μM, 0.5 μM-10 μM, 0.5 μM-15 μM, 0.5 μM-20 μM, 0.5 μM-30 μM, 0.5 μM-50 μM, 2.5 μM-5 μM, 2.5 μM-10 μM, 2.5 μM-15 μM, 2.5 μM-20 μM, 2.5 μM-30 μM, 2.5 μM-50 μM, 5 μM-10 μM, 5 μM-15 μM, 5 μM-20 μM, 5 μM-30 μM, 5 μM-50 μM, 10 μM-15 μM, 10 μM-20 μM, 10 μM-30 μM, 10 μM-50 μM, each inclusive. In some embodiments, the concentration of Y27632 in the media is at least about 0.5 μM, 2.5 μM, 5 μM, 10 μM, 15 μM, 20 μM, 30 μM, or 50 μM, In some embodiments, the concentration of Y27632 in the media is about 10 μM.

In some embodiments, the concentration of the UM729 in the media is from about 0.5 μM-1 μM, 0.5 μM-5 μM, 0.5 μM-10 μM, 0.5 μM-15 μM, 0.5 μM-20 μM, 0.5 μM-30 μM, 0.5 μM-50 μM, 1 μM-5 μM, 1 μM-10 μM, 1 μM-15 μM, 1 μM-20 μM, 1 μM-30 μM, 1 μM-50 μM, 5 μM-10 μM, 5 μM-15 μM, 5 μM-20 μM, 5 μM-30 μM, 5 μM-50 μM, 10 μM-15 μM, 10 μM-20 μM, 10 μM-30 μM, 10 μM-50 μM, each inclusive. In some embodiments, the concentration of UM729 in the media is at least about 0.5 μM, 1 μM, 5 μM, 10 μM, 15 μM, 20 μM, 30 μM, or 50 μM, In some embodiments, the concentration of UM729 in the media is about 1 μM.

In some embodiments, the concentration of the M-CSF in the media is from about 0.5 ng/mL-2.5 ng/mL, 0.5 ng/mL-5 ng/mL, 0.5 ng/mL-10 ng/mL, 0.5 ng/mL-15 ng/mL, 0.5 ng/mL-20 ng/mL, 0.5 ng/mL-30 ng/mL, 0.5 ng/mL-50 ng/mL, 0.5 ng/mL-100 ng/mL, 0.5 ng/mL-200 ng/mL, 2.5 ng/mL-5 ng/mL, 2.5 ng/mL-10 ng/mL, 2.5 ng/mL-15 ng/mL, 2.5 ng/mL-20 ng/mL, 2.5 ng/mL-30 ng/mL, 2.5 ng/mL-50 ng/mL, 2.5 ng/mL-100 ng/mL, 2.5 ng/mL-200 ng/mL, 5 ng/mL-10 ng/mL, 5 ng/mL-15 ng/mL, 5 ng/mL-20 ng/mL, 5 ng/mL-30 ng/mL, 5 ng/mL-50 ng/mL, 5 ng/mL-100 ng/mL, 5 ng/mL-200 ng/mL, 10 ng/mL-15 ng/mL, 10 ng/mL-20 ng/mL, 10 ng/mL-30 ng/mL, 10 ng/mL-50 ng/mL, 10 ng/mL-100 ng/mL, 10 ng/mL-200 ng/mL, each inclusive. In some embodiments, the concentration of M-CSF in the media is at least about 0.5 ng/mL, 2.5 ng/mL, 5 ng/mL, 10 ng/mL, 15 ng/mL, 20 ng/mL, 30 ng/mL, 50 ng/mL, 100 ng/mL, or 200 ng/mL. In some embodiments, the concentration of M-CSF in the media is about 50 ng/mL. In some embodiments, the concentration of M-CSF is increased in the media to 100 ng/mL.

In some embodiments, the concentration of the GM-CSF in the media is from about 0.5 ng/mL-2.5 ng/mL, 0.5 ng/mL-5 ng/mL, 0.5 ng/mL-10 ng/mL, 0.5 ng/mL-15 ng/mL, 0.5 ng/mL-20 ng/mL, 0.5 ng/mL-30 ng/mL, 0.5 ng/mL-50 ng/mL, 0.5 ng/mL-100 ng/mL, 0.5 ng/mL-200 ng/mL, 2.5 ng/mL-5 ng/mL, 2.5 ng/mL-10 ng/mL, 2.5 ng/mL-15 ng/mL, 2.5 ng/mL-20 ng/mL, 2.5 ng/mL-30 ng/mL, 2.5 ng/mL-50 ng/mL, 2.5 ng/mL-100 ng/mL, 2.5 ng/mL-200 ng/mL, 5 ng/mL-10 ng/mL, 5 ng/mL-15 ng/mL, 5 ng/mL-20 ng/mL, 5 ng/mL-30 ng/mL, 5 ng/mL-50 ng/mL, 5 ng/mL-100 ng/mL, 5 ng/mL-200 ng/mL, 10 ng/mL-15 ng/mL, 10 ng/mL-20 ng/mL, 10 ng/mL-30 ng/mL, 10 ng/mL-50 ng/mL, 10 ng/mL-100 ng/mL, 10 ng/mL-200 ng/mL, each inclusive. In some embodiments, the concentration of GM-CSF in the media is at least about 0.5 ng/mL, 2.5 ng/mL, 5 ng/mL, 10 ng/mL, 15 ng/mL, 20 ng/mL, 30 ng/mL, 50 ng/mL, 100 ng/mL, or 200 ng/mL. In some embodiments, the concentration of GM-CSF in the media is about 50 ng/mL. In some embodiments, the concentration of GM-CSF is increased in the media to 100 ng/mL.

In some embodiments, the concentration of the PI3K inhibitor in the media is from about 0.5 μM-1 μM, 0.5 μM-5 μM, 0.5 μM-10 μM, 0.5 μM-15 μM, 0.5 μM-20 μM, 0.5 μM-30 μM, 0.5 μM-50 μM, 1 μM-5 μM, 1 μM-10 μM, 1 μM-15 μM, 1 μM-20 μM, 1 μM-30 μM, 1 μM-50 μM, 5 μM-10 μM, 5 μM-15 μM, 5 μM-20 μM, 5 μM-30 μM, 5 μM-50 μM, 10 μM-15 μM, 10 μM-20 μM, 10 μM-30 μM, 10 μM-50 μM, each inclusive. In some embodiments, the concentration of PI3K inhibitor in the media is at least about 0.5 μM, 1 μM, 5 μM, 10 μM, 15 μM, 20 μM, 30 μM, or 50 μM, In some embodiments, the concentration of PI3K inhibitor in the media is about 4 μM. In some embodiments, the PI3K inhibitor is LY294002.

In some embodiments, the concentration of the AHR antagonist in the media is from about 0.5 μM-1 μM, 0.5 μM-5 μM, 0.5 μM-10 μM, 0.5 μM-15 μM, 0.5 μM-20 μM, 0.5 μM-30 μM, 0.5 μM-50 μM, 1 μM-5 μM, 1 μM-10 μM, 1 μM-15 μM, 1 μM-20 μM, 1 μM-30 μM, 1 μM-50 μM, 5 μM-10 μM, 5 μM-15 μM, 5 μM-20 μM, 5 μM-30 μM, 5 μM-50 μM, 10 μM-15 μM, 10 μM-20 μM, 10 μM-30 μM, 10 μM-50 μM, each inclusive. In some embodiments, the concentration of AHR antagonist in the media is at least about 0.5 μM, 1 μM, 5 μM, 10 μM, 15 μM, 20 μM, 30 μM, or 50 μM, In some embodiments, the concentration of AHR antagonist in the media is about 1 μM. In some embodiments, the AHR antagonist is StemRegenin-1.

In some embodiments, the concentration of the IL-3 in the media is from about 0.5 ng/mL-2.5 ng/mL, 0.5 ng/mL-5 ng/mL, 0.5 ng/mL-10 ng/mL, 0.5 ng/mL-15 ng/mL, 0.5 ng/mL-20 ng/mL, 0.5 ng/mL-30 ng/mL, 0.5 ng/mL-50 ng/mL, 0.5 ng/mL-100 ng/mL, 0.5 ng/mL-200 ng/mL, 2.5 ng/mL-5 ng/mL, 2.5 ng/mL-10 ng/mL, 2.5 ng/mL-15 ng/mL, 2.5 ng/mL-20 ng/mL, 2.5 ng/mL-30 ng/mL, 2.5 ng/mL-50 ng/mL, 2.5 ng/mL-100 ng/mL, 2.5 ng/mL-200 ng/mL, 5 ng/mL-10 ng/mL, 5 ng/mL-15 ng/mL, 5 ng/mL-20 ng/mL, 5 ng/mL-30 ng/mL, 5 ng/mL-50 ng/mL, 5 ng/mL-100 ng/mL, 5 ng/mL-200 ng/mL, 10 ng/mL-15 ng/mL, 10 ng/mL-20 ng/mL, 10 ng/mL-30 ng/mL, 10 ng/mL-50 ng/mL, 10 ng/mL-100 ng/mL, 10 ng/mL-200 ng/mL, each inclusive. In some embodiments, the concentration of IL-3 in the media is at least about 0.5 ng/mL, 2.5 ng/mL, 5 ng/mL, 10 ng/mL, 15 ng/mL, 20 ng/mL, 30 ng/mL, 50 ng/mL, 100 ng/mL, or 200 ng/mL. In some embodiments, the concentration of IL-3 in the media is 20 ng/mL. In some embodiments, the concentration of IL-3 in the media is about 25 ng/mL. In some embodiments, the concentration of IL-3 is increased in the media to 40 ng/mL.

In some embodiments, the concentration of the SCF in the media is from about 0.5 ng/mL-2.5 ng/mL, 0.5 ng/mL-5 ng/mL, 0.5 ng/mL-10 ng/mL, 0.5 ng/mL-15 ng/mL, 0.5 ng/mL-20 ng/mL, 0.5 ng/mL-30 ng/mL, 0.5 ng/mL-50 ng/mL, 0.5 ng/mL-100 ng/mL, 2.5 ng/mL-5 ng/mL, 2.5 ng/mL-10 ng/mL, 2.5 ng/mL-15 ng/mL, 2.5 ng/mL-20 ng/mL, 2.5 ng/mL-30 ng/mL, 2.5 ng/mL-50 ng/mL, 2.5 ng/mL-100 ng/mL, 5 ng/mL-10 ng/mL, 5 ng/mL-15 ng/mL, 5 ng/mL-20 ng/mL, 5 ng/mL-30 ng/mL, 5 ng/mL-50 ng/mL, 5 ng/mL-100 ng/mL, 10 ng/mL-15 ng/mL, 10 ng/mL-20 ng/mL, 10 ng/mL-30 ng/mL, 10 ng/mL-50 ng/mL, 10 ng/mL-100 ng/mL, each inclusive. In some embodiments, the concentration of SCF in the media is at least about 0.5 ng/mL, 2.5 ng/mL, 5 ng/mL, 10 ng/mL, 15 ng/mL, 20 ng/mL, 30 ng/mL, 50 ng/mL, or 100 ng/mL, In some embodiments, the concentration of SCF in the media is about 20 ng/mL.

In some embodiments, the concentration of the UM729 in the media is from about 0.5 μM-1 μM, 0.5 μM-5 μM, 0.5 μM-10 μM, 0.5 μM-15 μM, 0.5 μM-20 μM, 0.5 μM-30 μM, 0.5 μM-50 μM, 1 μM-5 μM, 1 μM-10 μM, 1 μM-15 μM, 1 μM-20 μM, 1 μM-30 μM, 1 μM-50 μM, 5 μM-10 μM, 5 μM-15 μM, 5 μM-20 μM, 5 μM-30 μM, 5 μM-50 μM, 10 μM-15 μM, 10 μM-20 μM, 10 μM-30 μM, 10 μM-50 μM, each inclusive. In some embodiments, the concentration of UM729 in the media is at least about 0.5 μM, 1 μM, 5 μM, 10 μM, 15 μM, 20 μM, 30 μM, or 50 μM. In some embodiments, the concentration of UM729 in the media is about 1 μM.

In some embodiments, one or more of the above steps of producing myeloid cells can include addition of a non-physiological ligand of the synthetic cytokine receptor (e.g. rapamycin or analog) to the culture medium to induce differentiation.

In some embodiments, the non-physiological ligand is rapamycin or a rapamycin analog. In some embodiments, the rapamycin analog is rapalog. In some embodiments, the non-physiological ligand (e.g., rapamycin or a rapamycin analog) is added to the media at a concentration of 2.5 nM and 200 nM, 2.5 nM and 150 nM, 2.5 nM and 100 nM, 2.5 nM and 50 nM, 2.5 nM and 20 nM, 2.5 nM and 10 nM, In some embodiments, the non-physiological ligand (e.g., rapamycin or a rapamycin analog) is added to the media at a concentration of between 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM, 10 nM and 50 nM, 10 nM and 20 nM, 20 nM and 200 nM, 20 nM and 150 nM 20 nM and 100 nM, 20 nM and 50 nM, 50 nM and 200 nM, 50 nM and 150 nM, 50 nM and 100 nM, 100 nM and 200 nM, 100 nM and 150 nM and 150 nM and 200 nM.

In some embodiments, the non-physiological ligand (e.g., rapamycin or a rapamycin analog) is added to the media at a concentration of at or about 10 nM. In some embodiments, the non-physiological ligand (e.g., rapamycin or a rapamycin analog)is added to the media at a concentration of at or about 100 nM. In some embodiments, rapamycin is added to the media at a concentration of at or about 100 nM. In some embodiments, rapalog is added to the media at a concentration of at or about 100 nM.

In some embodiments, it is surprisingly found that low concentrations of the non-physiological ligand (e.g., rapamycin or a rapamycin analog) is able to support differentiation and/or expansion. In some embodiments, the non-physiological ligand (e.g., rapamycin or a rapamycin analog) is added to the media at a concentration at or less than 10 nM. In some embodiments, the non-physiological ligand (e.g., rapamycin or a rapamycin analog) is added to the media at a concentration from 2.5 nM to 10 nM, such as 3 nM to 7 nM. In some embodiments, the non-physiological ligand (e.g., rapamycin or a rapamycin analog) is added to the media at a concentration of at or about 3 nM, at or about 4 nM, at or about 5 nM, at or about 6 nM, at or about 7 nM, at or about 8 nM, at or about 9 nM, or at or about 10 nM, or any value between any of the foregoing. In some embodiments, the non-physiological ligand (e.g., rapamycin or a rapamycin analog) is added to the media at a concentration of at or about 3.1 nM. In some embodiments, the non-physiological ligand (e.g., rapamycin or a rapamycin analog) is added to the media at a concentration of at or about 10 nM. In some embodiments, rapamycin is added to the media at a concentration of at or about 6.2 nM. In some embodiments, rapamycin is added to the media at a concentration of at or about 3.1 nM. In some embodiments, rapamycin is added to the media at a concentration of at or about 6.2 nM. In some embodiments, rapalog is added to the media at a concentration of at or about 3.1 nM. In some embodiments, rapalog is added to the media at a concentration of at or about 6.2 nM.

In some embodiments, the culturing of the cell populations can be transferred to a suitable vessel to promote cellular aggregation. In some embodiments, pluripotent aggregates may be formed in a bioreactor by culturing the engineered iPSCs in suspension in the bioreactor. A conventional strategy utilizes the formation of embryoid bodies as a common and critical intermediate to initiate the lineage-specific differentiation. Embryoid bodies are aggregates of stem cells that are induced to differentiate by changes in environmental stimuli (e.g., exposure and/or removal of specific molecular/chemical factors; and/or exposure/interaction with three-dimensional structures). Formation of embryoid bodies induces the cells to differentiate cells to a mesoderm specification.

Hematopoietic cells may be generated from embryoid bodies derived from pluripotent cells. Pluripotent cells may be allowed to form embryoid bodies or aggregates as a part of the differentiation process. The formation of “embryoid bodies” (EBs), or clusters of growing cells, in order to induce differentiation generally involves in vitro aggregation of human pluripotent stem cells into EBs and allows for the spontaneous and random differentiation of human pluripotent stem cells into multiple tissue types that represent endoderm, ectoderm, and mesoderm origins. Without specific culture conditions, it may take about two weeks for EBs to differentiate toward any of the three germ layers, and the differentiation process is performed in a random pattern.

In some embodiments, the culturing of the cell populations can be transferred to a suitable vessel to promote cellular aggregation. The vessel may be a 2D or a 3D vessel. Examples of suitable 2D vessels for culturing source cells include any petri dish or culture dish regularly used in the laboratory for culturing cells.

In some embodiments, the vessel is suitable for 3-Dimensional (3D) culture. Without being bound to a particular theory or mechanism, it is believed that 3D culture may be more effective for providing a scaffold for cell differentiation than two dimensional (2D) culture. Suitable 3D culture systems may include, for example, a hanging drop 3D culture, e.g., hanging drop plates, a 3D microwell culture, e.g., ultra-low attachment multiwell plates, a 3D culture on a hydrophobic surface, a rotational culture, a static 3D suspension culture, or a bioreactor. Hanging drop plates are commercially available such as, for example, the PERFECTA3D hanging drop plate, available from Biospherix, Parish, N.Y. Ultra-low attachment multiwell plates (in some cases also referred to as non-adherent culture vessels) are also commercially available such as, for example, AGGREWELL™ ultra-low attachment, multi-well plate, available from Stemcell Technologies, Vancouver, Canada.

In some embodiments, the vessel is not treated to promote cell adhesion and growth. In some embodiments, the vessel is a standard tissue culture plate but is not treated to promote cell adhesion and growth. In some embodiments, the cells do not adhere or substantially adhere during the culturing. In some embodiments, the culturing is in suspension.

In some embodiments, the vessels are multi-well plates. The multi-well plates may be 96-well plates, 24-well plates or 6-well plates.

In some embodiments, the vessel is a bioreactor. In some embodiments, bioreactors are used for the process of myeloid cell generation and proliferation after the development of EBs. Bioreactors allow for the optimization of cell culture conditions to achieve optimal hydrogen production yields and process robustness. Environmental conditions that can be adjusted or monitored in a bioreactor include gas composition (e.g., air, oxygen, nitrogen, carbon dioxide), gas flow rates, temperature, pH, dissolved oxygen levels, and the agitation speed/circulation rate within the cell culture. Any type of bioreactor known in the art may be used for cell culture for the differentiation and expansion of cultured myeloid cells, including, but not limited to, a stirred-tank bioreactor, a pneumatic bioreactor (e.g., a bubble column or airlift bioreactor), a membrane bioreactor, a hollow-fiber bioreactor, a wave bioreactor, a vertical wheel bioreactor, a gas permeable rapid expansion (G-Rex) bioreactor, or a disposable bioreactor. In some embodiments, the bioreactor is a gas permeable rapid expansion (G-Rex) bioreactor. In some embodiments, the bioreactor is a vertical wheel bioreactor. In some embodiments, the bioreactor is a stirred-tank bioreactor. In some embodiments, the stirred-tank bioreactor is the Sartorius Ambr250 stirred tank bioreactor.

The differentiation and expansion of the iMyeloid cells may be scaled to any desired volume to suit various purposes. For example, for high to medium throughput screening of various culture conditions, the process may be scaled to take place in a microbioreactor (e.g., about 15 mL to about 500 mL) or a benchtop scale bioreactor e.g., ranging from about 0.5 L to about 15 L). Alternatively, the process may be scaled up to a pilot scale bioreactor (e.g., ranging from about 15 L to about 15,000 L), or a manufacturing scale bioreactor (e.g., about 15,000 L to about 75,000 L or greater). In some embodiments, the bioreactor is a vertical wheel bioreactor with a volume of about 10 mL to about 1000 mL. In some embodiments, the bioreactor used to differentiate iPSC cells to HPs is a vertical wheel bioreactor with a volume of about 100 mL. In some embodiments, the bioreactor used to differentiate HPs to iMACs is a vertical wheel bioreactor with a volume of about 500 mL.

Methods of dissociating the cells are known to a skilled artisan. Any of a variety of methods can be used. In some embodiments, dissociation is with Gentle Cell Dissociation Reagent (GDCR; Stem Cell Technologies). In some embodiments, dissociation is with EDTA.

In some embodiments, one or more of the above steps of producing HP cells can include addition of a non-physiological ligand of the synthetic cytokine receptor (e.g. rapamycin or analog) to the culture medium to induce differentiation. In some embodiments, the non-physiological ligand is rapamycin or a rapamycin analog. In some embodiments, the rapamycin analog is rapalog. In some embodiments, the non-physiological ligand (e.g., rapamycin or a rapamycin analog) is added to the media at a concentration of 2.5 nM and 200 nM, 2.5 nM and 150 nM, 2.5 nM and 100 nM, 2.5 nM and 50 nM, 2.5 nM and 20 nM, 2.5 nM and 10 nM, In some embodiments, the non-physiological ligand (e.g., rapamycin or a rapamycin analog) is added to the media at a concentration of between 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM, 10 nM and 50 nM, 10 nM and 20 nM, 20 nM and 200 nM, 20 nM and 150 nM 20 nM and 100 nM, 20 nM and 50 nM, 50 nM and 200 nM, 50 nM and 150 nM, 50 nM and 100 nM, 100 nM and 200 nM, 100 nM and 150 nM and 150 nM and 200 nM.

In some embodiments, the non-physiological ligand (e.g., rapamycin or a rapamycin analog)is added to the media at a concentration of at or about 10 nM. In some embodiments, the non-physiological ligand (e.g., rapamycin or a rapamycin analog) is added to the media at a concentration of at or about 100 nM. In some embodiments, rapamycin is added to the media at a concentration of at or about 100 nM. In some embodiments, rapalog is added to the media at a concentration of at or about 100 nM.

In some embodiments, it is surprisingly found that low concentrations of the non-physiological ligand (e.g., rapamycin or a rapamycin analog) is able to support differentiation and/or expansion. In some embodiments, the non-physiological ligand (e.g., rapamycin or a rapamycin analog) is added to the media at a concentration at or less than 10 nM. In some embodiments, the non-physiological ligand (e.g., rapamycin or a rapamycin analog) is added to the media at a concentration from 2.5 nM to 10 nM, such as 3 nM to 7 nM. In some embodiments, the non-physiological ligand (e.g., rapamycin or a rapamycin analog) is added to the media at a concentration of at or about 3 nM, at or about 4 nM, at or about 5 nM, at or about 6 nM, at or about 7 nM, at or about 8 nM, at or about 9 nM, or at or about 10 nM, or any value between any of the foregoing. In some embodiments, the non-physiological ligand (e.g., rapamycin or a rapamycin analog) is added to the media at a concentration of at or about 3.1 nM. In some embodiments, the non-physiological ligand (e.g., rapamycin or a rapamycin analog) is added to the media at a concentration of at or about 10 nM. In some embodiments, the non-physiological ligand (e.g., rapamycin or a rapamycin analog) is added to the media at a concentration of at or about 6.2 nM. In some embodiments, rapamycin is added to the media at a concentration of at or about 3.1 nM. In some embodiments, rapamycin is added to the media at a concentration of at or about 6.2 nM. In some embodiments, rapalog is added to the media at a concentration of at or about 3.1 nM. In some embodiments, rapalog is added to the media at a concentration of at or about 6.2 nM.

In some embodiments, the provided method includes culturing engineered stem cells, e.g. engineered with a synthetic cytokine receptor and/or a CAR, with the non-physiological ligand for a first period of time sufficient to generate HPs, and contacting the HPs with a differentiation media for a second period of time sufficient to generate iMACs.

In some embodiments, conditions in addition to or other than activation with the synthetic cytokine receptor can be used in methods to differentiate the engineered stem cells to macrophages.

In some embodiments, the provided stem cells, such as iPSCs, engineered with a synthetic cytokine receptor may instead or alternatively be differentiated via any other method known to differentiate macrophages. In some embodiments, one or more growth factor or cytokine customarily used in connection with differentiation and/or activation of macrophages may be used in the provided methods in addition to the non-physiological ligand engagement of the synthetic cytokine receptor.

Various differentiation protocols for macrophages are known in the art.

In some embodiments, the stem cells are adapted for feeder-free culture. As used herein, a “feeder-free” (FF) environment refers to an environment such as a culture condition, cell culture or culture media which is essentially free of feeder or stromal cells, and/or which has not been pre-conditioned by the cultivation of feeder cells. “Pre-conditioned” medium refers to a medium harvested after feeder cells have been cultivated within the medium for a period of time, such as for at least one day. Pre-conditioned medium contains many mediator substances, including growth factors and cytokines secreted by the feeder cells cultivated in the medium.

E. Expansion and Activation of iMC (e.g, iMAC Cells)

In some embodiments, after induced myeloid (iMC) cells (e.g., iMac cells) have been transduced with a CAR and/or a synthetic cytokine receptor, the cells are cultured under conditions that promote the activation and expansion of the cells.

Culture conditions may be such that the cells can be administered to a patient without concern for reactivity against components of the culture medium. For example, the culture conditions may omit bovine serum products, such as bovine serum albumin. In one illustrative aspect, the activation can be achieved by introducing known activators into the culture medium. In one aspect, the population of cells can be cultured under conditions promoting activation for about 1 to about 4 days. In one embodiment, the appropriate level of activation can be determined by cell size, proliferation rate, or activation markers determined by flow cytometry. In some embodiments, any of the culturing methods disclosed herein may be used to promote activation of the myeloid cells.

In some embodiments, provided herein is a method of making and/or expanding a population of engineered cells comprising a synthetic cytokine receptor for a non-physiological ligand. In some embodiments, the method comprises: providing engineered cells comprising a synthetic cytokine receptor, optionally by introducing into source cells a polynucleotide encoding a synthetic cytokine receptor, and incubating the engineered cells in media comprising a non-physiological ligand.

In this embodiment, the cytokine receptor comprises a synthetic gamma chain polypeptide comprising a first dimerization domain, a first transmembrane domain, and an interleukin-2 receptor subunit gamma (IL-2RG) intracellular domain, and a synthetic beta chain polypeptide comprising a second dimerization domain, a second transmembrane domain, and an intracellular domain selected from an interleukin-2 receptor subunit beta (IL-2RB) intracellular domain; wherein the non-physiological ligand activates the synthetic cytokine receptor in the engineered cells to induce expansion and/or activation of the engineered cells.

In some embodiments, myeloid cells are engineered to express a synthetic cytokine receptor, such as RACR, and activated with a rapalog in the medium without recombinant cytokines in the medium.

Generation of myeloid cells (e.g., macrophages) from iPSC using conventional approaches is generally inefficient and is a current bottleneck in the manufacture of effector immune cell therapies. For example, in some cases conventional approaches achieve only ~1× yield of macrophage from iPSC (1 macrophage for every 1 iPSC). In some embodiments, the provided methods can result in increased yields greater than at or about 10-fold (10×), 20-fold, 40-fold, 50-fold, 60-fold, 70-fold, 80-fold, 90-fold, 100-fold, 150-fold, 200-fold, 250-fold, 300-fold of macrophage from iPSC. In some embodiments, the provided methods can result in increased yields greater than at or about 350-fold, 400-fold, 450-fold, 500-fold, 550-fold, 600-fold, 650-fold, 1,000-fold, 1,500-fold, 2,000-fold, 2,500-fold, 3,000-fold, 3,500-fold, 4,000-fold, 4,500-fold, 5,000-fold, 5,500-fold, 6,000-fold, 6,500-fold, 7,000-fold, 7,500-fold, 8,000-fold, 8,500-fold, 9,000-fold, 9,500-fold, 10,000-fold of macrophage from iPSC. In some embodiments, the provided methods can result in increased yields greater than at or about 7,000-fold of myeloid cells from iPSC. This result represents a substantial improvement over other approaches to differentiation iPSC to myeloid cell differentiation and expansion. Furthermore, generation of iMAC from HP using the provided embodiments result in iMAC that are highly potent as demonstrated by killing and/or phagocytosis of tumor target cells.

In some embodiments, the expanding step is performed in a feeder-free cell culture.

In some embodiments, provided is a method of administering an effective amount of non-physiological ligand to the subject, wherein the non-physiological ligand causes the iPSC cells to differentiate to myeloid cells according to any of the foregoing embodiments.

In some embodiments, provided are methods of differentiating and expanding iMAC cells by culturing in a suitable vessel. The vessel may be a 2D or a 3D vessel. Examples of suitable 2D vessels for culturing source cells include any petri dish or culture dish regularly used in the laboratory for culturing cells. The culturing vessel may be coated with a suitable culturing medium, for example an extracellular medium for the attachment and/or differentiation of cultured cells. In some embodiments, the vessel is treated to promote cell adhesion and growth. An example of a suitable medium for use in the inventive method is MATRIGEL™ membrane matrix (BD Biosciences, Franklin Lakes, N.J.).

In some embodiments, the vessel is suitable for 3-Dimensional (3D) culture. Without being bound to a particular theory or mechanism, it is believed that 3D culture may be more effective for providing a scaffold for cell differentiation than two dimensional (2D) culture. Suitable 3D culture systems may include, for example, a hanging drop 3D culture, e.g., hanging drop plates, a 3D microwell culture, e.g., ultra-low attachment multiwell plates, a 3D culture on a hydrophobic surface, a rotational culture, a static 3D suspension culture, or a bioreactor. Hanging drop plates are commercially available such as, for example, the PERFECTA3D hanging drop plate, available from Biospherix, Parish, N.Y. Ultra-low attachment multiwell plates (in some cases also referred to as non-adherent culture vessels) are also commercially available such as, for example, AGGREWELL™ ultra-low attachment, multi-well plate, available from Stemcell Technologies, Vancouver, Canada.

In some embodiments, the vessel is not treated to promote cell adhesion and growth. In some embodiments, the vessel is a standard tissue culture plate but is not treated to promote cell adhesion and growth. In some embodiments, the cells do not adhere or substantially adhere during the culturing. In some embodiments, the culturing is in suspension.

In some embodiments, the vessels are multi-well plates. The multi-well plates may be 96-well plates, 24-well plates or 6-well plates.

In some embodiments, the vessel is a bioreactor. In some embodiments, bioreactors are used for the process of iMC generation and proliferation after the development of EBs. Bioreactors allow for the optimization of cell culture conditions to achieve optimal hydrogen production yields and process robustness. Environmental conditions that can be adjusted or monitored in a bioreactor include gas composition (e.g., air, oxygen, nitrogen, carbon dioxide), gas flow rates, temperature, pH, dissolved oxygen levels, and the agitation speed/circulation rate within the cell culture. Any type of bioreactor known in the art may be used for cell culture for the differentiation and expansion of cultured iMC cells, including, but not limited to, a stirred-tank bioreactor, a pneumatic bioreactor (e.g., a bubble column or airlift bioreactor), a membrane bioreactor, a hollow-fiber bioreactor, a wave bioreactor, a vertical wheel bioreactor, a gas permeable rapid expansion (G-Rex) bioreactor, or a disposable bioreactor. In some embodiments, the bioreactor is a gas permeable rapid expansion (G-Rex) bioreactor. In some embodiments, the bioreactor is a vertical wheel bioreactor. In some embodiments, the bioreactor is a stirred-tank bioreactor. In some embodiments, the vertical wheel bioreactor is the PBS bioreactor. In some embodiments, the stirred-tank bioreactor is the Sartorius Ambr250 stirred tank bioreactor.

The expansion of the iMC cells may be scaled to any desired volume to suit various purposes. For example, for high to medium throughput screening of various culture conditions, the process may be scaled to take place in a microbioreactor (e.g., about 15 mL to about 500 mL) or a benchtop scale bioreactor e.g., ranging from about 0.5 L to about 15 L). Alternatively, the process may be scaled up to a pilot scale bioreactor (e.g., ranging from about 15 L to about 15,000 L), or a manufacturing scale bioreactor (e.g., about 15,000 L to about 75,000 L or greater). In some embodiments, the bioreactor is a vertical wheel bioreactor with a volume of about 10 mL to about 1000 mL. In some embodiments, the bioreactor is a vertical wheel bioreactor with a volume of about 100 mL. In some embodiments, the bioreactor is a vertical wheel bioreactor with a volume of about 500 mL.

In some embodiments, one or more vessels may be used for iMC expansion. In some embodiments, the cells may be cultured in the vessel from days 0-3, days 0-10, days 0-15, days 0-20, days 0-25, days 0-30, days 0-35, days 0-40, days 0-50, days 0-60, days 0-100, days 3-10, days 3-15, days 3-20, days 3-25, days 3-30, days 3-35, days 3-40, days 3-50, days 3-60, days 3-100, days 10-20, days 10-25, days 10-30, days 10-35, days 10-40, days 10-50, days 10-60, days 10-100, days 15-20, days 15-25, days 15-30, days 15-35, days 15-40, days 15-50, days 15-60, days 15-100, days 20-25, days 20-30, days 20-35, days 20-40, days 20-50, days 20-60, days 20-100, days 25-30, days 25-35, days 25-40, days 25-50, days 25-60, days 25-100, days 30-35, days 30-40, days 30-50, days 30-60, days 30-100, days 35-40, days 35-50, days 35-60, days 35-100, days 40-50, days 40-60, days 40-100, each inclusive. In some embodiments, the cells may be cultured in the vessel from days 0-35. In some embodiments, the cells may be cultured in the vessel from days 3-35. In some embodiments, the vessel is a bioreactor. In some embodiments, one or more bioreactors may be used for iAC expansion. In some embodiments, the one or more bioreactors are different types of bioreactors. In some embodiments, the one or more bioreactors are different sizes of bioreactors.

F. Myeloid Cells Differentiated from Engineered Stem Cells (e.g., iMAC Cells)

In some embodiments, macrophages may be derived from iPSCs by sequentially differentiating the iPSCs into hematopoietic progenitor cells (HPCs); the HPCs into macrophages—termed “iMAC” cells. In a variation, macrophages may be derived from HPCs by sequentially differentiating the HPCs into myeloid progenitor cells; and then the myeloid progenitor cells into iMAC cells. In a further variation, macrophages may be derived by differentiating myeloid progenitor cells into iMAC cells. Engineering of the cells to express the synthetic cytokine receptor may be performed at the iPSC, HPC, myeloid progenitor cells, or iMAC cell step of the differentiation process.

In some embodiments, the hematopoietic stem cells are characterized as being CD34+ and/or CD45+; common myeloid progenitor cells, characterized as being CD34+, CD90−, and CD45RA+, and/or macrophage cells, characterized as being CD45+CD14+ and optionally express CD11b, CD68, CD163, F4/80, CD16, CD54, CD49e, CD38, Egr2, CD71, TLR2, and/or TLR4. In some embodiments, myeloid progenitor cells are characterized as being CD34+, CD90−, and CD45RA+. In some embodiments, myeloid progenitor cells further express CD123.

In some embodiments, the myeloid progenitor cells comprise one or more of: Common Myeloid Progenitor Cells (“CMPs”) (CD34+, CD90−, CD123+, CD45RA−), Megakaryocyte/Erythroid Progenitor Cells (“MEPs”) (CD34+, CD90−, CD123−, CD45RA−) and Granulocyte/Monocyte Progenitor Cells (“GMPs”) (CD34+, CD90−, CD123+, CD45RA+). In another embodiment the cell population is substantially free of cells of lymphoid lineage, e.g., Common Lymphoid Progenitor Cells (“CLPs”) (CD34+, CD7+, CD10+), T cells (CD2+, CD3+), and B cells (CD19+, CD20+, CD33−), e.g., less than 5%. In another embodiment the cell population comprises Multi-Potent Progenitor Cells (“MPPs”) (CD34+, CD90+). In another embodiment the cell population comprises Granulocyte-macrophage progenitors that can differentiate into monocytes (CD34+, CD14+), which, in turn, differentiate into macrophages (CD34−, CD11b+, CD68+). They also can differentiate into granulocytes (CD34+, CD15+), which, in turn, differentiate into neutrophils (CD34−, CD15+, CD66b+, CD16+), basophils (CD34−, CD15+, CD123+), and eosinophils (CD34−, CD15+, CD66b+, CD11b+).

In some embodiments, the myeloid cells derived from the genetically engineered myeloid progenitor cell is a macrophage, a neutrophil, a megakaryocyte, a monocyte, a basophil, an eosinophil, and/or an erythrocyte cell. In some embodiments, the myeloid cell is a macrophage. In some embodiments, the myeloid cell derived from the genetically engineered myeloid progenitor cell is a macrophage. In some embodiments, the macrophages are CD34−, CD11b+, CD68+. In some embodiments, the myeloid cell derived from the genetically engineered myeloid progenitor cell is a granulocyte. In some embodiments, the granulocytes are CD34+, CD15+. In some embodiments, the myeloid cell derived from the genetically engineered myeloid progenitor cell is a neutrophil. In some embodiments, the neutrophils are CD34−, CD15+, CD66b+, CD16+. In some embodiments, the myeloid cell derived from the genetically engineered myeloid progenitor cell is a basophil. In some embodiments, the basophils are CD34−, CD15+, CD123+. In some embodiments, the myeloid cell derived from the genetically engineered myeloid progenitor cell is a eosinophil. In some embodiments, the eosinophils are CD34−, CD15+, CD66b+, CD11b+.

In some embodiments, a cell is “derived from an induced pluripotent stem cell” or is an “iPSC-derived” cell, if it is differentiated beyond pluripotent stem cell stage and comprises genetic modifications consistent with those used to produce an induced pluripotent stem cell, e.g., presence of recombinant DNA comprising Oct4, Sox2, Klf4, and/or cMyc genes. Recombinant DNA is DNA in which two DNA sequences, not normally connected in nature, have been combined. For example, connection of a heterologous promoter to a gene to form an expression construct represents a recombinant DNA molecule.

Myeloid progenitor cells, as well as terminally differentiated myeloid cells that develop from them, can be produced by a process that involves providing mature cells, reprogramming the mature cells to produce induced pluripotent stem cells, optionally rendering induced pluripotent stem cells hypoimmunogenic (e.g., by knocking out HLA Class I/II genes and up-regulating CD47), differentiating the pluripotent stem cells into hematopoietic stem cells, differentiating hematopoietic stem cells into myeloid progenitor cells and differentiating myeloid progenitor cells into mature cells of the myeloid line, such as granulocytes (precursors to neutrophils, eosinophils, basophils), monocytes (precursors to macrophages), megakaryocytes (precursors to platelets) and erythroid progenitors (precursors to erythrocytes). At each stage, the cells also may be hypoimmunogenic.

In some embodiments, the iMAC cells are characterized by being CD45+CD14^{high}

In some embodiments, the macrophages express one or more of CD14, CD11b, CD68, CD163, F4/80, CD16, CD54, CD49e, CD38, Egr2, CD71, TLR2, TLR4. In some embodiments, the population of engineered cells is 40% to 60% CD14+, 50% to 70% CD14+, 60% to 80% CD14+, 70% to 90% CD14+, 80% to 100% CD14+, or any percentage within a range defined by any two aforementioned values.

In some embodiments, the population of engineered macrophages is CD14lo. In some embodiments, the population of engineered macrophages is CD14high.

In some embodiments, the population of engineered macrophages is 40% to 60% CD45+, 50% to 70% CD45+, 60% to 80% CD45+, 70% to 90% CD45+, 80% to 100% CD45+, or any percentage within a range defined by any two aforementioned values.

In some embodiments, the population of engineered macrophages is 60% to 80% CD45+CD56+, 65% to 85% CD45+CD56+, 70% to 90% CD45+CD56+, 75% to 95% CD45+CD56+, 80% to 99% CD45+CD14+, or any percentage within a range defined by any two aforementioned values.

In some embodiments, the population of engineered macrophages is at least 40% CD45+, at least 50% CD45+, at least 60% CD45+, at least 70% CD45+, at least 80% CD45+, at least 90% CD45+, or 100% CD45+.

In some embodiments, the iMACs express a CAR in the engineered stem cell, as described in Section IID.

Tumor cells are a highly sensitive target for an in vitro macrophage cytotoxic activity assay. In the assay, macrophages are co-incubated at different ratios with target tumor cells known to be sensitive to macrophage-mediated tumor killing. The target cells are pre-labeled with a fluorescent dye to allow their discrimination from the effector cells (macrophages). After the incubation period, killed target cells are identified by a nucleic acid stain, which specifically permeates dead cells. % Dead is calculated by comparing the total number of viable cells in each experimental assay well to a non-effector control well. As used herein, the term “activity” refers to a measurement of the macrophages' cytotoxic capacity against target cells.

In some embodiments, the engineered macrophages may be fresh or frozen. In some embodiments, the engineered macrophages are fresh. In some embodiments, the engineered macrophages are frozen. In some embodiments, when the frozen macrophages are thawed, they retain viability and cytotoxic function compared to fresh macrophages. In some embodiments, frozen/thawed macrophages perform as well as controlling tumors as fresh macrophages.

In some embodiments, the cells can be frozen by methods of cryopreservation. In some embodiments, iMCs are subjected to cryopreservation after their differentiation in accord with provided methods. In some embodiments, iMCs are subjected to cryopreservation after their engineering. In some embodiments, engineered iMCs produced according to provided methods are subjected to cryopreservation. In some embodiments, the method includes cryopreserving the cells in the presence of a cryoprotectant, thereby producing a cryopreserved composition. Any of a variety of known freezing solutions and parameters in some aspects may be used. In some embodiments, the cryoprotectant is DMSO. In some embodiments, the cells are frozen, e.g., cryopreserved, in a solution with a final concentration of between 1% and 15%, between 6% and 12%, between 5% and 10%, or between 6% and 8% DMSO. In some embodiments, the cryopreservation medium is between at or about 5% and at or about 10% DMSO (v/v). In some embodiments, the cryopreservation media contains one or more additional excipients, such as plasmalyte A or human serum albumin (HSA). In some embodiments, the solution for cryopreservation may also include human serum albumin (HSA). In particular embodiments, the cells are frozen, e.g., cryopreserved, in a solution with a final concentration of between 0.1% and 5%, between 0.25% and 4%, between 0.5% and 2%, or between 1% and 2% HSA. In some embodiments, the cryopreservation medium contains a commercially available cryopreservation solution (CryoStor™ CS10 or CS5). CryoStor™ CS10 is a cryopreservation medium containing 10% dimethyl sulfoxide (DMSO). CryoStor™ CS5 is a cryopreservation medium containing 5% dimethyl sulfoxide (DMSO). In some embodiments, the cells are generally then frozen to or to about −80 °C at a rate of or of about 1° per minute and stored in the vapor phase of a liquid nitrogen storage tank. In some aspects, the engineered iMCs are thawed prior to their use, such as in connection with methods of treatment described herein. In some embodiments, after thawing the cells the method includes washing the cryopreserved composition under conditions to reduce or remove the cyroprotectant.

### III. GENE EDITING AND ENGINEERING

In some embodiments the pluripotent stems cells (e.g. iPSCs) or iMCs may be modified by gene editing. In some embodiments the pluripotent stems cells (e.g. iPSCs) or iMACs may be modified by genetic engineering, such as by introducing an exogenous nucleic acid encoding a transgene, such as a chimeric antigen receptor (CAR). In some embodiments, the gene edited iPSCs as described may be used as source cells for differentiation into iMCs (e.g., iMACs).

Genome editing generally refers to the process of editing or changing the nucleotide sequence of a genome, preferably in a precise, desirable and/or pre-determined manner. Examples of compositions, systems, and methods of genome editing described herein use site-directed nucleases to cut or cleave DNA at precise target locations in the genome, thereby creating a double-strand break (DSB) in the DNA. Such breaks can be repaired by endogenous DNA repair pathways, such as homology directed repair (HDR) and/or non-homologous end-joining (NHEJ) repair (see e.g., Cox et al., (2015) Nature Medicine 21 (2):121-31).

In some embodiments, the cells described herein (e.g., stem cells, macrophages) are genetically modified. In some embodiments, the modification involves knocking out one or more endogenous genes using a DNA-targeted protein and a nuclease or an RNA-guided nuclease and/or knocking in one or more exogenous genes of interest. In some embodiments, a gene of interest is knocked into a particular locus of interest. In some embodiments, the gene of interest is a synthetic cytokine receptor complex. In some embodiments, the synthetic cytokine receptor complex is activated by rapamycin. In some embodiments, the synthetic cytokine receptor complex is a rapamycin activated cytokine receptor (RACR). In some embodiments, a RACR is knocked into a locus of interest. In some embodiments, the gene of interest is a chimeric antigen receptor.

In some embodiments, the modification comprises contacting a cell with a DNA-targeted protein and a nuclease or an RNA-guided nuclease. In some embodiments, a DNA-targeted protein and a nuclease or an RNA-guided nuclease includes zinc finger protein (ZFP), a clustered regularly interspaced short palindromic nucleic acid (CRISPR), or a TAL-effector nuclease (TALEN). In some embodiments, CRISPR-Cas9 is used. In some embodiments, CRISPR-Mad7 is used.

Rejection of cellular therapeutics (e.g., CAR T cells) is due at least to mismatches of human leukocyte antigen (HLA) between donor and recipient. One solution recently identified is disrupting expression of genes involved in this rejection, such as beta-2-microglobulin (B2M). Accordingly, in some embodiments, the cells described herein (e.g., iPSCs, macrophages), are genetically engineered to knockout a B2M locus.

In some embodiments, the cells described herein are genetically engineered to be rapamycin resistant. Rapamycin is small molecule drug that inhibits the mTOR pathway, which is a pathway that is essential for cell growth and expansion. Thus, contacting a cell with rapamycin could, in some cases, inhibit or reduce cell growth and expansion. In some embodiments, to eliminate or reduce rapamycin-mediated growth inhibition of a source cell or macrophage using the provided methods, the provided cells are disrupted in an endogenous gene involved in rapamycin function, thereby rendering such cells “rapamycin resistant.” It is understood that reference to a “rapamycin resistant” cell refers to the ability of a cell's endogenous mTOR pathway not to be affected or impacted by the presence of rapamycin or a rapamycin analog. However, it is further understood that a “rapamycin resistant” cell may nevertheless be responsive to rapamycin via a pathway that does not involve mTOR, such as due to activation of a synthetic RACR as described herein.

In some embodiments, the cells are genetically engineered to disrupt a gene associated with rapamycin recognition. In some embodiments, the cells are genetically engineered to disrupt the mTOR gene. In some embodiments, the mTOR gene is FKBP-12 (also known as FKBP-1A, FKBP1, FKBP12, PKC12, PKCI2, PPIASE). FKBP12 is an essential binder of rapamycin and required for its function. In some embodiments, the cells are genetically engineered to disrupt the FKBP12 gene. In some embodiments, the cells are genetically engineered to knockout the FKB12 gene to induce rapamycin resistance. In some embodiments, the disruption of the endogenous FKBP12 gene of the source stem cell (e.g. iPSC) is through genetic knock out with CRISPR-Cas system. In a normal cell without a genetic disruption of FKBP12, FKBP12 is the primary binder of rapamycin, and the FKBP12-rapamycin complex then binds to the FRB subunit of mTOR and blocks mTOR signaling. By disrupting expression of the FKBP12 gene such as by FKBP12 knockout, results herein demonstrate successful rapamycin suppression activity because rapamycin has no function without first complexing with FKBP1A. Thus, genetic disruption of FKBP12, such as by gene knock out, renders the stem cells (e.g. iPSCs) highly resistant to rapamycin-mediated mTOR inhibition, enabling robust growth of the stem cells (e.g. iPSC) even in the presence of high doses of rapamycin. In some embodiments, the ability to render cells resistant to rapamycin growth suppression permits engagement of the RACR by rapamycin during cell differentiation without deleterious effects. Further, knock out of FKBP12 avoids competition of FKBP12 with the RACR for binding to rapamycin. Thus, in some embodiments, the ability to render cells resistant to rapamycin growth by FKBP12 knock out also permits activation of RACR-containing cells in vivo and suppresses potential allogeneic anti-graft responses through mTOR suppression of the host immune system. In some embodiments, the gRNA used to knock out FKBP12 was a pool of gRNA molecules. In some embodiments, the gRNAs comprise one or more gRNA selected from a gRNA comprising the sequence set forth in SEQ ID NO: 19, SEQ ID NO:20 or SEQ ID NO:21. In some embodiments, the one or more gRNA is a pool of gRNA comprising 2 or 3 gRNA.

In some embodiments, the cells described herein are genetically engineered to comprise a nucleotide sequence encoding a synthetic cytokine receptor in an endogenous gene. In some embodiments, the synthetic cytokine receptor is engineered into a gene such that expression of the endogenous gene is not disrupted. In some embodiments, the cells described herein are genetically engineered to comprise a nucleotide sequence encoding a synthetic cytokine receptor complex in a disrupted gene, such as a gene that has been inactivated or knocked-out in the cell.

In some embodiments, the cells described herein are genetically engineered to comprise a nucleotide sequence encoding a synthetic cytokine receptor in a target endogenous gene. In some embodiments, the synthetic cytokine receptor is engineered into a safe-harbor locus. In some embodiments, the target endogenous gene is a housekeeping gene. In some embodiments, the housekeeping gene is eukaryotic translation elongation factor 1 alpha (EEF1A), glylceraldehyde-3-phosphate dehydrogenase (GAPDH), ubiquitin C (UBC), or actin beta (ACTB). In some embodiments, the target endogenous gene is a B2M.

In some embodiments, the gene of interest inserted into an endogenous locus is a synthetic cytokine receptor complex. In some embodiments, the endogenous promoter of the particular locus is used.

In some embodiments, an exogenous promoter is operably connected to the gene encoding the synthetic cytokine receptor complex to drive expression. In some embodiments, the promoter is an EF1A promoter (also known as EEF1A promoter). In some embodiments, the promoter is an MND promoter. In some embodiments, additional promoter(s) may be included such that two or more promoters drive expression of the exogenous gene of interest. In some embodiments, the two or more promoters may be the same or different. In some embodiments, the promoter is a dual promoter in which the synthetic cytokine receptor is under the operable control of two promoters. In some embodiments, the dual promoter is a dual EF1α promoter.

For example, in some embodiments the cells comprise a disrupted B2M gene and a nucleotide sequence encoding the synthetic cytokine receptor in the disrupted B2M gene.

In some embodiments, the cells described herein (e.g., iPSCs, macrophages) comprise (i) a disrupted B2M locus, and (ii) a nucleotide sequence encoding a synthetic cytokine receptor complex (e.g., a RACR) under control of the endogenous B2M promoter and an EEF1A promoter.

In some embodiments, the cells described herein (e.g., iPSCs, macrophages) comprise (i) a disrupted B2M locus, and (ii) a nucleotide sequence encoding a synthetic cytokine receptor complex (e.g., a RACR) inserted into the endogenous B2M gene and under control of the endogenous B2M promoter and an EEF1A promoter.

In some embodiments, cells comprising (i) a disrupted B2M locus and (ii) a nucleotide sequence encoding a synthetic cytokine receptor complex (e.g., a RACR) are produced by any of the methods described below.

In some embodiments, the cells described herein (e.g., iPSCs, macrophages) further comprise (i) a disrupted AAVS1 locus, and (ii) a nucleotide sequence encoding CAR (e.g., a CD19-CAR) under control of the endogenous AAVS1 promoter.

### A. Systems for Genome Editing

In some embodiments, a system for editing a cell described herein comprises a site-directed nuclease, such as a CRISPR/Cas system and optionally a gRNA. In some embodiments, the system comprises an engineered nuclease. In some embodiments, the system comprises a site-directed nuclease. In some embodiments, the site-directed nuclease comprises a CRISPR/Cas nuclease system. In some embodiments, the Cas nuclease is Cas9. In some embodiments, the nuclease is Mad7. In some embodiments, the guide RNA comprising the CRISPR/Cas system is a single guide RNA (sgRNA).

Sequences herein sets forth exemplary gRNA targeting sequences. In some embodiments, the gRNA targeting sequence may contain one or more thymines in the complementary portion sequence substituted with uracil. It will be understood by one of ordinary skill in the art that uracil and thymine can both be represented by ‘t’, instead of ‘u’ for uracil and ‘t’ for thymine; in the context of a ribonucleic acid, it will be understood that ‘t’ is used to represent uracil unless otherwise indicated.

### 1. Nuclease

a. CRISPR/Cas Nuclease Systems

Naturally-occurring CRISPR/Cas systems are genetic defense systems that provides a form of acquired immunity in prokaryotes. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see e.g., Koonin et al., (2017) Curr Opin Microbiol 37:67-78).

Engineered versions of CRISPR/Cas systems has been developed in numerous formats to mutate or edit genomic DNA of cells from other species. The general approach of using the CRISPR/Cas system involves the heterologous expression or introduction of a site-directed nuclease (e.g., Cas nuclease) in combination with a guide RNA (gRNA) into a cell, resulting in a DNA cleavage event (e.g., the formation a single-strand or double-strand break (SSB or DSB)) in the backbone of the cell's genomic DNA at a precise, targetable location. The manner in which the DNA cleavage event is repaired by the cell provides the opportunity to edit the genome by the addition, removal, or modification (substitution) of DNA nucleotide(s) or sequences (e.g., genes).

In some embodiments, a system for editing a cell described herein comprises a nuclease capable of inducing a DNA break within an endogenous target gene in the cell. In some embodiments, the DNA break comprises a double stranded break (DSB), which is induced by a nuclease capable of inducing a DSB by cleaving both strands of double stranded DNA at a cleavage site. In some embodiments, the DNA break comprises a single strand break (SSB) at a cleavage site in the sense strand or the antisense strand of the endogenous target gene. In some embodiments, the DNA break comprises a SSB at a cleavage site in the sense strand, and a SSB at a cleavage site in the antisense strand, thereby resulting in a DSB. In some embodiments, the DSB is induced by a pair of recombinant nucleases, e.g., nickases, that are each capable of inducing a single strand break (SSB) in opposite DNA strands at different cleavage sites, e.g., at a cleavage site upstream of the gene variant in one strand and at a cleavage site downstream of the gene variant in the other strand of the target gene. In some embodiments, a first of the pair of nickases forms a complex with a first guide RNA, e.g., a first sgRNA, for targeting cleavage to one strand, e.g., the sense strand, and the second of the pair of nickases forms a complex with a second guide RNA, e.g., a second sgRNA, for targeting cleavage to the other strand, e.g., the antisense strand. In some embodiments, a DSB is induced through a SSB on each of the opposite strands, i.e., the sense strand and the antisense strand, of an endogenous target gene in the cell.

In general, genes are located in double stranded DNA that includes a sense strand and an antisense strand, which are complementary to one another. The sense strand is also referred to as the coding strand because its sequence is the DNA version of the RNA sequence that is transcribed. The antisense strand is also referred to as the template strand because its sequence is complementary to the RNA sequence that is transcribed.

i. Guide RNAs (gRNAs)

Engineered CRISPR/Cas systems comprise at least two components: 1) a guide RNA (gRNA) molecule and 2) a Cas nuclease, which interact to form a gRNA/Cas nuclease complex. A gRNA comprises at least a user-defined targeting domain termed a “spacer” comprising a nucleotide sequence and a CRISPR repeat sequence. In engineered CRISPR/Cas systems, a gRNA/Cas nuclease complex is targeted to a specific target sequence of interest within a target nucleic acid (e.g., a genomic DNA molecule) by generating a gRNA comprising a spacer with a nucleotide sequence that is able to bind to the specific target sequence in a complementary fashion (See Jinek et al., Science, 337, 816-821 (2012) and Deltcheva et al., Nature, 471, 602-607 (2011)). Thus, the spacer provides the targeting function of the gRNA/Cas nuclease complex.

In naturally-occurring type II-CRISPR/Cas systems, the “gRNA” is comprised of two RNA strands: 1) a CRISPR RNA (crRNA) comprising the spacer and CRISPR repeat sequence, and 2) a trans-activating CRISPR RNA (tracrRNA). In Type II-CRISPR/Cas systems, the portion of the crRNA comprising the CRISPR repeat sequence and a portion of the tracrRNA hybridize to form a crRNA:tracrRNA duplex, which interacts with a Cas nuclease (e.g., Cas9). As used herein, the terms “split gRNA” or “modular gRNA” refer to a gRNA molecule comprising two RNA strands, wherein the first RNA strand incorporates the crRNA function(s) and/or structure and the second RNA strand incorporates the tracrRNA function(s) and/or structure, and wherein the first and second RNA strands partially hybridize.

Accordingly, in some embodiments, a gRNA comprises two RNA molecules. In some embodiments, the gRNA comprises a CRISPR RNA (crRNA) and a trans-activating CRISPR RNA (tracrRNA). In some embodiments, the gRNA is a split gRNA. In some embodiments, the gRNA is a modular gRNA. In some embodiments, the split gRNA comprises a first strand comprising, from 5′ to 3′, a spacer, and a first region of complementarity; and a second strand comprising, from 5′ to 3′, a second region of complementarity; and optionally a tail domain.

In some embodiments, the crRNA comprises a spacer comprising a nucleotide sequence that is complementary to and hybridizes with a sequence that is complementary to the target sequence on a target nucleic acid (e.g., a genomic DNA molecule). In some embodiments, the crRNA comprises a region that is complementary to and hybridizes with a portion of the tracrRNA.

In some embodiments, the target nucleic acid (e.g., endogenous gene) is B2M. In some embodiments, the crRNA comprises the nucleotide sequence set forth in SEQ ID NO:18, or a nucleotide sequence having at least 80%, 85%, 90%, or 95% sequence identity to the nucleotide sequence set forth in SEQ ID NO:18. In some embodiments, the crRNA comprises the nucleotide sequence set forth in SEQ ID NO:18.

In some embodiments, the target nucleic acid (e.g., endogenous gene) is FKBP12. In some embodiments, the crRNA comprises the nucleotide sequence set forth in any one of SEQ ID NOS:19, 20, and 21, or a nucleotide sequence having at least 80%, 85%, 90%, or 95% sequence identity to a nucleotide sequence set forth in any one of SEQ ID NOS: 19, 20, and 21. In some embodiments, the crRNA comprises the nucleotide sequence set forth in any one of SEQ ID NOS:19, 20, and 21. In some embodiments, the crRNA comprises the nucleotide sequence set forth in SEQ ID NO:19, or a nucleotide sequence having at least 80%, 85%, 90%, or 95% sequence identity to the nucleotide sequence set forth in SEQ ID NO:19. In some embodiments, the crRNA comprises the nucleotide sequence set forth in SEQ ID NO: 19 In some embodiments, the crRNA comprises the nucleotide sequence set forth in SEQ ID NO:20, or a nucleotide sequence having at least 80%, 85%, 90%, or 95% sequence identity to the nucleotide sequence set forth in SEQ ID NO:20. In some embodiments, the crRNA comprises the nucleotide sequence set forth in SEQ ID NO:20. In some embodiments, the crRNA comprises the nucleotide sequence set forth in SEQ ID NO:21, or a nucleotide sequence having at least 80%, 85%, 90%, or 95% sequence identity to the nucleotide sequence set forth in SEQ ID NO:21. In some embodiments, the crRNA comprises the nucleotide sequence set forth in SEQ ID NO:21.

In some embodiments, the target nucleic acid (e.g., endogenous gene) is AAVS1. In some embodiments, the gRNA comprises the nucleotide sequence set forth in SEQ ID NO:52, or a nucleotide sequence having at least 80%, 85%, 90%, or 95% sequence identity to the nucleotide sequence set forth in SEQ ID NO:52. In some embodiments, the gRNA comprises the nucleotide sequence set forth in SEQ ID NO:52.

In some embodiments, the tracrRNA may comprise all or a portion of a wild-type tracrRNA sequence from a naturally-occurring CRISPR/Cas system. In some embodiments, the tracrRNA may comprise a truncated or modified variant of the wild-type tracr RNA. The length of the tracr RNA may depend on the CRISPR/Cas system used. In some embodiments, the tracrRNA may comprise 5, 6, 7, 8, 9, 10, 11, 12, 13, 14, 15, 16, 17, 18, 19, 20, 25, 30, 40, 50, 60, 70, 80, 90, 100, or more than 100 nucleotides in length. In certain embodiments, the tracrRNA is at least 26 nucleotides in length. In additional embodiments, the tracrRNA is at least 40 nucleotides in length. In some embodiments, the tracrRNA may comprise certain secondary structures, such as, e.g., one or more hairpins or stem-loop structures, or one or more bulge structures.

a) Single Guide RNA (sgRNA)

Engineered CRISPR/Cas nuclease systems often combine a crRNA and a tracrRNA into a single RNA molecule, referred to herein as a “single guide RNA” (sgRNA), by adding a linker between these components. Without being bound by theory, similar to a duplexed crRNA and tracrRNA, an sgRNA will form a complex with a Cas nuclease (e.g., Cas9), guide the Cas nuclease to a target sequence and activate the Cas nuclease for cleavage the target nucleic acid (e.g., genomic DNA). Accordingly, in some embodiments, the gRNA may comprise a crRNA and a tracrRNA that are operably linked. In some embodiments, the sgRNA may comprise a crRNA covalently linked to a tracrRNA. In some embodiments, the crRNA and the tracrRNA is covalently linked via a linker. In some embodiments, the sgRNA may comprise a stem-loop structure via base pairing between the crRNA and the tracrRNA. In some embodiments, a sgRNA comprises, from 5′ to 3′, a spacer, a first region of complementarity, a linking domain, a second region of complementarity, and, optionally, a tail domain.

The sgRNA can be unmodified or modified. For example, modified sgRNAs can comprise one or more 2′-O-methyl phosphorothioate nucleotides.

By way of illustration, guide RNAs used in the CRISPR/Cas system, or other smaller RNAs can be readily synthesized by chemical means, as illustrated herein and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

### b) Spacers

In some embodiments, the gRNAs comprise a spacer sequence. A spacer sequence is a sequence that defines the target site of a target nucleic acid (e.g. DNA). The target nucleic acid is a double-stranded molecule: one strand comprises the target sequence adjacent to a PAM sequence and is referred to as the “PAM strand,” and the second strand is referred to as the “non-PAM strand” and is complementary to the PAM strand and target sequence. Both gRNA spacer and the target sequence are complementary to the non-PAM strand of the target nucleic acid. In some embodiments, a spacer sequence corresponding to a target sequence adjacent to a PAM sequence is complementary to the non-PAM strand of the target nucleic acid. Thus, in some embodiments, a spacer sequence which corresponds to a target sequence adjacent to a PAM sequence is identical to the PAM strand. The gRNA spacer sequence hybridizes to the complementary strand (e.g.: the non-PAM strand of the target nucleic acid/target site). In some embodiments, the spacer is sufficiently complementary to the complementary strand of the target sequence (e.g.: non-PAM strand), as to target a Cas nuclease to the target nucleic acid. In some embodiments, the spacer is at least 80%, 85%, 90% or 95% complementary to the non-PAM strand of the target nucleic acid. In some embodiments, the spacer is 100% complementary to the non-PAM strand of the target nucleic acid. In some embodiments, the spacer comprises 1, 2, 3, 4, 5, 6 or more nucleotides that are not complementary with the non-PAM strand of the target nucleic acid. In some embodiments, the spacer comprises 1 nucleotide that is not complementary with the non-PAM strand of the target nucleic acid. In some embodiments, the spacer comprises 2 nucleotides that are not complementary with the non-PAM strand of the target nucleic acid.

In some embodiments, the 5′ most nucleotide of gRNA comprises the 5′ most nucleotide of the spacer. In some embodiments, the spacer is located at the 5′ end of the crRNA. In some embodiments, the spacer is located at the 5′ end of the sgRNA. In some embodiments, the spacer is about 15-50, about 20-45, about 25-40 or about 30-35 nucleotides in length. In some embodiments, the spacer is about 19-22 nucleotides in length. In some embodiments the spacer is about 15, 16, 17, 18, 19, 20, 21, 22, 23, 24, 25, 26, 27, 28, 29 or 30 nucleotides in length. In some embodiments the spacer is 19 nucleotides in length. In some embodiments, the spacer is 20 nucleotides in length, in some embodiments, the spacer is 21 nucleotides in length.

In some embodiments, the nucleotide sequence of the spacer is designed or chosen using a computer program. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, and/or presence of SNPs.

In some embodiments, the spacer comprise at least one or more modified nucleotide(s) such as those described herein. The disclosure provides gRNA molecules comprising a spacer which may comprise the nucleobase uracil (U), while any DNA encoding a gRNA comprising a spacer comprising the nucleobase uracil (U) will comprise the nucleobase thymine (T) in the corresponding position(s).

ii. Methods of Making gRNAs

Methods for making gRNAs are known to those of skill in the art and include but not limited to in vitro transcription (IVT), synthetic and/or chemical synthesis methods, or a combination thereof. Enzymatic (IVT), solid-phase, liquid-phase, combined synthetic methods, small region synthesis, and ligation methods are utilized. In one embodiment, the gRNAs are made using IVT enzymatic synthesis methods. Methods of making polynucleotides by IVT are known in the art and are described in International Application PCT/US2013/30062. Accordingly, the present disclosure also includes polynucleotides, e.g., DNA, constructs and vectors are used to in vitro transcribe a gRNA described herein.

In some embodiments, non-natural modified nucleobases are introduced into polynucleotides, e.g., gRNA, during synthesis or post-synthesis. In certain embodiments, modifications are on internucleoside linkages, purine or pyrimidine bases, or sugar. In some embodiments, the modification is introduced at the terminal of a polynucleotide; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

In some embodiments, enzymatic or chemical ligation methods are used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

In some embodiments, the disclosure provides nucleic acids, e.g., vectors, encoding gRNAs described herein. In some embodiments, the nucleic acid is a DNA molecule. In other embodiments, the nucleic acid is an RNA molecule. In some embodiments, the nucleic acid comprises a nucleotide sequence encoding a crRNA. In some embodiments, the nucleotide sequence encoding the crRNA comprises a spacer flanked by all or a portion of a repeat sequence from a naturally-occurring CRISPR/Cas system. In some embodiments, the nucleic acid comprises a nucleotide sequence encoding a tracrRNA. In some embodiments, the crRNA and the tracrRNA is encoded by two separate nucleic acids. In other embodiments, the crRNA and the tracrRNA is encoded by a single nucleic acid. In some embodiments, the crRNA and the tracrRNA is encoded by opposite strands of a single nucleic acid. In other embodiments, the crRNA and the tracrRNA is encoded by the same strand of a single nucleic acid.

In some embodiments, the gRNAs provided by the disclosure are chemically synthesized by any means described in the art (see e.g., WO/2005/01248). While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together.

In some embodiments, more than one guide RNA can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

The guide RNA may target any sequence of interest via the targeting sequence (e.g.: spacer sequence) of the crRNA. In some embodiments, the degree of complementarity between the targeting sequence of the guide RNA and the target sequence on the target nucleic acid molecule is about 60%, 65%, 70%, 75%, 80%, 85%, 90%, 95%, 97%, 98%, 99%, or 100%. In some embodiments, the targeting sequence of the guide RNA and the target sequence on the target nucleic acid molecule is 100% complementary. In other embodiments, the targeting sequence of the guide RNA and the target sequence on the target nucleic acid molecule may contain at least one mismatch. For example, the targeting sequence of the guide RNA and the target sequence on the target nucleic acid molecule may contain 1, 2, 3, 4, 5, 6, 7, 8, 9, or 10 mismatches. In some embodiments, the targeting sequence of the guide RNA and the target sequence on the target nucleic acid molecule may contain 1-6 mismatches. In some embodiments, the targeting sequence of the guide RNA and the target sequence on the target nucleic acid molecule may contain 5 or 6 mismatches.

The length of the targeting sequence may depend on the CRISPR-Cas system and components used. For example, different Cas9 proteins from different bacterial species have varying optimal targeting sequence lengths. Accordingly, the targeting sequence may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the targeting sequence may comprise 18-24 nucleotides in length. In some embodiments, the targeting sequence may comprise 19-21 nucleotides in length. In some embodiments, the targeting sequence may comprise 20 nucleotides in length.

In some embodiments of the present disclosure, a CRISPR/Cas nuclease system includes at least one guide RNA. In some embodiments, the guide RNA and the Cas protein may form a ribonucleoprotein (RNP), e.g., a CRISPR/Cas complex. The guide RNA may guide the Cas protein to a target sequence on a target nucleic acid molecule (e.g., a genomic DNA molecule), where the Cas protein cleaves the target nucleic acid. In some embodiments, the CRISPR/Cas complex is a Cpf1/guide RNA complex. In some embodiments, the CRISPR complex is a Type-II CRISPR/Cas9 complex. In some embodiments, the Cas protein is a Cas9 protein. In some embodiments, the CRISPR/Cas9 complex is a Cas9/guide RNA complex. In some embodiments, the CRISPR/Cas complex is an engineered Class 2 Type V CRISPR system. In some embodiments, the endonuclease is Mad7.

iii. Cas Nuclease

In some embodiments, the disclosure provides compositions and systems (e.g., an engineered CRISPR/Cas system) comprising a site-directed nuclease, wherein the site-directed nuclease is a Cas nuclease. The Cas nuclease may comprise at least one domain that interacts with a guide RNA (gRNA). Additionally, the Cas nuclease are directed to a target sequence by a guide RNA. The guide RNA interacts with the Cas nuclease as well as the target sequence such that, once directed to the target sequence, the Cas nuclease is capable of cleaving the target sequence. In some embodiments, the guide RNA provides the specificity for the cleavage of the target sequence, and the Cas nuclease are universal and paired with different guide RNAs to cleave different target sequences.

In some embodiments, the CRISPR/Cas system comprise components derived from a Type-I, Type-II, or Type-III system. Updated classification schemes for CRISPR/Cas loci define Class 1 and Class 2 CRISPR/Cas systems, having Types I to V or VI (Makarova et al., (2015) Nat Rev Microbiol, 13(11):722-36; Shmakov et al., (2015) Mol Cell, 60:385-397). Class 2 CRISPR/Cas systems have single protein effectors. Cas proteins of Types II, V, and VI are single-protein, RNA-guided endonucleases, herein called “Class 2 Cas nucleases.” Class 2 Cas nucleases include, for example, Cas9, Cpf1, C2c1, C2c2, and C2c3 proteins. The Cpf1 nuclease (Zetsche et al., (2015) Cell 163:1-13) is homologous to Cas9, and contains a RuvC-like nuclease domain.

In some embodiments, the Cas nuclease are from a Type-II CRISPR/Cas system (e.g., a Cas9 protein from a CRISPR/Cas9 system). In some embodiments, the Cas nuclease are from a Class 2 CRISPR/Cas system (a single-protein Cas nuclease such as a Cas9 protein or a Cpf1 protein). The Cas9 and Cpf1 family of proteins are enzymes with DNA endonuclease activity, and they can be directed to cleave a desired nucleic acid target by designing an appropriate guide RNA, as described further herein.

A Type-II CRISPR/Cas system component are from a Type-IIA, Type-IIB, or Type-IIC system. Cas9 and its orthologs are encompassed. Non-limiting exemplary species that the Cas9 nuclease or other components are from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonfex degensii, Caldicelulosiruptor becscii, Candidatus Desulforudis, Clostridium botulinum, Clostridium diffiMACe, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, or Acaryochloris marina. In some embodiments, the Cas9 protein are from Streptococcus pyogenes (SpCas9). In some embodiments, the Cas9 protein are from Streptococcus thermophilus (StCas9). In some embodiments, the Cas9 protein are from Neisseria meningitides (NmCas9). In some embodiments, the Cas9 protein are from Staphylococcus aureus (SaCas9). In some embodiments, the Cas9 protein are from Campylobacter jejuni (CjCas9).

In some embodiments, a Cas nuclease may comprise more than one nuclease domain. For example, a Cas9 nuclease may comprise at least one RuvC-like nuclease domain (e.g., Cpf1) and at least one HNH-like nuclease domain (e.g., Cas9). In some embodiments, the Cas9 nuclease introduces a DSB in the target sequence. In some embodiments, the Cas9 nuclease is modified to contain only one functional nuclease domain. For example, the Cas9 nuclease is modified such that one of the nuclease domains is mutated or fully or partially deleted to reduce its nucleic acid cleavage activity. In some embodiments, the Cas9 nuclease is modified to contain no functional RuvC-like nuclease domain. In other embodiments, the Cas9 nuclease is modified to contain no functional HNH-like nuclease domain. In some embodiments in which only one of the nuclease domains is functional, the Cas9 nuclease is a nickase that is capable of introducing a single-stranded break (a “nick”) into the target sequence. In some embodiments, a conserved amino acid within a Cas9 nuclease domain is substituted to reduce or alter a nuclease activity. In some embodiments, the Cas nuclease nickase comprises an amino acid substitution in the RuvC-like nuclease domain. Exemplary amino acid substitutions in the RuvC-like nuclease domain include D10A (based on the S. pyogenes Cas9 nuclease). In some embodiments, the nickase comprises an amino acid substitution in the HNH-like nuclease domain. Exemplary amino acid substitutions in the HNH-like nuclease domain include E762A, H840A, N863A, H983A, and D986A (based on the S. pyogenes Cas9 nuclease). In some embodiments, the nuclease system described herein comprises a nickase and a pair of guide RNAs that are complementary to the sense and antisense strands of the target sequence, respectively. The guide RNAs directs the nickase to target and introduce a DSB by generating a nick on opposite strands of the target sequence (i.e., double nicking). Chimeric Cas9 nucleases are used, where one domain or region of the protein is replaced by a portion of a different protein. For example, a Cas9 nuclease domain is replaced with a domain from a different nuclease such as Fok1. A Cas9 nuclease is a modified nuclease.

In some embodiments, the Cas nuclease is from a Type-I CRISPR/Cas system. In some embodiments, the Cas nuclease is a component of the Cascade complex of a Type-I CRISPR/Cas system. For example, the Cas nuclease is a Cas3 nuclease. In some embodiments, the Cas nuclease is derived from a Type-III CRISPR/Cas system. In some embodiments, the Cas nuclease is derived from Type-IV CRISPR/Cas system. In some embodiments, the Cas nuclease is derived from a Type-V CRISPR/Cas system. In some embodiments, the Cas nuclease is derived from a Type-VI CRISPR/Cas system.

In some embodiments, the Cas nuclease is a Mad endonuclease. CRISPR/Mad systems are closely related to the Type V (Cpf1-like) of Class-2 family of Cas enzymes. In some embodiments, the CRISPR-Mad system employs an Eubacterium rectale Mad7 endonuclease or variant thereof. The Mad7-crRNA complex cleaves target DNA by identification of a PAM 5′-YTTN.

b. Engineered Nucleases

In some embodiments, the cells described herein are genetically engineered with a site-directed nuclease, wherein the site-directed nuclease is an engineered nuclease. Exemplary engineered nucleases are meganuclease (e.g., homing endonucleases), ZFN, TALEN, and megaTAL.

Naturally-occurring meganucleases may recognize and cleave double-stranded DNA sequences of about 12 to 40 base pairs and are commonly grouped into five families. In some embodiments, the meganuclease are chosen from the LAGLIDADG family, the GIY-YIG family, the HNH family, the His-Cys box family, and the PD-(D/E)XK family. In some embodiments, the DNA binding domain of the meganuclease are engineered to recognize and bind to a sequence other than its cognate target sequence. In some embodiments, the DNA binding domain of the meganuclease are fused to a heterologous nuclease domain. In some embodiments, the meganuclease, such as a homing endonuclease, are fused to TAL modules to create a hybrid protein, such as a “megaTAL” protein. The megaTAL protein have improved DNA targeting specificity by recognizing the target sequences of both the DNA binding domain of the meganuclease and the TAL modules.

ZFNs are fusion proteins comprising a zinc-finger DNA binding domain (“zinc fingers” or “ZFs”) and a nuclease domain. Each naturally-occurring ZF may bind to three consecutive base pairs (a DNA triplet), and ZF repeats are combined to recognize a DNA target sequence and provide sufficient affinity. Thus, engineered ZF repeats are combined to recognize longer DNA sequences, such as, e.g., 9-, 12-, 15-, or 18-bp, etc. In some embodiments, the ZFN comprise ZFs fused to a nuclease domain from a restriction endonuclease. For example, the restriction endonuclease is FokI. In some embodiments, the nuclease domain comprises a dimerization domain, such as when the nuclease dimerizes to be active, and a pair of ZFNs comprising the ZF repeats and the nuclease domain is designed for targeting a target sequence, which comprises two half target sequences recognized by each ZF repeats on opposite strands of the DNA molecule, with an interconnecting sequence in between (which is sometimes called a spacer in the literature). For example, the interconnecting sequence is 5 to 7 bp in length. When both ZFNs of the pair bind, the nuclease domain may dimerize and introduce a DSB within the interconnecting sequence. In some embodiments, the dimerization domain of the nuclease domain comprises a knob-into-hole motif to promote dimerization. For example, the ZFN comprises a knob-into-hole motif in the dimerization domain of FokI.

The DNA binding domain of TALENs usually comprises a variable number of 34 or 35 amino acid repeats (“modules” or “TAL modules”), with each module binding to a single DNA base pair, A, T, G, or C. Adjacent residues at positions 12 and 13 (the “repeat-variable di-residue” or RVD) of each module specify the single DNA base pair that the module binds to. Though modules used to recognize G may also have affinity for A, TALENs benefit from a simple code of recognition—one module for each of the 4 bases—which greatly simplifies the customization of a DNA-binding domain recognizing a specific target sequence. In some embodiments, the TALEN may comprise a nuclease domain from a restriction endonuclease. For example, the restriction endonuclease is FokI. In some embodiments, the nuclease domain may dimerize to be active, and a pair of TALENS is designed for targeting a target sequence, which comprises two half target sequences recognized by each DNA binding domain on opposite strands of the DNA molecule, with an interconnecting sequence in between. For example, each half target sequence is in the range of 10 to 20 bp, and the interconnecting sequence is 12 to 19 bp in length. When both TALENs of the pair bind, the nuclease domain may dimerize and introduce a DSB within the interconnecting sequence. In some embodiments, the dimerization domain of the nuclease domain may comprise a knob-into-hole motif to promote dimerization. For example, the TALEN may comprise a knob-into-hole motif in the dimerization domain of FokI.

c. Target Sites

In some embodiments, the site-directed nucleases described herein are directed to and cleave (e.g., introduce a DSB) a target nucleic acid molecule (e.g. endogenous gene). In some embodiments, the target nucleic acid molecule is a housekeeping gene. In some embodiments, the housekeeping gene is eukaryotic translation elongation factor 1 alpha (EEF1A), glylceraldehyde-3-phosphate dehydrogenase (GAPDH), ubiquitin C (UBC), or actin beta (ACTB). In some embodiments, the target nucleic acid is a gene associated with rapamycin response. In some embodiments, the target nucleic acid is FKBP12. In some embodiments, the target nucleic acid is B2M.

The target nucleic acid molecule is any DNA molecule that is endogenous or exogenous to a cell. As used herein, the term “endogenous sequence” refers to a sequence that is native to the cell. In some embodiments, the target nucleic acid molecule is a genomic DNA (gDNA) molecule or a chromosome from a cell or in the cell. In some embodiments, the target sequence of the target nucleic acid molecule is a genomic sequence from a cell or in the cell. In some embodiments, the target sequence may be located in a coding sequence of a gene, an intron sequence of a gene, a transcriptional control sequence of a gene, a translational control sequence of a gene, or a non-coding sequence between genes. In some embodiments, the gene may be a protein coding gene. In other embodiments, the gene may be a non-coding RNA gene. In some embodiments, the target sequence may comprise all or a portion of a disease-associated gene.

In some embodiments, the target sequence may be located in a non-genic functional site in the genome that controls aspects of chromatin organization, such as a scaffold site or locus control region. In some embodiments, the target sequence may be a genetic safe harbor site, i.e., a locus that facilitates safe genetic modification.

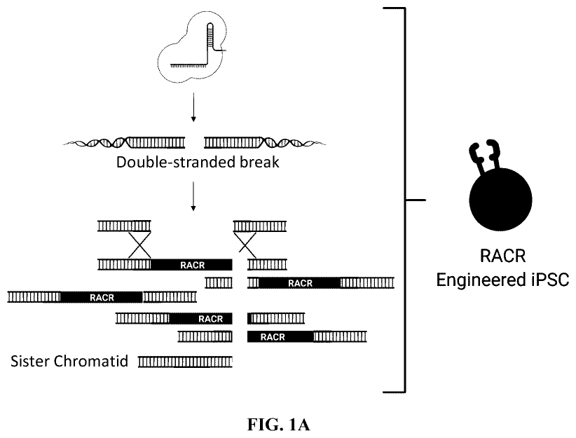

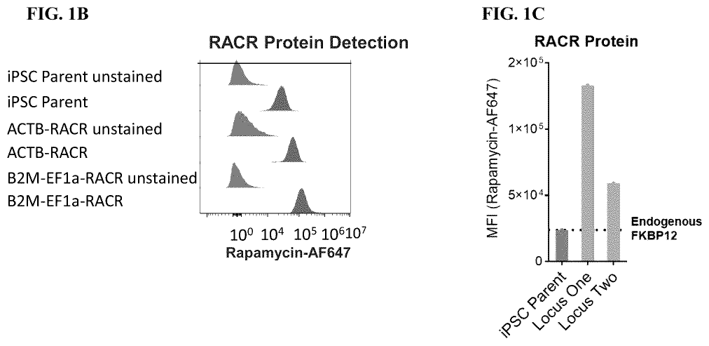

In some embodiments, the target sequence may be adjacent to a protospacer adjacent motif (PAM), a short sequence recognized by a CRISPR/Cas complex. In some embodiments, the PAM may be adjacent to or within 1, 2, 3, or 4, nucleotides of the 3′ end of the target sequence. In some embodiments, the target sequence may include the PAM. The length and the sequence of the PAM may depend on the Cas protein used. For example, the PAM may be selected from a consensus or a particular PAM sequence for a specific Cas nuclease or Cas ortholog, including those disclosed in FIG. 1 of Ran et al., (2015) Nature, 520:186-191 (2015), which is incorporated herein by reference. In some embodiments, the PAM may comprise 2, 3, 4, 5, 6, 7, 8, 9, or 10 nucleotides in length. Non-limiting exemplary PAM sequences include NGG (SpCas9 WT, SpCas9 nickase, dimeric dCas9-Fok1, SpCas9-HF1, SpCas9 K855A, eSpCas9 (1.0), eSpCas9 (1.1)), NGAN or NGNG (SpCas9 VQR variant), NGAG (SpCas9 EQR variant), NGCG (SpCas9 VRER variant), NAAG (SpCas9 QQR1 variant), NNGRRT or NNGRRN (SaCas9), NNNRRT (KKH SaCas9), NNNNRYAC (CjCas9), NNAGAAW (St1Cas9), NAAAAC (TdCas9), NGGNG (St3Cas9), NG (FnCas9), NAAAAN (TdCas9), NNAAAAW (StCas9), NNNNACA (CjCas9), GNNNCNNA (PmCas9), and NNNNGATT (NmCas9) (see e.g., Cong et al., (2013) Science 339:819-823; Kleinstiver et al., (2015) Nat Biotechnol 33:1293-1298; Kleinstiver et al., (2015) Nature 523:481-485; Kleinstiver et al., (2016) Nature 529:490-495; Tsai et al., (2014) Nat Biotechnol 32:569-576; Slaymaker et al., (2016) Science 351:84-88; Anders et al., (2016) Mol Cell 61:895-902; Kim et al., (2017) Nat Comm 8:14500; Fonfara et al., (2013) Nucleic Acids Res 42:2577-2590; Garneau et al., (2010) Nature 468:67-71; Magadan et al., (2012) PLoS ONE 7:e40913; Esvelt et al., (2013) Nat Methods 10(11):1116-1121(wherein N is defined as any nucleotide, W is defined as either A or T, R is defined as a purine (A) or (G), and Y is defined as a pyrimidine (C) or (T)). In some embodiments, the PAM sequence is NGG. In some embodiments, the PAM sequence is NGAN. In some embodiments, the PAM sequence is NGNG. In some embodiments, the PAM is NNGRRT. In some embodiments, the PAM sequence is NGGNG. In some embodiments, the PAM sequence may be NNAAAAW.

In some embodiments, the PAM sequence that is recognized by a nuclease, e.g., Cas9, differs depending on the particular nuclease and the bacterial species it is from. In some embodiments, the PAM sequence recognized by SpCas9 is the nucleotide sequence 5′-NGG-3′, where “N” is any nucleotide. In some embodiments, a PAM sequence recognized by SaCas9 is the nucleotide sequence 5′-NGRRT-3′ or the nucleotide sequence 5′-NGRRN-3′, where “N” is any nucleotide and “R” is a purine (e.g., guanine or adenine). In some embodiments, a PAM sequence recognized by NmeCas9 is the nucleotide sequence 5′-NNNNGATT-3′, where “N” is any nucleotide. In some embodiments, a PAM sequence recognized by CjCas9 is the nucleotide sequence 5′-NNNNRYAC-3′, where “N” is any nucleotide, “R” is a purine (e.g., guanine or adenine), and “Y” is a pyrimidine (e.g., cytosine or thymine). In some embodiments, a PAM sequence recognized by StCas9 is the nucleotide sequence 5′-NNAGAAW-3′, where “N” is any nucleotide and “W” is adenine or thymine.

In some embodiments, the recombinant nuclease is Cas9 and the PAM sequence is the nucleotide sequence: (a) 5′-NGG-3′; (b) 5′-NGRRT-3′ or 5′-NGRRN-3′; (c) 5′-NNNNGATT-3′; (d) 5′-NNNNRYAC-3′; or (e) 5′-NNAGAAW-3′; where “N” is any nucleotide, “R” is a purine (e.g., guanine or adenine), “Y” is a pyrimidine (e.g., cytosine or thymine), and “W” is adenine or thymine. In some embodiments, the recombinant nuclease is Cas9, e.g., SpCas9, and the PAM sequence is 5′-NGG-3′, where “N” is any nucleotide. In some embodiments, the recombinant nuclease is Cas9, e.g., SaCas9, and the PAM sequence is 5′-NGRRT-3′ or 5′-NGRRN-3′, where “N” is any nucleotide and “R” is a purine, such as guanine or adenine. In some embodiments, the recombinant nuclease is Cas9, e.g., NmeCas9, and the PAM sequence is 5′-NNNNGATT-3′, where “N” is any nucleotide. In some embodiments, the recombinant nuclease is Cas9, e.g., CjCas9, and the PAM sequence is 5′-NNNNRYAC-3′, where “N” is any nucleotide, “R” is a purine, such as guanine or adenine, and “Y” is a pyrimidine, such as cytosine or thymine. In some embodiments, the recombinant nuclease is Cas9, e.g., StCas9, and the PAM sequence is 5′-NNAGAAW-3′, where “N” is any nucleotide and “W” is adenine or thymine.

d. Ribonucleoproteins

In some embodiments, the site-directed polypeptide (e.g., Cas nuclease) and genome-targeting nucleic acid (e.g., gRNA or sgRNA) may each be administered separately to a cell or a subject. In some embodiments, the site-directed polypeptide may be pre-complexed with one or more guide RNAs, or one or more sgRNAs. Such pre-complexed material is known as a ribonucleoprotein particle (RNP). In some embodiments, the nuclease system comprises a ribonucleoprotein (RNP). In some embodiments, the nuclease system comprises a Cas9 RNP comprising a purified Cas9 protein in complex with a gRNA. In some embodiments, the nuclease system comprises a Mad7 RNP comprising a purified Mad7 protein in complex with a gRNA. Cas9 and Mad7 protein can be expressed and purified by any means known in the art. Ribonucleoproteins are assembled in vitro and can be delivered directly to cells using standard electroporation or transfection techniques known in the art.

### B. Targeted Gene Insertions

In some embodiments, the synthetic cytokine receptor (e.g. RACR) is integrated into a target nucleic acid molecule (e.g. an endogenous gene). In some embodiments, the integration into a target endogenous gene can disrupt expression of the target endogenous gene in the cells. In some embodiments, the target nucleic acid molecule is a housekeeping gene. In some embodiments, the housekeeping gene is eukaryotic translation elongation factor 1 alpha (EEF1A), glylceraldehyde-3-phosphate dehydrogenase (GAPDH), ubiquitin C (UBC), or actin beta (ACTB). In some embodiments, the target nucleic acid is B2M. In some embodiment, a nucleic acid encoding the engineered cytokine receptor is integrated into a disrupted B2M locus, such as by HDR or other methods. In some embodiments, HDR can be used to integrate a donor template comprising a nucleic acid encoding a synthetic cytokine receptor (e.g., a RACR) into a target nucleic acid molecule (e.g. an endogenous gene). For instance, by HDR methods a construct encoding the synthetic cytokine receptor further comprises a first homology arm and a second homology arm homologous to a target gene locus for CRISPR-based homology directed repair.

In some embodiments, one or more additional genes can be knocked-in or inserted into the genome of a cell. In some embodiments, a gene encoding a chimeric antigen receptor (CAR), such as described in Section IV, is inserted into the genome of a cell. In some embodiments, a gene encoding FRB, such as described in Section C, is inserted into the genome of a cell. In some embodiments, each of the one or more additional gene may be individual integrated into an endogenous gene. In some embodiments, the integration into a target endogenous gene can disrupt expression of the target endogenous gene in the cells. In some embodiments, the target nucleic acid molecule is a housekeeping gene. In some embodiments, the housekeeping gene is eukaryotic translation elongation factor 1 alpha (EEF1A), glylceraldehyde-3-phosphate dehydrogenase (GAPDH), ubiquitin C (UBC), or actin beta (ACTB). In some embodiments, the target nucleic acid molecule is a safe harbor gene. In some embodiments, the safe harbor gene is AAVS1. In some embodiments, the target nucleic acid is B2M. In some embodiments, HDR can be used to integrate a donor template comprising a nucleic acid encoding an additional gene (e.g. CAR or FRB) into an endogenous gene. For instance, by HDR methods a construct encoding the additional gene (e.g. nucleic acid encoding CAR or FRB) further comprises a first homology arm and a second homology arm homologous to a target gene locus for CRISPR-based homology directed repair. In some embodiments, a nucleic acid construct encoding the synthetic cytokine receptor is integrated into the B2M locus and a nucleic acid encoding FRB or a CAR is integrated into the AAVS1 locus. In some embodiments, a nucleic acid construct encoding the synthetic cytokine receptor is integrated into the B2M locus, a nucleic acid encoding FRB is integrated into one of the ACTB or EF1A locus, and a nucleic acid encoding a CAR is integrated into the other of the AAVS1 locus.

In some embodiments, transient BCL-XL overexpression is carried out in a cell that is disrupted for certain endogenous genes that are essential genes (Li et al. (2018) Nucleic Acids Research, 46:10195-10215). For instance, in some cases, editing essential genes requires anti-apoptotic support to enable clone selection and this can be achieved by providing transient overexpression of BCL-2 during editing. In some embodiments, transient BCL-XL overexpression can be achieved by introduction of a BCL-XL mRNA in the cell.

In some of embodiments, a stem cell, such as an iPSC, is engineered with the targeted gene insertion or insertions. In some embodiments, a progenitor cells, such as an HP, is engineered with the targeted gene insertion or insertions. In some embodiments, an iMAC is engineered with the targeted gene insertion or insertions.

Methods of introducing an exogenous gene, such as the synthetic cytokine receptor, into a target nucleic acid molecule (e.g. endogenous gene) are well known in the art (see for example Menke D. Genesis (2013) 51: 618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties) and include targeted homologous recombination, site specific recombinases, PB transposases and genome editing by engineered nucleases. Agents for introducing the synthetic cytokine receptor into a target nucleic acid molecule can be designed via publicly available sources or obtained commercially from Transposagen, Addgene and Sangamo Biosciences.

In some embodiments the gene editing technologies can be used for knock-out or knock-down of genes. In some embodiments, the gene-editing technologies can be used for knock-in or integration of DNA into a region of the genome. In some embodiments, the gene editing technology mediates double-strand breaks (DSB), including in connection with non-homologous end-joining (NHEJ) or homology-directed repair (HDR). In some embodiments, the a DNA base editing or prime-editing gene editing technology can be used. In some embodiments, a Programmable Addition via Site-specific Targeting Elements (PASTE) gene editing technology can be used.

Exemplary methods used to introduce the synthetic cytokine receptor into a target nucleic acid molecule include genome editing using endonucleases, meganucleases, zinc-finger nucleases and transcriptional activator-like effector nucleases (TALENs).

In some embodiments, methods to introduce an exogenous gene, such as a gene encoding the synthetic cytokine receptor. into a target nucleic acid molecule involves genome editing using engineered endonucleases. In some embodiments, this approach involves a reverse genetics method using artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a donor template for regenerating the missing DNA sequence at the break point. In order to introduce specific nucleotide modifications (e.g., mutations, such as amino acid substitutions) to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and RNA-guided nucleases (RGNs) such as Type II and Type V RGNs.

It will be apparent to one skilled in the art upon reading the present disclosure that various editing mechanisms can be used to create the cells, systems and methods of manufacture disclosed. Multiple different nuclease-based systems exist for providing edits into an organism's genome, and each can be used in either single editing systems, sequential editing systems (e.g., using different nuclease-directed systems sequentially to provide two or more genome edits in a cell) and/or recursive editing systems, (e.g., utilizing a single nuclease-directed system to introduce two or more genome edits in a cell). Thus, a person of skill in the art would recognize upon reading the present disclosure that various enzyme-directed editing systems are useful for the disclosed embodiments.

In some embodiments, the targeted insertion may be by target-primed reverse transcription (TPRT) or “prime editing”. In some embodiments, prime editing mediates targeted insertions in human cells without requiring DSBs or donor DNA templates. Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit

In some embodiments, targeted insertion is by Programmable Addition via Site-specific Targeting Elements (PASTE). In some aspects, PASTE is platform in which genomic insertion is directed via a CRISPR-Cas9 nickase fused to both a reverse transcriptase and serine integrase. As described in Ioannidi et al. (doi.org/10.1101/2021.11.01.466786), PASTE does not generate double stranded breaks, but allowed for integration of sequences as large as ~36 kb. In some embodiments, the serine integrase can be any known in the art. In some embodiments, the serine integrase has sufficient orthogonality such that PASTE can be used for multiplexed gene integration, simultaneously integrating at least two different genes at at least two genomic loci. In some embodiments, PASTE has editing efficiencies comparable to or better than those of homology directed repair or non-homologous end joining based integration, with activity in nondividing cells and fewer detectable off-target events.

### 1. Homology-Directed Repair (HDR)

In some aspects, the provided embodiments involve targeted integration of a nucleic acid sequence, such as a donor template, at a target nucleic acid sequence, e.g. an endogenous gene. In some embodiments, the target nucleic acid molecule is a housekeeping gene. In some embodiments, the housekeeping gene is eukaryotic translation elongation factor 1 alpha (EEF1A), glylceraldehyde-3-phosphate dehydrogenase (GAPDH), ubiquitin C (UBC), or actin beta (ACTB). In some embodiments, the target nucleic acid is a safe harbor gene. In some embodiments, the safe harbor gene is AAVS1. In some embodiments, the target nucleic acid is B2M.

In some embodiments, DNA repair mechanisms can be induced by a nuclease after (i) two SSBs, where there is a SSB on each strand, thereby inducing single strand overhangs; or (ii) a DSB occurring at the same cleavage site on both strands, thereby inducing a blunt end break.

In some embodiments, HDR is utilized for targeted integration or insertion of a nucleic acid sequence(s), e.g., a donor template, in one or more target nucleic acid molecules (e.g., endogenous gene(s)). In some embodiments, HDR can be used to integrate a donor template comprising a synthetic cytokine receptor (e.g., a RACR) and/or a CAR into a target nucleic acid molecule (e.g. an endogenous gene). For example, HDR can be used to integrate a donor template encoding a RACR into the B2M gene locus. For example, HDR can be used to further integrate a donor template encoding a CAR into the AAVS1 gene locus.

Agents capable of inducing a DSB, such as Cas nucleases (e.g. Cas9), TALENs, and ZFNs, promote genomic editing by inducing a DSB at a cleavage site within a target nucleic acid molecule such as an endogenous gene, e.g., B2M, as discussed in preceding sections.

Agents capable of inducing a SSB, also sometimes referred to as a nick, include recombinant nucleases, e.g., Cas9, having nickase activity, such as, e.g., those described in preceding sections. Examples of agents having nickase activity includes, e.g., a Cas9 from Streptococcus pyogenes that comprises a mutation selected from the group consisting of D10A, H840A, H854A, and H863A.

Upon cleavage by one of these agents, the target endogenous gene, e.g., B2M, with the SSBs or the DSB undergoes one of two major pathways for DNA damage repair: (1) the error-prone non-homologous end joining (NHEJ), or (2) the high-fidelity homology-directed repair (HDR) pathway.

In some embodiments, cells in which SSBs or a DSB was previously induced by one or more agent(s) comprising a nuclease, are obtained, and a donor template, e.g., ssODN, is introduced to result in HDR and integration of the donor template into the target endogenous gene, e.g., B2M.

In general, in the absence of a repair template, e.g., a donor template, such as a ssODN, the NHEJ process re-ligates the ends of the cleaved DNA strands, which frequently results in nucleotide deletions and insertions at the cleavage site.

Alteration of nucleic acid sequences at target endogenous gene locus, such as the B2M gene locus, can occur by HDR by integrating an exogenously provided donor template that encodes for a synthetic cytokine receptor (e.g., a RACR). The HDR pathway can occur by way of the canonical HDR pathway or the alternative HDR pathway. Unless otherwise indicated, the term “HDR” or “homology-directed repair” as used herein encompasses both canonical HDR and alternative HDR.

Canonical HDR or “canonical homology-directed repair” or cHDR,” are used interchangeably, and refers to the process of repairing DNA damage using a homologous nucleic acid (e.g., an endogenous homologous sequence, such as a sister chromatid; or an exogenous nucleic acid, such as a donor template). Canonical HDR typically acts when there has been a significant resection at the DSB, forming at least one single-stranded portion of DNA. In a normal cell, canonical HDR typically involves a series of steps such as recognition of the break, stabilization of the break, resection, stabilization of single-stranded DNA, formation of a DNA crossover intermediate, resolution of the crossover intermediate, and ligation. The canonical HDR process requires RAD51 and BRCA2, and the homologous nucleic acid, e.g., donor template, is typically double-stranded. In canonical HDR, a double-stranded polynucleotide, e.g., a double stranded donor template, is introduced, which comprises a sequence that is homologous to the targeting sequence within the target endogenous gene locus, and which will either be directly integrated into the targeting sequence or will be used as a template to insert the sequence, or a portion the sequence, of the donor template into the target endogenous gene, e.g., B2M After resection at the break, repair can progress by different pathways, e.g., by the double Holliday junction model (also referred to as the double strand break repair, or DSBR, pathway), or by the synthesis-dependent strand annealing (SDSA) pathway.

In the double Holliday junction model, strand invasion occurs by the two single stranded overhangs of the targeting sequence to the homologous sequences in the double-stranded polynucleotide, e.g., double stranded donor template, which results in the formation of an intermediate with two Holliday junctions. The junctions migrate as new DNA is synthesized from the ends of the invading strand to fill the gap resulting from the resection. The end of the newly synthesized DNA is ligated to the resected end, and the junctions are resolved, resulting in the insertion at the targeting sequence, or a portion of the targeting sequence that includes the gene variant. Crossover with the polynucleotide, e.g., donor template, may occur upon resolution of the junctions.

In the SDSA pathway, only one single stranded overhang invades the polynucleotide, e.g., donor template, and new DNA is synthesized from the end of the invading strand to fill the gap resulting from resection. The newly synthesized DNA then anneals to the remaining single stranded overhang, new DNA is synthesized to fill in the gap, and the strands are ligated to produce the modified DNA duplex.

Alternative HDR, or “alternative homology-directed repair,” or “alternative HDR,” are used interchangeably, and refers, in some embodiments, to the process of repairing DNA damage using a homologous nucleic acid (e.g., an endogenous homologous sequence, such as a sister chromatid; or an exogenous nucleic acid, such as a donor template). Alternative HDR is distinct from canonical HDR in that the process utilizes different pathways from canonical HDR, and can be inhibited by the canonical HDR mediators, RAD51 and BRCA2. Moreover, alternative HDR is also distinguished by the involvement of a single-stranded or nicked homologous nucleic acid template, e.g., donor template, whereas canonical HDR generally involves a double-stranded homologous template. In the alternative HDR pathway, a single strand template polynucleotide, e.g., donor template, is introduced. A nick, single strand break, or DSB at the cleavage site, for altering a desired target site, e.g., a target endogenous gene, e.g., B2M, is mediated by a nuclease molecule, e.g., any of the nucleases as described herein, and resection at the break occurs to reveal single stranded overhangs. Incorporation of the sequence of the template polynucleotide, e.g., donor template, to alter the target site of the DNA typically occurs by the SDSA pathway, as described herein.

In some embodiments, HDR is carried out by introducing, into a cell, one or more agent(s) capable of inducing a DSB, such as any of those as described herein, and a donor template, e.g., ssODN, such as any of those described herein. The introducing can be carried out by any suitable delivery means, such as any of those as described herein. The conditions under which HDR is allowed to occur can be any conditions suitable for carrying out HDR in a cell.

In some embodiments, HDR is carried out by introducing, into a cell, one or more agent(s) capable of inducing a SSB in each stand, such as any of those as described herein, and a donor template, e.g., ssODN, such as any of those described herein. The introducing can be carried out by any suitable delivery means, such as any of those as described herein. The conditions under which HDR is allowed to occur can be any conditions suitable for carrying out HDR in a cell.

a. Donor Templates

In some embodiments, the provided methods include the use of a donor template, e.g., a donor template encoding a synthetic cytokine receptor, e.g., a RACR and/or a CAR, that is homologous to a portion(s) of the targeting sequence in the target gene, e.g., B2M and/or AAVS1. In some embodiments, the targeting sequence is comprised within the sense strand. In some embodiments, the targeting sequence is comprised within the antisense strand. Also provided, in some embodiments, are donor templates for use in the methods provided herein, e.g., as templates for HDR-mediated integration of a nucleic acid sequence encoding a RACR.

In some embodiments, the donor template is used in conjunction with the one or more agent(s) capable of inducing a DNA break, e.g., a SSB or a DSB. In some embodiments, the donor template is used in conjunction with the one or more agent(s) capable of inducing a DSB and a guide RNA, e.g., sgRNA, to knock in a nucleic acid sequence encoding a synthetic cytokine receptor (e.g., a RACR) at a target endogenous gene locus (e.g., B2M). In some embodiments, the donor template is used in conjunction with the one or more agent(s) capable of inducing a SSB; the first guide RNA, e.g., the first sgRNA; and the second guide RNA, e.g., the second sgRNA, to knock in a nucleic acid sequence encoding a synthetic cytokine receptor (e.g., a RACR) at a target endogenous gene locus (e.g., B2M).

In some embodiments, the donor template comprises a nucleic acid sequence that is homologous to the cleavage site in the target gene, e.g., B2M. In some embodiments, the donor template comprises a nucleic acid sequence that is homologous to the cleavage site in the sense strand of the target gene, e.g., B2M. In some embodiments, the donor template comprises a nucleic acid sequence that is homologous to the cleavage site in the antisense strand of the target gene, e.g., B2M. In some embodiments, the target gene, e.g., B2M, includes a sense strand and an antisense strand, and the sense strand comprises the targeting sequence. In some embodiments, the target gene, e.g., B2M, includes a sense strand and an antisense strand, and the antisense strand comprises the targeting sequence.

In some embodiments, the donor template, e.g., ssODN, comprises a nucleic acid sequence comprising a PAM sequence that is homologous to the PAM sequence in the targeting sequence.

In some embodiments, the donor template is single-stranded. In some embodiments, the donor template is a single-stranded DNA oligonucleotide (ssODN). In some embodiments, the donor template is double-stranded.

In some embodiments, the ssODN comprises a 5′ ssODN arm and a 3′ ssODN arm. In some embodiments, the 5′ ssODN arm is directly linked to the 3′ ssODN arm. In some embodiments, the 5′ ssODN arm is homologous to the sequence of the target gene, e.g., B2M, that is immediately upstream of the cleavage site, and the 3′ ssODN arm is homologous to the sequence of the target gene that is immediately downstream of the cleavage site.

In some embodiments, the 5′ ssODN arm and/or the 3′ ssODN arm has a length that is between 250 and 750 nucleotides in length. In some embodiments, the 5′ ssODN arm has a length that is between 250 and 750 nucleotides in length. In some embodiments, the 3′ ssODN arm has a length that is between 250 and 750 nucleotides in length. In some embodiments, each of the 5′ ssODN arm and the 3′ ssODN arm has a length that is between 250 and 750 nucleotides in length. In some embodiments, the 5′ ssODN arm and/or the 3′ ssODN arm has a length that is about 500 nucleotides in length. In some embodiments, the 5′ ssODN arm has a length that is about 500 nucleotides in length. In some embodiments, the 3′ ssODN arm has a length that is about 500 nucleotides in length. In some embodiments, each of the 5′ ssODN arm and the 3′ ssODN arm has a length that is about 500 nucleotides in length.

In some embodiments, the target gene is B2M and the donor template comprises a nucleic acid sequence that is homologous to the cleavage site in the B2M gene. In some embodiments, the donor template comprises a nucleic acid sequence that is homologous to the cleavage site in the sense strand of the B2M target gene. In some embodiments, the donor template comprises a nucleic acid sequence that is homologous to the cleavage site in the antisense strand of the B2M target gene. In some embodiments, the donor template is a ssODN and the 5′ ssODN arm is homologous to the sequence of the B2M target gene that is immediately upstream of the cleavage site, and the 3′ ssODN arm is homologous to the sequence of the B2M target gene that is immediately downstream of the cleavage site.

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO:22. In some embodiments, the 5′ ssODN arm comprises the nucleic acid sequence set forth in SEQ ID NO:22.

In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 23 In some embodiments, the 3′ ssODN arm comprises the nucleic acid sequence set forth in SEQ ID NO:23.

In some embodiments, the 5′ ssODN arm comprises the nucleic acid sequence as set forth in SEQ ID NO: 22, and the 3′ ssODN arm comprises the nucleic acid sequence as set forth in SEQ ID NO: 23.

In some embodiments, the target gene is AAVS1 and the donor template comprises a nucleic acid sequence that is homologous to the cleavage site in the AAVS1 gene. In some embodiments, the donor template comprises a nucleic acid sequence that is homologous to the cleavage site in the sense strand of the AAVS1 target gene. In some embodiments, the donor template comprises a nucleic acid sequence that is homologous to the cleavage site in the antisense strand of the AAVS1 target gene. In some embodiments, the donor template is a ssODN and the 5′ ssODN arm is homologous to the sequence of the AAVS1 target gene that is immediately upstream of the cleavage site, and the 3′ ssODN arm is homologous to the sequence of the AAVS1 target gene that is immediately downstream of the cleavage site.

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO:53. In some embodiments, the 5′ ssODN arm comprises the nucleic acid sequence set forth in SEQ ID NO:53.

In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 54. In some embodiments, the 3′ ssODN arm comprises the nucleic acid sequence set forth in SEQ ID NO:54.

In some embodiments, the 5′ ssODN arm comprises the nucleic acid sequence as set forth in SEQ ID NO: 53, and the 3′ ssODN arm comprises the nucleic acid sequence as set forth in SEQ ID NO: 54.

Also provided herein is an isolated nucleic acid, e.g., an isolated nucleic acid for use in a method of knocking in a synthetic cytokine receptor (e.g., a RACR or CAR) into a target gene (e.g., B2M or AAVS1), comprising the nucleic acid sequence of any of the donor templates, e.g., ssODNs, or portions thereof, e.g., or 5′ ssODN arms, or 3′ ssODN arms, described herein.

In some embodiments, the crRNA comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 18; the 5′ ssODN arm comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 22; and the 3′ ssODN arm comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 23. In some embodiments, the crRNA comprises the nucleic acid sequence set forth in any one of SEQ ID NOS: 18; the 5′ ssODN comprises the nucleic acid sequence set forth in SEQ ID NO: 22; and the 3′ ssODN comprises the nucleic acid sequence set forth in SEQ ID NO: 23.

Also provided herein is an isolated nucleic acid, e.g., an isolated nucleic acid for use in a method of knocking in a synthetic cytokine receptor (e.g., a CAR) into a target gene (e.g., AAVS1), comprising the nucleic acid sequence of any of the donor templates, e.g., ssODNs, or portions thereof, e.g., or 5′ ssODN arms, or 3′ ssODN arms, described herein.

In some embodiments, the crRNA comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 52; the 5′ ssODN arm comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 53; and the 3′ ssODN arm comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 54. In some embodiments, the crRNA comprises the nucleic acid sequence set forth in any one of SEQ ID NOS: 52; the 5′ ssODN comprises the nucleic acid sequence set forth in SEQ ID NO: 53; and the 3′ ssODN comprises the nucleic acid sequence set forth in SEQ ID NO: 54.

In some embodiments, the donor template, e.g., ssODN, comprises a nucleic acid sequence encoding a transgene sequence encoding the synthetic cytokine receptor. In some embodiments, the synthetic cytokine receptor is a rapamycin-activated cytokine receptor (RACR) that is responsive to rapamycin or an analog (e.g. rapalog). In some embodiments, the transgene sequence is a tandem cassette that encodes both polypeptides of the synthetic cytokine receptor.

In some embodiments, the transgene encoding the synthetic cytokine receptor (e.g. RACR) can be inserted so that its expression is driven by the endogenous promoter at the integration site, for example the promoter that drives expression of the endogenous B2M gene. In some embodiments in which the polypeptide encoding sequences are promoterless, expression of the integrated transgene is then ensured by transcription driven by an endogenous promoter or other control element in the region of interest. For example, the transgene encoding a portion of the synthetic cytokine receptor (e.g. RACR) can be inserted without a promoter, but in-frame with the coding sequence of the endogenous locus (e.g. B2M locus) such that expression of the integrated transgene is controlled by the transcription of the endogenous promoter and/or other regulatory elements at the integration site. In some embodiments, a multi-cistronic element such as a ribosome skipping element/self-cleavage element (e.g., a 2A element or an internal ribosome entry site (IRES)), is placed upstream of the transgene, such that the multi-cistronic element is placed in-frame with one or more exons of the endogenous open reading frame at the endogenous locus (e.g. B2M locus), such that the expression of the transgene is operably linked to the endogenous promoter.

In some embodiments, each nucleic acid encoding a polypeptide of the synthetic cytokine receptor in the “tandem” cassettes is independently controlled by a regulatory element or all controlled as a multi-cistronic (e.g. bicistronic) expression system. In other embodiments, each nucleic acid encoding a polypeptide of the synthetic cytokine receptor in the “tandem” cassettes can be operatively linked to a promoter, which can be the same or different. In some embodiments, the nucleic acid molecule can contain a promoter that drives the expression of two or more different polypeptide chains. In some embodiments, such nucleic acid molecules can be multi-cistronic (bicistronic or tricistronic, see e.g., U.S. Pat. No. 6,060,273). In some embodiments, transcription units can be engineered as a bicistronic unit containing an IRES (internal ribosome entry site), which allows coexpression of gene products by a message from a single promoter. Alternatively, in some cases, a single promoter may direct expression of an RNA that contains, in a single open reading frame (ORF), two polypeptides separated from one another by sequences encoding a cleavable linker as described herein. The ORF thus encodes a single polypeptide, which, either during or after translation, is processed into the individual polypeptide chains. In some embodiments, the promoter is selected from among human elongation factor 1 alpha (EF1α) promoter (such as set forth in SEQ ID NO:24, 25 or 26). In some embodiments, the promoter is an MND promoter (such as set forth in SEQ ID NO:27).

In some embodiments, the donor template, e.g., ssODN, comprises a nucleic acid sequence encoding a synthetic cytokine receptor (e.g. RACR). In some embodiments, the nucleic acid sequence encoding the synthetic cytokine receptor (e.g. RACR) is located between the 5′ ssODN arm and the 3′ ssODN arm. In some embodiments, the nucleic acid sequence encoding the synthetic cytokine receptor (e.g. RACR) comprises an EF1-alpha promoter (e.g., SEQ ID NO:24, 25 or 26). In some embodiments, the nucleic acid sequence encoding the synthetic cytokine receptor (e.g. RACR) comprises a MND promoter (e.g., SEQ ID NO:27). In some embodiments, the synthetic cytokine receptor is a rapamycin-activated cytokine receptor (RACR). The RACR can be any as described, such as in Section II.B. In some embodiments, the nucleic acid molecule is a tandem cassette encoding the first polypeptide sequence of RACR and the second polypeptide sequence of RACR.

In some embodiments, the first nucleic acid sequence encoding the RACR comprises a nucleic acid sequence encoding a RACR-gamma chain (e.g., SEQ ID NO:28), and a nucleic acid sequence encoding a RACR-beta chain (e.g., SEQ ID NO:33). In some embodiments, the first nucleic acid sequence encodes a RACR-gamma chain that has at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 28. In some embodiments, the first nucleic acid sequence encodes the RACR-gamma chain sequence set forth in SEQ ID NO:28. In some embodiments, the nucleic acid sequence encoding the RACR-gamma chain further encodes a signal peptide at the N-terminus of the nascent protein to prompt transport of the protein when expressed. In some embodiments, the signal peptide has the sequence set forth in SEQ ID NO: 29. In some embodiments, the second nucleic acid sequence encodes a RACR-beta chain that has at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 33. In some embodiments, the second nucleic acid sequence encodes the RACR-beta chain set forth in SEQ ID NO:33. In some embodiments, the nucleic acid sequence encoding the RACR-beta chain further encodes a signal peptide at the N-terminus of the nascent protein to prompt transport of the protein when expressed. In some embodiments, the signal peptide has the sequence set forth in SEQ ID NO: 34.

In some embodiments, the first nucleic acid sequence encoding the RACR-gamma chain has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 37. In some embodiments, the first nucleic acid sequence encoding the RACR-gamma chain has the sequence set forth in SEQ ID NO:37. In some embodiments, the second nucleic acid sequence encoding the RACR-beta chain has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 38. In some embodiments, the second nucleic acid sequence encoding the RACR-beta chain is set forth in SEQ ID NO:38.

In some embodiments, the nucleic acid sequence encoding the RACR-gamma chain and the nucleic acid sequence encoding the RACR-beta chain are separated by a nucleic acid sequence encoding a cleavable linker. In some embodiments, a further nucleic acid sequence encoding a cleavable linker is located downstream of the nucleic acid sequence encoding the RACR-beta chain

In some embodiments, the linker is a protein quantitation reporter linker (PQR; e.g., SEQ ID NO:42), including any as described in Canadian Patent Application No. CA2970093, incorporated by reference in its entirety herein. In some embodiments, the PQR linker has the sequence set forth in SEQ ID NO:42. In some embodiments, the PQR linker is encoded by a sequence of nucleotides set forth in SEQ ID NO:41.

In some embodiments, the cleavable linker is a self-cleaving peptide, such as a 2A ribosomal skip element. In some cases, the cleavable linker, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and nucleic acids disclosed herein, without limitation, 2A sequences from the foot-and-mouth disease virus (F2A, e.g., SEQ ID NO: 43), equine rhinitis A virus (E2A, e.g., SEQ ID NO: 44), Thosea asigna virus (T2A, e.g., SEQ ID NO: 45 or 46), and porcine teschovirus-1 (P2A, e.g., SEQ ID NO: 47 or 48) as described in U.S. Patent Publication No. 20070116690.

In some embodiments, by virtue of the cleavable element located between the first nucleic acid sequence and the second nucleic acid sequence, expression of a nucleic acid sequence encoding a RACR yields a first peptide (i.e., the RACR-gamma chain) and a separate, second peptide (i.e., the RACR-beta chain).

In some embodiments, the transgene sequences may also include sequences required for transcription termination and/or polyadenylation signal. In some aspects, exemplary polyadenylation signal is selected from SV40, hGH, BGH, and rbGlob transcription termination sequence and/or polyadenylation signal. In some embodiments, the transgene includes an SV40 polyadenylation signal. In some embodiments, if present within the transgene, the transcription termination sequence and/or polyadenylation signal is typically the most 3′ sequence within the transgene, and is linked to one of the homology arm. In some embodiments, transgene sequence includes the polyadenylation sequence set forth in SEQ ID NO:39.

In some embodiments, the ssODN comprises, in order: a 5′ ssODN arm, a EF1-alpha promoter, a nucleic acid sequence encoding the RACR-gamma chain, a nucleic acid sequence encoding a cleavable linker (e.g., a PQR linker), a nucleic acid sequence encoding the RACR-beta chain, a polyA sequence, and the 3′ ssODN arm.

In some embodiments, the ssODN comprises the sequence set forth in SEQ ID NO:40 or a sequence that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 40. In some embodiments, the ssODN is set forth in SEQ ID NO:40.

In some embodiments, after the integration of the ssODN into the target gene, the target gene is knocked out. In some embodiments, the target gene is human B2M and/or AAVS1, and, after the integration of the ssODN, B2M and/or AAVS1 is knocked out. In some embodiments, a nucleic acid sequence encoding the synthetic cytokine receptor is integrated into the B2M locus. In some embodiments, a nucleic acid sequence encoding the CAR is integrated into the AAVS1 locus. In some embodiments, the engineered iPSC and iMAC has a modified B2M locus in which the endogenous B2M gene is genetically disrupted by knockout of the B2M gene and knock in by targeted integration of a nucleic acid encoding the synthetic cytokine receptor. In some embodiments, the engineered iPSC and iMAC has a modified AAVS1 locus in which the endogenous AAVS1 gene is genetically disrupted by knockout of the AAVS1 gene and knock in by targeted integration of a nucleic acid encoding the CAR. In some embodiments, the synthetic cytokine receptor is a RACR encoded by a nucleic acid sequence that contains in order: a EF1-alpha promoter, a nucleic acid sequence encoding the RACR-gamma chain, a nucleic acid sequence encoding a cleavable linker (e.g., a PQR linker), a nucleic acid sequence encoding the RACR-beta chain, and a polyA sequence. In some embodiments, the nucleic acid sequence encoding RACR that is integrated into the B2M locus has the sequence set forth in SEQ ID NO:32 or a sequence that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 32. In some embodiments, the nucleic acid encoding RACR that is integrated into the B2M locus is set forth in SEQ ID NO:32.

### IV. CHIMERIC ANTIGEN RECEPTOR

In some cases, the stem cells or macrophages of the present disclosure comprise a polynucleotide encoding a chimeric antigen receptor (CAR), thereby generating stem cells or macrophages expressing the CAR.

In some embodiments, the disclosure contemplates a chimeric antigen receptor (CAR) system for use in the treatment of subjects with cancer. In some embodiments, the macrophages of the disclosure comprise a CAR sequence (CAR-macrophages or CAR-iMAC cells).

In some embodiments, stem cells or macrophages are engineered to express CAR constructs by transfecting a population of cells with an expression vector encoding the CAR construct. Illustrative examples of populations of cells that may be transfected include HSCs, blood progenitor cells, myeloid progenitor cells, or macrophages. Appropriate means for preparing a transduced population of macrophages expressing a selected CAR construct will be well known to the skilled artisan, and includes retrovirus, lentivirus (viral mediated CAR gene delivery system), sleeping beauty, and piggyback (transposon/transposase systems that include a non-viral mediated CAR gene delivery system), to name a few examples. In some embodiments, any of the transduction methods contemplated in the disclosure may be used to generate CAR-expressing stem cells or macrophages.

In some embodiments, stem cells or macrophages are engineered to express CAR constructs by genetically engineering (e.g., via CRISPR) a population of cells to express the CAR construct. In some embodiments, a nucleic acid molecule encoding a CAR, such as by introduction of a vector construct encoding the CAR, is introduced into the cell. In some embodiments, the construct is designed for insertion of the nucleic acid encoding the CAR into an endogenous locus in the cell. Methods of gene insertion or knock-in are known, including any of the methods described in Section III. In some embodiments, insertion of a CAR-encoding construct is by homology directed repair, such as by using a CRISPR-Cas system.

In some embodiments, the CAR construct contains an extracellular binding portion, a transmembrane domain and an intracellular signaling domain. In some embodiments, the intracellular signaling domain contains a costimulatory signaling domain and/or an activation signaling domain. In some embodiments, the CAR construct contains an extracellular binding portion, a transmembrane domain and an intracellular signaling domain comprising a costimulatory signaling domain. In some embodiments, the CAR construct contains an extracellular binding portion, a transmembrane domain and an intracellular signaling domain comprising an activation signaling domain. In some embodiments, the CAR construct contains an extracellular binding portion, a transmembrane domain and an intracellular signaling domain comprising a costimulatory signaling domain and an activation signaling domain.

In some embodiments, the CARs may include additional elements, such a signal peptide to ensure proper export of the fusion protein to the cells surface, a transmembrane domain to ensure the fusion protein is maintained as an integral membrane protein, and a hinge domain that imparts flexibility to the recognition region and allows strong binding to the targeted moiety.

### A. Extracellular Binding Portion

Conventionally, CARs are generated by fusing a polynucleotide encoding a VL, VH, or scFv to the 5′ end of a polynucleotide encoding transmembrane and intracellular domains, and transducing cells with that polynucleotide as well as with the corresponding VH or VL, if needed. Numerous variations on CARs well known in the art and the disclosure contemplates using any of the known variations. Additionally, VL/VH pairs and scFv's for innumerable haptens are known in the art or can be generated by conventional methods routinely. Accordingly, the present disclosure contemplates using any known hapten-binding domain.

In any embodiments described herein, the binding portion of the CAR can be, for example, a single chain fragment variable region (scFv) of an antibody, a Fab, Fv, Fc, or (Fab′)2 fragment, and the like. The use of unaltered (i.e., full size) antibodies, such as IgG, IgM, IgA, IgD or IgE, in the CAR or as the CAR is excluded from the scope of the invention.

In some embodiments, the binding portion of the CAR can be directed to any antigen that is desired to be targeted, such as due to its overexpression on cells or association with a disease or conditions like cancer.

In some embodiments, the binding portion of the CAR is specific to a tumor antigen. The selection of the antigen binding domain will depend on the particular type of cancer to be treated. Tumor antigens are well known in the art and include, for example, a glioma-associated antigen, carcinoembryonic antigen (CEA), EGFRvIII, IL-11Ra, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, CD123, cyclin B1, lectin-reactive AFP, Fos-related antigen 1, ADRB3, thyroglobulin, EphA2, RAGE-1, RU1, RU2, SSX2, AKAP-4, LCK, OY-TES1, PAXS, SART3, CLL-1, fucosyl GM1, GloboH, MN-CA IX, EPCAM, EVT6-AML, TGS5, human telomerase reverse transcriptase, plysialic acid, PLAC1, RU1, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-1a, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6, E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRCSD, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, IL13Ra2, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG1 6, TA-90\Mac-2 binding proteincyclophilm C-associated protein, TAAL6, TAG72, TLP, TPS, GPC3, MUC16, LMP1, EBMA-1, BARF-1, CS1, CD319, HER1, B7H6, LiCAM, IL6, and MET.

In some embodiments, a CAR comprises an extracellular domain comprising a FMC63 scFv binding domain for CD19 binding. In some embodiments, the CAR is a second-generation CAR comprised of the FMC63 mouse anti-human CD19 scFv linked to the 4-1BB costimulatory domain and the CD3zeta intracellular signaling domain. In some embodiments, a CAR comprises a binding domain for CD19, a CD8a hinge, a CD8a transmembrane domain, a 4-1BB costimulatory domain, and a CD3zeta signaling domain. In some embodiments, a CAR comprises a binding domain for CD19, an IgG4 hinge, a CD28 transmembrane domain, a 4-1BB costimulatory domain, and a CD3zeta signaling domain. In some embodiments, a CAR comprises a binding domain for CD19, a CD28 hinge, a CD28 transmembrane domain, a CD28 costimulatory domain, and CD3zeta signaling domain. In some embodiments, a CAR comprises an extracellular domain comprising a FMC63 scFv binding domain for CD19 binding, a CD8a hinge, a CD8a transmembrane domain, a 4-1BB costimulatory domain, and a CD3zeta signaling domain. In some embodiments, a CAR comprises an extracellular domain comprising a FMC63 scFv binding domain for CD19 binding, an IgG4 hinge, a CD28 transmembrane domain, a 4-1BB costimulatory domain, and a CD3zeta signaling domain. In some embodiments, a CAR comprises an extracellular domain comprising a FMC63 scFv binding domain for CD19 binding, a CD28 hinge, a CD28 transmembrane domain, a CD28 costimulatory domain, and CD3zeta signaling domain.

In some embodiments, the CAR is a second-generation CAR comprised of the FMC63 mouse anti-human CD19 scFv linked to the CD28 costimulatory domain and the CD3zeta intracellular signaling domain. In some embodiments, the CAR is a second-generation CAR comprised of the FMC63 mouse anti-human CD19 scFv linked to a CD8 transmembrane domain, 4-1BB costimulatory domain, and the CD3zeta intracellular signaling domain.

In some embodiments, the antigen is BCMA. CAR T therapies targeting BCMA have been approved by the FDA and include Abecma and Carvykti. CARs targeting BCMA are described, for example, in US Publication No. 2020/0246381; U.S. Pat. No. 10,918,665; US Publication No. 2019/0161553, each of which is herein incorporated by reference. In some embodiments, a CAR comprises a binding domain for BCMA, a CD8a hinge, a CD8a transmembrane domain, a 4-1BB costimulatory domain, and a CD3zeta signaling domain. In some embodiments, a CAR comprises a binding domain for BCMA, an IgG4 hinge, a CD28 transmembrane domain, a 4-1BB costimulatory domain, and a CD3zeta signaling domain. In some embodiments, a CAR comprises a binding domain for BCMA, a CD28 hinge, a CD28 transmembrane domain, a CD28 costimulatory domain, and CD3zeta signaling domain.

In some embodiments, the antigen is G protein-coupled receptor class C group 5 member D (GPRC5D). CARs targeting GRC5D are described, for example, in US Publication Nos. 2018/0118803 and 2021/10393689, each of which is herein incorporated by reference. In some embodiments, a CAR comprises a binding domain for GRC5D, a CD8a hinge, a CD8a transmembrane domain, a 4-1BB costimulatory domain, and a CD3zeta signaling domain. In some embodiments, a CAR comprises a binding domain for GRC5D, an IgG4 hinge, a CD28 transmembrane domain, a 4-1BB costimulatory domain, and a CD3zeta signaling domain. In some embodiments, a CAR comprises a binding domain for GRC5D, a CD28 hinge, a CD28 transmembrane domain, a CD28 costimulatory domain, and CD3zeta signaling domain.

In some embodiments, the antigen is Fe Receptor-like 5 (FcRL5). CARs targeting FcRL5 are described, for example, in US Publication No. US 2017/0275362, which is herein incorporated by reference. In some embodiments, a CAR comprises a binding domain for FcRL5, a CD8a hinge, a CD8a transmembrane domain, a 4-1BB costimulatory domain, and a CD3zeta signaling domain. In some embodiments, a CAR comprises a binding domain for FcRL5, an IgG4 hinge, a CD28 transmembrane domain, a 4-1BB costimulatory domain, and a CD3zeta signaling domain. In some embodiments, a CAR comprises a binding domain for FcRL5, a CD28 hinge, a CD28 transmembrane domain, a CD28 costimulatory domain, and CD3zeta signaling domain.

In some embodiments, the antigen is receptor tyrosine kinase-like orphan receptor 1 (ROR1). CARs targeting ROR1 are described, for example, in US Publication No. 2022/0096651, which is herein incorporated by reference. In some embodiments, a CAR comprises a binding domain for ROR1, a CD8a hinge, a CD8a transmembrane domain, a 4-1BB costimulatory domain, and a CD3zeta signaling domain. In some embodiments, a CAR comprises a binding domain for ROR1, an IgG4 hinge, a CD28 transmembrane domain, a 4-1BB costimulatory domain, and a CD3zeta signaling domain. In some embodiments, a CAR comprises a binding domain for ROR1, a CD28 hinge, a CD28 transmembrane domain, a CD28 costimulatory domain, and CD3zeta signaling domain.

In some embodiments, the CAR is a second-generation CAR comprised an anti-BCMA scFv linked to the 4-1BB costimulatory domain and the CD3zeta intracellular signaling domain. In some embodiments, the CAR is a second-generation CAR comprised an anti-GPRC5D scFv linked to the 4-1BB costimulatory domain and the CD3zeta intracellular signaling domain. In some embodiments, the CAR is a second-generation CAR comprised an anti-ROR1 scFv linked to the 4-1BB costimulatory domain and the CD3zeta intracellular signaling domain.

A skilled artisan is readily familiar with CARs against diverse tumor antigens. Any one of such CARs can be employed as the CAR. Numerous CARs have been approved by the FDA and include, but are not limited to, anti-CD19 and anti-BCMA CAR T cells such as tisagenlecleucel (Kymriah), axicabtagene ciloleucel (Yescarta), brexucabtagene autoleucel (Tecartus), lisocabtagene maraleucel (Breyanzi), or idecabtagene vicleucel (Abecma). It is within the level of a skilled artisan to generate similar constructs for specific targeting of a desired tumor antigen.

In some embodiments, the binding portion of the CAR can be directed to a universal antigen to target a wide variety of tumors without the need to prepare separate CAR constructs. The targeted moiety recognized by the CAR may also remain constant. In some embodiments, a ligand may be administered to the subject to allow interaction with target cells and interaction with the binding portion of the CAR. It is only the ligand portion of the small conjugate molecule that needs to be altered to allow the system to target cancer cells of different identity. Exemplary universal CAR systems are described in the Section above.

In some embodiments, the CAR is an anti-hapten CAR, such as any described in Section IV.A. above. In some embodiments, the anti-hapten CAR can be selectively targeted to a target cell labeled by a small molecule conjugate composed of a hapten and a cell-targeting moiety, such as any described above. In some embodiments, the CAR is an anti-fluorescein/FITC chimeric antigen receptor that can be selectively targeted to a target cell labeled by a small molecule conjugate composed of fluorescein or fluorescein isothiocyanate (FITC) and a cell-targeting moiety. In variations, other haptens recognized by CARs may be used in place of fluorescein/FITC. The CAR may be generated using various scFv sequences known in the art, or scFv sequences generated by conventional and routine methods. Further illustrative scFv sequences for fluorescein/FITC and for other haptens are provided in, for example, WO 2021/076788, the disclosure of which is incorporated by reference herein.

### Targeting Agents for Universal CARs

In some embodiments, the CAR system of the disclosure makes use of CARs that target a moiety that is not produced or expressed by cells of the subject being treated. This CAR system thus allows for focused targeting of the macrophages to target cells, such as cancer cells. By administration of a small conjugate molecule along with the CAR-expressing macrophages, the macrophage cell response can be targeted to only those cells expressing the tumor receptor, thereby reducing off-target toxicity, and the activation of macrophages can be more easily controlled due to the rapid clearance of the small conjugate molecule. As an added advantage, the CAR-expressing macrophages can be used as a “universal” cytotoxic cell to target a wide variety of tumors without the need to prepare separate CAR constructs. The targeted moiety recognized by the CAR may also remain constant. It is only the ligand portion of the small conjugate molecule that needs to be altered to allow the system to target cancer cells of different identity.

Various methods to target CARs and CAR-expressing cells have been described in the art, including, for example in US 2020/0123224, the disclosure of which is incorporated by reference herein. For example, a fluorescein or fluorescein isothiocyanate (FITC) moiety may be conjugated to an agent that binds to a desired target cell (such as a cancer cell), and thereby a CAR-macrophage cell expressing an anti-fluorescein/FITC chimeric antigen receptor may be selectively targeted to the target cell labeled by the conjugate. In variations, other haptens recognized by CARs may be used in place of fluorescein/FITC. The CAR may be generated using various scFv sequences known in the art, or scFv sequences generated by conventional and routine methods. Further illustrative scFv sequences for fluorescein/FITC and for other haptens are provided in, for example, WO 2021/076788, the disclosure of which is incorporated by reference herein.

In one embodiment, the disclosure provides an illustration of this conjugate molecule/CAR system.

In some embodiments, the CAR system of the disclosure utilizes conjugate molecules as the bridge between CAR-expressing cells and targeted cancer cells. The conjugate molecules are conjugates comprising a hapten and a cell-targeting moiety, such as any suitable tumor cell-specific ligand. Illustrative haptens that can be recognized and bound by CARs, include small molecular weight organic molecules such as DNP (2,4-dinitrophenol), TNP (2,4,6-trinitrophenol), biotin, and digoxigenin, along with fluorescein and derivatives thereof, including FITC (fluorescein isothiocyanate), NHS-fluorescein, and pentafluorophenyl ester (PFP) and tetrafluorophenyl ester (TFP) derivatives, a knottin, a centyrin, and a DARPin. Suitable cell-targeting moiety that may themselves act as a hapten for a CAR include knottins (see Kolmar H. et al., The FEBS Journal. 2008. 275(11):26684-90), centyrins, and DARPins (see Reichert, J. M. MAbs 2009. 1(3):190-209).

In some embodiments, the cell-targeting moiety is DUPA (DUPA-(99m) Tc), a ligand bound by PSMA-positive human prostate cancer cells with nanomolar affinity (K_{D}=14 nM; see Kularatne, S. A. et al., Mol Pharm. 2009. 6(3):780-9). In one embodiment, a DUPA derivative can be the ligand of the small molecule ligand linked to a targeting moiety, and DUPA derivatives are described in WO 2015/057852, incorporated herein by reference.

In some embodiments, the cell-targeting moiety is CCK2R ligand, a ligand bound by CCK2R-positive cancer cells (e.g., cancers of the thyroid, lung, pancreas, ovary, brain, stomach, gastrointestinal stroma, and colon; see Wayua. C. et al., Molecular Pharmaceutics. 2013. ePublication).

In some embodiments, the cell-targeting moiety is folate, folic acid, or an analogue thereof, a ligand bound by the folate receptor on cells of cancers that include cancers of the ovary, cervix, endometrium, lung, kidney, brain, breast, colon, and head and neck cancers; see Sega, E. I. et al., Cancer Metastasis Rev. 2008. 27(4):655-64).

In some embodiments, the cell-targeting moiety is an NK-1R ligand. Receptors for NK-1R the ligand are found, for example, on cancers of the colon and pancreas. In some embodiments, the NK-1R ligand may be synthesized according the method disclosed in Int'l Patent Appl. No. PCT/US2015/044229, incorporated herein by reference.

In some embodiments, the cell-targeting moiety may be a peptide ligand, for example, the ligand may be a peptide ligand that is the endogenous ligand for the NK1 receptor. In some embodiments, the small conjugate molecule ligand may be a regulatory peptide that belongs to the family of tachykinins which target tachykinin receptors. Such regulatory peptides include Substance P (SP), neurokinin A (substance K), and neurokinin B (neuromedin K), (see Hennig et al., International Journal of Cancer: 61, 786-792).

In some embodiments, the cell-targeting moiety is a CAIX ligand. Receptors for the CAIX ligand found, for example, on renal, ovarian, vulvar, and breast cancers. The CAIX ligand may also be referred to herein as CA9.

In some embodiments, the cell-targeting moiety is a ligand of gamma glutamyl transpeptidase. The transpeptidase is overexpressed, for example, in ovarian cancer, colon cancer, liver cancer, astrocytic gliomas, melanomas, and leukemias.

In some embodiments, the cell-targeting moiety is a CCK2R ligand. Receptors for the CCK2R ligand found on cancers of the thyroid, lung, pancreas, ovary, brain, stomach, gastrointestinal stroma, and colon, among others.

In one embodiment, the cell-targeting moiety may have a mass of less than about 10,000 Daltons, less than about 9000 Daltons, less than about 8,000 Daltons, less than about 7000 Daltons, less than about 6000 Daltons, less than about 5000 Daltons, less than about 4500 Daltons, less than about 4000 Daltons, less than about 3500 Daltons, less than about 3000 Daltons, less than about 2500 Daltons, less than about 2000 Daltons, less than about 1500 Daltons, less than about 1000 Daltons, or less than about 500 Daltons. In another embodiment, the small molecule ligand may have a mass of about 1 to about 10,000 Daltons, about 1 to about 9000 Daltons, about 1 to about 8,000 Daltons, about 1 to about 7000 Daltons, about 1 to about 6000 Daltons, about 1 to about 5000 Daltons, about 1 to about 4500 Daltons, about 1 to about 4000 Daltons, about 1 to about 3500 Daltons, about 1 to about 3000 Daltons, about 1 to about 2500 Daltons, about 1 to about 2000 Daltons, about 1 to about 1500 Daltons, about 1 to about 1000 Daltons, or about 1 to about 500 Daltons.

In one illustrative embodiment, the linkage in a conjugate described herein can be a direct linkage or the linkage can be through an intermediary linker. In one embodiment, if present, an intermediary linker can be any biocompatible linker known in the art, such as a divalent linker. In one illustrative embodiment, the divalent linker can comprise about 1 to about 30 carbon atoms. In another illustrative embodiment, the divalent linker can comprise about 2 to about 20 carbon atoms. In other embodiments, lower molecular weight divalent linkers (i.e., those having an approximate molecular weight of about 30 to about 300 Da) are employed. In another embodiment, linkers lengths that are suitable include, but are not limited to, linkers having 2, 3, 4, 5, 6, 7, 8, 9. 10, 11, 12, 13, 14, 15, 16, 17, 18, 19, 20, 21, 22, 23, 24, 25, 26, 27, 28, 29, 30, 31, 32, 33, 34, 35, 36, 37. 38, 39 or 40, or more atoms.

In some embodiments, the hapten and the cell-targeting moiety can be directly conjugated through such means as reaction between the isothiocyanate group of FITC and free amine group of small ligands (e.g., folate, DUPA, and CCK2R ligand). However, the use of a linking domain to connect the two molecules may be helpful as it can provide flexibility and stability. Examples of suitable linking domains include: 1) polyethylene glycol (PEG); 2) polyproline; 3) hydrophilic amino acids; 4) sugars; 5) unnatural peptideoglycans; 6) polyvinylpyrrolidone; 7) pluronic F-127. Linker lengths that are suitable include, but are not limited to, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

In some embodiments, the linker may be a divalent linker that may include one or more spacers.

An illustrative conjugate of the disclosure is FITC-Folate

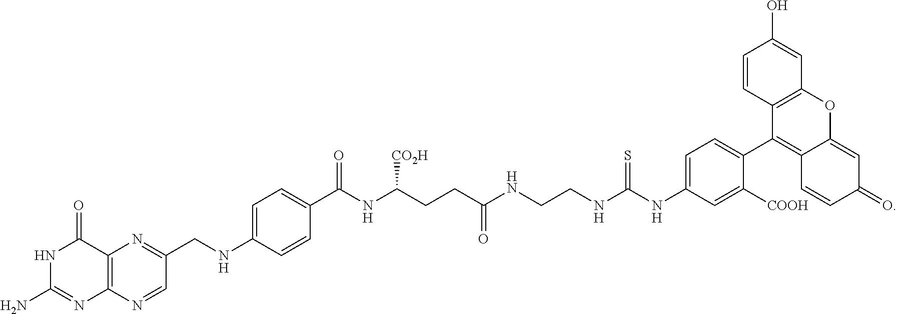 [SMILES: Nc1nc2ncc(CNc3ccc(C(=O)N[C@@H](CCC(=O)NCCNC(=S)Nc4ccc(-c5c6ccc(=O)cc-6oc6cc(O)ccc56)c(C(=O)O)c4)C(=O)O)cc3)nc2c(=O)[nH]1]

An illustrative conjugate of the disclosure is FITC-CA9

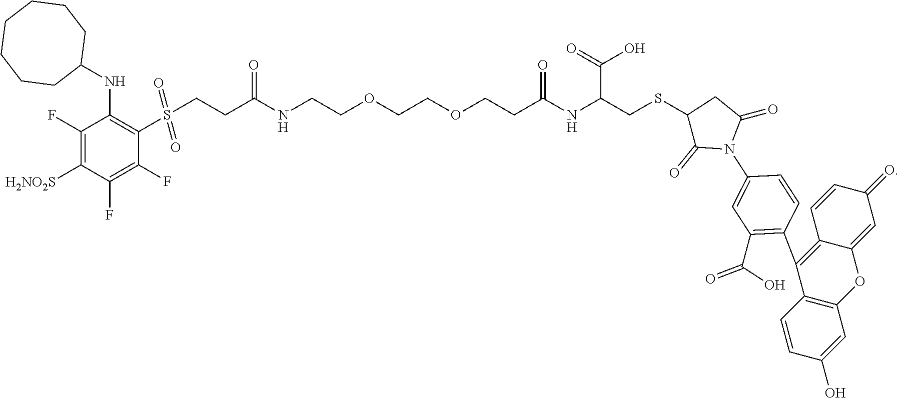 [SMILES: NS(=O)(=O)c1c(F)c(F)c(S(=O)(=O)CCC(=O)NCCOCCOCCC(=O)NC(CSC2CC(=O)N(c3ccc(-c4c5ccc(=O)cc-5oc5cc(O)ccc45)c(C(=O)O)c3)C2=O)C(=O)O)c(NC2CCCCCCC2)c1F]

Illustrative conjugates of the disclosure include the following molecules: FITC-(PEG)₁₂-Folate, FITC-(PEG)₂₀-Folate, FITC-(PEG)₁₀₈-Folate, FITC-DUPA, FITC-(PEG)₁₂-DUPA, FITC-CCK2R ligand, FITC-(PEG)₁₂-CCK2R ligand, FITC-(PEG)₁₁-NK1R ligand and FITC-(PEG)₂-CA9.

While the affinity at which the ligands and cancer cell receptors bind can vary, and in some cases low affinity binding may be preferable (such as about 1 μM), the binding affinity of the ligands and cancer cell receptors will generally be at least about 100 μM, 1 nM, 10 nM, or 100 nM, preferably at least about 1 μM or 10 μM, even more preferably at least about 100 μM.

Examples of conjugates and methods of making them are provided in U.S. patent applications US 2017/0290900, US 2019/0091308, and US 2020/0023009, all of which are incorporated herein by reference.

### B. Co-Stimulatory Domain

In some embodiments, a co-stimulation domain serves to enhance the proliferation and survival of the lymphocytes upon binding of the CAR to a targeted moiety. The identity of the co-stimulation domain is limited only in that it has the ability to enhance cellular proliferation and survival activation upon binding of the targeted moiety by the CAR. Suitable co-stimulation domains include, but are not limited to: CD28 (see, e.g., Alvarez-Vallina, L. et al., Eur J Immunol. 1996. 26(10):2304-9); CD137 (4-1BB), a member of the tumor necrosis factor (TNF) receptor family (see, e.g., Imai, C. et al., Leukemia. 2004. 18:676-84); and CD134 (OX40), a member of the TNFR-superfamily of receptors (see, e.g., Latza, U. et al., Eur. J. Immunol. 1994. 24:677). A skilled artisan will understand that sequence variants of these co-stimulation domains can be used, where the variants have the same or similar activity as the domain on which they are modeled. In various embodiments, such variants have at least about 80%, at least about 90%, at least about 95%, at least about 97%, at least about 98%, at least about 99%, or at least about 99.5% sequence identity to the amino acid sequence of the domain from which they are derived.

In some embodiments of the invention, the CAR constructs comprise two co-stimulation domains. While the particular combinations include all possible variations of the four noted domains, specific examples include: 1) CD28+CD137 (4-1BB) and 2) CD28+CD134 (OX40).

### C. Activation Signaling Domain

In some embodiments, the activation signaling domain serves to activate cells upon binding of the CAR to a targeted moiety. The identity of the activation signaling domain is limited only in that it has the ability to induce activation of the selected cell upon binding of the targeted moiety by the CAR. The skilled artisan will understand that sequence variants of these noted activation signaling domains can be used without adversely impacting the invention, where the variants have the same or similar activity as the domain on which they are modeled. Such variants may have at least about 80%, at least about 90%, at least about 95%, at least about 97%, at least about 98%, at least about 99%, or at least about 99.5% sequence identity to the amino acid sequence of the domain from which they are derived.

### D. Exemplary CARs

In some embodiments, the CAR is an anti-CD19 CAR. Exemplary ligands are described in the Section above. In some embodiments, the ligand is CD19.

An illustrative nucleotide sequence encoding a CAR may comprise SEQ ID NO: 50:

CTGCTGCTGGTGACCTCCCTGCTGCTGTGCGAGCTGCCTCACCCA
GCCTTTCTGCTGATCCCCGACATCCAGATGACACAGACCACAAGC
TCCCTGTCTGCCAGCCTGGGCGACAGAGTGACCATCTCCTGTAGG
GCCTCTCAGGATATCAGCAAGTACCTGAACTGGTATCAGCAGAAG
CCAGATGGCACAGTGAAGCTGCTGATCTACCACACCTCCAGGCTG
CACTCTGGAGTGCCAAGCCGGTTCTCCGGATCTGGAAGCGGCACC
GACTATTCCCTGACAATCTCTAACCTGGAGCAGGAGGATATCGCC
ACATACTTTTGCCAGCAGGGCAATACCCTGCCATATACATTCGGC
GGAGGAACCAAGCTGGAGATCACCGGATCCACATCTGGAAGCGGC
AAGCCAGGAAGCGGAGAGGGATCCACAAAGGGAGAGGTGAAGCTG
CAGGAGAGCGGACCAGGACTGGTGGCACCATCCCAGTCTCTGAGC
GTGACCTGTACAGTGTCCGGCGTGTCTCTGCCTGACTACGGCGTG
TCCTGGATCAGGCAGCCACCTAGGAAGGGACTGGAGTGGCTGGGC
GTGATCTGGGGCTCTGAGACCACATACTATAATTCTGCCCTGAAG
AGCCGCCTGACCATCATCAAGGACAACTCCAAGTCTCAGGTGTTT
CTGAAGATGAATAGCCTGCAGACCGACGATACAGCCATCTACTAT
TGCGCCAAGCACTACTATTACGGCGGCTCCTACGCCATGGATTAT
TGGGGCCAGGGCACCTCCGTGACAGTGTCTAGCGGCGCTGTGCAC
ACCAGAGGACTGGATTTCGCCTGCGACTTCTGGGTGCTGGTGGTG
GTGGGAGGCGTGCTGGCCTGTTACTCCCTGCTGGTGACCGTGGCC
TTTATCATCTTCTGGGTGAAGAGAGGCAGGAAGAAGCTGCTGTAT
ATCTTTAAGCAGCCCTTCATGCGCCCTGTGCAGACCACACAGGAG
GAGGACGGCTGCAGCTGTCGGTTTCCAGAGGAGGAGGAGGGAGGA
TGCGAGCTGCGCGTGAAGTTCAGCCGGTCCGCCGATGCCCCTGCC
TACCAGCAGGGCCAGAACCAGCTGTATAACGAGCTGAATCTGGGC
CGGAGAGAGGAGTACGACGTGCTGGATAAGAGGAGGGGAAGGGAC
CCAGAGATGGGAGGCAAGCCTCGGAGAAAGAACCCACAGGAGGGC
CTGTACAATGAGCTGCAGAAGGACAAGATGGCCGAGGCCTATTCT
GAGATCGGCATGAAGGGAGAGAGGCGCCGGGGCAAGGGACACGAT
GGCCTGTACCAGGGCCTGAGCACCGCCACAAAGGACACATATGAT
GCCCTGCACATGCAGGCCCTGCCACCTAGG.

An illustrative amino acid sequence encoding a CAR may comprise SEQ ID NO: 51:

LLLVTSLLLCELPHPAFLLIPDIQMTQTTSSLSASLGDRVTISCR
ASQDISKYLNWYQQKPDGTVKLLIYHTSRLHSGVPSRFSGSGSGT
DYSLTISNLEQEDIATYFCQQGNTLPYTFGGGTKLEITGSTSGSG
KPGSGEGSTKGEVKLQESGPGLVAPSQSLSVTCTVSGVSLPDYGV
SWIRQPPRKGLEWLGVIWGSETTYYNSALKSRLTIIKDNSKSQVF
LKMNSLQTDDTAIYYCAKHYYYGGSYAMDYWGQGTSVTVSSGAVH
TRGLDFACDFWVLVVVGGVLACYSLLVTVAFIIFWVKRGRKKLLY
IFKQPFMRPVQTTQEEDGCSCRFPEEEEGGCELRVKFSRSADAPA
YQQGQNQLYNELNLGRREEYDVLDKRRGRDPEMGGKPRRKNPQEG
LYNELQKDKMAEAYSEIGMKGERRRGKGHDGLYQGLSTATKDTYD
ALHMQALPPR.

An illustrative nucleotide sequence encoding a CAR may comprise SEQ ID NO: 13:

(SEQ ID NO: 13)
ATGGCCTTACCAGTGACCGCCTTGCTCCTGCCGCTGGCCTTGCTG
CTCCACGCCGCCAGGCCGGATGTCGTGATGACCCAGACCCCCCTC
AGCCTCCCAGTGTCCCTCGGTGACCAGGCTTCTATTAGTTGCAGA
TCCAGCCAGTCCCTCGTGCACTCTAACGGTAATACCTACCTGAGA
TGGTATCTCCAGAAGCCCGGACAGAGCCCTAAGGTGCTGATCTAC
AAAGTCTCCAACCGGGTGTCTGGAGTCCCTGACCGCTTCTCAGGG
AGCGGTTCCGGCACCGACTTCACCCTGAAGATCAACCGGGTGGAG
GCCGAAGACCTCGGCGTCTATTTCTGCTCTCAGAGTACACATGTG
CCCTGGACCTTCGGCGGAGGGACCAAGCTGGAGATCAAAAGCTCC
GCAGACGATGCCAAGAAAGATGCCGCTAAGAAAGACGATGCTAAG
AAAGACGATGCAAAGAAAGACGGTGGCGTGAAGCTGGATGAAACC
GGAGGAGGTCTCGTCCAGCCAGGAGGAGCCATGAAGCTGAGTTGC
GTGACCAGCGGATTCACCTTTGGGCACTACTGGATGAACTGGGTG
CGACAGTCCCCAGAGAAGGGGCTCGAATGGGTCGCTCAGTTCAGG
AACAAACCCTACAATTATGAGACATACTATTCAGACAGCGTGAAG
GGCAGGTTTACTATCAGTAGAGACGATTCCAAATCTAGCGTGTAC
CTGCAGATGAACAATCTCAGGGTCGAAGATACAGGCATCTACTAT
TGCACAGGGGCATCCTATGGTATGGAGTATCTCGGTCAGGGGACA
AGCGTCACAGTCAGTTTCGTGCCGGTCTTCCTGCCAGCGAAGCCC
ACCACGACGCCAGCGCCGCGACCACCAACACCGGCGCCCACCATC
GCGTCGCAGCCCCTGTCCCTGCGCCCAGAGGCGTGCCGGCCAGCG
GCGGGGGGCGCAGTGCACACGAGGGGGCTGGACTTCGCCTGTGAT
ATCTACATCTGGGCGCCCTTGGCCGGGACTTGTGGGGTCCTTCTC
CTGTCACTGGTTATCACCCTTTACTGCAACCACAGGAACCGTTTC
TCTGTTGTTAAACGGGGCAGAAAGAAACTCCTGTATATATTCAAA
CAACCATTTATGAGACCAGTACAAACTACTCAAGAGGAAGATGGC
TGTAGCTGCCGATTTCCAGAAGAAGAAGAAGGAGGATGTGAACTG
AGAGTGAAGTTCAGCAGGAGCGCAGACGCCCCCGCGTACCAGCAG
GGCCAGAACCAGCTCTATAACGAGCTCAATCTAGGACGAAGAGAG
GAGTACGATGTTTTGGACAAGAGACGTGGCCGGGACCCTGAGATG
GGGGGAAAGCCGAGAAGGAAGAACCCTCAGGAAGGCCTGTACAAT
GAACTGCAGAAAGATAAGATGGCGGAGGCCTACAGTGAGATTGGG
ATGAAAGGCGAGCGCCGGAGGGGCAAGGGGCACGATGGCCTTTAC
CAGGGTCTCAGTACAGCCACCAAGGACACCTACGACGCCCTTCAC
ATGCAGGCCCTGCCCCCTCGCTAA

An illustrative CAR amino acid sequence may comprise SEQ ID NO: 14:

(SEQ ID NO: 14)
MALPVTALLLPLALLLHAARPDVVMTQTPLSLPVSLGDQASISCR
SSQSLVHSNGNTYLRWYLQKPGQSPKVLIYKVSNRVSGVPDRFSG
SGSGTDFTLKINRVEAEDLGVYFCSQSTHVPWTFGGGTKLEIKSS
ADDAKKDAAKKDDAKKDDAKKDGGVKLDETGGGLVQPGGAMKLSC
VTSGFTFGHYWMNWVRQSPEKGLEWVAQFRNKPYNYETYYSDSVK
GRFTISRDDSKSSVYLQMNNLRVEDTGIYYCTGASYGMEYLGQGT
SVTVSFVPVFLPAKPTTTPAPRPPTPAPTIASQPLSLRPEACRPA
AGGAVHTRGLDFACDIYIWAPLAGTCGVLLLSLVITLYCNHRNRF
SVVKRGRKKLLYIFKQPFMRPVQTTQEEDGCSCRFPEEEEGGCEL
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR

An illustrative nucleotide insert may comprise SEQ ID NO: 15:

(SEQ ID NO: 15)
GCCACCATGGCCTTACCAGTGACCGCCTTGCTCCTGCCGCTGGCC
TTGCTGCTCCACGCCGCCAGGCCGGATGTCGTGATGACCCAGACC
CCCCTCAGCCTCCCAGTGTCCCTCGGTGACCAGGCTTCTATTAGT
TGCAGATCCAGCCAGTCCCTCGTGCACTCTAACGGTAATACCTAC
CTGAGATGGTATCTCCAGAAGCCCGGACAGAGCCCTAAGGTGCTG
ATCTACAAAGTCTCCAACCGGGTGTCTGGAGTCCCTGACCGCTTC
TCAGGGAGCGGTTCCGGCACCGACTTCACCCTGAAGATCAACCGG
GTGGAGGCCGAAGACCTCGGCGTCTATTTCTGCTCTCAGAGTACA
CATGTGCCCTGGACCTTCGGCGGAGGGACCAAGCTGGAGATCAAA
AGCTCCGCAGACGATGCCAAGAAAGATGCCGCTAAGAAAGACGAT
GCTAAGAAAGACGATGCAAAGAAAGACGGTGGCGTGAAGCTGGAT
GAAACCGGAGGAGGTCTCGTCCAGCCAGGAGGAGCCATGAAGCTG
AGTTGCGTGACCAGCGGATTCACCTTTGGGCACTACTGGATGAAC
TGGGTGCGACAGTCCCCAGAGAAGGGGCTCGAATGGGTCGCTCAG
TTCAGGAACAAACCCTACAATTATGAGACATACTATTCAGACAGC
GTGAAGGGCAGGTTTACTATCAGTAGAGACGATTCCAAATCTAGC
GTGTACCTGCAGATGAACAATCTCAGGGTCGAAGATACAGGCATC
TACTATTGCACAGGGGCATCCTATGGTATGGAGTATCTCGGTCAG
GGGACAAGCGTCACAGTCAGTTTCGTGCCGGTCTTCCTGCCAGCG
AAGCCCACCACGACGCCAGCGCCGCGACCACCAACACCGGCGCCC
ACCATCGCGTCGCAGCCCCTGTCCCTGCGCCCAGAGGCGTGCCGG
CCAGCGGCGGGGGGCGCAGTGCACACGAGGGGGCTGGACTTCGCC
TGTGATATCTACATCTGGGCGCCCTTGGCCGGGACTTGTGGGGTC
CTTCTCCTGTCACTGGTTATCACCCTTTACTGCAACCACAGGAAC
CGTTTCTCTGTTGTTAAACGGGGCAGAAAGAAACTCCTGTATATA
TTCAAACAACCATTTATGAGACCAGTACAAACTACTCAAGAGGAA
GATGGCTGTAGCTGCCGATTTCCAGAAGAAGAAGAAGGAGGATGT
GAACTGAGAGTGAAGTTCAGCAGGAGCGCAGACGCCCCCGCGTAC
CAGCAGGGCCAGAACCAGCTCTATAACGAGCTCAATCTAGGACGA
AGAGAGGAGTACGATGTTTTGGACAAGAGACGTGGCCGGGACCCT
GAGATGGGGGGAAAGCCGAGAAGGAAGAACCCTCAGGAAGGCCTG
TACAATGAACTGCAGAAAGATAAGATGGCGGAGGCCTACAGTGAG
ATTGGGATGAAAGGCGAGCGCCGGAGGGGCAAGGGGCACGATGGC
CTTTACCAGGGTCTCAGTACAGCCACCAAGGACACCTACGACGCC
CTTCACATGCAGGCCCTGCCCCCTCGCTAA

In some embodiments, the CAR may be encoded by a nucleic acid sequence that encodes a signal peptide to signal transport of the CAR in the cell. It is understood that typically the signal peptide is removed from the protein.

An illustrative CAR amino acid sequence without a signal peptide may comprise SEQ ID NO: 16:

(SEQ ID NO: 16)
DVVMTQTPLSLPVSLGDQASISCRSSQSLVHSNGNTYLRWYLQKP
GQSPKVLIYKVSNRVSGVPDRFSGSGSGTDFTLKINRVEAEDLGV
YFCSQSTHVPWTFGGGTKLEIKSSADDAKKDAAKKDDAKKDDAKK
DGGVKLDETGGGLVQPGGAMKLSCVTSGFTFGHYWMNWVRQSPEK
GLEWVAQFRNKPYNYETYYSDSVKGRFTISRDDSKSSVYLQMNNL
RVEDTGIYYCTGASYGMEYLGQGTSVTVSFVPVFLPAKPTTTPAP
RPPTPAPTIASQPLSLRPEACRPAAGGAVHTRGLDFACDIYIWAP
LAGTCGVLLLSLVITLYCNHRNRFSVVKRGRKKLLYIFKQPFMRP
VQTTQEEDGCSCRFPEEEEGGCELRVKFSRSADAPAYQQGQNQLY
NELNLGRREEYDVLDKRRGRDPEMGGKPRRKNPQEGLYNELQKDK
MAEAYSEIGMKGERRRGKGHDGLYQGLSTATKDTYDALHMQALPP
R

An illustrative CAR amino acid sequence signal peptide may comprise SEQ ID NO: 17:

(SEQ ID NO: 17)
MALPVTALLLPLALLLHAARP

In various embodiments, CAR-expressing cells comprising the nucleic acid of SEQ ID NO: 13 or 15 are provided. In some embodiments, a chimeric antigen receptor polypeptide comprising SEQ ID NO: 14 is contemplated. In some embodiments, a chimeric antigen receptor polypeptide comprising SEQ ID NO: 16 is contemplated. In some embodiments, a vector is contemplated comprising SEQ ID NO: 13 or 15. In some embodiments, a lentiviral vector is contemplated comprising SEQ ID NO: 13 or 15. In some embodiments, SEQ ID NO: 14 can comprise or consist of human or humanized amino acid sequences. In some embodiments, SEQ ID NO: 16 can comprise or consist of human or humanized amino acid sequences.

In some embodiments, variant nucleic acid sequences or amino acid sequences having at least about 80%, at least about 90%, at least about 95%, at least about 97%, at least about 98%, at least about 99%, or at least about 99.5% sequence identity to SEQ ID NO: 13, SEQ ID NO: 14, SEQ ID NO: 15 or SEQ ID NO: 16 are contemplated.

In various embodiments, CAR-expressing cells comprising the nucleic acid of SEQ ID NO: 50 are provided. In some embodiments, SEQ ID NO: 51 can comprise or consist of human or humanized amino acid sequences.

In some embodiments, nucleic acid sequences or amino acid sequences encoding the CAR having at least about 80%, at least about 90%, at least about 95%, at least about 97%, at least about 98%, at least about 99%, or at least about 99.5% sequence identity to SEQ ID NO:50 and 51, respectively.

While the affinity at which the CARs, expressed by the lymphocytes, bind to the targeted moiety can vary, and in some cases low affinity binding may be preferable (such as about 50 nM), the binding affinity of the CARs to the targeted ligand will generally be at least about 100 nM, 1 μM, or 10 μM, preferably at least about 100 μM, 1 fM or 10 fM, even more preferably at least about 100 fM.

### V. NUCLEIC ACID VECTORS

As used herein, the term “nucleic acid vector” is intended to mean any nucleic acid that functions to carry, harbor or express a nucleic acid of interest. Nucleic acid vectors can have specialized functions such as expression, packaging, pseudotyping, transduction or sequencing, for example. Nucleic acid vectors also can have, for example, manipulatory functions such as a cloning or shuttle vector. The structure of the vector can include any desired form that is feasible to make and desirable for a particular use. Such forms include, for example, circular forms such as plasmids and phagemids, as well as linear or branched forms. A nucleic acid vector can be composed of, for example, DNA or RNA, as well as contain partially or fully, nucleotide derivatives, analogs and mimetics. Such nucleic acid vectors can be obtained from natural sources, produced recombinantly or chemically synthesized.

Non-limiting examples of vector systems of the present disclosure include a retrovirus, a lentivius, a foamy virus, and a Sleeping Beauty transposon.

### Retroviral Particles

Retroviruses include lentiviruses, gamma-retroviruses, and alpha-retroviruses, each of which may be used to deliver polynucleotides to cells using methods known in the art. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV-1 and HIV-2) and the Simian Immunodeficiency Virus (SIV). Retroviral vectors have been generated by multiply attenuating the HIV virulence genes, for example, the genes env, vif, vpr, vpu and nef are deleted, making the vector biologically safe.

Illustrative lentiviral vectors include those described in Naldini et al. (1996) Science 272:263-7; Zufferey et al. (1998) J. Virol. 72:9873-9880; Dull et al. (1998) J. Virol. 72:8463-8471; U.S. Pat. Nos. 6,013,516; and 5,994,136, which are each incorporated herein by reference in their entireties. In general, these vectors are configured to carry the essential sequences for selection of cells containing the vector, for incorporating foreign nucleic acid into a lentiviral particle, and for transfer of the nucleic acid into a target cell.

A commonly used lentiviral vector system is the so-called third-generation system. Third-generation lentiviral vector systems include four plasmids. The “transfer plasmid” encodes the polynucleotide sequence that is delivered by the lentiviral vector system to the target cell. The transfer plasmid generally has one or more transgene sequences of interest flanked by long terminal repeat (LTR) sequences, which facilitate integration of the transfer plasmid sequences into the host genome. For safety reasons, transfer plasmids are generally designed to make the resulting vector replication incompetent. For example, the transfer plasmid lacks gene elements necessary for generation of infective particles in the host cell. In addition, the transfer plasmid may be designed with a deletion of the 3′ LTR, rendering the virus “self-inactivating” (SIN). See Dull et al. (1998) J. Virol. 72:8463-71; Miyoshi et al. (1998) J. Virol. 72:8150-57. The viral particle may also comprise a 3′ untranslated region (UTR) and a 5′ UTR. The UTRs comprise retroviral regulatory elements that support packaging, reverse transcription and integration of a proviral genome into a cell following contact of the cell by the retroviral particle.

Third-generation systems also generally include two “packaging plasmids” and an “envelope plasmid.” The “envelope plasmid” generally encodes an Env gene operatively linked to a promoter. In an illustrative third-generation system, the Env gene is VSV-G and the promoter is the CMV promoter. The third-generation system uses two packaging plasmids, one encoding gag and pol and the other encoding rev as a further safety feature; an improvement over the single packaging plasmid of so-called second-generation systems. Although safer, the third-generation system can be more cumbersome to use and result in lower viral titers due to the addition of an additional plasmid. Illustrative packing plasmids include, without limitation, pMD2.G, pRSV-rev, pMDLG-pRRE, and pRRL-GOI.

Many retroviral vector systems rely on the use of a “packaging cell line.” In general, the packaging cell line is a cell line whose cells are capable of producing infectious retroviral particles when the transfer plasmid, packaging plasmid(s), and envelope plasmid are introduced into the cells. Various methods of introducing the plasmids into the cells may be used, including transfection or electroporation. In some cases, a packaging cell line is adapted for high-efficiency packaging of a retroviral vector system into retroviral particles.

As used herein, the terms “retroviral vector” or “lentiviral vector” is intended to mean a nucleic acid that encodes a retroviral or lentiviral cis nucleic acid sequence required for genome packaging and one or more polynucleotide sequence to be delivered into the target cell. Retroviral particles and lentiviral particles generally include an RNA genome (derived from the transfer plasmid), a lipid-bilayer envelope in which the Env protein is embedded, and other accessory proteins including integrase, protease, and matrix protein. As used herein, the terms “retroviral particle” and “lentiviral particle” refers a viral particle that includes an envelope, has one or more characteristics of a lentivirus, and is capable of invading a target host cell. Such characteristics include, for example, infecting non-dividing host cells, transducing non-dividing host cells, infecting or transducing host immune cells, containing a retroviral or lentiviral virion including one or more of the gag structural polypeptides, containing a retroviral or lentiviral envelope including one or more of the env encoded glycoproteins, containing a genome including one or more retrovirus or lentivirus cis-acting sequences functioning in replication, proviral integration or transcription, containing a genome encoding a retroviral or lentiviral protease, reverse transcriptase or integrase, or containing a genome encoding regulatory activities such as Tat or Rev. The transfer plasmids may comprise a cPPT sequence, as described in U.S. Pat. No. 8,093,042.

The efficiency of the system is an important concern in vector engineering. The efficiency of a retroviral or lentiviral vector system may be assessed in various ways known in the art, including measurement of vector copy number (VCN) or vector genomes (vg) such as by quantitative polymerase chain reaction (qPCR), or titer of the virus in infectious units per milliliter (IU/mL). For example, the titer may be assessed using a functional assay performed on the cultured tumor cell line HT1080 as described in Humbert et al. Development of third-generation Cocal Envelope Producer Cell Lines for Robust Retroviral Gene Transfer into Hematopoietic Stem Cells and T-cells. Molecular Therapy 24:1237-1246 (2016). When titer is assessed on a cultured cell line that is continually dividing, no stimulation is required and hence the measured titer is not influenced by surface engineering of the retroviral particle. Other methods for assessing the efficiency of retroviral vector systems are provided in Gaererts et al. Comparison of retroviral vector titration methods. BMC Biotechnol. 6:34 (2006).

In some embodiments, the retroviral particles and/or lentiviral particles of the disclosure comprise a polynucleotide comprising a sequence encoding a receptor that specifically binds to the gating adaptor. In some embodiments, a sequence encoding a receptor that specifically binds to the gating adaptor is operatively linked to a promoter. Illustrative promoters include, without limitation, a cytomegalovirus (CMV) promoter, a CAG promoter, an SV40 promoter, an SV40/CD43 promoter, and a MND promoter.

In some embodiments, the retroviral particles comprise transduction enhancers. In some embodiments, the retroviral particles comprise tagging proteins.

In some embodiments, each of the retroviral particles comprises a polynucleotide comprising, in 5′ to 3′ order: (i) a 5′ long terminal repeat (LTR) or untranslated region (UTR), (ii) a promoter, (iii) a sequence encoding a receptor that specifically binds to a ligand, and (iv) a 3′ LTR or UTR.

In some embodiments, the retroviral particles comprise a cell surface receptor that binds to a surface marker on a target host cell, allowing host cell transduction. The viral vector may comprise a heterologous viral envelope glycoprotein giving a pseudotyped viral vector. For example, the viral envelope glycoprotein may be derived from RD 114 or one of its variants, VSV-G, Gibbon-ape leukaemia virus (GALV), or is the Amphotropic envelope, Measles envelope or baboon retroviral envelope glycoprotein. In some embodiments, the cell-surface receptor is a VSV G protein from the Cocal strain or a functional variant thereof.

Various fusion glycoproteins can be used to pseudotype lentiviral vectors. While the most commonly used example is the envelope glycoprotein from vesicular stomatitis virus (VSVG), many other viral proteins have also been used for pseudotyping of lentiviral vectors. See Joglekar et al. Human Gene Therapy Methods 28:291-301 (2017). The present disclosure contemplates substitution of various fusion glycoproteins. Notably, some fusion glycoproteins result in higher vector efficiency.

In some embodiments, pseudotyping a fusion glycoprotein or functional variant thereof facilitates targeted transduction of specific cell types, including, but not limited to, innate lymphoid cells, cytotoxic innate lymphoid cells, or NK cells. In some embodiments, the fusion glycoprotein or functional variant thereof is/are full-length polypeptide(s), functional fragment(s), homolog(s), or functional variant(s) of Human immunodeficiency virus (HIV) gp160, Murine leukemia virus (MLV) gp70, Gibbon ape leukemia virus (GALV) gp70, Feline leukemia virus (RD 114) gp70, Amphotropic retrovirus (Ampho) gp70, 10A1 MLV (10A1) gp70, Ecotropic retrovirus (Eco) gp70, Baboon ape leukemia virus (BaEV) gp70, Measles virus (MV) H and F, Nipah virus (NiV) H and F, Rabies virus (RabV) G, Mokola virus (MOKV) G, Ebola Zaire virus (EboZ) G, Lymphocytic choriomeningitis virus (LCMV) GP1 and GP2, Baculovirus GP64, Chikungunya virus (CHIKV) E1 and E2, Ross River virus (RRV) E1 and E2, Semliki Forest virus (SFV) E1 and E2, Sindbis virus (SV) E1 and E2, Venezualan equine encephalitis virus (VEEV) E1 and E2, Western equine encephalitis virus (WEEV) E1 and E2, Influenza A, B, C, or D HA, Fowl Plague Virus (FPV) HA, Vesicular stomatitis virus VSV-G, or Chandipura virus and Piry virus CNV-G and PRV-G.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

The disclosure further provides various retroviral vectors, including but not limited to gamma-retroviral vectors, alpha-retroviral vectors, and lentiviral vectors. In some embodiments, the vector may be a viral vector, a retroviral vector, a lentiviral vector, a gamma-retroviral vector. In some embodiments, the viral vector comprises a VSV G-protein or functional variant thereof. In some embodiments, the viral vector comprises a Cocal G-protein or functional variant thereof.

### VI. METHODS OF TREATMENT

The present disclosure provides methods of treating a subject in need thereof with the compositions, therapeutic compositions, cells, vectors, and polynucleotides disclosed herein. In some embodiments, the disclosure provides a method of treating cancer and/or killing cancer cells in a subject, comprising administering a therapeutically effective amount of the disclosed cells to the subject. Also provided is a method of treating a tumor and/or killing tumor cells in a subject, comprising administering an effective amount of non-physiological ligand to the subject, wherein the non-physiological ligand causes the cells to proliferate according to any of the foregoing embodiments.

Also provided are uses and compositions for use in in any of the provided methods. The methods and uses may include use of any composition as described herein, including those produced by methods herein or a pharmaceutical composition provided herein, such as described below. In some embodiments, provided herein is a method of treating a condition in an individual, comprising administering any of the provided engineered myeloid cells, e.g., iMACs, to an individual in need thereof. Uses include uses of the cells or pharmaceutical compositions thereof in such methods and treatments, and in the preparation of a medicament in order to carry out such therapeutic methods.

Such methods and uses include therapeutic methods and uses, for example, involving administration of the therapeutic cells, or compositions containing the same, to a subject having a disease, condition, or disorder. In some cases, the disease or disorder is a tumor or cancer. In some embodiments, the disease or disorder is a virus infection. In some embodiments, the cells or pharmaceutical composition thereof is administered in an effective amount to effect treatment of the disease or disorder. In some embodiments, the methods thereby treat the disease or condition or disorder in the subject.

Accumulating evidence suggests that phagocytotic cells are abundant in the tumor microenvironment of numerous cancers. Macrophages, for example, in the tumor microenvironment can adopt a classically activated (M1, antitumor) or an alternatively activated (M2, pro-tumor) phenotype. Macrophages and neutrophils are potent effectors of the innate immune system and are capable of at least three distinct anti-tumor functions: phagocytosis, cellular cytotoxicity, and antigen presentation to orchestrate an adaptive immune response. While T cells require antigen-dependent activation via the T cell receptor or the chimeric immunoreceptor, phagocytotic cells can be activated in a variety of ways. Direct macrophage activation is antigen-independent, relying on mechanisms such as pathogen associated molecular pattern recognition by Toll-like receptors (TLRs). Immune-complex mediated activation is antigen dependent but requires the presence of antigen-specific antibodies and absence of the inhibitory CD47-SIRPa interaction.

Compared with T cells, the macrophage has the advantages of being easier to enter the solid tumor and less likely to be inhibited by other types of cells, and therefore can play a better role in tumor immunotherapy. Since the expressed chimeric antigen receptor is located on the surface of the macrophage, the macrophage can accurately target tumor cells.

The present invention includes compositions and methods for treating a disease or disorder in a subject. The invention includes expression of a chimeric antigen receptor in myeloid cells, such as a monocyte, macrophage or neutrophil. Such a modified cell is recruited to the tumor microenvironment where it acts as a potent immune effector by infiltrating the tumor and killing target cells.

Some approaches to cancer immunotherapy include the use of cytokines or chemokines to recruit activated macrophages and other immune cells (e.g., neutrophils) to the tumor site which allow for recognition and targeted destruction of the tumor site. IFN-αα and IFN-β have been shown to inhibit tumor progression by inducing cell differentiation and apoptosis.

Among cells in the compositions or for use in the methods or uses herein are engineered iMAC cells that comprise a heterologous nucleic acid encoding an antigen receptor (e.g. CAR). In some embodiments, the CAR is able to target an antigen expressed by a cell associated with a disease or condition, such as a tumor cell. Targeting to the antigen directs the iMAC to the cell to trigger target cell death by phagocytosis or cytotoxic killing, thereby treating the disease or condition. In some embodiments, the provided methods can be used to treat any disease or disorder in which targeted cell killing mediates a treatment of the disease or condition. For instance, in the case of a CAR, the disease or condition to be treated is any disease or condition that is associated with expression of an antigen that is recognized or targeted by the CAR. In some embodiments as described, the CAR may be a “universal” CAR that can be targeted to a target cell by labeling with a separately administered conjugate molecule that acts as a bridge between the CAR-expressing cells and targeted cancer cells. In some embodiments, the conjugate molecules are conjugates comprising a hapten and a cell-targeting moiety, such as any suitable tumor cell-specific ligand.

In some embodiments, provided is a method of administering an effective amount of non-physiological ligand (e.g. rapamycin or rapalog) to the subject, wherein the non-physiological ligand causes the cells to proliferate according to any of the foregoing embodiments. In some embodiments, the non-physiological ligand is administered in combination with the macrophages. The non-physiological ligand can be administered concurrently or sequentially with administration of macrophages to the subject. In some embodiments, the non-physiological ligand is first administered before administration of the macrophages or concurrently with administration of the macrophages. In some embodiments, the non-physiological ligand is administration after administration of the macrophages. In some embodiments, the non-physiological ligand may be administered intermittently or at various intervals, such as for a period of time after administration of macrophages, such as CAR-macrophages, to a subject.

In some embodiments, the non-physiological ligand is present or provided at an amount effective to cause expansion of the macrophages ex vivo. In some embodiments, the non-physiological ligand is present or provided at an amount effective to cause expansion of the macrophages in vivo. In some embodiments, the non-physiological ligand is present or provided at an amount effective to cause macrophage cytokine secretion and/or phagocytosis ex vivo. In some embodiments, the non-physiological ligand is present or provided at an amount effective cause macrophage cytokine secretion and/or phagocytosis in vivo. In some embodiments, the non-physiological ligand is present or provided at an amount effective to cause macrophage secretion of tumor necrosis factor alpha (TNF-α) and/or nitric acid (NO) ex vivo. In some embodiments, the non-physiological ligand is present or provided at an amount effective to cause macrophage secretion of tumor necrosis factor alpha (TNF-α) and/or nitric acid (NO) in vivo. In some embodiments, the non-physiological ligand is present or provided at an amount effective to cause tumor cell killing.

In some embodiments, the non-physiological ligand is present or provided at a therapeutically effective amount.

In some embodiments, the non-physiological ligand is rapamycin or a rapamycin analog. In some embodiments, the rapamycin analog is rapalog.

In some embodiments, the non-physiological ligand (e.g. rapamycin or an analog) is administered at a dose of 1 mg to 100 mg, optionally between 10-100 mg, optionally at or about 10 mg, 20 mg, 25 mg, 30 mg, 40 mg, 50 mg or any value between any of the foregoing.

In some embodiments, the malignancy is a solid tumor, sarcoma, carcinoma, lymphoma, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), chronic or acute leukemia, acute myeloid leukemia, chronic myeloid leukemia, acute lymphoblastic leukemia (ALL) (including non T-cell ALL), chronic lymphocytic leukemia (CLL), T-cell lymphoma, one or more of B-cell acute lymphoid leukemia (“BALL”), T-cell acute lymphoid leukemia (“TALL”), acute lymphoid leukemia (ALL), chronic myelogenous leukemia (CML), B cell prolymphocytic leukemia, blastic plasmacytoid dendritic cell neoplasm, Burkitf's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, myelodysplasia and myelodysplastic syndrome, plasmablastic lymphoma, plasmacytoid dendritic cell neoplasm, Waldenstrom macroglobulinemia, a plasma cell proliferative disorder (e.g., asymptomatic myeloma (smoldering multiple myeloma or indolent myeloma)), monoclonal gammapathy of undetermined significance (MGUS), plasmacytomas (e.g., plasma cell dyscrasia, solitary myeloma, solitary plas acyto a, extramedullary plasmacytoma, and multiple plasmacytoma), systemic amyloid light chain amyloidosis, POEMS syndrome (also known as Crow-Fukase syndrome, Takatsuki disease, and PEP syndrome), or a combination thereof.

In some embodiments, a method disclosed herein may be used to treat cancer and/or kill cancer cells in a subject by administering a therapeutically effective amount of the cells according to any of the foregoing embodiments.

The present disclosure also provides a method of treating cancer and/or killing cancer cells in a subject, comprising administering the system of any of the foregoing embodiments to the subject.

In some embodiments, the present disclosure provides a method of treating cancer with any of the compositions provided herein. “Cancer” has its plain and ordinary meaning when read in light of the specification, and may include but is not limited to, for example, a group of diseases involving abnormal cell growth with the potential to invade or spread to other parts of the body. Subjects that can be addressed using the methods described herein include subjects identified or selected as having cancer, including but not limited to colon, lung, liver, breast, renal, prostate, ovarian, skin (including melanoma), bone, and brain cancer, etc. Such identification and/or selection can be made by clinical or diagnostic evaluation. In some embodiments, the tumor associated antigens or molecules are known, such as melanoma, breast cancer, brain cancer, squamous cell carcinoma, colon cancer, leukemia, myeloma, and/or prostate cancer. Examples include but are not limited to B cell lymphoma, breast cancer, brain cancer, prostate cancer, and/or leukemia. In some embodiments, one or more oncogenic polypeptides are associated with kidney, uterine, colon, lung, liver, breast, renal, prostate, ovarian, skin (including melanoma), bone, brain cancer, adenocarcinoma, pancreatic cancer, chronic myelogenous leukemia or leukemia. In some embodiments, a method of treating, ameliorating, or inhibiting a cancer in a subject is provided. In some embodiments, the cancer is breast, ovarian, lung, pancreatic, prostate, melanoma, renal, pancreatic, glioblastoma, neuroblastoma, medulloblastoma, sarcoma, liver, colon, skin (including melanoma), bone or brain cancer.

In some embodiments, the target cell is a tumor cell. In some embodiments, the target cell exists in a tumor microenvironment.

In some embodiments, the present disclosure provides a method of treating cancer with any of the compositions provided herein without prior conditioning of the subject. In some embodiments, a subject of the present disclosure does not need to receive a lymphodepleting therapy. A person skilled in the art will understand the common lymphodepleting therapies available, such as chemotherapy. In some embodiments, a subject of the present disclosure has not received a lymphodepleting therapy. In some embodiments, a differentiated cell is provided to a subject that has not received a lymphodepleting therapy. In some embodiments, the subject has not received a lymphodepleting therapy for 1, 2, 3, 4, 5, 6, 7, 8, 9 or 10 days prior to administration of the differentiated cell.

In some embodiments, a differentiated cell is provided to the subject 1, 2, 3, 4, 5, 6, 7, 8, 9, 10, 11, 12, 15, 20, 24, 36, 48, 60 or 72 hours after administration of a ligand composition, or any time within a range defined by any two aforementioned values. In some embodiments, the cell is provided to the subject 1, 2, 3, 4, 5, 6, 7, 8, 9, 10, 11, 12, 15, 20, 24, 36 or 48 hours before administration of the ligand composition, or any time within a range defined by any two aforementioned values. In some embodiments, the cell is provided to the subject within seconds or minutes, such as less than an hour, of providing the composition to the subject. In some embodiments, a boost of the cell and/or the composition is provided to the subject.

In some aspects, the present disclosure provides a method of treating a cancer as described in WO 2019/144095, which is incorporated herein by reference in its entirety.

In some embodiments, the present disclosure provides a method of treating cancer comprising administering a chimeric antigen receptor cell (e.g., a macrophage), wherein the CAR comprises an E2 anti-fluorescein antibody fragment. In some embodiments, the method of treating cancer further comprises administering a small molecule linked to a targeting moiety by a linker.

In some embodiments, the targeting moiety is determined by the type of cancer being treated in the subject. As an example, folate receptor is highly expressed on the surface of a wide variety of solid tumor cells including breast (e.g., triple negative breast cancer), ovarian, endometrial, kidney, lung, brain, pancreatic, gastric, prostate, acute myelocytic leukemia, and non small cell lung cancers. Thus, in some embodiments, the targeting moiety comprises a folate, which would bind folate receptor expressed on a cancer or tumor cell. In other embodiments, the folate can be folic acid, a folic acid analog or any folate-receptor binding molecule. In some embodiments, the small molecule comprises fluorescein, fluorescein isothiocyanate (FITC), NHS-fluorescein, or any other fluorophore. In some embodiments, the small molecule linked to a targeting moiety is FITC-folate.

In some embodiments, the disclosure contemplates engraftment of engineered stem cells for use in the treatment of subjects with cancer. In some embodiments, the iPSC-derived engineered macrophages stably engraft in the subject. In some embodiments, the iPSC-derived engineered macrophages display long-term engraftment in the subject. In some embodiments, administration of the iPSC-derived engineered macrophages allowed for engraftment and further differentiation within the subject. In some embodiments, the iPSC-derived engineered macrophages display long-term persistence in the subject.

### A. Combination Therapy

In some embodiments, the myeloid cells, such as engineered macrophages and engineered neutrophils, can be used in connection with adoptive cell therapy methods.

In some embodiments, the provided methods can be used in a combination with an additional cancer therapy.

In some embodiments, the additional cancer therapy is a small molecule, e.g., a chemical compound, an antibody therapy, e.g., a humanized monoclonal antibody with or without conjugation to a radionuclide, toxin, or drug, surgery, and/or radiation.

In some embodiments, the subject is selected to receive an additional cancer therapy, which can include a cancer therapeutic, radiation, chemotherapy, or a drug for the treatment of cancer. In some embodiments, the drugs comprise Abiraterone, Alemtuzumab, Anastrozole, Aprepitant, Arsenic trioxide, Atezolizumab, Azacitidine, Bevacizumab, Bleomycin, Bortezomib, Cabazitaxel, Capecitabine, Carboplatin, Cetuximab, Chemotherapy drug combinations, Cisplatin, Crizotinib, Cyclophosphamide, Cytarabine, Denosumab, Docetaxel, Doxorubicin, Eribulin, Erlotinib, Etoposide, Everolimus, Exemestane, Filgrastim, Fluorouracil, Fulvestrant, Gemcitabine, Imatinib, Imiquimod, Ipilimumab, Ixabepilone, Lapatinib, Lenalidomide, Letrozole, Leuprolide, Mesna, Methotrexate, Nivolumab, Oxaliplatin, Paclitaxel, Palonosetron, Pembrolizumab, Pemetrexed, Prednisone, Radium-223, Rituximab, Sipuleucel-T, Sorafenib, Sunitinib, Talc Intrapleural, Tamoxifen, Temozolomide, Temsirolimus, Thalidomide, Trastuzumab, Vinorelbine or Zoledronic acid.

In some embodiments, the subject is selected to receive an antibody therapy. In some embodiments, the antibody therapy comprises an anti-CD38 therapy. In some embodiments, the anti-CD38 antibody is daratumumab (Darzalex), isatuximab (Sarclisa) or MOR202. In some embodiments, the anti-CD38 antibody is daratumumab (Darzalex). In some embodiments, the anti-CD38 antibody is isatuximab (Sarclisa). In some embodiments, the anti-CD38 antibody is MOR202. In some embodiments, the subject is selected to receive a composition comprising any of the iMAC cells provided herein and daratumumab. In some embodiments, the subject is selected to receive a composition comprising any of the iMAC cells provided herein and isatuximab. In some embodiments, the subject is selected to receive a composition comprising any of the iMAC cells provided herein and MOR202.

In some embodiments, the myeloid cells, such as engineered macrophages and engineered neutrophils, can be used in connection with adoptive cell therapy methods, including any of the cell therapy methods described in, for example, in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

### B. Modes of Administration and Dosing

In some embodiments, transduced myeloid cells may be grown in conditions that are suitable for a population of cells that will be introduced into a subject such as a human. Specific considerations include the use of culture media that lacks any animal products, such as bovine serum. Other considerations include sterilized-condition to avoid contamination of bacteria, fungi and mycoplasma. In some embodiments, any of the cell culturing methods contemplated in the disclosure may be used to grow CAR-expressing myeloid cells (e.g., macrophages).

In some embodiments, after transfection, the cells can be immediately administered to the patient or the cells can be cultured for at least about 1, 2, 3, 4, 5, 6, 7, 8, 9, 10, 11, 12, 13, 14, 15, 16, 17, 18 or more days, or between about 5 and about 12 days, between about 6 and about 13 days, between about 7 and about 14 days, or between about 8 and about 15 days, for example, to allow time for the cells to recover from the transfection. Suitable culture conditions can be similar to the conditions under which the cells were cultured for activation either with or without the agent that was used to promote activation. In some embodiments, any of the methods of administering macrophages contemplated in the disclosure may be used to administer CAR-expressing macrophages to the patient.

The disclosed cells may be administered in a number of ways depending upon whether local or systemic treatment is desired.

In the case of adoptive cell therapy, methods for administration of cells for adoptive cell therapy are known and may be used in connection with the provided methods and compositions.

In general, administration may be topical, parenteral, or enteral. The compositions of the disclosure are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrastemal, intravenous, intraarterial, intrathecal, intraventricular, intraurethral, intracranial, intratumoral, intrasynovial injection or infusions; and kidney dialytic infusion techniques. In an embodiment, parenteral administration of the compositions of the present disclosure comprises intravenous administration.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Illustrative parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

The present compositions of viral particles, adaptor molecules, and/or immune cells may be administered in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of infusing differentiated cells or transgenic differentiated cells according to the disclosure, a subject is administered the range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges, and/or such a number of cells per kilogram of body weight of the subject. For example, in some embodiments the administration of the cells or population of cells can comprise administration of about 10³ to about 10⁹ cells per kg body weight including all integer values of cell numbers within those ranges. In some embodiments, the dose is administered one time. In some embodiments, administration of the dose is repeated a plurality of times in a multi-dose administration regimen.

In some embodiments, greater than at or about 5×10⁹ iMACs (e.g. iMAC-CAR) is administered per dose. In some embodiments, from about 5×10⁹ iMACs to about 100×10⁹ iMACs is administered per dose, from about 5×10⁹ iMACs to about 50×10⁹ iMACs is administered per dose, from about 5×10⁹ iMACs to about 25×10⁹ iMACs is administered per dose, from about 5×10⁹ iMACs to 10×10⁹ iMACs is administered per dose, from about 10×10⁹ iMACs to about 100×10⁹ iMACs is administered per dose, from about 10×10⁹ iMACs to about 50×10⁹ iMACs is administered per dose, from about 10×10⁹ iMACs to about 25×10⁹ iMACs is administered per dose, from about 25×10⁹ iMACs to about 100×10⁹ iMACs is administered per dose, from about 25×10⁹ iMACs to about 50×10⁹ iMACs is administered per dose, or from about 50×10⁹ iMACs to about 100×10⁹ iMACs is administered per dose. In some embodiments, at or about 5×10⁹ iMACs, at or about 10×10⁹ iMACs, at or about 20×10⁹ iMACs, at or about 30×10⁹ iMACs, at or about 40×10⁹ iMACs, at or about 50×10⁹ iMACs, at or about 60×10⁹ iMACs, at or about 70×10⁹ iMACs, at or about 80×10⁹ iMACs, at or about 90×10⁹ iMACs, at or about 100×10⁹ iMACs, or any value between any of the foregoing is administered per dose. In some embodiments, the dose is administered one time. In some embodiments, administration of the dose is repeated a plurality of times in a multi-dose administration regimen.

In some embodiments, the therapeutic use of the iMAC cells is a single-dose treatment.

In some embodiments, the therapeutic use of the iMAC cells is a multi-dose treatment. In some embodiments, the multi-dose treatment is one dose every day, every 3 days, every 5 days, every 7 days, every 10 days, every 15 days, every 20 days, every 25 days, every 30 days, every 35 days, every 40 days, every 45 days, or every 50 days, or any number of days in-between. In some embodiments, the composition containing iMAC cells may be administered once weekly. The frequency and duration of the multi-dose therapy can be empirically determined by a skilled physician or clinician, such as based on factors that include signs or symptoms of disease or symptoms in the subject or the pharmacokinetics or expansion of iMACs in the subject.

In some embodiments, the composition containing iMACs cells may be administered as a multi-dose treatment for a predetermined number of doses. In some embodiments, the composition containing iMACs may be administered as two doses, three doses, four doses, five doses, six doses, seven doses, eight doses, nine doses, ten doses, eleven doses or twelve doses. In some embodiments, the doses are administered for 4 weeks, 6 weeks, 8 weeks, 10 weeks, 12 weeks, 16 weeks, 20 weeks, 24 weeks, 28 weeks, 32 weeks, 36 weeks or more.

In some embodiments, the provided compositions containing iMACs can be administered to a subject by any convenient route including parenteral routes such as subcutaneous, intramuscular, intravenous, and/or epidural routes of administration. In particular embodiments, the provided compositions are administered by intravenous infusion.

In some embodiments, the compositions containing iMACs is administered in combination with a non-physiological ligand of the synthetic cytokine receptor. In some embodiments, the non-physiological ligand is rapamycin or a rapamycin analog. In some embodiments, the rapamycin analog is rapalog. In some embodiments, the non-physiological ligand (e.g. rapamycin or an analog) is administered at a dose of 1 mg to 100 mg, optionally between 10-100 mg, optionally at or about 10 mg, 20 mg, 25 mg, 30 mg, 40 mg, 50 mg or any value between any of the foregoing.

In some embodiments, the dose of the non-physiological ligand is administered as a single dose or as multiple doses.

In some embodiments, the non-physiological ligand is administered in multiple doses. In some embodiments, each dose of the multiple doses are for administration every day, every 3 days, every 5 days, every 7 days, every 10 days, every 15 days, every 20 days, every 25 days, every 30 days, every 35 days, every 40 days, every 45 days, or every 50 days, or any number of days in-between. In some embodiments, the non-physiological ligand may be administered once weekly. The frequency and duration of administration of the non-physiological ligand can be empirically determined by a skilled physician or clinician, such as based on the pharmacokinetics or expansion of iMACs in the subject.

In some embodiments, the non-physiological ligand may be administered as a multi-dose treatment for a predetermined number of doses. In some embodiments, the non-physiological ligand may be administered as two doses, three doses, four doses, five doses, six doses, seven doses, eight doses, nine doses, ten doses, eleven doses or twelve doses. In some embodiments, the doses are administered for 4 weeks, 6 weeks, 8 weeks, 10 weeks, 12 weeks, 16 weeks, 20 weeks, 24 weeks, 28 weeks, 32 weeks, 36 weeks or more.

In some embodiments, provided is a method of administering an effective amount of non-physiological ligand to the subject, wherein the non-physiological ligand causes the myeloid progenitor cells to differentiate to macrophages according to any of the provided embodiments.

In some embodiments, the pharmacokinetics of the macrophages following administration to a subject can be monitored. In come embodiments, the concentration of macrophages in the plasma following administration can be measured using any method known in the art suitable for assessing concentrations of cells or of transgenes (e.g. CAR or synthetic cytokine receptor) expressed by such cells in samples of blood, or any methods described herein. For example, nucleic acid-based methods, such as quantitative PCR (qPCR) or flow cytometry-based methods, or other assays, such as an immunoassay, ELISA, or chromatography/mass spectrometry-based assays can be used. In some embodiments, qPCR can be used to detect copy number of nucleic acid encoding the transgene (e.g. CAR or synthetic cytokine receptor) compared to total amount of nucleic acid or DNA in the particular sample, e.g., blood, serum, plasma or tissue, such as a tumor sample. In some embodiments, flow cytometric assays can be used for detecting cells expressing an engineered surface protein, such as a CAR or the synthetic cytokine receptor, generally using antibodies specific for the protein. Cell-based assays may also be used to detect the number or percentage or concentration of functional cells, such as cells capable of binding to and/or neutralizing and/or inducing responses, e.g., cytotoxic responses, against target cells of the disease or condition or expressing the antigen targeted by the CAR.

### VII. EXEMPLARY EMBODIMENTS

Among the provided embodiments are:
1. An engineered cell that is a myeloid progenitor cell comprising a synthetic cytokine receptor for a non-physiological ligand,
wherein the synthetic cytokine receptor comprises:
a synthetic gamma chain polypeptide comprising a first dimerization domain, a first transmembrane domain, and an interleukin-2 receptor subunit gamma (IL-2RG) intracellular domain, and
a synthetic beta chain polypeptide comprising a second dimerization domain, a second transmembrane domain, and beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.
2. The engineered cell of embodiment 1, wherein the myeloid progenitor cell is a Granulocyte/Monocyte Progenitor Cell (“GMP”).
3. The engineered cell of embodiment 1 or embodiment 2, wherein the myeloid progenitor cell is characterized with a surface phenotype CD34+, CD90−, and CD45RA+.
4. The engineered cell of embodiment 1 or embodiment 2, wherein the myeloid progenitor cell is characterized with a surface phenotype CD34+, CD90−, CD123+, and CD45RA+.
5. An engineered cell that is a myeloid cell comprising a synthetic cytokine receptor for a non-physiological ligand,
wherein the cytokine synthetic receptor comprises:
a synthetic gamma chain polypeptide comprising a first dimerization domain, a first transmembrane domain and an interleukin-2 receptor subunit gamma (IL-2RG) intracellular domain, and
a synthetic beta chain polypeptide comprising a second dimerization domain, a second transmembrane domain, and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.
6. The engineered cell of embodiment 5, wherein the myeloid cell is a macrophage, a neutrophil, a megakaryocyte, a monocyte, a basophil, an eosinophil, and/or an erythrocyte cell.
7. An engineered cell that is a macrophage comprising a synthetic cytokine receptor for a non-physiological ligand,
wherein the synthetic cytokine receptor comprises:
a synthetic gamma chain polypeptide comprising a first dimerization domain a first transmembrane domain, and an interleukin-2 receptor subunit gamma (IL-2RG) intracellular domain, and
a synthetic beta chain polypeptide comprising a second dimerization domain, a second transmembrane domain, and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.
8. The engineered cell of embodiment 6 or embodiment 7, wherein the macrophage is a mature macrophage that expresses CD14.
9. An engineered cell that is a neutrophil comprising a synthetic cytokine receptor for a non-physiological ligand,
wherein the synthetic cytokine receptor comprises:
   a synthetic gamma chain polypeptide comprising a first dimerization domain, a first transmembrane domain, and an interleukin-2 receptor subunit gamma (IL-2RG) intracellular domain, and
a synthetic beta chain polypeptide comprising a second dimerization domain, a second transmembrane domain, and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.
10. The engineered cell of any of embodiments 1-9, wherein the engineered cell has been differentiated from a stem cell.
11. The engineered cell of embodiment 10, wherein the stem cell is a pluripotent stem cell.
12. The engineered cell of embodiment 10 or embodiment 11, wherein the stem cell is an induced pluripotent stem cell.
13. The engineered cell of any of embodiments 1-6 and 10-12, wherein the myeloid cell is an induced myeloid cell (iMC) differentiated from a stem cell engineered with the synthetic cytokine receptor.
14. The engineered cell of any of embodiments 7-8 and 10-12, wherein the macrophage is an induced macrophage (iMAC) differentiated from an stem cell engineered with the synthetic cytokine receptor.
15. The engineered cell of any of embodiments 9-12, wherein the neutrophil is an induced neutrophil (iNEU) differentiated from a stem cell engineered with the synthetic cytokine receptor.
16. The engineered cell of any of embodiments 1-15, wherein:
the synthetic gamma chain polypeptide comprises, in N- to C-terminal order, the first dimerization domain, the first transmembrane domain, and the IL-2RG intracellular domain, and the synthetic beta chain polypeptide comprises, in N- to C-terminal order, the second dimerization domain, the second transmembrane domain, and the beta chain intracellular domain.
17. The engineered macrophage of any of embodiments 1-16, wherein the first dimerization domain and the second dimerization domain are extracellular domains
18. The engineered cell of any of embodiments 1-17, wherein the IL-2RG intracellular domain comprises a polypeptide sequence at least 95% identical to SEQ ID NO: 1, or a polypeptide sequence as set forth in SEQ ID NO: 1.
19. The engineered cell of any of embodiments 1-18, wherein the first transmembrane domain comprises the IL-2RG transmembrane domain.
20. The engineered cell of embodiment 19, wherein the IL-2RG transmembrane domain comprises a polypeptide sequence at least 95% identical to SEQ ID NO: 8 or 31, or a polypeptide sequence as set forth in SEQ ID NO: 8 or 31.
21. The engineered cell of any of embodiments 1-20, wherein the beta chain intracellular domain is an the IL-2RB intracellular domain.
22. The engineered cell of embodiment 21, wherein the IL-2RB intracellular domain comprises a polypeptide sequence at least 95% identical to SEQ ID NO: 2, or a polypeptide sequence as set forth in SEQ ID NO: 2.
23. The engineered cell of any of embodiments 1-20, wherein the beta chain intracellular domain is anIL-7RB intracellular domain
24. The engineered cell of embodiment 23, wherein the IL-7RB intracellular domain comprises a polypeptide sequence at least 95% identical to SEQ ID NO: 3, or a polypeptide sequence as set forth in SEQ ID NO: 3
25. The engineered cell of any of embodiments 1-24, wherein the beta chain intracellular domain is an IL-21RB intracellular domain.
26. The engineered cell of any of embodiments 25, wherein the IL-21RB intracellular domain comprises a polypeptide sequence at least 95% identical to SEQ ID NO: 4, or a polypeptide sequence as set forth in SEQ ID NO: 4.
27. The engineered cell of any of embodiments 1-26, wherein the second transmembrane domain comprises a transmembrane domain from the same polypeptide as the beta chain intracellular domain.
28. The engineered cell of any one of embodiments 1-27, wherein the second transmembrane domain is a transmembrane domain of IL-2RB comprising a polypeptide sequence at least 95% identical to SEQ ID NO: 35 or 36, or a polypeptide sequence as set forth in SEQ ID NO: 35 or 36.
29. The engineered cell of any of embodiments 1-22, 27 and 28, wherein:
the first transmembrane domain of the synthetic gamma chain polypeptide is an IL-2RG transmembrane domain comprising the sequence set forth in SEQ ID NO: 8 or 31, and the IL-2RG intracellular domain comprises the sequence set forth in SEQ ID NO:1; and
the second transmembrane domain of the synthetic beta chain polypeptide is an IL-2RB transmembrane domain comprising the sequence set forth in SEQ ID NO: 35 or 36, and the beta chain intracellular domain is an IL-2RB intracellular domain comprising the sequence set forth in SEQ ID NO:2.
30. The engineered cell of any of embodiments 1-29, wherein:
the first transmembrane domain and the IL-2RG intracellular domain of the synthetic gamma chain polypeptide comprise the sequences set forth in SEQ ID NOs: 31 and SEQ ID NO: 1; and
the second transmembrane domain and the beta chain intracellular domain of the synthetic beta chain polypeptide comprise the sequences set forth in SEQ ID NOs: 35 and SEQ ID NO: 2.
31. The engineered cell of any one of embodiments 1-30, wherein the first dimerization domain and the second dimerization domain are heterodimerization domains selected from FKBP12-rapamycin binding (FRB) domain and a FK506-Binding Protein of size 12 kD (FKBP); and/or
wherein the non-physiological ligand is rapamycin or a rapalog
32. The engineered cell of any one of embodiments 1-31, wherein the first dimerization domain is FKBP and the second dimerization domain is FRB.
33. The engineered cell of any one of embodiments 1-31, wherein the first dimerization domain is FRB and the second dimerization domain is FKBP.
34. The engineered cell of any of embodiments 31-33, wherein the FRB domain comprises a polypeptide sequence at least 95% identical to SEQ ID NO: 6 or SEQ ID NO: 7.
35. The engineered cell of any of embodiments 31-34, wherein the FRB domain comprises the polypeptide sequence set forth in SEQ ID NO: 6 or SEQ ID NO: 7.
36. The engineered cell of any one of embodiments 31-35, wherein the FKBP domain comprises a polypeptide sequence at least 95% identical to SEQ ID NO: 5 or SEQ ID NO:30.
37. The engineered cell of any one of embodiments 31-36, wherein the FKBP domain comprises the polypeptide sequence set forth in SEQ ID NO: 5 or SEQ ID NO:30
38. The engineered cell of any of embodiments 1-37, wherein the synthetic gamma chain polypeptide has the sequence of amino acids set forth in SEQ ID NO: 28 or a sequence of amino acids that is at least 90%, at least 95%, at least 96%, at least 97%, at least 98%, at least 99%, or 100% identical to SEQ ID NO: 28, and the synthetic beta chain polypeptide has the sequence of amino acids set forth in SEQ ID NO: 55 or a sequence of amino acids that is at least 90%, at least 95%, at least 96%, at least 97%, at least 98%, at least 99%, or 100% identical to SEQ ID NO: 55.
39. The engineered cell of any of embodiments 1-38, wherein the synthetic gamma chain polypeptide has the sequence of amino acids set forth in SEQ ID NO: 56 or a sequence of amino acids that is at least 90%, at least 95%, at least 96%, at least 97%, at least 98%, at least 99%, or 100% identical to SEQ ID NO: 56, and the synthetic beta chain polypeptide has the sequence of amino acids set forth in SEQ ID NO: 57 or a sequence of amino acids that is at least 90%, at least 95%, at least 96%, at least 97%, at least 98%, at least 99%, or 100% identical to SEQ ID NO: 57.
40. The engineered cell of any of embodiments 1-30, wherein the first dimerization domain and the second dimerization domain are homodimerization domains selected from:
   i) FK506-Binding Protein of size 12 kD (FKBP);
   ii) cyclophiliA (CypA); or
   iii) gyrase B (CyrB);
and the non-physiological ligand is, respectively:
   i) FK1012, AP1510, AP1903, or AP20187 or an analog thereof,
   ii) cyclosporin-A (CsA) or an analog thereof, or
   iii) coumermycin or an analog thereof.
41. The engineered cell of any of embodiments 1-40, wherein the engineered cell is resistant to rapamycin-mediated mTOR inhibition.
42. The engineered cell of any of embodiments 1-41, wherein the engineered cell expresses a cytosolic polypeptide that binds to the non-physiological ligand, optionally wherein the cytosolic polypeptide is a cytosolic FRB domain.
43. The engineered cell of any of embodiments 1-42, wherein the non-physiological ligand is rapamycin or a rapalog, and the engineered cell expresses a cytosolic FRB domain or variant thereof.
44. The engineered cell of embodiment 42 or embodiment 43, wherein the cytosolic FRB domain comprises a polypeptide sequence at least 95% identical to SEQ ID NO: 6 or SEQ ID NO: 7.
45. The engineered cell of embodiment 42 or embodiment 43, wherein the cytosolic FRB domain comprises a polypeptide sequence at least 98% identical to SEQ ID NO: 6 or SEQ ID NO: 7.
46. The engineered cell of any of embodiments 1-45, wherein the engineered cell comprises a disrupted FKBP12 gene that reduces expression of FKBP12 in the cell.
47. The engineered cell of any of embodiments 1-46, wherein the engineered cell comprises knock out of the FKBP12 gene.
48. The engineered cell of any one of embodiments 1-47, wherein the engineered cell comprises a nucleotide sequence encoding the synthetic cytokine receptor inserted into the genome of the cell.
49. The engineered cell of embodiment 48, wherein the nucleotide sequence encoding the synthetic cytokine receptor is inserted into a non-target locus in the genome of the cell.
50. The engineered cell of embodiment 48, wherein the nucleotide sequence encoding the synthetic cytokine receptor is inserted into an endogenous gene of the cell.
51. The engineered cell of embodiment 50, wherein the insertion reduces expression of the endogenous gene in the locus.
52. The engineered cell of embodiment 50 or embodiment 51, wherein the insertion knocks out the endogenous gene in the locus.
53. The engineered cell of any of embodiments 50-52, wherein the insertion is by homology-directed repair.
54. The engineered cell of any of embodiments 50-53, wherein the endogenous gene is a housekeeping gene, a blood-lineage specific loci or an immune-related gene.
55. The engineered cell of embodiment 54, wherein the housekeeping gene is selected from eukaryotic translation elongation factor 1 alpha (EEF1A), glylceraldehyde-3-phosphate dehydrogenase (GAPDH), ubiquitin C (UBC), and actin beta (ACTB).
56. The engineered cell of embodiment 54, wherein the immune-related gene is selected from a beta-2-microglobulin (B2M) gene, and a signal regulatory protein alpha (SIRPA) gene.
57. The engineered cell of any one of embodiments 1-56, wherein the engineered cell comprises a B2M knockout.
58. The engineered cell of any of embodiments 1-57, wherein the engineered cell comprises a B2M knockout and a FKBP12 knockout.
59. The engineered cell of any one of embodiments 1-58, comprising a chimeric antigen receptor (CAR).
60. The engineered cell of any of embodiments 1-59, wherein binding of the non-physiological ligand to the synthetic cytokine receptor activates the synthetic cytokine receptor in the engineered cell to induce expansion and/or activation of the engineered cell in a cell population.
61. A population comprising engineered myeloid progenitor cells of any of embodiments 1-4 and 10-12 and 16-60.
62. A population comprising engineered myeloid cells of any of embodiments 5, 6 and 10-13 and 16-60.
63. A population comprising engineered macrophages of any of embodiments 7, 8 and 10-12, and 16-60.
64. A population comprising engineered neutrophils of any of embodiments 9-12 and 15-60.
65. A method of generating genetically engineered myeloid cells differentiated from stem cells, the method comprising:
a) culturing a population of stem cells engineered with a synthetic cytokine receptor under conditions to differentiate cells into a population of hematopoietic progenitors (HP),
wherein the synthetic cytokine receptor comprises:
a synthetic gamma chain polypeptide comprising a first dimerization domain, a first transmembrane domain and an interleukin-2 receptor subunit gamma (IL-2RG) intracellular domain, and
a synthetic beta chain polypeptide comprising a second dimerization domain a second transmembrane domain and and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain; and
b) culturing the cells produced in a) by incubation under conditions to generate myeloid cells.
wherein at least a portion of one or both of steps a) andb) are carried out in the presence of a non-physiological ligand of the synthetic cytokine receptor.
66. The method of embodiment 65, wherein the myeloid cell is a macrophage or a neutrophil.
67. A method of generating genetically engineered macrophage differentiated from stem cells, the method comprising:
a) culturing a population of stem cells engineered with a synthetic cytokine receptor under conditions to differentiate cells into a population of hematopoietic progenitors (HP),
wherein the synthetic cytokine receptor comprises:
a synthetic gamma chain polypeptide comprising a first dimerization domain, a first transmembrane domain, and an interleukin-2 receptor subunit gamma (IL-2RG) intracellular domain, and
a synthetic beta chain polypeptide comprising a second dimerization domain, a second transmembrane domain and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain; and
b) culturing the cells produced in a) by incubation under conditions to generate macrophages,
wherein at least a portion of one or both of steps a) andb) are carried out in the presence of a non-physiological ligand of the synthetic cytokine receptor.
68. The method of any of embodiments 65-67, wherein the culturing in a) is carried out by a first incubation under conditions to produce an Embryoid Body (EB) followed by one or more further incubations in the presence of the non-physiological ligand and optionally one or more myeloid cell differentiation factors selected from one or more of IL-3, M-CSF and GM-CSF.
69. The method of embodiment 68, wherein the one or more myeloid cell differentiation factors is IL-3, M-CSF and GM-CSF.
70. The method of embodiment 68 or embodiment 69, wherein the one or more further incubations comprises a second incubation in a second media comprising one or more of IL-3, GM-CSF, and M-CSF, and a third incubation in a third media comprising one or more of IL-3, GM-CSF, and M-CSF, wherein one or both of the second media and the third media comprises the non-physiological ligand.
71. The method of any of embodiments 65-70, wherein step a) comprises:
(i) performing a first incubation comprising culturing the population of stem cells engineered with the synthetic cytokine receptor under conditions to form a first aggregate in a first media;
(ii) contacting the aggregate with a dissociating agent to form a population of dissociated cells;
(iii) performing a second incubation comprising culturing the population of dissociated cells under conditions to form a second aggregate in a second media, optionally wherein the second media comprises one or more of IL-3, GM-CSF, and M-CSF; and
(iv) performing a third incubation comprising culturing the population of cells in (iii) in a third media, optionally wherein the third media comprises one or more of IL-3, GM-CSF, and M-CSF.
72. The method of any of embodiments 68-71, wherein the first incubation is in a first media comprising one or more of BMP4, FGF2, VEGF-165 and a Rock Inhibitor.
73. The method of any of embodiments 68-72, wherein the first incubation is in a first media comprising BMP4, FGF2, VEGF-165 and a Rock Inhibitor.
74. the method of embodiment 72 or embodiment 73, wherein the Rock Inhibitor is Y27632.
75. The method of any one of embodiments 70-74, wherein the second media further comprises the non-physiological ligand of the synthetic cytokine receptor.
76. The method of any one of embodiment 70-74, wherein the second media does not comprise the non-physiological ligand of the synthetic cytokine receptor.
77. The method of any of embodiments 70-76, wherein the culturing in the first media is for 1 to 3 days.
78. The method of any one of embodiments 70-77, wherein the second media comprises one or more of BMP4, FGF2, VEGF, LY294002, IL-3, GM-CSF, and M-CSF.
79. The method of any one of embodiments 70-77, wherein the second media comprises BMP4, FGF2, VEGF, LY294002, IL-3, and M-CSF.
80. The method of any one of embodiments 70-79, wherein the second media further comprises a non-physiological ligand of the synthetic cytokine receptor.
81. The method of any one of embodiments 70-79, wherein the second media does not comprise a non-physiological ligand of the synthetic cytokine receptor.
82. The method of any one of embodiments 70-81, wherein the culturing in the second media is for 3 to 6 days.
83. The method of any one of embodiments 70-82, wherein the third media comprises one or more of UM729, StemRegenin-1, BMP4, FGF2, VEGF, LY294002, IL-3, GM-CSF, and M-CSF.
84. The method of any one of embodiments 70-82, wherein the third media comprises UM729, StemRegenin-1, BMP4, FGF2, VEGF, LY294002, IL-3, GM-CSF, and M-CSF.
85. The method of any one of embodiments 70-84, wherein the third media further comprises a non-physiological ligand of the synthetic cytokine receptor.
86. The method of any one of embodiments 70-84, wherein the third media does not comprise a non-physiological ligand of the synthetic cytokine receptor.
87. The method of any one of embodiments 70-86, wherein the culturing in the third media is for 6 to 12 days.
88. The method of any of embodiments 67-87, wherein the culturing in a) produces myeloid progenitor cells.
89. The method of embodiment 88, wherein the myeloid progenitor cell is a Granulocyte/Monocyte Progenitor Cell (“GMP”).
90. The method of embodiment 88 or embodiment 89, wherein the myeloid progenitor cells are characterized with a surface phenotype CD34+, CD90−, and CD45RA+.
91. The method of embodiment 88 or embodiment 89, wherein the myeloid progenitor cells are characterized with a surface phenotype CD34+, CD90−, CD123+, and CD45RA+.
92. The method of any one of embodiments 65-91, wherein the culturing in b) is in a media comprising one or more of UM729, SCF, StemRegenin1, IL-3, GM-CSF, and M-CSF.
93. The method of any one of embodiments 65-91, wherein the culturing in b) is in a media comprising UM729, SCF, StemRegenin1, IL-3, GM-CSF, and M-CSF.
94. The method of any one of embodiments 65-93, wherein the culturing in b) is in a media further comprising a non-physiological ligand of the synthetic cytokine receptor.
95. The method of any one of embodiments 65-93, wherein the culturing in b) is in a media that does not comprise a non-physiological ligand of the synthetic cytokine receptor.
96. The method of any one of embodiments 67-95, wherein the culturing in b) is for 12 to 24 days.
97. A method of generating genetically engineered myeloid cells to express a synthetic cytokine receptor, comprising: culturing a population of hematopoietic progenitors (HP) cells derived from stem cells engineered with a synthetic cytokine receptor under conditions to differentiate cells into a population of myeloid cells, wherein at least a portion of the culturing is carried out in the presence of a non-physiological ligand of the synthetic cytokine receptor, and
wherein the synthetic cytokine receptor comprises:
a synthetic gamma chain polypeptide comprising a first dimerization domain, a first transmembrane domain and an interleukin-2 receptor subunit gamma (IL-2RG) intracellular domain, and
a synthetic beta chain polypeptide comprising a second dimerization domain, a second transmembrane domain, and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.
98. A method of generating genetically engineered myeloid cells to express a synthetic cytokine receptor, comprising: culturing a population of myeloid progenitor cells derived from stem cells engineered with a synthetic cytokine receptor under conditions to differentiate cells into a population of myeloid cells, wherein at least a portion of the culturing is carried out in the presence of a non-physiological ligand of the synthetic cytokine receptor, and
wherein the synthetic cytokine receptor comprises:
a synthetic gamma chain polypeptide comprising a first dimerization domain, a first transmembrane domain and an interleukin-2 receptor subunit gamma (IL-2RG) intracellular domain, and
a synthetic beta chain polypeptide comprising a second dimerization domain, a second transmembrane domain and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.
99. The method of embodiment 97 or embodiment 98, wherein the myeloid cells are macrophages or neutrophils.
100. A method of generating genetically engineered macrophages to express a synthetic cytokine receptor, comprising: culturing a population of hematopoietic progenitors (HP) cells derived from stem cells engineered with a synthetic cytokine receptor under conditions to differentiate cells into a population of macrophages, wherein at least a portion of the culturing is carried out in the presence of a non-physiological ligand of the synthetic cytokine receptor, and
wherein the synthetic cytokine receptor comprises:
a synthetic gamma chain polypeptide comprising a first dimerization domain, a first transmembrane domain and an interleukin-2 receptor subunit gamma (IL-2RG) intracellular domain, and
a synthetic beta chain polypeptide comprising a second dimerization domain, a a second transmembrane domain and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.
101. A method of generating genetically engineered macrophages to express a synthetic cytokine receptor, comprising: culturing a population of myeloid progenitor cells derived from stem cells engineered with a synthetic cytokine receptor under conditions to differentiate cells into a population of macrophages, wherein at least a portion of the culturing is carried out in the presence of a non-physiological ligand of the synthetic cytokine receptor, and
wherein the synthetic cytokine receptor comprises:
a synthetic gamma chain polypeptide comprising a first dimerization domain, a first transmembrane domain, and an interleukin-2 receptor subunit gamma (IL-2RG) intracellular domain, and
a synthetic beta chain polypeptide comprising a second dimerization domain, a second transmembrane domain a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain and intracellular domain.
102. The method of any of embodiments 97-101, wherein the culturing is in a media comprising one or more of UM729, SCF, StemRegenin1, IL-3, GM-CSF, and M-CSF.
103. The method of any one of embodiments 97-101, wherein the culturing is in a media comprising UM729, SCF, StemRegenin1, IL-3, GM-CSF, and M-CSF.
104. The method of any one of embodiments 97-103, wherein the culturing is for 12 to 24 days.
105. The method of any of embodiments 67-96, wherein the culturing of one or both steps a) and b) is carried out in a bioreactor.
106. The method of any of embodiments 97-104, wherein the culturing is carried out in a bioreactor.
107. The method of embodiment 105 or embodiment 106, wherein the bioreactor is a vertical wheel bioreactor.
108. The method of any of embodiments 105-107, wherein the bioreactor is a vertical wheel bioreactor with a volume of about 10 mL to about 1000 mL.
109. The method of embodiment 105, wherein the culturing in a) is carried out in a bioreactor and wherein the bioreactor is a vertical wheel bioreactor with a volume of about 100 mL.
110. The method of any embodiment 105 or embodiment 109, wherein the culturing in b) is carried out in a bioreactor and the bioreactor is a vertical wheel bioreactor with a volume of about 500 mL.
111. The method of embodiment 106, wherein the bioreactor is a vertical wheel bioreactor with a volume of about 500 mL.
112. The method of any one of embodiments 65-111, wherein the stem cells are pluripotent stem cells.
113. The method of embodiment 112, wherein the pluripotent stem cells are induced pluripotent stem cells.
114. The method of any of embodiments 65-112, wherein the synthetic gamma chain polypeptide comprises, in N- to C-terminal order, the first dimerization domain, the first transmembrane domain, and the IL-2RG intracellular domain, and the synthetic beta chain polypeptide comprises, in N- to C-terminal order, the second dimerization domain, the second transmembrane domain, and the intracellular domain.
115. The method of any of embodiments 65-114, wherein the first dimerization domain and the second dimerization domain are extracellular domains.
116. The method of any of embodiments 65-115, wherein the IL-2RG intracellular domain comprises a polypeptide sequence at least 95% identical to SEQ ID NO: 1, or a polypeptide sequence as set forth in SEQ ID NO: 1.
117. The method of any of embodiments 65-116, wherein the first transmembrane domain comprises the IL-2RG transmembrane domain.
118. The method of embodiment 117, wherein the IL-2RG transmembrane domain comprises a polypeptide sequence at least 95% identical to SEQ ID NO: 8 or 31, or a polypeptide sequence as set forth in SEQ ID NO: 8 or 31.
119. The method of any of embodiments 65-118, wherein the beta chain intracellular domain comprises the IL-2RB intracellular domain.
120. The method of embodiment 119, wherein the IL-2RB intracellular domain comprises a polypeptide sequence at least 95% identical to SEQ ID NO: 2, or a polypeptide sequence as set forth in SEQ ID NO: 2.
121. The method of any of embodiments 65-118, wherein the beta chain intracellular domain comprises the IL-7RB intracellular domain.
122. The method of embodiment 121, wherein the IL-7RB intracellular domain comprises a polypeptide sequence at least 95% identical to SEQ ID NO: 3, or a polypeptide sequence as set forth in SEQ ID NO: 3.
123. The method of embodiment 122, wherein the beta chain intracellular domain comprises the IL-21RB intracellular domain.
124. The method of embodiment 123, wherein the IL-21RB intracellular domain comprises a polypeptide sequence at least 95% identical to SEQ ID NO: 4, or a polypeptide sequence as set forth in SEQ ID NO: 4.
125. The method of any of embodiments 65-124, wherein the second transmembrane domain comprises a transmembrane domain from the same polypeptide as the intracellular domain.
126. The method of any one of embodiments 65-125, wherein the second transmembrane domain is a transmembrane domain of IL-2RB comprising a polypeptide sequence at least 95% identical to SEQ ID NO: 35 or 36, or a polypeptide sequence as set forth in SEQ ID NO: 35 or 36.
127. The method of any of embodiments 65-120, 125 and 126, wherein:
the first transmembrane domain of the synthetic gamma chain polypeptide is an IL-2RG transmembrane domain comprising the sequence set forth in SEQ ID NO: 8 or 31 and the IL-2RG intracellular domain comprises the sequence set forth in SEQ ID NO:1; and
the second transmembrane domain of the synthetic beta chain polypeptide is an IL-2RB transmembrane domain comprising the sequence set forth in SEQ ID NO: 35 or 36 and the beta chainintracellular domain comprising the sequence set forth in SEQ ID NO:2.
128. The method of any of embodiments 65-127, wherein:
the first transmembrane domain and the IL-2RG intracellular domain of the synthetic gamma chain polypeptide comprises the sequences set forth in SEQ ID NOs: 31 and SEQ ID NO: 1; and
the second transmembrane domain and the beta chain intracellular domain of the synthetic beta chain polypeptide comprises the sequences set forth in SEQ ID NOs: 35 and SEQ ID NO: 2.
129. The method of any one of embodiments 65-128, wherein the first dimerization domain and the second dimerization domain are heterodimerization domains selected from selected from FKBP12-rapamycin binding (FRB) domain and a FK506-Binding Protein of size 12 kD (FKBP); and/or
wherein the non-physiological ligand is rapamycin or a rapalog.
130. The method of any one of embodiments 65-129, wherein the first dimerization domain is FKBP and the second dimerization domain is FRB.
131. The method of any one of embodiments 65-129, wherein the first dimerization domain is FRB and the second dimerization domain is FKBP.
132. The method of any of embodiments 129-131, wherein the FRB domain comprises a polypeptide sequence at least 95% identical to SEQ ID NO: 6 or SEQ ID NO: 7.
133. The method of any of embodiments 129-132, wherein the FRB domain comprises the polypeptide sequence set forth in SEQ ID NO: 6 or SEQ ID NO: 7.
134. The engineered cell of any one of embodiments 129-133, wherein the FKBP domain comprises a polypeptide sequence at least 95% identical to SEQ ID NO: 5 or SEQ ID NO:30.
135. The method of any one of embodiments 129-134, wherein the FKBP domain comprises the polypeptide sequence set forth in SEQ ID NO: 5 or SEQ ID NO:30.
136. The method of any of embodiments 65-135, wherein the synthetic gamma chain polypeptide has the sequence of amino acids set forth in SEQ ID NO: 28 or a sequence of amino acids that is at least 90%, at least 95%, at least 96%, at least 97%, at least 98%, at least 99%, or 100% identical to SEQ ID NO: 28, and the synthetic beta chain polypeptide has the sequence of amino acids set forth in SEQ ID NO: 55 or a sequence of amino acids that is at least 90%, at least 95%, at least 96%, at least 97%, at least 98%, at least 99%, or 100% identical to SEQ ID NO: 55.
137. The method of any of embodiments 65-136, wherein the synthetic gamma chain polypeptide has the sequence of amino acids set forth in SEQ ID NO: 56 or a sequence of amino acids that is at least 90%, at least 95%, at least 96%, at least 97%, at least 98%, at least 99%, or 100% identical to SEQ ID NO: 56, and the synthetic beta chain polypeptide has the sequence of amino acids set forth in SEQ ID NO: 57 or a sequence of amino acids that is at least 90%, at least 95%, at least 96%, at least 97%, at least 98%, at least 99%, or 100% identical to SEQ ID NO: 57.
138. The method of any of embodiments 65-128, wherein the first dimerization domain and the second dimerization domain are homodimerization domains selected from:
   i) FK506-Binding Protein of size 12 kD (FKBP);
   ii) cyclophiliA (CypA); or
   iii) gyrase B (CyrB);
and the non-physiological ligand is, respectively:
   i) FK1012, AP1510, AP1903, or AP20187 or an analog thereof,
   ii) cyclosporin-A (CsA) or an analog thereof, or
   iii) coumermycin or an analog thereof.
139. The method of any of embodiments 65-138, wherein cells of the population of cells (the population of stem cells, the population of hematopoietic progenitor cells or the population of myeloid progenitor cells) are resistant to rapamycin-mediated mTOR inhibition.
140. The method of any of embodiments 65-139, wherein cells of the population of cells (the population of stem cells, the population of hematopoietic progenitor cells or the population of myeloid progenitor cells) express a cytosolic polypeptide that binds to the non-physiological ligand, optionally wherein the cytosolic polypeptide is a cytosolic FRB domain.
141. The method of any of embodiments 65-140, wherein the non-physiological ligand is rapamycin or a rapalog, and the cells of the population express a cytosolic FRB domain or variant thereof.
142. The method of embodiment 140 or embodiment 141, wherein the cytosolic FRB domain comprises a polypeptide sequence at least 95% identical to SEQ ID NO: 6 or SEQ ID NO: 7.
143. The method of embodiment 140 or embodiment 141, wherein the cytosolic FRB domain comprises a polypeptide sequence at least 98% identical to SEQ ID NO: 6 or SEQ ID NO: 7.
144. The method of any of embodiments 65-143, wherein cells of the population of cells (the population of stem cells, the population of hematopoietic progenitor cells or the population of myeloid progenitor cells) comprise a disrupted FKBP12 gene that reduces expression of FKBP12 in the cell.
145. The method of any of embodiments 65-144, wherein cells of the population of cells (the population of stem cells, the population of hematopoietic progenitor cells or the population of myeloid progenitor cells) comprise knock out of the FKBP12 gene.
146. The method of any of embodiments 65-145, wherein the synthetic cytokine receptor is integrated into an endogenous gene of cells of the population of cells (the population of stem cells, the population of hematopoietic progenitor cells or the population of myeloid progenitor cells) by targeted integration of the nucleotide sequence encoding the synthetic cytokine receptor into the endogenous gene.
147. The method of embodiment 146, wherein the targeted integration is by non-homologous end joining (NHEJ).
148. The method of embodiment 146, wherein the targeted integration is by homology directed repair.
149. The method of any of embodiments 146-148, wherein the insertion reduces expression of the endogenous gene in the locus.
150. The method of any of embodiments 146-149, wherein the insertion knocks out the endogenous gene in the locus.
151. The method of any of embodiments 146-150, wherein the insertion is by homology-directed repair.
152. The method of any of embodiments 146-151, wherein the endogenous gene is a housekeeping gene, a blood-lineage specific loci or an immune-related gene.
153. The method of embodiment 152, wherein the housekeeping gene is selected from eukaryotic translation elongation factor 1 alpha (EEF1A), glylceraldehyde-3-phosphate dehydrogenase (GAPDH), ubiquitin C (UBC), and actin beta (ACTB).
154. The method of embodiment 152, wherein the immune-related gene is selected from a beta-2-microglobulin (B2M) gene, and a signal regulatory protein alpha (SIRPA) gene.
155. The method of any one of embodiments 65-154, wherein cells of the population of cells (the population of stem cells, the population of hematopoietic progenitor cells or the population of myeloid progenitor cells) comprises a B2M knockout.
156. The method of any of embodiments 65-155, wherein cells of the population of cells (the population of stem cells, the population of hematopoietic progenitor cells or the population of myeloid progenitor cells) comprise a B2M knockout and a FKBP12 knockout.
157. The method of any one of embodiments 65-156, wherein cells of the population of cells (the population of stem cells, the population of hematopoietic progenitor cells or the population of myeloid progenitor cells) comprising a chimeric antigen receptor (CAR).
158. The method of any one of embodiments 66-96 and 99-157, wherein macrophages are mature macrophages that express CD14.
159. The method of any one of embodiments 65-158, wherein the non-physiological ligand is rapamycin or a rapamycin analog.
160. The method of embodiment 159, wherein the rapamycin analog is rapalog.
161. The method of any one of embodiments 65-160, wherein the non-physiological ligand is added to the media at a concentration of between 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM, 10 nM and 50 nM, 10 nM and 20 nM, 20 nM and 200 nM, 20 nM and 150 nM 20 nM and 100 nM, 20 nM and 50 nM, 50 nM and 200 nM, 50 nM and 150 nM, 50 nM and 100 nM, 100 nM and 200 nM, 100 nM and 150 nM and 150 nM and 200 nM.
162. The method of any one of embodiments 65-161, wherein the non-physiological ligand is added to the media at a concentration of at or about 10 nM.
163. The method of any of embodiments 65-162, wherein the non-physiological ligand is added to the media at a concentration of at or about 100 nM.
164. A population of myeloid cells produced by the method of any of embodiments 65, 66, 68-99 and 102-163.
165. A population of macrophages produced by the method of any of embodiments 65-96 and 99-163.
166. The population of embodiment 165, wherein the population of macrophages express CD14.
167. A pharmaceutical composition comprising the population of engineered myeloid cells of embodiment 62 or embodiment 164.
168. A pharmaceutical composition comprising the population of engineered macrophages of embodiment 63, embodiment 165 or embodiment 166.
169. The pharmaceutical composition of any of embodiments 167-168 comprising a pharmaceutically acceptable excipient.
170. The pharmaceutical composition of any of embodiments 167-169 comprising a cryoprotectant.
171. A method of expanding myeloid cells, the method comprising contacting the population of myeloid cells of embodiment 62 or embodiment 164, or the pharmaceutical composition of embodiment 167, 169 or 170 with the non-physiological ligand of the synthetic cytokine receptor.
172. A method of expanding macrophages, the method comprising contacting the population of macrophages of embodiment 63, embodiment 165 or embodiment 166, or the pharmaceutical composition of embodiment 168, 169 or 170 with the non-physiological ligand of the synthetic cytokine receptor.
173. The method of embodiment 171 or embodiment 172 that is performed in vitro or ex vivo.
174. The method of any one of embodiments 171-173, wherein the non-physiological ligand is rapamycin or a rapamycin analog.
175. The method of embodiment 174, wherein the rapamycin analog is rapalog.
176. The method of one any of embodiments 171-175, wherein the non-physiological ligand is contacted at a concentration of between 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM, 10 nM and 50 nM, 10 nM and 20 nM, 20 nM and 200 nM, 20 nM and 150 nM 20 nM and 100 nM, 20 nM and 50 nM, 50 nM and 200 nM, 50 nM and 150 nM, 50 nM and 100 nM, 100 nM and 200 nM, 100 nM and 150 nM and 150 nM and 200 nM.
177. The method of any one of embodiments 171-176, wherein the non-physiological ligand is contacted at a concentration of at or about 10 nM.
178. The method of any one of embodiments 171-177, wherein the non-physiological ligand is contacted at a concentration of at or about 100 nM.
179. The method of any one of embodiments 171-178, wherein the method is performed in vivo in a subject and the non-physiological ligand is administered to the subject.
180. A method of treating a disease or condition in a subject, comprising administering to the subject an effective amount of the population of myeloid cells of embodiment 62 or embodiment 164, or the pharmaceutical composition of embodiment 167, 169 or 170.
181. A method of treating a disease or condition in a subject, comprising administering to the subject an effective amount of the population of macrophages of embodiment 63, embodiment 165 or embodiment 166, or the pharmaceutical composition of embodiment 168, 169 or 170 with the non-physiological ligand of the synthetic cytokine receptor.
182. The method of embodiment 180 or embodiment 181, wherein the disease or condition is a cancer.
183. The method of any one of embodiments 171-182, wherein the cells express a CAR directed against an antigen expressed by cells of the disease or condition.
184. The method of embodiment 183, wherein the CAR targets a cancer antigen.
185. The method of any one of embodiments 171-184, comprising administering to the subject a non-physiological ligand of the synthetic cytokine receptor.
186. The method of any one of embodiments 171-185, wherein the non-physiological ligand is rapamycin or a rapamycin analog.
187. The method of embodiment 186, wherein the rapamycin analog is rapalog.
188. The method of any one of embodiments 171-187, wherein the non-physiological ligand is administered at a dose of 1 mg to 100 mg, optionally between 10-100 mg, optionally at or about 10 mg, 20 mg, 25 mg, 30 mg, 40 mg, 50 mg or any value between any of the foregoing.
189. The method of any one of embodiments 171-188, wherein multiple doses of the non-physiological ligand are administered to the subject.
190. The method of embodiment 189, wherein the multiple doses are administered intermittently or at regular intervals after administration of the macrophage population or composition thereof to the subject, optionally for a predetermined period of time.
191. The method of any one of embodiments 171-190, wherein 2 to 8 doses of the non-physiological ligand are administered to the subject.
192. The method of any one of embodiments 171-190, wherein a single dose of the non-physiological ligand is administered to the subject.
193. A kit comprising the engineered cell of any one of embodiments 1-60, the population of engineered myeloid cells of claim 62 or claim 164, the population of macrophages of embodiment 63, embodiment 165 or embodiment 166, or the pharmaceutical composition of any one of embodiments 168-170 and instructions for administering to a subject in need thereof.
194. The kit of embodiment 193, further comprising a container comprising the non-physiological ligand and instructions for administering the non-physiological ligand to the subject after administration of the cell population.
195. The kit of any of embodiment 193 or embodiment 194, wherein the subject has a cancer.

### VIII. EXAMPLES

The following examples are included for illustrative purposes only and are not intended to limit the scope of the invention.

### Example 1 Gene Editing of iPSCs

This example demonstrates transduction of a Rapamycin-Activated Cytokine Receptor (RACR) (SEQ ID NO: 28 and 33) in induced pluripotent stem cells (iPSCs). RACR is activated via the addition of its synthetic ligand rapamycin, which induces a JAK/STAT signal. In this example, experiments were carried out to assess if addition of the synthetic ligand is able to drive differentiation and expansion of cells into hematopoietic progenitors (HPs) and then into natural killer cells or macrophages (termed, iMacs).

The RACR construct was designed as a synthetic cytokine polypeptide comprising a first nucleotide sequence FKBP12:IL2Rγ (amino acid sequence set forth in SEQ ID NO:28) and a second nucleotide sequence encoding FRB:IL2Rβ (amino acid sequence set forth in SEQ ID NO:33). Alternatively, RACR comprises a first nucleotide sequence FRB:IL2Rγ (amino acid sequence set forth in SEQ ID NO:56) and a second nucleotide sequence encoding FKBP12:IL2Rβ (amino acid sequence set forth in SEQ ID NO:57).

CRISPR-Cas9 was used to generate site-specific knock-ins of constructs encoding RACR via homology-directed repair (HDR) and is shown schematically in FIG. 1A. Specifically, RACR was knocked-in at the B2M locus with the addition of an EF1a promoter (B2M homology arms SEQ ID NO: 22-23; B2M gRNA SEQ ID NO: 18; EF1a promoter SEQ ID NO: 24).

iPSCs were genetically engineered using methods well known in the art, such as in Ran et al. Nature Protocols, Vol. 8, pgs. 2281-2308 (2013), Liu et al. The CRISPR Journal, Vol. 3, Issue 3 (2020), and General CRISPR RNP Transfection Guidelines by Thermo Fisher Scientific, which are incorporated by reference in their entirety. In this example, electroporation was used. The cells were electroporated with ribonucleoprotein (RNP) complexes comprising a Cas9 and a guide RNA that targeted the specific locus indicated.

Protein levels of RACR were detected through flow cytometry after staining with rapamycin-AF647 (FIG. 1B). The stained signal in the iPSC parent is likely due to Rapa-AF647 binding to endogenous FKBP12, with further increases in signal observed in cells expressing RACR from either actin B (ACTB) or B2M-EF1a modified loci (FIG. 1C).

The expressed levels of RACR were normalized to actin B (ACTB) mRNA expression for gene-edited iPSCs. RACR was first knocked into the B2M locus with simultaneous B2M knock out and expressed using a using a dual promoter system containing both the endogenous B2M promoter as well as an exogenously provided EF1a promoter. The endogenous B2M promoter alone was insufficient to produce high levels of RACR. This dual promoter system resulted in RACR levels that were equivalent or higher than what can be reached through lentiviral transduction of RACR with a strong exogenous MND promoter.

Simultaneously, CRISPR-Cas9 was used to generate site-specific knock-ins of constructs encoding an anti-CD19 chimeric antigen receptor (CAR) via homology-directed repair (HDR). The nucleotide sequence encoding the CAR is set forth in SEQ ID NO: 50 and encodes the amino acid sequence set forth in SEQ ID NO: 51. Specifically, the CAR was knocked-in at the AAVS1 locus (AAVS1 homology arms SEQ ID NOs: 53-54; gRNA SEQ ID NO: 52).

### Example 2 Generation of FKBP12 Knock-Out iPSCs

This example demonstrates the knock-out of the FKBP12 gene in RACR-iPSC cells. RACR requires rapamycin for activation; however, rapamycin has the potential to inhibit cell growth. To generate rapamycin resistant cells, the FKBP12 gene was knocked-out, using Cas9 and a pool of gRNAs set forth in SEQ ID NOS: 19-21.

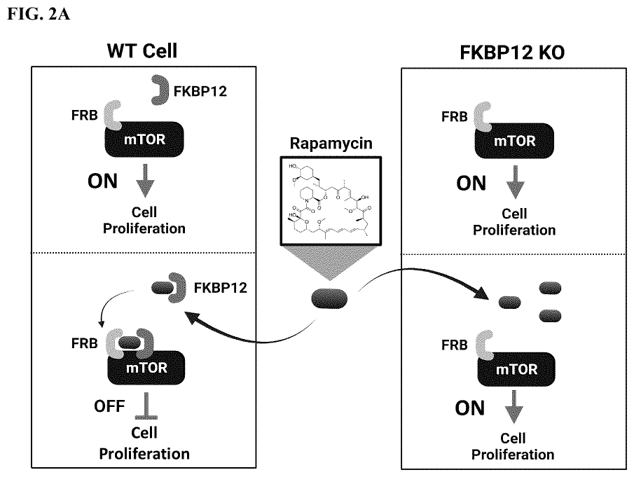

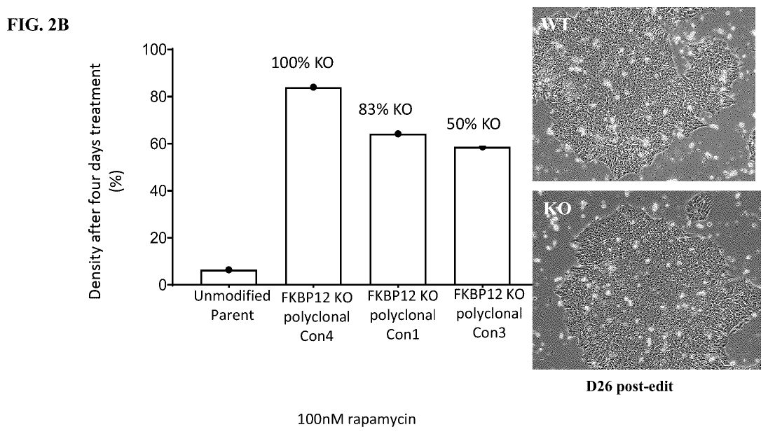

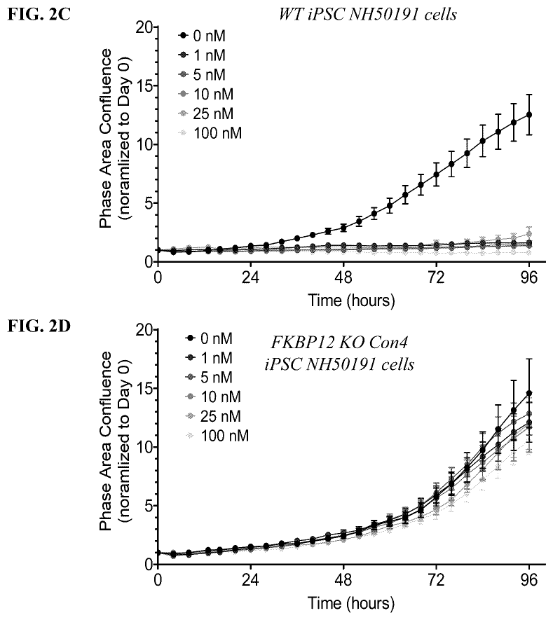

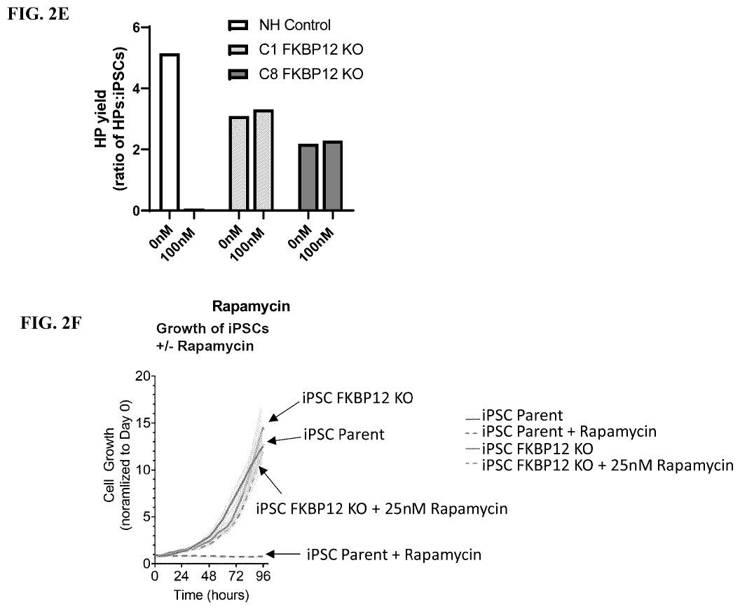

A schematic depicting the role of FKBP12 in the inhibition of proliferation by rapamycin via mTOR is shown in FIG. 2A. In wildtype cells, FKBP12 binds to rapamycin creating a novel binding surface that enables FKBP12-rapamycin to dimerize with the FRB domain of mTOR and subsequently inhibit mTOR. In the absence of FKBP12, rapamycin cannot interact with the mTOR complex and thus loses its inhibitory capacity. Rapamycin-mediated inhibition of iPSC proliferation in polyclonal FKBP12 knock-out (KO) lines is shown in FIG. 2B, left panel. The efficiency of the knockout was determined by Sanger sequencing. Con1, Con2, or Con3 indicate different electroporation conditions for CRISPR-Cas9 based editing resulting in differing levels of knockout. Phase-contrast images reveal normal iPSC morphology in both wild type and FKBP12 KO cells, FIG. 2B, right panel. The confluency of wildtype iPSCs after four days of treatment with varying doses of rapamycin with inhibition of growth at 1 nM rapamycin or higher is shown in FIG. 2C. The growth of FKBP12 KO iPSCs during four days of treatment with varying doses of rapamycin demonstrated robust growth even at doses up to 100 nM rapamycin, confirming rapamycin resistance of FKBP12 KO lines is shown in FIG. 2D and FIG. 2F. The ability of clonal FKBP12 KO iPSCs to differentiate to hematopoietic progenitors (HPs) in the presence of rapamycin is shown in FIG. 2E. In contrast, rapamycin fully inhibits HP differentiation of the parental wildtype iPSCs.

This experiment demonstrates that FKBP12 KO cell lines have enhanced HP differentiation compared to parental WT iPSCs.

### Example 3 Generation of RACR and CAR-Engineered Macrophages and Functionality

RACR CAR iPSCs (edited B2M-EF1a-RACR and knocked out for FKBP12), generated as described in Example 1 and 2, were generated. RACR+ CAR+ iPSC cells were then differentiated into RACR+ CAR+ macrophages and assessed for functional activity.

Embryoid Body iPSC Formation (Days 0-3)

Confluent RACR+ CD19CAR+ iPSCs (edited B2M-EF1a-RACR and knocked out for FKBP12) were passaged from Matrigel-coated 6-well plates using 0.5 mM EDTA and were added to 60 mL in a vertical wheel bioreactor. At day 0, embryoid bodies (EBs) were generated from a single cell suspension of iPSCs in STEMdiff APEL 2 Medium (STEMCELL Technologies) supplemented with 10 ng/mL BMP4, 10 ng/mL FGF2, 50 ng/mL VEGF-165, 10 μM Y27632, and rapamycin (10 nM) to initiate mesoderm differentiation.

### Hematopoietic Progenitor Cell Differentiation (Days 3-15)

Mesoderm was differentiated to hematopoietic progenitors from days 3-15. 50 mL macarophage medium 1 (MM1) was added to the vertical wheel bioreactor for a total of 110 mL. MM1 included SFEM II (STEMCEll Technologies), 10 ng/mL BMP4, 50 ng/mL FGF2, 50 ng/mL VEGF, 4 μM LY294002, 10 μM rapamycin, 25 ng/mL IL-3, and 50 ng/mL M-CSF. From days 6-15, cells were cultured in macrophage medium 2 (MM2), which added 50 ng/mL GM-CSF, 1 μM UM729, and 1 μM StemRegenin-1 to the MM1 formulation. Cells were passaged 1:3 on day 6 (20 mL of the 110 mL EB suspension was added to 40 mL MM2) and continued in the vertical wheel bioreactors. On day 9, 50 mL MM2 was added to the vessel for a total of 110 mL. Starting on day 9, IL-3 concentration was increased to 40 ng/mL and the M-CSF and GM-CSF concentrations were increased to 100 ng/mL.

RACR+ CAR+ Macrophage (iMac) Differentiation and Maturation (Days 12-24)

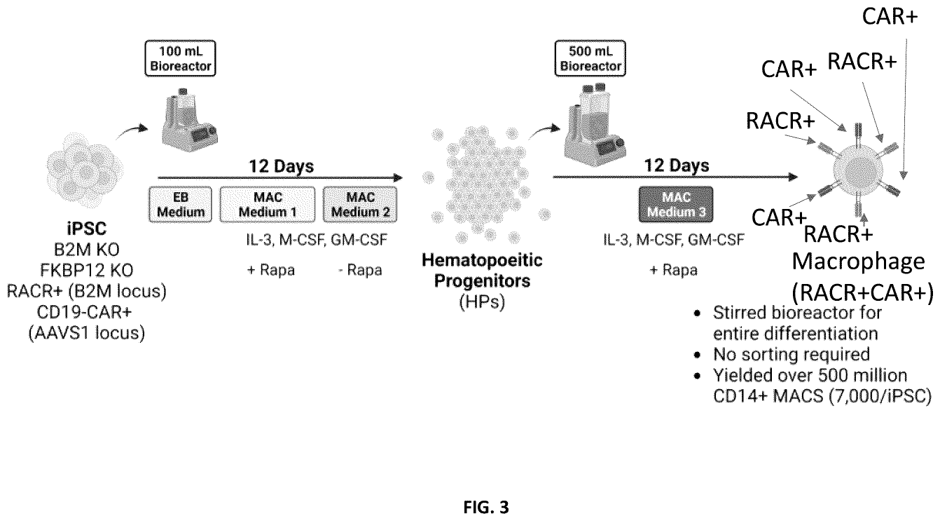

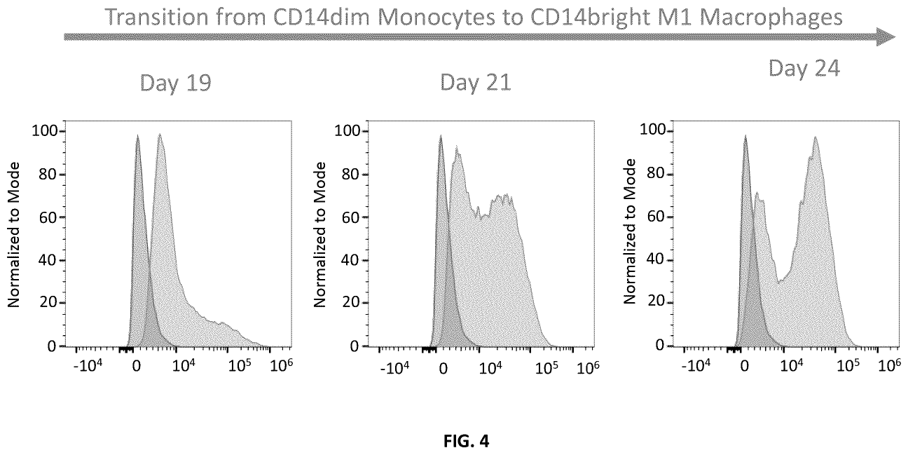

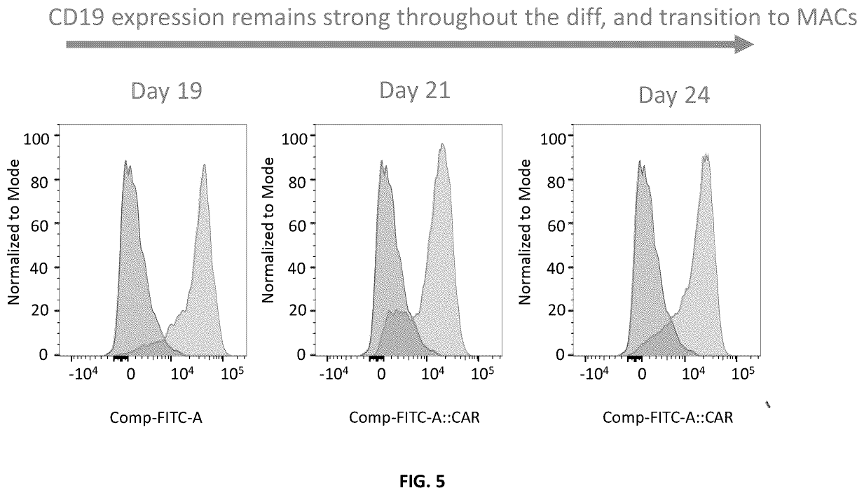

At Day 12 of culture, the hematopoietic progenitor (HP) cells were moved to a 500 mL vertical wheel bioreactor and continued in culture for up to 24 days for RACR macrophage cell generation. As shown in FIG. 3, the cells were cultured in macrophage medium 3 (MM3), which consisted of AIM V Medium (ThermoFisher), 10% Serum, 1× GlutaMAX, 100 ng/mL M-CSF, 100 ng/mL GM-CSF, 20 ng/mL SCF, 1 μM UM729, 1 μM StemRegenin1, and 20 ng/mL IL-3. 10 nM rapamycin was again included from days 16-24. In one experiment, the development of mature CD14+ macrophages was measured via flow cytometry on days 19, 21 and 24 (FIG. 4). As shown in FIG. 4, CD14 expression increased over time, developing from CD14-dim monocytes to mature CD14+ macrophages. Further, CD19CAR expression remained strong throughout the macrophage development, as measured by flow cytometry on days 19, 21, and 24 (FIG. 5). After 24 total days, the differentiation protocol yielded over 500 million CD14-expressing macrophages, for an approximate yield of 7,000 macrophages per 1 iPSC cell.

Next, functionality of the RACR+ CAR+ macrophages was examined.

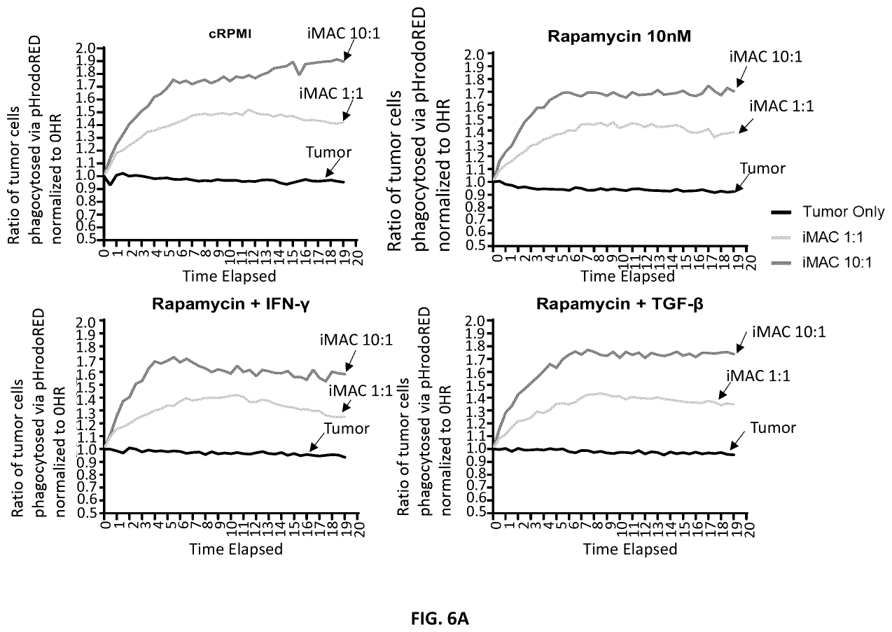

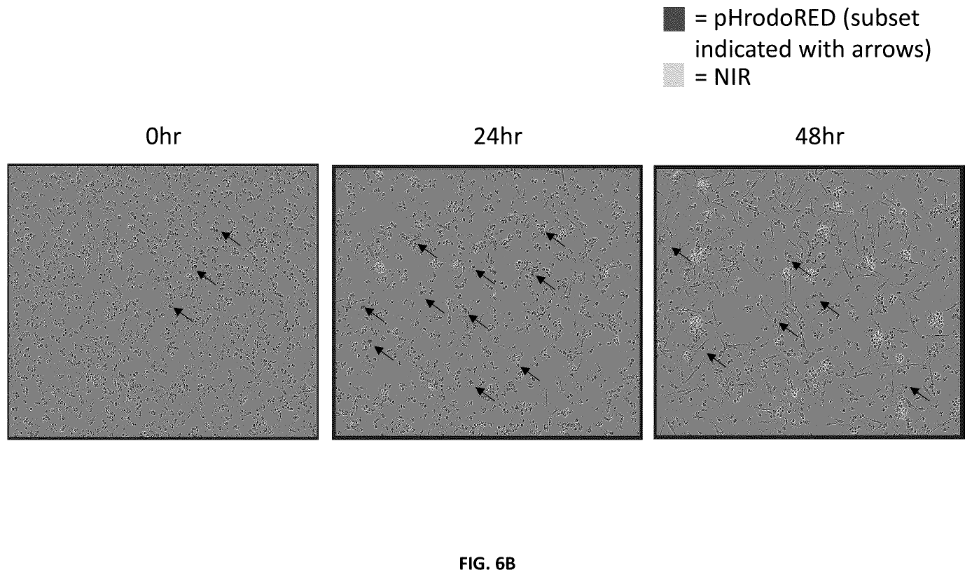

RACR+ CAR+ macrophages were co-cultured with tumor cells at various ratios in the presence or absence of rapamycin (10 nM), IFN-γ and/or TGF-β over the span of 48 hours (FIG. 6A). Tumor cells were stained with pHrodo™ Red ester and the cell culture was imaged by fluorescent microscopy (FIG. 6B). Tumor cells cultured with the RACR+ CAR+ macrophages at a 10:1 ratio had the highest ratio of phagocytosis as compared to the tumor only control (FIG. 6A). Inclusion of IFN-γ or TGF-β in the cell culture did not drastically affect the rate of phagocytosis.

This experiment demonstrates the functionality of macrophages transduced with RACR and a CD19 CAR.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ | | |
|---|---|---|
| ID NO: | Sequence | Description |
| 1 | ERTMPRIPTLKNLEDLVTEYHGNFSAWSGVSKGLAESLQPDYSERL | IL2RG Common |
| | CLVSEIPPKGGALG | Gamma Chain |
| | EGPGASPCNQHSPYWAPPCYTLKPET | Intracellular |
| | | domain |
| 2 | NCRNTGPWLKKVLKCNTPDPSKFFSQLSSEHGGDVQKWLSSPFPSS | IL2RB |
| | SFSPGGLAPEISPL | intracellular |
| | EVLERDKVTQLLLQQDKVPEPASLSSNHSLTSCFTNQGYFFFHLPD | domain |
| | ALEIEACQVYFTYD | |
| | PYSEEDPDEGVAGAPTGSSPQPLQPLSGEDDAYCTFPSRDDLLLFS | |
| | PSLLGGPSPPSTAP | |
| | GGSGAGEERMPPSLQERVPRDWDPQPLGPPTPGVPDLVDFQPPPEL | |
| | VLREAGEEVPDAGP | |
| | REGVSFPWSRPPGQGEFRALNARLPLNTDAYLSLQELQGQDPTHLV | |
| 3 | KKRIKPIVWPSLPDHKKTLEHLCKKPRKNLNVSFNPESFLDCQIHR | IL7RB |
| | VDDIQARDEVEGFL | intracellular |
| | QDTFPQQLEESEKQRLGGDVQSPNCPSEDVVITPESFGRDSSLTCL | domain |
| | AGNVSACDAPILSS | |
| | SRSLDCRESGKNGPHVYQDLLLSLGTTNSTLPPPFSLQSGILTLNP | |
| | VAQGQPILTSLGSN | |
| | QEEAYVTMSSFYQNQ | |
| 4 | SLKTHPLWRLWKKIWAVPSPERFFMPLYKGCSGDFKKWVGAPFTGS | IL21RB |
| | SLELGPWSPEVPST | intracellular |
| | LEVYSCHPPRSPAKRLQLTELQEPAELVESDGVPKPSFWPTAQNSG | domain |
| | GSAYSEERDRPYGL | |
| | VSIDTVTVLDAEGPCTWPCSCEDDGYPALDLDAGLEPSPGLEDPLL | |
| | DAGTTVLSCGCVSA | |
| | GSPGLGGPLGSLLDRLKPPLADGEDWAGGLPWGGRSPGGVSESEAG | |
| | SPLAGLDMDTFDSG | |
| | FVGSDCSSPVECDFTSPGDEGPPRSYLRQWVVIPPPLSSPGPQAS | |
| 5 | GVQVETISPGDGRTFPKRGQTCVVHYTGMLEDGKKFDSSRDRNKPF | FKBP |
| | KFMLGKQEVIRGWE | domain |
| | EGVAQMSVGQRAKLTISPDYAYGATGHPGIIPPHATLVFDVELLKL | (FKBP12) |
| | E | |
| 6 | ILWHEMWHEGLEEASRLYFGERNVKGMFEVLEPLHAMMERGPQTLK | FRB domain |
| | ETSFNQAYGRDLME | |
| | AQEWCRKYMKSGNVKDLTQAWDLYYHVFRRISK | |
| 7 | ILWHEMWHEGLEEASRLYFGERNVKGMFEVLEPLHAMMERGPQTLK | variant |
| | ETSFNQAYGRDLME | FRB domain |
| | AQEWCRKYMKSGNVKDLLQAWDLYYHVFRRISK | (FRB mutant |
| | | domain) |
| 8 | VVISVGSMGLIISLLCVYFWL | transmembrane |
| | | (TM) domain |
| 9 | VAVAGCVFLLISVLLLSGL | TM domain |
| 10 | PILLTISILSFFSVALLVILACVLW | TM domain |
| 11 | GWNPHLLLLLLLVIVFIPAFW | TM domain |
| 12 | MALPVTALLLPLALLLHAARP | CD8a |
| | | signal |
| | | sequence |
| 13 | ATGGCCTTACCAGTGACCGCCTTGCTCCTGCCGCTGGCCTTGCTGC | nucleotide |
| | TCCACGCCGCCAGGCCGGATGTCGTGATGACCCAGACCCCCCTCAG | sequence |
| | CCTCCCAGTGTCCCTCGGTGACCAGGCTTCTATTAGTTGCAGATCC | encoding a |
| | AGCCAGTCCCTCGTGCACTCTAACGGTAATACCTACCTGAGATGGT | CAR |
| | ATCTCCAGAAGCCCGGACAGAGCCCTAAGGTGCTGATCTACAAAGT | |
| | CTCCAACCGGGTGTCTGGAGTCCCTGACCGCTTCTCAGGGAGCGGT | |
| | TCCGGCACCGACTTCACCCTGAAGATCAACCGGGTGGAGGCCGAAG | |
| | ACCTCGGCGTCTATTTCTGCTCTCAGAGTACACATGTGCCCTGGAC | |
| | CTTCGGCGGAGGGACCAAGCTGGAGATCAAAAGCTCCGCAGACGAT | |
| | GCCAAGAAAGATGCCGCTAAGAAAGACGATGCTAAGAAAGACGATG | |
| | CAAAGAAAGACGGTGGCGTGAAGCTGGATGAAACCGGAGGAGGTCT | |
| | CGTCCAGCCAGGAGGAGCCATGAAGCTGAGTTGCGTGACCAGCGGA | |
| | TTCACCTTTGGGCACTACTGGATGAACTGGGTGCGACAGTCCCCAG | |
| | AGAAGGGGCTCGAATGGGTCGCTCAGTTCAGGAACAAACCCTACAA | |
| | TTATGAGACATACTATTCAGACAGCGTGAAGGGCAGGTTTACTATC | |
| | AGTAGAGACGATTCCAAATCTAGCGTGTACCTGCAGATGAACAATC | |
| | TCAGGGTCGAAGATACAGGCATCTACTATTGCACAGGGGCATCCTA | |
| | TGGTATGGAGTATCTCGGTCAGGGGACAAGCGTCACAGTCAGTTTC | |
| | GTGCCGGTCTTCCTGCCAGCGAAGCCCACCACGACGCCAGCGCCGC | |
| | GACCACCAACACCGGCGCCCACCATCGCGTCGCAGCCCCTGTCCCT | |
| | GCGCCCAGAGGCGTGCCGGCCAGCGGCGGGGGGCGCAGTGCACACG | |
| | AGGGGGCTGGACTTCGCCTGTGATATCTACATCTGGGCGCCCTTGG | |
| | CCGGGACTTGTGGGGTCCTTCTCCTGTCACTGGTTATCACCCTTTA | |
| | CTGCAACCACAGGAACCGTTTCTCTGTTGTTAAACGGGGCAGAAAG | |
| | AAACTCCTGTATATATTCAAACAACCATTTATGAGACCAGTACAAA | |
| | CTACTCAAGAGGAAGATGGCTGTAGCTGCCGATTTCCAGAAGAAGA | |
| | AGAAGGAGGATGTGAACTGAGAGTGAAGTTCAGCAGGAGCGCAGAC | |
| | GCCCCCGCGTACCAGCAGGGCCAGAACCAGCTCTATAACGAGCTCA | |
| | ATCTAGGACGAAGAGAGGAGTACGATGTTTTGGACAAGAGACGTGG | |
| | CCGGGACCCTGAGATGGGGGGAAAGCCGAGAAGGAAGAACCCTCAG | |
| | GAAGGCCTGTACAATGAACTGCAGAAAGATAAGATGGCGGAGGCCT | |
| | ACAGTGAGATTGGGATGAAAGGCGAGCGCCGGAGGGGCAAGGGGCA | |
| | CGATGGCCTTTACCAGGGTCTCAGTACAGCCACCAAGGACACCTAC | |
| | GACGCCCTTCACATGCAGGCCCTGCCCCCTCGCTAA | |
| 14 | MALPVTALLLPLALLLHAARPDVVMTQTPLSLPVSLGDQASISCRS | CAR amino |
| | SQSLVHSNGNTYLR | acid |
| | WYLQKPGQSPKVLIYKVSNRVSGVPDRFSGSGSGTDFTLKINRVEA | sequence |
| | EDLGVYFCSQSTHV | |
| | PWTFGGGTKLEIKSSADDAKKDAAKKDDAKKDDAKKDGGVKLDETG | |
| | GGLVQPGGAMKLSC | |
| | VTSGFTFGHYWMNWVRQSPEKGLEWVAQFRNKPYNYETYYSDSVKG | |
| | RFTISRDDSKSSVY | |
| | LQMNNLRVEDTGIYYCTGASYGMEYLGQGTSVTVSFVPVFLPAKPT | |
| | TTPAPRPPTPAPTI | |
| | ASQPLSLRPEACRPAAGGAVHTRGLDFACDIYIWAPLAGTCGVLLL | |
| | SLVITLYCNHRNRF | |
| | SVVKRGRKKLLYIFKQPFMRPVQTTQEEDGCSCRFPEEEEGGCELR | |
| | VKFSRSADAPAYQQ | |
| | GQNQLYNELNLGRREEYDVLDKRRGRDPEMGGKPRRKNPQEGLYNE | |
| | LQKDKMAEAYSEIG | |
| | MKGERRRGKGHDGLYQGLSTATKDTYDALHMQALPPR | |
| 15 | GCCACCATGGCCTTACCAGTGACCGCCTTGCTCCTGCCGCTGGCCT | nucleotide |
| | TGCTGCTCCACGCCGCCAGGCCGGATGTCGTGATGACCCAGACCCC | insert |
| | CCTCAGCCTCCCAGTGTCCCTCGGTGACCAGGCTTCTATTAGTTGC | |
| | AGATCCAGCCAGTCCCTCGTGCACTCTAACGGTAATACCTACCTGA | |
| | GATGGTATCTCCAGAAGCCCGGACAGAGCCCTAAGGTGCTGATCTA | |
| | CAAAGTCTCCAACCGGGTGTCTGGAGTCCCTGACCGCTTCTCAGGG | |
| | AGCGGTTCCGGCACCGACTTCACCCTGAAGATCAACCGGGTGGAGG | |
| | CCGAAGACCTCGGCGTCTATTTCTGCTCTCAGAGTACACATGTGCC | |
| | CTGGACCTTCGGCGGAGGGACCAAGCTGGAGATCAAAAGCTCCGCA | |
| | GACGATGCCAAGAAAGATGCCGCTAAGAAAGACGATGCTAAGAAAG | |
| | ACGATGCAAAGAAAGACGGTGGCGTGAAGCTGGATGAAACCGGAGG | |
| | AGGTCTCGTCCAGCCAGGAGGAGCCATGAAGCTGAGTTGCGTGACC | |
| | AGCGGATTCACCTTTGGGCACTACTGGATGAACTGGGTGCGACAGT | |
| | CCCCAGAGAAGGGGCTCGAATGGGTCGCTCAGTTCAGGAACAAACC | |
| | CTACAATTATGAGACATACTATTCAGACAGCGTGAAGGGCAGGTTT | |
| | ACTATCAGTAGAGACGATTCCAAATCTAGCGTGTACCTGCAGATGA | |
| | ACAATCTCAGGGTCGAAGATACAGGCATCTACTATTGCACAGGGGC | |
| | ATCCTATGGTATGGAGTATCTCGGTCAGGGGACAAGCGTCACAGTC | |
| | AGTTTCGTGCCGGTCTTCCTGCCAGCGAAGCCCACCACGACGCCAG | |
| | CGCCGCGACCACCAACACCGGCGCCCACCATCGCGTCGCAGCCCCT | |
| | GTCCCTGCGCCCAGAGGCGTGCCGGCCAGCGGCGGGGGGCGCAGTG | |
| | CACACGAGGGGGCTGGACTTCGCCTGTGATATCTACATCTGGGCGC | |
| | CCTTGGCCGGGACTTGTGGGGTCCTTCTCCTGTCACTGGTTATCAC | |
| | CCTTTACTGCAACCACAGGAACCGTTTCTCTGTTGTTAAACGGGGC | |
| | AGAAAGAAACTCCTGTATATATTCAAACAACCATTTATGAGACCAG | |
| | TACAAACTACTCAAGAGGAAGATGGCTGTAGCTGCCGATTTCCAGA | |
| | AGAAGAAGAAGGAGGATGTGAACTGAGAGTGAAGTTCAGCAGGAGC | |
| | GCAGACGCCCCCGCGTACCAGCAGGGCCAGAACCAGCTCTATAACG | |
| | AGCTCAATCTAGGACGAAGAGAGGAGTACGATGTTTTGGACAAGAG | |
| | ACGTGGCCGGGACCCTGAGATGGGGGGAAAGCCGAGAAGGAAGAAC | |
| | CCTCAGGAAGGCCTGTACAATGAACTGCAGAAAGATAAGATGGCGG | |
| | AGGCCTACAGTGAGATTGGGATGAAAGGCGAGCGCCGGAGGGGCAA | |
| | GGGGCACGATGGCCTTTACCAGGGTCTCAGTACAGCCACCAAGGAC | |
| | ACCTACGACGCCCTTCACATGCAGGCCCTGCCCCCTCGCTAA | |
| 16 | DVVMTQTPLSLPVSLGDQASISCRSSQSLVHSNGNTYLRWYLQKPG | CAR amino |
| | QSPKVLIYKVSNRVSGVPDRFSGSGSGTDFTLKINRVEAEDLGVYF | acid |
| | CSQSTHV | sequence |
| | PWTFGGGTKLEIKSSADDAKKDAAKKDDAKKDDAKKDGGVKLDETG | without |
| | GGLVQPGGAMKLSC | the signal |
| | VTSGFTFGHYWMNWVRQSPEKGLEWVAQFRNKPYNYETYYSDSVKG | peptide |
| | RFTISRDDSKSSVY | |
| | LQMNNLRVEDTGIYYCTGASYGMEYLGQGTSVTVSFVPVFLPAKPT | |
| | TTPAPRPPTPAPTI | |
| | ASQPLSLRPEACRPAAGGAVHTRGLDFACDIYIWAPLAGTCGVLLL | |
| | SLVITLYCNHRNRF | |
| | SVVKRGRKKLLYIFKQPFMRPVQTTQEEDGCSCRFPEEEEGGCELR | |
| | VKFSRSADAPAYQQ | |
| | GQNQLYNELNLGRREEYDVLDKRRGRDPEMGGKPRRKNPQEGLYNE | |
| | LQKDKMAEAYSEIG | |
| | MKGERRRGKGHDGLYQGLSTATKDTYDALHMQALPPR | |
| 17 | MALPVTALLLPLALLLHAARP | CAR amino |
| | | acid sequence |
| | | signal |
| | | peptide |
| 18 | GGCCGAGATGTCTCGCTCCG | B2M gRNA |
| 19 | CACGCAGGTCTGGCCGCGCT | FKBP12 |
| | | gRNA |
| 20 | TGCTGAGCCGATGCGCGCGG | FKBP12 |
| | | gRNA |
| 21 | CTCACCGTCTCCTGGGGAGA | FKBP12 |
| | | gRNA |
| 22 | TACCCAGAGAATGGAGAAACCCTGCAGGGAATTCCCAAGCTGTAGT | B2M 5′ |
| | TATAAACAGAAGTTCTCCTTCTGCTAGGTAGCATTCAAAGATCTTA | homology |
| | ATCTTCTGGGTTTCCGTTTTCTCGAATGAAAAATGCAGGTCCGAGC | arm |
| | AGTTAACTGGCTGGGGCACCATTAGCAAGTCACTTAGCATCTCTGG | |
| | GGCCAGTCTGCAAAGCGAGGGGGCAGCCTTAATGTGCCTCCAGCCT | |
| | GAAGTCCTAGAATGAGCGCCCGGTGTCCCAAGCTGGGGCGCGCACC | |
| | CCAGATCGGAGGGCGCCGATGTACAGACAGCAAACTCACCCAGTCT | |
| | AGTGCATGCCTTCTTAAACATCACGAGACTCTAAGAAAAGGAAACT | |
| | GAAAACGGGAAAGTCCCTCTCTCTAACCTGGCACTGCGTCGCTGGC | |
| | TTGGAGACAGGTGACGGTCCCTGCGGGCCTTGTCCTGATTGGCTGG | |
| | GCACGCGTTTAATATAAGTGGAGGCGTCGCGCTGGCGGGC | |
| 23 | TGGCCTTAGCTGTGCTCGCGCTACTCTCTCTTTCTGGCCTGGAGGC | B2M 3′ |
| | TATCCAGCGTGAGTCTCTCCTACCCTCCCGCTCTGGTCCTTCCTCT | homology |
| | CCCGCTCTGCACCCTCTGTGGCCCTCGCTGTGCTCTCTCGCTCCGT | arm |
| | GACTTCCCTTCTCCAAGTTCTCCTTGGTGGCCCGCCGTGGGGCTAG | |
| | TCCAGGGCTGGATCTCGGGGAAGCGGCGGGGTGGCCTGGGAGTGGG | |
| | GAAGGGGGTGCGCACCCGGGACGCGCGCTACTTGCCCCTTTCGGCG | |
| | GGGAGCAGGGGAGACCTTTGGCCTACGGCGACGGGAGGGTCGGGAC | |
| | AAAGTTTAGGGCGTCGATAAGCGTCAGAGCGCCGAGGTTGGGGGAG | |
| | GGTTTCTCTTCCGCTCTTTCGCGGGGCCTCTGGCTCCCCCAGCGCA | |
| | GCTGGAGTGGGGGACGGGTAGGCTCGTCCCAAAGGCGCGGCGCTGA | |
| | GGTTTGTGAACGCGTGGAGGGGCGCTTGGGGTCTGGGGGA | |
| 24 | cgtgaggctccggtgcccgtcagtgggcagagcgcacatcgcccac | EF1alpha |
| | agtccccgagaagttggggggaggggtcggcaattgaaccggtgcc | promoter |
| | tagagaaggtggcgcggggtaaactgggaaagtgatgtcgtgtact | |
| | ggctccgcctttttcccgagggtgggggagaaccgtatataagtgc | |
| | agtagtcgccgtgaacgttctttttcgcaacgggtttgccgccaga | |
| | acacaggtaagtgccgtgtgtggttcccgcgggcctggcctcttta | |
| | cgggttatggcccttgcgtgccttgaattacttccacgcccctggc | |
| | tgcagtacgtgattcttgatcccgagcttcgggttggaagtgggtg | |
| | ggagagttcgaggccttgcgcttaaggagccccttcgcctcgtgct | |
| | tgagttgaggcctggcctgggcgctggggccgccgcgtgcgaatct | |
| | ggtggcaccttcgcgcctgtctcgctgctttcgataagtctctagc | |
| | catttaaaatttttgatgacctgctgcgacgctttttttctggcaa | |
| | gatagtcttgtaaatgcgggccaagatctgcacactggtatttcgg | |
| | tttttggggccgcgggcggcgacggggcccgtgcgtcccagcgcac | |
| | atgttcggcgaggcggggcctgcgagcgcggccaccgagaatcgga | |
| | cgggggtagtctcaagctggccggcctgctctggtgcctggcctcg | |
| | cgccgccgtgtatcgccccgccctgggcggcaaggctggcccggtc | |
| | ggcaccagttgcgtgagcggaaagatggccgcttcccggccctgct | |
| | gcagggagctcaaaatggaggacgcggcgctcgggagagcgggcgg | |
| | gtgagtcacccacacaaaggaaaagggcctttccgtcctcagccgt | |
| | cgcttcatgtgactccacggagtaccgggcgccgtccaggcacctc | |
| | gattagttctcgagcttttggagtacgtcgtctttaggttgggggg | |
| | aggggttttatgcgatggagtttccccacactgagtgggtggagac | |
| | tgaagttaggccagcttggcacttgatgtaattctccttggaattt | |
| | gccctttttgagtttggatcttggttcattctcaagcctcagacag | |
| | tggttcaaagtttttttcttccatttcaggtgtcgtgaa | |
| 25 | CGTGAGGCTCCGGTGCCCGTCAGTGGGCAGAGCGCACATCGCCCAC | EF1alpha |
| | AGTCCCCGAGAAGTTGGGGGGAGGGGTCGGCAATTGAACCGGTGCC | promoter |
| | TAGAGAAGGTGGCGCGGGGTAAACTGGGAAAGTGATGTCGTGTACT | |
| | GGCTCCGCCTTTTTCCCGAGGGTGGGGGAGAACCGTATATAAGTGC | |
| | AGTAGTCGCCGTGAACGTTCTTTTTCGCAACGGGTTTGCCGCCAGA | |
| | ACACAGGTAAGTGCCGTGTGTGGTTCCCGCGGGCCTGGCCTCTTTA | |
| | CGGGTTATGGCCCTTGCGTGCCTTGAATTACTTCCACGCCCCTGGC | |
| | TGCAGTACGTGATTCTTGATCCCGAGCTTCGGGTTGGAAGTGGGTG | |
| | GGAGAGTTCGAGGCCTTGCGCTTAAGGAGCCCCTTCGCCTCGTGCT | |
| | TGAGTTGAGGCCTGGCTTGGGCGCTGGGGCCGCCGCGTGCGAATCT | |
| | GGTGGCACCTTCGCGCCTGTCTCGCTGCTTTCGATAAGTCTCTAGC | |
| | CATTTAAAATTTTTGATGACCTGCTGCGACGCTTTTTTTCTGGCAA | |
| | GATAGTCTTGTAAATGCGGGCCAAGATCTGCACACTGGTATTTCGG | |
| | TTTTTGGGGCCGCGGGCGGCGACGGGGCCCGTGCGTCCCAGCGCAC | |
| | ATGTTCGGCGAGGCGGGGCCTGCGAGCGCGGCCACCGAGAATCGGA | |
| | CGGGGGTAGTCTCAAGCTGGCCGGCCTGCTCTGGTGCCTGGCCTCG | |
| | CGCCGCCGTGTATCGCCCCGCCCTGGGCGGCAAGGCTGGCCCGGTC | |
| | GGCACCAGTTGCGTGAGCGGAAAGATGGCCGCTTCCCGGCCCTGCT | |
| | GCAGGGAGCTCAAAATGGAGGACGCGGCGCTCGGGAGAGCGGGCGG | |
| | GTGAGTCACCCACACAAAGGAAAAGGGCCTTTCCGTCCTCAGCCGT | |
| | CGCTTCATGTGACTCCACGGAGTACCGGGCGCCGTCCAGGCACCTC | |
| | GATTAGTTCTCGAGCTTTTGGAGTACGTCGTCTTTAGGTTGGGGGG | |
| | AGGGGTTTTATGCGATGGAGTTTCCCCACACTGAGTGGGTGGAGAC | |
| | TGAAGTTAGGCCAGCTTGGCACTTGATGTAATTCTCCTTGGAATTT | |
| | GCCCTTTTTGAGTTTGGATCTTGGTTCATTCTCAAGCCTCAGACAG | |
| | TGGTTCAAAGTTTTTTTCTTCCATTTCAGGTGTCGTGA | |
| 26 | CGTGAGGCTCCGGTGCCCGTCAGTGGGCAGAGCGCACATCGCCCAC | EF1alpha |
| | AGTCCCCGAGAAGTTGGGGGGAGGGGTCGGCAATTGAACCGGTGCC | promoter |
| | TAGAGAAGGTGGCGCGGGGTAAACTGGGAAAGTGATGTCGTGTACT | |
| | GGCTCCGCCTTTTTCCCGAGGGTGGGGGAGAACCGTATATAAGTGC | |
| | ACTAGTCGCCGTGAACGTTCTTTTTCGCAACGGGTTTGCCGCCAGA | |
| | ACACAGGTAAGTGCCGTGTGTGGTTCCCGCGGGCCTGGCCTCTTTA | |
| | CGGGTTATGGCCCTTGCGTGCCTTGAATTACTTCCACCTGGCTGCA | |
| | GTACGTGATTCTTGATCCCGAGCTTCGGGTTGGAAGTGGGTGGGAG | |
| | AGTTCGTGGCCTTGCGCTTAAGGAGCCCCTTCGCCTCGTGCTTGAG | |
| | TTGTGGCCTGGCCTGGGCGCTGGGGCCGCCGCGTGCGAATCTGGTG | |
| | GCACCTTCGCGCCTGTCTCGCTGCTTTCGATAAGTCTCTAGCCATT | |
| | TAAAATTTTTGATGACCTGCTGCGACGCTTTTTTTCTGGCAAGATA | |
| | GTCTTGTAAATGCGGGCCAAGATCAGCACACTGGTATTTCGGTTTT | |
| | TGGGGCCGCGGGCGGCGACGGGGCCCGTGCGTCCCAGCGCACATGT | |
| | TCGGCGAGGCGGGGCCTGCGAGCGCGGCCACCGAGAATCGGACGGG | |
| | GGTAGTCTCAAGCTGCCCGGCCTGCTCTGGTGCCTGGCCTCGCGCC | |
| | GCCGTGTATCGCCCCGCCCTGGGCGGCAAGGCTGGCCCGGTCGGCA | |
| | CCAGTTGCGTGAGCGGAAAGATGGCCGCTTCCCGGCCCTGCTGCAG | |
| | GGAGCACAAAATGGAGGACGCGGCGCTCGGGAGAGCGGGCGGGTGA | |
| | GTCACCCACACAAAGGAAAAGGGCCTTTCCGTCCTCAGCCGTCGCT | |
| | TCATGTGACTCCACGGAGTACCGGGCGCCGTCCAGGCACCTCGATT | |
| | AGTTCTCCAGCTTTTGGAGTACGTCGTCTTTAGGTTGGGGGGAGGG | |
| | GTTTTATGCGATGGAGTTTCCCCACACTGAGTGGGTGGAGACTGAA | |
| | GTTAGGCCAGCTTGGCACTTGATGTAATTCTCCTTGGAATTTGCCC | |
| | TTTTTGAGTTTGGATCTTGGTTCATTCTCAAGCCTCAGACAGTGGT | |
| | TCAAAGTTTTTTTCTTCCATTTCAGGTGTCGTGAAAACTACCCCTA | |
| | AAAGCCAAA | |
| 27 | gaacagagaaacaggagaatatgggccaaacaggatatctgtggta | MND |
| | agcagttcctgccccggctcagggccaagaacagttggaacagcag | promoter |
| | aatatgggccaaacaggatatctgtggtaagcagttcctgccccgg | |
| | ctcagggccaagaacagatggtccccagatgcggtcccgccctcag | |
| | cagtttctagagaaccatcagatgtttccagggtgccccaaggacc | |
| | tgaaatgaccctgtgccttatttgaactaaccaatcagttcgcttc | |
| | tcgcttctgttcgcgcgcttctgctccccgagctctatataagcag | |
| | agctcgtttagtgaaccgtcagatc | |
| 28 | GVQVETISPGDGRTFPKRGQTCVVHYTGMLEDGKKFDSSRDRNKPF | RACR (g) |
| | KFMLGKQEVIRGWEEGVAQMSVGQRAKLTISPDYAYGATGHPGIIP | |
| | PHATLVFDVELLKLGEGSNTSKENPFLFALEAVVISVGSMGLIISL | |
| | LCVYFWLERTMPRIPTLKNLEDLVTEYHGNFSAWSGVSKGLAESLQ | |
| | PDYSERLCLVSEIPPKGGALGEGPGASPCNQHSPYWAPPCYTLKPE | |
| | T | |
| 29 | MPLGLLWLGLALLGALHAQA | Signal |
| | | peptide |
| 30 | GVQVETISPGDGRTFPKRGQTCVVHYTGMLEDGKKFDSSRDRNKPF | FKBP |
| | KFMLGKQEVIRGWEEGVAQMSVGQRAKLTISPDYAYGATGHPGIIP | domain |
| | PHATLVFDVELLKLGE | (FKBP12) |
| 31 | GSNTSKENPFLFALEAVVISVGSMGLIISLLCVYFWL | IL2RG |
| | | transmembrane |
| | | domain and |
| | | ectodomain |
| | | portion |
| 32 | CGTGAGGCTCCGGTGCCCGTCAGTGGGCAGAGCGCACATCGCCCAC | RACR |
| | AGTCCCCGAGAAGTTGGGGGGAGGGGTCGGCAATTGAACCGGTGCC | |
| | TAGAGAAGGTGGCGCGGGGTAAACTGGGAAAGTGATGTCGTGTACT | |
| | GGCTCCGCCTTTTTCCCGAGGGTGGGGGAGAACCGTATATAAGTGC | |
| | AGTAGTCGCCGTGAACGTTCTTTTTCGCAACGGGTTTGCCGCCAGA | |
| | ACACAGGTAAGTGCCGTGTGTGGTTCCCGCGGGCCTGGCCTCTTTA | |
| | CGGGTTATGGCCCTTGCGTGCCTTGAATTACTTCCACGCCCCTGGC | |
| | TGCAGTACGTGATTCTTGATCCCGAGCTTCGGGTTGGAAGTGGGTG | |
| | GGAGAGTTCGAGGCCTTGCGCTTAAGGAGCCCCTTCGCCTCGTGCT | |
| | TGAGTTGAGGCCTGGCTTGGGCGCTGGGGCCGCCGCGTGCGAATCT | |
| | GGTGGCACCTTCGCGCCTGTCTCGCTGCTTTCGATAAGTCTCTAGC | |
| | CATTTAAAATTTTTGATGACCTGCTGCGACGCTTTTTTTCTGGCAA | |
| | GATAGTCTTGTAAATGCGGGCCAAGATCTGCACACTGGTATTTCGG | |
| | TTTTTGGGGCCGCGGGCGGCGACGGGGCCCGTGCGTCCCAGCGCAC | |
| | ATGTTCGGCGAGGCGGGGCCTGCGAGCGCGGCCACCGAGAATCGGA | |
| | CGGGGGTAGTCTCAAGCTGGCCGGCCTGCTCTGGTGCCTGGCCTCG | |
| | CGCCGCCGTGTATCGCCCCGCCCTGGGCGGCAAGGCTGGCCCGGTC | |
| | GGCACCAGTTGCGTGAGCGGAAAGATGGCCGCTTCCCGGCCCTGCT | |
| | GCAGGGAGCTCAAAATGGAGGACGCGGCGCTCGGGAGAGCGGGCGG | |
| | GTGAGTCACCCACACAAAGGAAAAGGGCCTTTCCGTCCTCAGCCGT | |
| | CGCTTCATGTGACTCCACGGAGTACCGGGCGCCGTCCAGGCACCTC | |
| | GATTAGTTCTCGAGCTTTTGGAGTACGTCGTCTTTAGGTTGGGGGG | |
| | AGGGGTTTTATGCGATGGAGTTTCCCCACACTGAGTGGGTGGAGAC | |
| | TGAAGTTAGGCCAGCTTGGCACTTGATGTAATTCTCCTTGGAATTT | |
| | GCCCTTTTTGAGTTTGGATCTTGGTTCATTCTCAAGCCTCAGACAG | |
| | TGGTTCAAAGTTTTTTTCTTCCATTTCAGGTGTCGTGA | |
| | GCCACC | |
| | ATGCCACTGGGACTGCTGTGGCTGGGACTGGCCCTGCTGGGCGCCC | |
| | TGCACGCCCAGGCCGGCGTGCAGGTGGAGACAATCAGCCCAGGCGA | |
| | CGGACGCACCTTTCCTAAGCGGGGCCAGACATGCGTGGTGCACTAC | |
| | ACCGGCATGCTGGAGGATGGCAAGAAGTTCGACTCTAGCAGAGATA | |
| | GGAACAAGCCCTTTAAGTTCATGCTGGGCAAGCAGGAAGTGATCAG | |
| | AGGCTGGGAGGAGGGCGTGGCCCAGATGTCTGTGGGCCAGAGGGCC | |
| | AAGCTGACAATCAGCCCAGACTACGCATATGGAGCAACCGGACACC | |
| | CAGGAATCATCCCACCTCACGCCACACTGGTGTTCGATGTGGAGCT | |
| | GCTGAAGCTGGGCGAGGGCTCTAACACCAGCAAGGAGAATCCATTT | |
| | CTGTTCGCACTGGAGGCAGTGGTCATCTCCGTGGGCTCTATGGGCC | |
| | TGATCATCTCCCTGCTGTGCGTGTACTTTTGGCTGGAGAGAACAAT | |
| | GCCCAGGATCCCTACCCTGAAGAACCTGGAGGACCTGGTGACCGAG | |
| | TATCACGGCAATTTCAGCGCCTGGTCCGGCGTGTCTAAGGGACTGG | |
| | CAGAGTCCCTGCAGCCAGATTACTCTGAGCGGCTGTGCCTGGTGAG | |
| | CGAGATCCCACCCAAGGGAGGCGCCCTGGGAGAGGGACCTGGAGCA | |
| | AGCCCATGCAACCAGCACTCCCCATATTGGGCCCCTCCATGTTACA | |
| | CCCTGAAGCCTGAGACA | |
| | GGAAGCGGAGCGACGAATTTTAGTCTACTGAAACAAGCGGGAGACG | |
| | TGGAGGAAAACCCTGGACCT | |
| | ATGGCACTGCCAGTGACCGCCCTGCTGCTGCCACTGGCCCTGCTGC | |
| | TGCACGCAGCACGCCCCATCCTGTGGCACGAAATGTGGCATGAAGG | |
| | CCTGGAGGAGGCAAGCAGACTGTATTTTGGCGAGAGAAATGTGAAA | |
| | GGAATGTTTGAGGTGCTGGAGCCACTGCACGCAATGATGGAGAGGG | |
| | GACCTCAGACCCTGAAGGAGACATCCTTTAACCAGGCCTATGGCAG | |
| | AGACCTGATGGAGGCCCAGGAGTGGTGCAGGAAGTACATGAAGAGC | |
| | GGAAATGTGAAAGACCTGCTGCAGGCCTGGGATCTGTATTATCACG | |
| | TGTTCAGAAGGATCAGCAAGGGCAAGGATACCATCCCTTGGCTGGG | |
| | ACACCTGCTGGTGGGACTGTCCGGAGCCTTTGGCTTCATCATCCTG | |
| | GTGTACCTGCTGATCAACTGCCGCAATACAGGCCCATGGCTGAAGA | |
| | AGGTGCTGAAGTGTAACACCCCAGACCCCTCCAAGTTCTTTTCTCA | |
| | GCTGTCCTCTGAGCACGGCGGCGATGTGCAGAAGTGGCTGAGCTCC | |
| | CCTTTTCCATCTAGCTCCTTCAGCCCTGGAGGACTGGCACCAGAGA | |
| | TCTCCCCTCTGGAGGTGCTGGAGCGGGACAAGGTGACCCAGCTGCT | |
| | GCTGCAGCAGGATAAGGTGCCCGAGCCTGCCTCCCTGTCTAGCAAC | |
| | CACAGCCTGACCTCCTGCTTTACAAATCAGGGCTATTTCTTTTTCC | |
| | ACCTGCCAGACGCACTGGAGATCGAGGCATGTCAGGTGTACTTCAC | |
| | ATATGATCCATACAGCGAGGAGGACCCAGATGAGGGAGTGGCCGGC | |
| | GCCCCTACCGGATCCTCTCCACAGCCACTGCAGCCACTGTCTGGCG | |
| | AGGACGATGCCTACTGTACATTTCCTAGCAGAGACGATCTGCTGCT | |
| | GTTCTCCCCTTCTCTGCTGGGAGGACCATCTCCACCTAGCACCGCA | |
| | CCTGGAGGATCCGGGGCAGGGGAGGAGAGGATGCCACCATCTCTGC | |
| | AGGAGAGGGTGCCAAGGGACTGGGATCCTCAGCCACTGGGACCTCC | |
| | AACCCCAGGAGTGCCCGACCTGGTGGATTTCCAGCCCCCTCCAGAG | |
| | CTGGTGCTGAGAGAGGCAGGAGAGGAGGTGCCAGACGCAGGACCAA | |
| | GAGAGGGCGTGAGCTTTCCATGGTCCAGGCCACCTGGACAGGGAGA | |
| | GTTCAGAGCCCTGAACGCCAGGCTGCCACTGAATACAGACGCCTAC | |
| | CTGTCTCTGCAGGAGCTGCAGGGCCAGGATCCCACCCACCTGGTG | |
| | TAAAC | |
| | AATAAAAGATCTTTATTTTCATTAGATCTGTGTGTTGGTTTTTTGT | |
| | GTG | |
| 33 | MALPVTALLLPLALLLHAARPILWHEMWHEGLEEASRLYFGERNVK | RACR (b) |
| | GMFEVLEPLHAMMERGPQTLKETSFNQAYGRDLMEAQEWCRKYMKS | With |
| | GNVKDLLQAWDLYYHVFRRISKGKDTIPWLGHLLVGLSGAFGFIIL | signal |
| | VYLLINCRNTGPWLKKVLKCNTPDPSKFFSQLSSEHGGDVQKWLSS | peptide |
| | PFPSSSFSPGGLAPEISPLEVLERDKVTQLLLQQDKVPEPASLSSN | |
| | HSLTSCFTNQGYFFFHLPDALEIEACQVYFTYDPYSEEDPDEGVAG | |
| | APTGSSPQPLQPLSGEDDAYCTFPSRDDLLLFSPSLLGGPSPPSTA | |
| | PGGSGAGEERMPPSLQERVPRDWDPQPLGPPTPGVPDLVDFQPPPE | |
| | LVLREAGEEVPDAGPREGVSFPWSRPPGQGEFRALNARLPLNTDAY | |
| | LSLQELQGQDPTHLV | |
| 34 | MALPVTALLLPLALLLHAARP | CD8a |
| | | Signal |
| | | peptide |
| 35 | GKDTIPWLGHLLVGLSGAFGFIILVYLLI | IL2RB |
| | | transmembrane |
| | | domain and |
| | | ectodomain |
| | | portion |
| 36 | IPWLGHLLVGLSGAFGFIILVYLLI | IL2RB |
| | | transmembrane |
| | | domain |
| 37 | ATGCCACTGGGACTGCTGTGGCTGGGACTGGCCCTGCTGGGCGCCC | RACR (g) |
| | TGCACGCCCAGGCCGGCGTGCAGGTGGAGACAATCAGCCCAGGCGA | Nucleotides |
| | CGGACGCACCTTTCCTAAGCGGGGCCAGACATGCGTGGTGCACTAC | |
| | ACCGGCATGCTGGAGGATGGCAAGAAGTTCGACTCTAGCAGAGATA | |
| | GGAACAAGCCCTTTAAGTTCATGCTGGGCAAGCAGGAAGTGATCAG | |
| | AGGCTGGGAGGAGGGCGTGGCCCAGATGTCTGTGGGCCAGAGGGCC | |
| | AAGCTGACAATCAGCCCAGACTACGCATATGGAGCAACCGGACACC | |
| | CAGGAATCATCCCACCTCACGCCACACTGGTGTTCGATGTGGAGCT | |
| | GCTGAAGCTGGGCGAGGGCTCTAACACCAGCAAGGAGAATCCATTT | |
| | CTGTTCGCACTGGAGGCAGTGGTCATCTCCGTGGGCTCTATGGGCC | |
| | TGATCATCTCCCTGCTGTGCGTGTACTTTTGGCTGGAGAGAACAAT | |
| | GCCCAGGATCCCTACCCTGAAGAACCTGGAGGACCTGGTGACCGAG | |
| | TATCACGGCAATTTCAGCGCCTGGTCCGGCGTGTCTAAGGGACTGG | |
| | CAGAGTCCCTGCAGCCAGATTACTCTGAGCGGCTGTGCCTGGTGAG | |
| | CGAGATCCCACCCAAGGGAGGCGCCCTGGGAGAGGGACCTGGAGCA | |
| | AGCCCATGCAACCAGCACTCCCCATATTGGGCCCCTCCATGTTACA | |
| | CCCTGAAGCCTGAGACA | |
| 38 | ATGGCACTGCCAGTGACCGCCCTGCTGCTGCCACTGGCCCTGCTGC | RACR (b) |
| | TGCACGCAGCACGCCCCATCCTGTGGCACGAAATGTGGCATGAAGG | nucleotides |
| | CCTGGAGGAGGCAAGCAGACTGTATTTTGGCGAGAGAAATGTGAAA | |
| | GGAATGTTTGAGGTGCTGGAGCCACTGCACGCAATGATGGAGAGGG | |
| | GACCTCAGACCCTGAAGGAGACATCCTTTAACCAGGCCTATGGCAG | |
| | AGACCTGATGGAGGCCCAGGAGTGGTGCAGGAAGTACATGAAGAGC | |
| | GGAAATGTGAAAGACCTGCTGCAGGCCTGGGATCTGTATTATCACG | |
| | TGTTCAGAAGGATCAGCAAGGGCAAGGATACCATCCCTTGGCTGGG | |
| | ACACCTGCTGGTGGGACTGTCCGGAGCCTTTGGCTTCATCATCCTG | |
| | GTGTACCTGCTGATCAACTGCCGCAATACAGGCCCATGGCTGAAGA | |
| | AGGTGCTGAAGTGTAACACCCCAGACCCCTCCAAGTTCTTTTCTCA | |
| | GCTGTCCTCTGAGCACGGCGGCGATGTGCAGAAGTGGCTGAGCTCC | |
| | CCTTTTCCATCTAGCTCCTTCAGCCCTGGAGGACTGGCACCAGAGA | |
| | TCTCCCCTCTGGAGGTGCTGGAGCGGGACAAGGTGACCCAGCTGCT | |
| | GCTGCAGCAGGATAAGGTGCCCGAGCCTGCCTCCCTGTCTAGCAAC | |
| | CACAGCCTGACCTCCTGCTTTACAAATCAGGGCTATTTCTTTTTCC | |
| | ACCTGCCAGACGCACTGGAGATCGAGGCATGTCAGGTGTACTTCAC | |
| | ATATGATCCATACAGCGAGGAGGACCCAGATGAGGGAGTGGCCGGC | |
| | GCCCCTACCGGATCCTCTCCACAGCCACTGCAGCCACTGTCTGGCG | |
| | AGGACGATGCCTACTGTACATTTCCTAGCAGAGACGATCTGCTGCT | |
| | GTTCTCCCCTTCTCTGCTGGGAGGACCATCTCCACCTAGCACCGCA | |
| | CCTGGAGGATCCGGGGCAGGGGAGGAGAGGATGCCACCATCTCTGC | |
| | AGGAGAGGGTGCCAAGGGACTGGGATCCTCAGCCACTGGGACCTCC | |
| | AACCCCAGGAGTGCCCGACCTGGTGGATTTCCAGCCCCCTCCAGAG | |
| | CTGGTGCTGAGAGAGGCAGGAGAGGAGGTGCCAGACGCAGGACCAA | |
| | GAGAGGGCGTGAGCTTTCCATGGTCCAGGCCACCTGGACAGGGAGA | |
| | GTTCAGAGCCCTGAACGCCAGGCTGCCACTGAATACAGACGCCTAC | |
| | CTGTCTCTGCAGGAGCTGCAGGGCCAGGATCCCACCCACCTGGTG | |
| 39 | AATAAAAGATCTTTATTTTCATTAGATCTGTGTGTTGGTTTTTTGT | Synthetic |
| | GTG | polyA |
| 40 | TACCCAGAGAATGGAGAAACCCTGCAGGGAATTCCCAAGCTGTAGT | RACR_B2M |
| | TATAAACAGAAGTTCTCCTTCTGCTAGGTAGCATTCAAAGATCTTA | ssODN |
| | ATCTTCTGGGTTTCCGTTTTCTCGAATGAAAAATGCAGGTCCGAGC | |
| | AGTTAACTGGCTGGGGCACCATTAGCAAGTCACTTAGCATCTCTGG | |
| | GGCCAGTCTGCAAAGCGAGGGGGCAGCCTTAATGTGCCTCCAGCCT | |
| | GAAGTCCTAGAATGAGCGCCCGGTGTCCCAAGCTGGGGCGCGCACC | |
| | CCAGATCGGAGGGCGCCGATGTACAGACAGCAAACTCACCCAGTCT | |
| | AGTGCATGCCTTCTTAAACATCACGAGACTCTAAGAAAAGGAAACT | |
| | GAAAACGGGAAAGTCCCTCTCTCTAACCTGGCACTGCGTCGCTGGC | |
| | TTGGAGACAGGTGACGGTCCCTGCGGGCCTTGTCCTGATTGGCTGG | |
| | GCACGCGTTTAATATAAGTGGAGGCGTCGCGCTGGCGGGC | |
| | CGTGAGGCTCCGGTGCCCGTCAGTGGGCAGAGCGCACATCGCCCAC | |
| | AGTCCCCGAGAAGTTGGGGGGAGGGGTCGGCAATTGAACCGGTGCC | |
| | TAGAGAAGGTGGCGCGGGGTAAACTGGGAAAGTGATGTCGTGTACT | |
| | GGCTCCGCCTTTTTCCCGAGGGTGGGGGAGAACCGTATATAAGTGC | |
| | AGTAGTCGCCGTGAACGTTCTTTTTCGCAACGGGTTTGCCGCCAGA | |
| | ACACAGGTAAGTGCCGTGTGTGGTTCCCGCGGGCCTGGCCTCTTTA | |
| | CGGGTTATGGCCCTTGCGTGCCTTGAATTACTTCCACGCCCCTGGC | |
| | TGCAGTACGTGATTCTTGATCCCGAGCTTCGGGTTGGAAGTGGGTG | |
| | GGAGAGTTCGAGGCCTTGCGCTTAAGGAGCCCCTTCGCCTCGTGCT | |
| | TGAGTTGAGGCCTGGCTTGGGCGCTGGGGCCGCCGCGTGCGAATCT | |
| | GGTGGCACCTTCGCGCCTGTCTCGCTGCTTTCGATAAGTCTCTAGC | |
| | CATTTAAAATTTTTGATGACCTGCTGCGACGCTTTTTTTCTGGCAA | |
| | GATAGTCTTGTAAATGCGGGCCAAGATCTGCACACTGGTATTTCGG | |
| | TTTTTGGGGCCGCGGGCGGCGACGGGGCCCGTGCGTCCCAGCGCAC | |
| | ATGTTCGGCGAGGCGGGGCCTGCGAGCGCGGCCACCGAGAATCGGA | |
| | CGGGGGTAGTCTCAAGCTGGCCGGCCTGCTCTGGTGCCTGGCCTCG | |
| | CGCCGCCGTGTATCGCCCCGCCCTGGGCGGCAAGGCTGGCCCGGTC | |
| | GGCACCAGTTGCGTGAGCGGAAAGATGGCCGCTTCCCGGCCCTGCT | |
| | GCAGGGAGCTCAAAATGGAGGACGCGGCGCTCGGGAGAGCGGGCGG | |
| | GTGAGTCACCCACACAAAGGAAAAGGGCCTTTCCGTCCTCAGCCGT | |
| | CGCTTCATGTGACTCCACGGAGTACCGGGCGCCGTCCAGGCACCTC | |
| | GATTAGTTCTCGAGCTTTTGGAGTACGTCGTCTTTAGGTTGGGGGG | |
| | AGGGGTTTTATGCGATGGAGTTTCCCCACACTGAGTGGGTGGAGAC | |
| | TGAAGTTAGGCCAGCTTGGCACTTGATGTAATTCTCCTTGGAATTT | |
| | GCCCTTTTTGAGTTTGGATCTTGGTTCATTCTCAAGCCTCAGACAG | |
| | TGGTTCAAAGTTTTTTTCTTCCATTTCAGGTGTCGTGA | |
| | GCCACC | |
| | ATGCCACTGGGACTGCTGTGGCTGGGACTGGCCCTGCTGGGCGCCC | |
| | TGCACGCCCAGGCCGGCGTGCAGGTGGAGACAATCAGCCCAGGCGA | |
| | CGGACGCACCTTTCCTAAGCGGGGCCAGACATGCGTGGTGCACTAC | |
| | ACCGGCATGCTGGAGGATGGCAAGAAGTTCGACTCTAGCAGAGATA | |
| | GGAACAAGCCCTTTAAGTTCATGCTGGGCAAGCAGGAAGTGATCAG | |
| | AGGCTGGGAGGAGGGCGTGGCCCAGATGTCTGTGGGCCAGAGGGCC | |
| | AAGCTGACAATCAGCCCAGACTACGCATATGGAGCAACCGGACACC | |
| | CAGGAATCATCCCACCTCACGCCACACTGGTGTTCGATGTGGAGCT | |
| | GCTGAAGCTGGGCGAGGGCTCTAACACCAGCAAGGAGAATCCATTT | |
| | CTGTTCGCACTGGAGGCAGTGGTCATCTCCGTGGGCTCTATGGGCC | |
| | TGATCATCTCCCTGCTGTGCGTGTACTTTTGGCTGGAGAGAACAAT | |
| | GCCCAGGATCCCTACCCTGAAGAACCTGGAGGACCTGGTGACCGAG | |
| | TATCACGGCAATTTCAGCGCCTGGTCCGGCGTGTCTAAGGGACTGG | |
| | CAGAGTCCCTGCAGCCAGATTACTCTGAGCGGCTGTGCCTGGTGAG | |
| | CGAGATCCCACCCAAGGGAGGCGCCCTGGGAGAGGGACCTGGAGCA | |
| | AGCCCATGCAACCAGCACTCCCCATATTGGGCCCCTCCATGTTACA | |
| | CCCTGAAGCCTGAGACA | |
| | GGAAGCGGAGCGACGAATTTTAGTCTACTGAAACAAGCGGGAGACG | |
| | TGGAGGAAAACCCTGGACCT | |
| | ATGGCACTGCCAGTGACCGCCCTGCTGCTGCCACTGGCCCTGCTGC | |
| | TGCACGCAGCACGCCCCATCCTGTGGCACGAAATGTGGCATGAAGG | |
| | CCTGGAGGAGGCAAGCAGACTGTATTTTGGCGAGAGAAATGTGAAA | |
| | GGAATGTTTGAGGTGCTGGAGCCACTGCACGCAATGATGGAGAGGG | |
| | GACCTCAGACCCTGAAGGAGACATCCTTTAACCAGGCCTATGGCAG | |
| | AGACCTGATGGAGGCCCAGGAGTGGTGCAGGAAGTACATGAAGAGC | |
| | GGAAATGTGAAAGACCTGCTGCAGGCCTGGGATCTGTATTATCACG | |
| | TGTTCAGAAGGATCAGCAAGGGCAAGGATACCATCCCTTGGCTGGG | |
| | ACACCTGCTGGTGGGACTGTCCGGAGCCTTTGGCTTCATCATCCTG | |
| | GTGTACCTGCTGATCAACTGCCGCAATACAGGCCCATGGCTGAAGA | |
| | AGGTGCTGAAGTGTAACACCCCAGACCCCTCCAAGTTCTTTTCTCA | |
| | GCTGTCCTCTGAGCACGGCGGCGATGTGCAGAAGTGGCTGAGCTCC | |
| | CCTTTTCCATCTAGCTCCTTCAGCCCTGGAGGACTGGCACCAGAGA | |
| | TCTCCCCTCTGGAGGTGCTGGAGCGGGACAAGGTGACCCAGCTGCT | |
| | GCTGCAGCAGGATAAGGTGCCCGAGCCTGCCTCCCTGTCTAGCAAC | |
| | CACAGCCTGACCTCCTGCTTTACAAATCAGGGCTATTTCTTTTTCC | |
| | ACCTGCCAGACGCACTGGAGATCGAGGCATGTCAGGTGTACTTCAC | |
| | ATATGATCCATACAGCGAGGAGGACCCAGATGAGGGAGTGGCCGGC | |
| | GCCCCTACCGGATCCTCTCCACAGCCACTGCAGCCACTGTCTGGCG | |
| | AGGACGATGCCTACTGTACATTTCCTAGCAGAGACGATCTGCTGCT | |
| | GTTCTCCCCTTCTCTGCTGGGAGGACCATCTCCACCTAGCACCGCA | |
| | CCTGGAGGATCCGGGGCAGGGGAGGAGAGGATGCCACCATCTCTGC | |
| | AGGAGAGGGTGCCAAGGGACTGGGATCCTCAGCCACTGGGACCTCC | |
| | AACCCCAGGAGTGCCCGACCTGGTGGATTTCCAGCCCCCTCCAGAG | |
| | CTGGTGCTGAGAGAGGCAGGAGAGGAGGTGCCAGACGCAGGACCAA | |
| | GAGAGGGCGTGAGCTTTCCATGGTCCAGGCCACCTGGACAGGGAGA | |
| | GTTCAGAGCCCTGAACGCCAGGCTGCCACTGAATACAGACGCCTAC | |
| | CTGTCTCTGCAGGAGCTGCAGGGCCAGGATCCCACCCACCTGGTG | |
| | TAAAC | |
| | AATAAAAGATCTTTATTTTCATTAGATCTGTGTGTTGGTTTTTTGT | |
| | GTG | |
| | TGGCCTTAGCTGTGCTCGCGCTACTCTCTCTTTCTGGCCTGGAGGC | |
| | TATCCAGCGTGAGTCTCTCCTACCCTCCCGCTCTGGTCCTTCCTCT | |
| | CCCGCTCTGCACCCTCTGTGGCCCTCGCTGTGCTCTCTCGCTCCGT | |
| | GACTTCCCTTCTCCAAGTTCTCCTTGGTGGCCCGCCGTGGGGCTAG | |
| | TCCAGGGCTGGATCTCGGGGAAGCGGCGGGGTGGCCTGGGAGTGGG | |
| | GAAGGGGGTGCGCACCCGGGACGCGCGCTACTTGCCCCTTTCGGCG | |
| | GGGAGCAGGGGAGACCTTTGGCCTACGGCGACGGGAGGGTCGGGAC | |
| | AAAGTTTAGGGCGTCGATAAGCGTCAGAGCGCCGAGGTTGGGGGAG | |
| | GGTTTCTCTTCCGCTCTTTCGCGGGGCCTCTGGCTCCCCCAGCGCA | |
| | GCTGGAGTGGGGGACGGGTAGGCTCGTCCCAAAGGCGCGGCGCTGA | |
| | GGTTTGTGAACGCGTGGAGGGGCGCTTGGGGTCTGGGGGA | |
| 41 | GGAAGCGGAGCGACGAATTTTAGTCTACTGAAACAAGCGGGAGACG | PQR linker |
| | TGGAGGAAAACCCTGGACCT | nucleotide |
| 42 | GSGATNFSLLKQAGDVEENPGP | PQR linker |
| 43 | VKQTLNFDLLKLAGDVESNPGP | F2A |
| 44 | QCTNYALLKLAGDVESNPGP | E2A |
| 45 | LEGGGEGRGSLLTCGDVEENPGPR | T2A |
| 46 | EGRGSLLTCGDVEENPGP | T2A |
| 47 | GSGATNFSLLKQAGDVEENPGP | P2A |
| 48 | ATNFSLLKQAGDVEENPGP | P2A |
| 49 | GVQVETISPGDGRTFPKRGQTCVVHYTGMLEDGKKFDSSRDRNKPF | FKBP |
| | KFMLGKQEVIRGWE | domain |
| | EGVAQMSVGQRAKLTISPDYAYGATGHPGIIPPHATLVFDVELLKL | (FKBP12) |
| 50 | CTGCTGCTGGTGACCTCCCTGCTGCTGTGCGAGCTGCCTCACCCAG | CD19 CAR |
| | CCTTTCTGCTGATCCCCGACATCCAGATGACACAGACCACAAGCTC | nucleotide |
| | CCTGTCTGCCAGCCTGGGCGACAGAGTGACCATCTCCTGTAGGGCC | |
| | TCTCAGGATATCAGCAAGTACCTGAACTGGTATCAGCAGAAGCCAG | |
| | ATGGCACAGTGAAGCTGCTGATCTACCACACCTCCAGGCTGCACTC | |
| | TGGAGTGCCAAGCCGGTTCTCCGGATCTGGAAGCGGCACCGACTAT | |
| | TCCCTGACAATCTCTAACCTGGAGCAGGAGGATATCGCCACATACT | |
| | TTTGCCAGCAGGGCAATACCCTGCCATATACATTCGGCGGAGGAAC | |
| | CAAGCTGGAGATCACCGGATCCACATCTGGAAGCGGCAAGCCAGGA | |
| | AGCGGAGAGGGATCCACAAAGGGAGAGGTGAAGCTGCAGGAGAGCG | |
| | GACCAGGACTGGTGGCACCATCCCAGTCTCTGAGCGTGACCTGTAC | |
| | AGTGTCCGGCGTGTCTCTGCCTGACTACGGCGTGTCCTGGATCAGG | |
| | CAGCCACCTAGGAAGGGACTGGAGTGGCTGGGCGTGATCTGGGGCT | |
| | CTGAGACCACATACTATAATTCTGCCCTGAAGAGCCGCCTGACCAT | |
| | CATCAAGGACAACTCCAAGTCTCAGGTGTTTCTGAAGATGAATAGC | |
| | CTGCAGACCGACGATACAGCCATCTACTATTGCGCCAAGCACTACT | |
| | ATTACGGCGGCTCCTACGCCATGGATTATTGGGGCCAGGGCACCTC | |
| | CGTGACAGTGTCTAGCGGCGCTGTGCACACCAGAGGACTGGATTTC | |
| | GCCTGCGACTTCTGGGTGCTGGTGGTGGTGGGAGGCGTGCTGGCCT | |
| | GTTACTCCCTGCTGGTGACCGTGGCCTTTATCATCTTCTGGGTGAA | |
| | GAGAGGCAGGAAGAAGCTGCTGTATATCTTTAAGCAGCCCTTCATG | |
| | CGCCCTGTGCAGACCACACAGGAGGAGGACGGCTGCAGCTGTCGGT | |
| | TTCCAGAGGAGGAGGAGGGAGGATGCGAGCTGCGCGTGAAGTTCAG | |
| | CCGGTCCGCCGATGCCCCTGCCTACCAGCAGGGCCAGAACCAGCTG | |
| | TATAACGAGCTGAATCTGGGCCGGAGAGAGGAGTACGACGTGCTGG | |
| | ATAAGAGGAGGGGAAGGGACCCAGAGATGGGAGGCAAGCCTCGGAG | |
| | AAAGAACCCACAGGAGGGCCTGTACAATGAGCTGCAGAAGGACAAG | |
| | ATGGCCGAGGCCTATTCTGAGATCGGCATGAAGGGAGAGAGGCGCC | |
| | GGGGCAAGGGACACGATGGCCTGTACCAGGGCCTGAGCACCGCCAC | |
| | AAAGGACACATATGATGCCCTGCACATGCAGGCCCTGCCACCTAGG | |
| 51 | LLLVTSLLLCELPHPAFLLIPDIQMTQTTSSLSASLGDRVTISCRA | CD19 CAR |
| | SQDISKYLNWYQQKPDGTVKLLIYHTSRLHSGVPSRFSGSGSGTDY | polypeptide |
| | SLTISNLEQEDIATYFCQQGNTLPYTFGGGTKLEITGSTSGSGKPG | |
| | SGEGSTKGEVKLQESGPGLVAPSQSLSVTCTVSGVSLPDYGVSWIR | |
| | QPPRKGLEWLGVIWGSETTYYNSALKSRLTIIKDNSKSQVFLKMNS | |
| | LQTDDTAIYYCAKHYYYGGSYAMDYWGQGTSVTVSSGAVHTRGLDF | |
| | ACDFWVLVVVGGVLACYSLLVTVAFIIFWVKRGRKKLLYIFKQPFM | |
| | RPVQTTQEEDGCSCRFPEEEEGGCELRVKFSRSADAPAYQQGQNQL | |
| | YNELNLGRREEYDVLDKRRGRDPEMGGKPRRKNPQEGLYNELQKDK | |
| | MAEAYSEIGMKGERRRGKGHDGLYQGLSTATKDTYDALHMQALPPR | |
| 52 | GGGGCCACTAGGGACAGGAT | AAVS1 gRNA |
| 53 | TTTCCTTCTCCTTCTGGGGCCTGTGCCATCTCTCGTTTCTTAGGAT | 5′ |
| | GGCCTTCTCCGACGGATGTCTCCCTTGCGTCCCGCCTCCCCTTCTT | homology |
| | GTAGGCCTGCATCATCACCGTTTTTCTGGACAACCCCAAAGTACCC | arm for |
| | CGTCTCCCTGGCTTTAGCCACCTCTCCATCCTCTTGCTTTCTTTGC | AAVS1 |
| | CTGGACACCCCGTTCTCCTGTGGATTCGGGTCACCTCTCACTCCTT | |
| | TCATTTGGGCAGCTCCCCTACCCCCCTTACCTCTCTAGTCTGTGCT | |
| | AGCTCTTCCAGCCCCCTGTCATGGCATCTTCCAGGGGTCCGAGAGC | |
| | TCAGCTAGTCTTCTTCCTCCAACCCGGGCCCCTATGTCCACTTCAG | |
| | GACAGCATGTTTGCTGCCTCCAGGGATCCTGTGTCCCCGAGCTGGG | |
| | ACCACCTTATATTCCCAGGGCCGGTTAATGTGGCTCTGGTTCTGGG | |
| | TACTTTTATCTGTCCCCTCCACCCCACAGTGGGGCCACTA | |
| 54 | GGGACAGGATTGGTGACAGAAAAGCCCCATCCTTAGGCCTCCTCCT | 3′ |
| | TCCTAGTCTCCTGATATTGGGTCTAACCCCCACCTCCTGTTAGGCA | homology |
| | GATTCCTTATCTGGTGACACACCCCCATTTCCTGGAGCCATCTCTC | arm for |
| | TCCTTGCCAGAACCTCTAAGGTTTGCTTACGATGGAGCCAGAGAGG | AAVSI |
| | ATCCTGGGAGGGAGAGCTTGGCAGGGGGTGGGAGGGAAGGGGGGGA | |
| | TGCGTGACCTGCCCGGTTCTCAGTGGCCACCCTGCGCTACCCTCTC | |
| | CCAGAACCTGAGCTGCTCTGACGCGGCCGTCTGGTGCGTTTCACTG | |
| | ATCCTGGTGCTGCAGCTTCCTTACACTTCCCAAGAGGAGAAGCAGT | |
| | TTGGAAAAACAAAATCAGAATAAGTTGGTCCTGAGTTCTAACTTTG | |
| | GCTCTTCACCTTTCTAGTCCCCAATTTATATTGTTCCTCCGTGCGT | |
| | CAGTTTTACCTGTGAGATAAGGCCAGTAGCCAGCCCCGTC | |
| 55 | ILWHEMWHEGLEEASRLYFGERNVKGMFEVLEPLHAMMERGPQTLK | RACR (b) |
| | ETSFNQAYGRDLMEAQEWCRKYMKSGNVKDLLQAWDLYYHVFRRIS | without |
| | KGKDTIPWLGHLLVGLSGAFGFIILVYLLINCRNTGPWLKKVLKCN | signal |
| | TPDPSKFFSQLSSEHGGDVQKWLSSPFPSSSFSPGGLAPEISPLEV | peptide |
| | LERDKVTQLLLQQDKVPEPASLSSNHSLTSCFTNQGYFFFHLPDAL | |
| | EIEACQVYFTYDPYSEEDPDEGVAGAPTGSSPQPLQPLSGEDDAYC | |
| | TFPSRDDLLLFSPSLLGGPSPPSTAPGGSGAGEERMPPSLQERVPR | |
| | DWDPQPLGPPTPGVPDLVDFQPPPELVLREAGEEVPDAGPREGVSF | |
| | PWSRPPGQGEFRALNARLPLNTDAYLSLQELQGQDPTHLV | |
| 56 | ILWHEMWHEGLEEASRLYFGERNVKGMFEVLEPLHAMMERGPQTLK | FRB: IL2RG |
| | ETSFNQAYGRDLMEAQEWCRKYMKSGNVKDLLQAWDLYYHVFRRIS | polypeptide |
| | KGSNTSKENPFLFALEAVVISVGSMGLIISLLCVYFWLERTMPRIP | |
| | TLKNLEDLVTEYHGNFSAWSGVSKGLAESLQPDYSERLCLVSEIPP | |
| | KGGALGEGPGASPCNQHSPYWAPPCYTLKPET | |
| 57 | GVQVETISPGDGRTFPKRGQTCVVHYTGMLEDGKKFDSSRDRNKPF | FKBP12: IL2RB |
| | KFMLGKQEVIRGWEEGVAQMSVGQRAKLTISPDYAYGATGHPGIIP | polypeptide |
| | PHATLVFDVELLKLGEGKDTIPWLGHLLVGLSGAFGFIILVYLLIN | |
| | CRNTGPWLKKVLKCNTPDPSKFFSQLSSEHGGDVQKWLSSPFPSSS | |
| | FSPGGLAPEISPLEVLERDKVTQLLLQQDKVPEPASLSSNHSLTSC | |
| | FTNQGYFFFHLPDALEIEACQVYFTYDPYSEEDPDEGVAGAPTGSS | |
| | PQPLQPLSGEDDAYCTFPSRDDLLLFSPSLLGGPSPPSTAPGGSGA | |
| | GEERMPPSLQERVPRDWDPQPLGPPTPGVPDLVDFQPPPELVLREA | |
| | GEEVPDAGPREGVSFPWSRPPGQGEFRALNARLPLNTDAYLSLQEL | |
| | QGQDPTHLV | |

1. An engineered cell that is a myeloid progenitor cell comprising a synthetic cytokine receptor for a non-physiological ligand,
wherein the synthetic cytokine receptor comprises:
a synthetic gamma chain polypeptide comprising a first dimerization domain, a first transmembrane domain, and an interleukin-2 receptor subunit gamma (IL-2RG) intracellular domain; and
a synthetic beta chain polypeptide comprising a second dimerization domain, a second transmembrane domain, and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

2. The engineered cell of claim 1, wherein the myeloid progenitor cell is a Granulocyte/Monocyte Progenitor Cell (“GMP”).

3. The engineered cell of claim 1 or claim 2, wherein the myeloid progenitor cell is characterized with a surface phenotype CD34+, CD90−, and CD45RA+.

4. The engineered cell of claim 1 or claim 2, wherein the myeloid progenitor cell is characterized with a surface phenotype CD34+, CD90−, CD123+, and CD45RA+.

5. An engineered cell that is a myeloid cell comprising a synthetic cytokine receptor for a non-physiological ligand,
wherein the synthetic cytokine receptor comprises:
a synthetic gamma chain polypeptide comprising a first dimerization domain, a first transmembrane domain and an interleukin-2 receptor subunit gamma (IL-2RG) intracellular domain, and
a synthetic beta chain polypeptide comprising a second dimerization domain, a second transmembrane domain, and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

6. The engineered cell of claim 5, wherein the myeloid cell is a macrophage, a neutrophil, a megakaryocyte, a monocyte, a basophil, an eosinophil, and/or an erythrocyte cell.

7. An engineered cell that is a macrophage comprising a synthetic cytokine receptor for a non-physiological ligand,
wherein the synthetic cytokine receptor comprises:
a synthetic gamma chain polypeptide comprising a first dimerization domain, a first transmembrane domain, and an interleukin-2 receptor subunit gamma (IL-2RG) intracellular domain; and
a synthetic beta chain polypeptide comprising a second dimerization domain, a second transmembrane domain, and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

8. The engineered cell of claim 6 or claim 7, wherein the macrophage is a mature macrophage that expresses CD14.

9. An engineered cell that is a neutrophil comprising a synthetic cytokine receptor for a non-physiological ligand,
wherein the synthetic cytokine receptor comprises:
a synthetic gamma chain polypeptide comprising a first dimerization domain, a first transmembrane domain, and an interleukin-2 receptor subunit gamma (IL-2RG) intracellular domain; and
a synthetic beta chain polypeptide comprising a second dimerization domain, a second transmembrane domain, and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

10. The engineered cell of any of claims 1-9, wherein the engineered cell has been differentiated from a stem cell.

11. The engineered cell of claim 10, wherein the stem cell is a pluripotent stem cell.

12. The engineered cell of claim 10 or claim 11, wherein the stem cell is an induced pluripotent stem cell.

13. The engineered cell of any of claims 1-6 and 10-12, wherein the myeloid cell is an induced myeloid cell (iMC) differentiated from a stem cell engineered with the synthetic cytokine receptor.

14. The engineered cell of any of claims 7-8 and 10-12, wherein the macrophage is an induced macrophage (iMAC) differentiated from a stem cell engineered with the synthetic cytokine receptor.

15. The engineered cell of any of claims 9-12, wherein the neutrophil is an induced neutrophil (iNEU) differentiated from a stem cell engineered with the synthetic cytokine receptor.

16. The engineered cell of any of claims 1-15, wherein:
the synthetic gamma chain polypeptide comprises, in N- to C-terminal order, the first dimerization domain, the first transmembrane domain, and the IL-2RG intracellular domain; and
the synthetic beta chain polypeptide comprises, in N- to C-terminal order, the second dimerization domain, the second transmembrane domain, and the beta chain intracellular domain.

17. The engineered macrophage of any of claims 1-16, wherein the first dimerization domain and the second dimerization domain are extracellular domains.

18. The engineered cell of any of claims 1-17, wherein the IL-2RG intracellular domain comprises a polypeptide sequence at least 95% identical to SEQ ID NO: 1, or a polypeptide sequence as set forth in SEQ ID NO: 1.

19. The engineered cell of any of claims 1-18, wherein the first transmembrane domain comprises the IL-2RG transmembrane domain.

20. The engineered cell of claim 19, wherein the IL-2RG transmembrane domain comprises a polypeptide sequence at least 95% identical to SEQ ID NO: 8 or 31, or a polypeptide sequence as set forth in SEQ ID NO: 8 or 31.

21. The engineered cell of any of claims 1-20, wherein the beta chain intracellular domain is an IL-2RB intracellular domain.

22. The engineered cell of claim 21, wherein the IL-2RB intracellular domain comprises a polypeptide sequence at least 95% identical to SEQ ID NO: 2, or a polypeptide sequence as set forth in SEQ ID NO: 2.

23. The engineered cell of any of claims 1-20, wherein the beta chain intracellular domain is an IL-7RB intracellular domain.

24. The engineered cell of claim 23, wherein the IL-7RB intracellular domain comprises a polypeptide sequence at least 95% identical to SEQ ID NO: 3, or a polypeptide sequence as set forth in SEQ ID NO: 3.

25. The engineered cell of any of claims 1-20, wherein the beta chain intracellular domain is an IL-21RB intracellular domain.

26. The engineered cell of claim 25, wherein the IL-21RB intracellular domain comprises a polypeptide sequence at least 95% identical to SEQ ID NO: 4, or a polypeptide sequence as set forth in SEQ ID NO: 4.

27. The engineered cell of any of claims 1-26, wherein the second transmembrane domain comprises a transmembrane domain from the same polypeptide as the beta chain intracellular domain.

28. The engineered cell of any one of claims 1-27, wherein the second transmembrane domain is a transmembrane domain of IL-2RB comprising a polypeptide sequence at least 95% identical to SEQ ID NO: 35 or 36, or a polypeptide sequence as set forth in SEQ ID NO: 35 or 36.

29. The engineered cell of any of claims 1-22, 27 and 28, wherein:
the first transmembrane domain of the synthetic gamma chain polypeptide is an IL-2RG transmembrane domain comprising the sequence set forth in SEQ ID NO: 8 or 31, and the IL-2RG intracellular domain comprises the sequence set forth in SEQ ID NO:1; and
the second transmembrane domain of the synthetic beta chain polypeptide is an IL-2RB transmembrane domain comprising the sequence set forth in SEQ ID NO: 35 or 36, and the beta chain intracellular domain is an IL-2RB intracellular domain comprising the sequence set forth in SEQ ID NO:2.

30. The engineered cell of any of claims 1-29, wherein:
the first transmembrane domain and the IL-2RG intracellular domain of the synthetic gamma chain polypeptide comprise the sequences set forth in SEQ ID NOs: 31 and SEQ ID NO:1; and
the second transmembrane domain and the beta chain intracellular domain of the synthetic beta chain polypeptide comprise the sequences set forth in SEQ ID NOs: 35 and SEQ ID NO:2.

31. The engineered cell of any one of claims 1-30, wherein the first dimerization domain and the second dimerization domain are heterodimerization domains selected from FKBP12-rapamycin binding (FRB) domain and a FK506-Binding Protein of size 12 kD (FKBP); and/or
wherein the non-physiological ligand is rapamycin or a rapalog.

32. The engineered cell of any one of claims 1-31, wherein the first dimerization domain is FKBP and the second dimerization domain is FRB.

33. The engineered cell of any one of claims 1-31, wherein the first dimerization domain is FRB and the second dimerization domain is FKBP.

34. The engineered cell of any of claims 31-33, wherein the FRB domain comprises a polypeptide sequence at least 95% identical to SEQ ID NO: 6 or SEQ ID NO: 7.

35. The engineered cell of any of claims 31-34, wherein the FRB domain comprises the polypeptide sequence set forth in SEQ ID NO: 6 or SEQ ID NO: 7.

36. The engineered cell of any one of claims 31-35, wherein the FKBP domain comprises a polypeptide sequence at least 95% identical to SEQ ID NO: 5 or SEQ ID NO:30.

37. The engineered cell of any one of claims 31-36, wherein the FKBP domain comprises the polypeptide sequence set forth in SEQ ID NO: 5 or SEQ ID NO:30.

38. The engineered cell of any of claims 1-37, wherein the synthetic gamma chain polypeptide has the sequence of amino acids set forth in SEQ ID NO: 28 or a sequence of amino acids that is at least 90%, at least 95%, at least 96%, at least 97%, at least 98%, at least 99%, or 100% identical to SEQ ID NO: 28, and the synthetic beta chain polypeptide has the sequence of amino acids set forth in SEQ ID NO: 55 or a sequence of amino acids that is at least 90%, at least 95%, at least 96%, at least 97%, at least 98%, at least 99%, or 100% identical to SEQ ID NO: 55.

39. The engineered cell of any of claims 1-38, wherein the synthetic gamma chain polypeptide has the sequence of amino acids set forth in SEQ ID NO: 56 or a sequence of amino acids that is at least 90%, at least 95%, at least 96%, at least 97%, at least 98%, at least 99%, or 100% identical to SEQ ID NO: 56, and the synthetic beta chain polypeptide has the sequence of amino acids set forth in SEQ ID NO: 57 or a sequence of amino acids that is at least 90%, at least 95%, at least 96%, at least 97%, at least 98%, at least 99%, or 100% identical to SEQ ID NO: 57.

40. The engineered cell of any of claims 1-30, wherein the first dimerization domain and the second dimerization domain are homodimerization domains selected from:
i) FK506-Binding Protein of size 12 kD (FKBP);
ii) cyclophiliA (CypA); or
iii) gyrase B (CyrB);
and the non-physiological ligand is, respectively:
i) FK1012, AP1510, AP1903, or AP20187 or an analog thereof,
ii) cyclosporin-A (CsA) or an analog thereof, or
iii) coumermycin or an analog thereof.

41. The engineered cell of any of claims 1-40, wherein the engineered cell is resistant to rapamycin-mediated mTOR inhibition.

42. The engineered cell of any of claims 1-41, wherein the engineered cell expresses a cytosolic polypeptide that binds to the non-physiological ligand, optionally wherein the cytosolic polypeptide is a cytosolic FRB domain.

43. The engineered cell of any of claims 1-42, wherein the non-physiological ligand is rapamycin or a rapalog, and the engineered cell expresses a cytosolic FRB domain or variant thereof.

44. The engineered cell of claim 42 or claim 43, wherein the cytosolic FRB domain comprises a polypeptide sequence at least 95% identical to SEQ ID NO: 6 or SEQ ID NO: 7.

45. The engineered cell of claim 42 or claim 43, wherein the cytosolic FRB domain comprises a polypeptide sequence at least 98% identical to SEQ ID NO: 6 or SEQ ID NO: 7.

46. The engineered cell of any of claims 1-45, wherein the engineered cell comprises a disrupted FKBP12 gene that reduces expression of FKBP12 in the cell.

47. The engineered cell of any of claims 1-46, wherein the engineered cell comprises knock out of the FKBP12 gene.

48. The engineered cell of any one of claims 1-47, wherein the engineered cell comprises a nucleotide sequence encoding the synthetic cytokine receptor inserted into the genome of the cell.

49. The engineered cell of claim 48, wherein the nucleotide sequence encoding the synthetic cytokine receptor is inserted into a non-target locus in the genome of the cell.

50. The engineered cell of claim 48, wherein the nucleotide sequence encoding the synthetic cytokine receptor is inserted into an endogenous gene of the cell.

51. The engineered cell of claim 50, wherein the insertion reduces expression of the endogenous gene in the locus.

52. The engineered cell of claim 50 or claim 51, wherein the insertion knocks out the endogenous gene in the locus.

53. The engineered cell of any of claims 50-52, wherein the insertion is by homology-directed repair.

54. The engineered cell of any of claims 50-53, wherein the endogenous gene is a housekeeping gene, a blood-lineage specific loci, or an immune-related gene.

55. The engineered cell of claim 54, wherein the housekeeping gene is selected from eukaryotic translation elongation factor 1 alpha (EEF1A), glylceraldehyde-3-phosphate dehydrogenase (GAPDH), ubiquitin C (UBC), and actin beta (ACTB).

56. The engineered cell of claim 54, wherein the immune-related gene is selected from a beta-2-microglobulin (B2M) gene, and a signal regulatory protein alpha (SIRPA) gene.

57. The engineered cell of any one of claims 1-56, wherein the engineered cell comprises a B2M knockout.

58. The engineered cell of any of claims 1-57, wherein the engineered cell comprises a B2M knockout and a FKBP12 knockout.

59. The engineered cell of any one of claims 1-58, comprising a chimeric antigen receptor (CAR).

60. The engineered cell of any of claims 1-59, wherein binding of the non-physiological ligand to the synthetic cytokine receptor activates the synthetic cytokine receptor in the engineered cell to induce expansion and/or activation of the engineered cell in a cell population.

61. A population comprising engineered myeloid progenitor cells of any of claims 1-4 and 10-12 and 16-60.

62. A population comprising engineered myeloid cells of any of claims 5, 6 and 10-13 and 16-60.

63. A population comprising engineered macrophages of any of claims 7, 8 and 10-12, 14 and 16-60.

64. A population comprising engineered neutrophils of any of claims 9-12 and 15-60.

65. A method of generating genetically engineered myeloid cells differentiated from stem cells, the method comprising:
a) culturing a population of stem cells engineered with a synthetic cytokine receptor under conditions to differentiate cells into a population of hematopoietic progenitors (HP),
wherein the synthetic cytokine receptor comprises:
a synthetic gamma chain polypeptide comprising a first dimerization domain, a first transmembrane domain and an interleukin-2 receptor subunit gamma (IL-2RG) intracellular domain, and
a synthetic beta chain polypeptide comprising a second dimerization domain, a second transmembrane domain and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain; and
b) culturing the cells produced in a) by incubation under conditions to generate myeloid cells,
wherein at least a portion of one or both of steps a) and b) are carried out in the presence of a non-physiological ligand of the synthetic cytokine receptor.

66. The method of claim 65, wherein the myeloid cell is a macrophage or a neutrophil.

67. A method of generating genetically engineered macrophages differentiated from stem cells, the method comprising:
a) culturing a population of stem cells engineered with a synthetic cytokine receptor under conditions to differentiate cells into a population of hematopoietic progenitors (HP),
wherein the synthetic cytokine receptor comprises:
a synthetic gamma chain polypeptide comprising a first dimerization domain, a first transmembrane domain and an interleukin-2 receptor subunit gamma (IL-2RG) intracellular domain, and
a synthetic beta chain polypeptide comprising a second dimerization domain, a second transmembrane domain and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain; and
b) culturing the cells produced in a) by incubation under conditions to generate macrophages,
wherein at least a portion of one or both of steps a) and b) are carried out in the presence of a non-physiological ligand of the synthetic cytokine receptor.

68. The method of any of claims 65-67, wherein the culturing in a) is carried out by a first incubation under conditions to produce an Embryoid Body (EB) followed by one or more further incubations in the presence of the non-physiological ligand and optionally one or more myeloid cell differentiation factors selected from one or more of IL-3, M-CSF and GM-CSF.

69. The method of claim 68, wherein the one or more myeloid cell differentiation factors is IL-3, M-CSF and GM-CSF.

70. The method of claim 68 or claim 69, wherein the one or more further incubations comprises a second incubation in a second media comprising one or more of IL-3, GM-CSF, and M-CSF, and a third incubation in a third media comprising one or more of IL-3, GM-CSF, and M-CSF, wherein one or both of the second media and the third media comprises the non-physiological ligand.

71. The method of any of claims 65-70, wherein step a) comprises:
(i) performing a first incubation comprising culturing the population of stem cells engineered with the synthetic cytokine receptor under conditions to form a first aggregate in a first media;
(ii) contacting the aggregate with a dissociating agent to form a population of dissociated cells;
(iii) performing a second incubation comprising culturing the population of dissociated cells under conditions to form a second aggregate in a second media, optionally wherein the second media comprises one or more of IL-3, GM-CSF, and M-CSF; and
(iv) performing a third incubation comprising culturing the population of cells in (iii) in a third media, optionally wherein the third media comprises one or more of IL-3, GM-CSF, and M-CSF.

72. The method of any of claims 68-71, wherein the first incubation is in a first media comprising one or more of BMP4, FGF2, VEGF-165, and a Rock Inhibitor.

73. The method of any of claims 68-72, wherein the first incubation is in a first media comprising BMP4, FGF2, VEGF-165, and a Rock Inhibitor.

74. The method of claim 72 or claim 73, wherein the Rock Inhibitor is Y27632.

75. The method of any one of claims 70-74, wherein the second media further comprises the non-physiological ligand of the synthetic cytokine receptor.

76. The method of any one of claim 70-74, wherein the second media does not comprise the non-physiological ligand of the synthetic cytokine receptor.

77. The method of any of claims 70-76, wherein the culturing in the first media is for 1 to 3 days.

78. The method of any one of claims 70-77, wherein the second media comprises one or more of BMP4, FGF2, VEGF, LY294002, IL-3, GM-CSF, and M-CSF.

79. The method of any one of claims 70-77, wherein the second media comprises BMP4, FGF2, VEGF, LY294002, IL-3, and M-CSF.

80. The method of any one of claims 70-79, wherein the second media further comprises a non-physiological ligand of the synthetic cytokine receptor.

81. The method of any one of claims 70-79, wherein the second media does not comprise a non-physiological ligand of the synthetic cytokine receptor.

82. The method of any one of claims 70-81, wherein the culturing in the second media is for 3 to 6 days.

83. The method of any one of claims 70-82, wherein the third media comprises one or more of UM729, StemRegenin-1, BMP4, FGF2, VEGF, LY294002, IL-3, GM-CSF, and M-CSF.

84. The method of any one of claims 70-82, wherein the third media comprises UM729, StemRegenin-1, BMP4, FGF2, VEGF, LY294002, IL-3, GM-CSF, and M-CSF.

85. The method of any one of claims 70-84, wherein the third media further comprises a non-physiological ligand of the synthetic cytokine receptor.

86. The method of any one of claims 70-84, wherein the third media does not comprise a non-physiological ligand of the synthetic cytokine receptor.

87. The method of any one of claims 70-86, wherein the culturing in the third media is for 6 to 12 days.

88. The method of any of claims 67-87, wherein the culturing in a) produces myeloid progenitor cells.

89. The method of claim 88, wherein the myeloid progenitor cell is a Granulocyte/Monocyte Progenitor Cells (“GMPs”).

90. The method of claim 88 or claim 89, wherein the myeloid progenitor cells are characterized with a surface phenotype CD34+, CD90−, and CD45RA+.

91. The method of claim 88 or claim 89, wherein the myeloid progenitor cells are characterized with a surface phenotype CD34+, CD90−, CD123+, and CD45RA+.

92. The method of any one of claims 65-91, wherein the culturing in b) is in a media comprising one or more of UM729, SCF, StemRegenin1, IL-3, GM-CSF, and M-CSF.

93. The method of any one of claims 65-91, wherein the culturing in b) is in a media comprising UM729, SCF, StemRegenin1, IL-3, GM-CSF, and M-CSF.

94. The method of any one of claims 65-93, wherein the culturing in b) is in a media further comprising a non-physiological ligand of the synthetic cytokine receptor.

95. The method of any one of claims 65-93, wherein the culturing in b) is in a media that does not comprise a non-physiological ligand of the synthetic cytokine receptor.

96. The method of any one of claims 67-95, wherein the culturing in b) is for 12 to 24 days.

97. A method of generating genetically engineered myeloid cells to express a synthetic cytokine receptor, comprising: culturing a population of hematopoietic progenitors (HP) cells derived from stem cells engineered with a synthetic cytokine receptor under conditions to differentiate cells into a population of myeloid cells, wherein at least a portion of the culturing is carried out in the presence of a non-physiological ligand of the synthetic cytokine receptor, and
wherein the synthetic cytokine receptor comprises:
a synthetic gamma chain polypeptide comprising a first dimerization domain, a transmembrane domain and an interleukin-2 receptor subunit gamma (IL-2RG) intracellular domain, and
a synthetic beta chain polypeptide comprising a second dimerization domain, a second transmembrane domain and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

98. A method of generating genetically engineered myeloid cells to express a synthetic cytokine receptor, comprising: culturing a population of myeloid progenitor cells derived from stem cells engineered with a synthetic cytokine receptor under conditions to differentiate cells into a population of myeloid cells, wherein at least a portion of the culturing is carried out in the presence of a non-physiological ligand of the synthetic cytokine receptor, and
wherein the synthetic cytokine receptor comprises:
a synthetic gamma chain polypeptide comprising a first dimerization domain, a first transmembrane domain and an interleukin-2 receptor subunit gamma (IL-2RG) intracellular domain, and
a synthetic beta chain polypeptide comprising a second dimerization domain, a second transmembrane domain and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

99. The method of claim 97 or claim 98, wherein the myeloid cells are macrophages or neutrophils.

100. A method of generating genetically engineered macrophages to express a synthetic cytokine receptor, comprising: culturing a population of hematopoietic progenitors (HP) cells derived from stem cells engineered with a synthetic cytokine receptor under conditions to differentiate cells into a population of macrophages, wherein at least a portion of the culturing is carried out in the presence of a non-physiological ligand of the synthetic cytokine receptor, and
wherein the synthetic cytokine receptor comprises:
a synthetic gamma chain polypeptide comprising a first dimerization domain, a first transmembrane domain and an interleukin-2 receptor subunit gamma (IL-2RG) intracellular domain, and
a synthetic beta chain polypeptide comprising a second dimerization domain, a second transmembrane domain and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

101. A method of generating genetically engineered macrophages to express a synthetic cytokine receptor, comprising: culturing a population of myeloid progenitor cells derived from stem cells engineered with a synthetic cytokine receptor under conditions to differentiate cells into a population of macrophages, wherein at least a portion of the culturing is carried out in the presence of a non-physiological ligand of the synthetic cytokine receptor, and
wherein the synthetic cytokine receptor comprises:
a synthetic gamma chain polypeptide comprising a first dimerization domain, a first transmembrane domain and an interleukin-2 receptor subunit gamma (IL-2RG) intracellular domain, and
a synthetic beta chain polypeptide comprising a second dimerization domain, a second transmembrane domain and a beta chain intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

102. The method of any of claims 97-101, wherein the culturing is in a media comprising one or more of UM729, SCF, StemRegenin1, IL-3, GM-CSF, and M-CSF.

103. The method of any one of claims 97-101, wherein the culturing is in a media comprising UM729, SCF, StemRegenin1, IL-3, GM-CSF, and M-CSF.

104. The method of any one of claims 97-103, wherein the culturing is for 12 to 24 days.

105. The method of any of claims 67-96, wherein the culturing of one or both steps a) and b) is carried out in a bioreactor.

106. The method of any of claims 97-104, wherein the culturing is carried out in a bioreactor.

107. The method of claim 105 or claim 106, wherein the bioreactor is a vertical wheel bioreactor.

108. The method of any of claims 105-107, wherein the bioreactor is a vertical wheel bioreactor with a volume of about 10 mL to about 1000 mL.

109. The method of claim 105, wherein the culturing in a) is carried out in a bioreactor and wherein the bioreactor is a vertical wheel bioreactor with a volume of about 100 mL.

110. The method of any claim 105 or claim 109, wherein the culturing in b) is carried out in a bioreactor and the bioreactor is a vertical wheel bioreactor with a volume of about 500 mL.

111. The method of acclaim 106, wherein the bioreactor is a vertical wheel bioreactor with a volume of about 500 mL.

112. The method of any one of claims 65-111, wherein the stem cells are pluripotent stem cells.

113. The method of claim 112, wherein the pluripotent stem cells are induced pluripotent stem cells.

114. The method of any of claims 65-112, wherein:
the synthetic gamma chain polypeptide comprises, in N- to C-terminal order, the first dimerization domain, the first transmembrane domain, and the IL-2RG intracellular domain, and the synthetic beta chain polypeptide comprises, in N- to C-terminal order, the second dimerization domain, the second transmembrane domain, and the intracellular domain.

115. The method of any of claims 65-114, wherein the first dimerization domain and the second dimerization domain are extracellular domains.

116. The method of any of claims 65-115, wherein the IL-2RG intracellular domain comprises a polypeptide sequence at least 95% identical to SEQ ID NO: 1, or a polypeptide sequence as set forth in SEQ ID NO: 1.

117. The method of any of claims 65-116, wherein the first transmembrane domain comprises the IL-2RG transmembrane domain.

118. The method of claim 117, wherein the IL-2RG transmembrane domain comprises a polypeptide sequence at least 95% identical to SEQ ID NO: 8 or 31, or a polypeptide sequence as set forth in SEQ ID NO: 8 or 31.

119. The method of any of claims 65-118, wherein the beta chain intracellular domain comprises the IL-2RB intracellular domain.

120. The method of claim 119, wherein the IL-2RB intracellular domain comprises a polypeptide sequence at least 95% identical to SEQ ID NO: 2, or a polypeptide sequence as set forth in SEQ ID NO: 2.

121. The method of any of claims 65-118, wherein the beta chain intracellular domain comprises the IL-7RB intracellular domain.

122. The method of claim 121, wherein the IL-7RB intracellular domain comprises a polypeptide sequence at least 95% identical to SEQ ID NO: 3, or a polypeptide sequence as set forth in SEQ ID NO: 3.

123. The method of any of claims 65-118, wherein the beta chain intracellular domain comprises the IL-21RB intracellular domain.

124. The method of claim 123, wherein the IL-21RB intracellular domain comprises a polypeptide sequence at least 95% identical to SEQ ID NO: 4, or a polypeptide sequence as set forth in SEQ ID NO: 4.

125. The method of any of claims 65-124, wherein the second transmembrane domain comprises a transmembrane domain from the same polypeptide as the intracellular domain.

126. The method of any one of claims 65-125, wherein the second transmembrane domain is a transmembrane domain of IL-2RB comprising a polypeptide sequence at least 95% identical to SEQ ID NO: 35 or 36, or a polypeptide sequence as set forth in SEQ ID NO: 35 or 36.

127. The method of any of claims 65-120, 125 and 126, wherein:
the first transmembrane domain of the synthetic gamma chain polypeptide is an IL-2RG transmembrane domain comprising the sequence set forth in SEQ ID NO: 8 or 31 and the IL-2RG intracellular domain comprises the sequence set forth in SEQ ID NO:1; and
the second transmembrane domain of the synthetic beta chain polypeptide is an IL-2RB transmembrane domain comprising the sequence set forth in SEQ ID NO: 35 or 36 andthe beta chain intracellular domain comprising the sequence set forth in SEQ ID NO:2.

128. The method of any of claims 65-127, wherein:
the first transmembrane domain and the IL-2RG intracellular domain of the synthetic gamma chain polypeptide comprises the sequence set forth in SEQ ID NOs: 31 and SEQ ID NO:
1; and
the second transmembrane domain and the beta chain intracellular domain of the synthetic beta chain polypeptide comprises the sequences set forth in SEQ ID NOs: 35 and SEQ ID NO:2.

129. The method of any one of claims 65-128, wherein the first dimerization domain and the second dimerization domain are heterodimerization domains selected from selected from FKBP12-rapamycin binding (FRB) domain and a FK506-Binding Protein of size 12 kD (FKBP); and/or
wherein the non-physiological ligand is rapamycin or a rapalog.

130. The method of any one of claims 65-129, wherein the first dimerization domain is FKBP and the second dimerization domain is FRB.

131. The method of any one of claims 65-129, wherein the first dimerization domain is FRB and the second dimerization domain is FKBP.

132. The method of any of claims 129-131, wherein the FRB domain comprises a polypeptide sequence at least 95% identical to SEQ ID NO: 6 or SEQ ID NO: 7.

133. The method of any of claims 129-132, wherein the FRB domain comprises the polypeptide sequence set forth in SEQ ID NO: 6 or SEQ ID NO: 7.

134. The engineered cell of any one of claims 129-133, wherein the FKBP domain comprises a polypeptide sequence at least 95% identical to SEQ ID NO: 5 or SEQ ID NO:30.

135. The method of any one of claims 129-134, wherein the FKBP domain comprises the polypeptide sequence set forth in SEQ ID NO: 5 or SEQ ID NO:30.

136. The method of any of claims 65-135, wherein the synthetic gamma chain polypeptide has the sequence of amino acids set forth in SEQ ID NO: 28 or a sequence of amino acids that is at least 90%, at least 95%, at least 96%, at least 97%, at least 98%, at least 99%, or 100% identical to SEQ ID NO: 28, and the synthetic beta chain polypeptide has the sequence of amino acids set forth in SEQ ID NO: 55 or a sequence of amino acids that is at least 90%, at least 95%, at least 96%, at least 97%, at least 98%, at least 99%, or 100% identical to SEQ ID NO: 55.

137. The method of any of claims 65-136, wherein the synthetic gamma chain polypeptide has the sequence of amino acids set forth in SEQ ID NO: 56 or a sequence of amino acids that is at least 90%, at least 95%, at least 96%, at least 97%, at least 98%, at least 99%, or 100% identical to SEQ ID NO: 56, and the synthetic beta chain polypeptide has the sequence of amino acids set forth in SEQ ID NO: 57 or a sequence of amino acids that is at least 90%, at least 95%, at least 96%, at least 97%, at least 98%, at least 99%, or 100% identical to SEQ ID NO: 57.

138. The method of any of claims 65-128, wherein the first dimerization domain and the second dimerization domain are homodimerization domains selected from:
i) FK506-Binding Protein of size 12 kD (FKBP);
ii) cyclophiliA (CypA); or
iii) gyrase B (CyrB);
and the non-physiological ligand is, respectively:
i) FK1012, AP1510, AP1903, or AP20187 or an analog thereof,
ii) cyclosporin-A (CsA) or an analog thereof, or
iii) coumermycin or an analog thereof.

139. The method of any of claims 65-138, wherein cells of the population of cells (the population of stem cells, the population of hematopoietic progenitor cells or the population of myeloid progenitor cells) are resistant to rapamycin-mediated mTOR inhibition.

140. The method of any of claims 65-139, wherein cells of the population of cells (the population of stem cells, the population of hematopoietic progenitor cells or the population of myeloid progenitor cells) express a cytosolic polypeptide that binds to the non-physiological ligand, optionally wherein the cytosolic polypeptide is a cytosolic FRB domain.

141. The method of any of claims 65-140, wherein the non-physiological ligand is rapamycin or a rapalog, and the cells of the population express a cytosolic FRB domain or variant thereof.

142. The method of claim 140 or claim 141, wherein the cytosolic FRB domain comprises a polypeptide sequence at least 95% identical to SEQ ID NO: 6 or SEQ ID NO: 7.

143. The method of claim 140 or claim 141, wherein the cytosolic FRB domain comprises a polypeptide sequence at least 98% identical to SEQ ID NO: 6 or SEQ ID NO: 7.

144. The method of any of claims 65-143, wherein cells of the population of cells (the population of stem cells, the population of hematopoietic progenitor cells or the population of myeloid progenitor cells) comprise a disrupted FKBP12 gene that reduces expression of FKBP12 in the cell.

145. The method of any of claims 65-144, wherein cells of the population of cells (the population of stem cells, the population of hematopoietic progenitor cells or the population of myeloid progenitor cells) comprise knock out of the FKBP12 gene.

146. The method of any of claims 65-145, wherein the synthetic cytokine receptor is integrated into an endogenous gene of cells of the population of cells (the population of stem cells, the population of hematopoietic progenitor cells or the population of myeloid progenitor cells) by targeted integration of the nucleotide sequence encoding the synthetic cytokine receptor into the endogenous gene.

147. The method of claim 146, wherein the targeted integration is by non-homologous end joining (NHEJ).

148. The method of claim 146, wherein the targeted integration is by homology directed repair.

149. The method of any of claims 146-148, wherein the insertion reduces expression ofthe endogenous gene in the locus.

150. The method of any of claims 146-149, wherein the insertion knocks out the endogenous gene in the locus.

151. The method of any of claims 146-150, wherein the insertion is by homology-directed repair.

152. The method of any of claims 146-151, wherein the endogenous gene is a housekeeping gene, a blood-lineage specific loci or an immune-related gene.

153. The method of claim 152, wherein the housekeeping gene is selected from eukaryotic translation elongation factor 1 alpha (EEF1A), glylceraldehyde-3-phosphate dehydrogenase (GAPDH), ubiquitin C (UBC), and actin beta (ACTB).

154. The method of claim 152, wherein the immune-related gene is selected from a beta-2-microglobulin (B2M) gene, and a signal regulatory protein alpha (SIRPA) gene.

155. The method of any one of claims 65-154, wherein cells of the population of cells (the population of stem cells, the population of hematopoietic progenitor cells or the population of myeloid progenitor cells) comprises a B2M knockout.

156. The method of any of claims 65-155, wherein cells of the population of cells (the population of stem cells, the population of hematopoietic progenitor cells or the population of myeloid progenitor cells) comprise a B2M knockout and a FKBP12 knockout.

157. The method of any one of claims 65-156, wherein cells of the population of cells (the population of stem cells, the population of hematopoietic progenitor cells or the population of myeloid progenitor cells) comprising a chimeric antigen receptor (CAR).

158. The method of any one of claims 66-96 and 99-157, wherein macrophages are mature macrophages that express CD14.

159. The method of any one of claims 65-158, wherein the non-physiological ligand is rapamycin or a rapamycin analog.

160. The method of claim 159, wherein the rapamycin analog is rapalog.

161. The method of any one of claims 65-160, wherein the non-physiological ligand is added to the media at a concentration of between 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM, 10 nM and 50 nM, 10 nM and 20 nM, 20 nM and 200 nM, 20 nM and 150 nM 20 nM and 100 nM, 20 nM and 50 nM, 50 nM and 200 nM, 50 nM and 150 nM, 50 nM and 100 nM, 100 nM and 200 nM, 100 nM and 150 nM and 150 nM and 200 nM.

162. The method of any one of claims 65-161, wherein the non-physiological ligand is added to the media at a concentration of at or about 10 nM.

163. The method of any of claims 65-162, wherein the non-physiological ligand is added to the media at a concentration of at or about 100 nM.

164. A population of myeloid cells produced by the method of any of claims 65, 66, 68-99 and 102-163.

165. A population of macrophages produced by the method of any of claims 65-96 and 99-163.

166. The population of claim 165, wherein the population of macrophages express CD14.

167. A pharmaceutical composition comprising the population of engineered myeloid cells of claim 62 or claim 164.

168. A pharmaceutical composition comprising the population of engineered macrophages of claim 63, claim 165 or claim 166.

169. The pharmaceutical composition of any of claims 167-168 comprising a pharmaceutically acceptable excipient.

170. The pharmaceutical composition of any of claims 167-169 comprising a cryoprotectant.

171. A method of expanding myeloid cells, the method comprising contacting the population of myeloid cells of claim 62 or claim 164, or the pharmaceutical composition of claim 167, 169 or 170 with the non-physiological ligand of the synthetic cytokine receptor.

172. A method of expanding macrophages, the method comprising contacting the population of macrophages of claim 63, claim 165 or claim 166, or the pharmaceutical composition of claim 168, 169 or 170 with the non-physiological ligand of the synthetic cytokine receptor.

173. The method of claim 171 or claim 172 that is performed in vitro or ex vivo.

174. The method of any one of claims 171-173, wherein the non-physiological ligand is rapamycin or a rapamycin analog.

175. The method of claim 174, wherein the rapamycin analog is rapalog.

176. The method of one any of claims 171-175, wherein the non-physiological ligand is contacted at a concentration of between 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM, 10 nM and 50 nM, 10 nM and 20 nM, 20 nM and 200 nM, 20 nM and 150 nM 20 nM and 100 nM, 20 nM and 50 nM, 50 nM and 200 nM, 50 nM and 150 nM, 50 nM and 100 nM, 100 nM and 200 nM, 100 nM and 150 nM and 150 nM and 200 nM.

177. The method of any one of claims 171-176, wherein the non-physiological ligand is contacted at a concentration of at or about 10 nM.

178. The method of any one of claims 171-177, wherein the non-physiological ligand is contacted at a concentration of at or about 100 nM.

179. The method of any one of claims 171-178, wherein the method is performed in vivo in a subject and the non-physiological ligand is administered to the subject.

180. A method of treating a disease or condition in a subject, comprising administering to the subject an effective amount of the population of myeloid cells of claim 62 or claim 164, or the pharmaceutical composition of claim 167, claim 169 or claim 170.

181. A method of treating a disease or condition in a subject, comprising administering to the subject an effective amount of the population of macrophages of claim 63, claim 165 or claim 166, or the pharmaceutical composition of claim 168, claim 169 or claim 170 with the non-physiological ligand of the synthetic cytokine receptor.

182. The method of claim 180 or claim 181, wherein the disease or condition is a cancer.

183. The method of any one of claims 171-182, wherein the cells express a CAR directed against an antigen expressed by cells of the disease or condition.

184. The method of claim 183, wherein the CAR targets a tumor antigen.

185. The method of any one of claims 171-184, comprising administering to the subject a non-physiological ligand of the synthetic cytokine receptor.

186. The method of any one of claims 171-185, wherein the non-physiological ligand is rapamycin or a rapamycin analog.

187. The method of claim 186, wherein the rapamycin analog is rapalog.

188. The method of any one of claims 171-187, wherein the non-physiological ligand is administered at a dose of 1 mg to 100 mg, optionally between 10-100 mg, optionally at or about 10 mg, 20 mg, 25 mg, 30 mg, 40 mg, 50 mg or any value between any of the foregoing.

189. The method of any one of claims 171-188, wherein multiple doses of the non-physiological ligand are administered to the subject.

190. The method of claim 189, wherein the multiple doses are administered intermittently or at regular intervals after administration of the macrophage population or composition thereof to the subject, optionally for a predetermined period of time.

191. The method of any one of claims 171-190, wherein 2 to 8 doses of the non-physiological ligand are administered to the subject.

192. The method of any one of claims 171-190, wherein a single dose of the non-physiological ligand is administered to the subject.

193. A kit comprising the engineered cell of any one of claims 1-60, the population of engineered myeloid cells of claim 62 or claim 164, the population of macrophages of claim 63, claim 165 or claim 166, or the pharmaceutical composition of any one of claims 168-170 and instructions for administering to a subject in need thereof.

194. The kit of claim 193, further comprising a container comprising the non-physiological ligand and instructions for administering the non-physiological ligand to the subject after administration of the cell population.

195. The kit of any of claim 193 or claim 194, wherein the subject has a cancer.


SEQUENCE LISTING (57 sequences)

SEQ ID NO: 1 - protein, 86 aa, synthetic construct
  REGION 1..86: note = IL2RG Common Gamma Chain Intracellular domain
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 2 - protein, 286 aa, synthetic construct
  REGION 1..286: note = IL2RB intracellular domain
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 3 - protein, 195 aa, synthetic construct
  REGION 1..195: note = IL7RB intracellular domain
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 4 - protein, 285 aa, synthetic construct
  REGION 1..285: note = IL21RB intracellular domain
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 5 - protein, 107 aa, synthetic construct
  REGION 1..107: note = FKBP domain (FKBP12)
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 6 - protein, 93 aa, synthetic construct [sequence omitted]

SEQ ID NO: 7 - protein, 93 aa, synthetic construct
  REGION 1..93: note = variant FRB domain (FRB mutant domain)
ILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 8 - protein, 21 aa, synthetic construct
  REGION 1..21: note = transmembrane (TM) domain
VVISVGSMGLIISLLCVYFWL

SEQ ID NO: 9 - protein, 19 aa, synthetic construct
  REGION 1..19: note = TM domain
VAVAGCVFLLISVLLLSGL

SEQ ID NO: 10 - protein, 25 aa, synthetic construct
  REGION 1..25: note = TM domain
PILLTISILSFFSVALLVILACVLW

SEQ ID NO: 11 - protein, 21 aa, synthetic construct
  REGION 1..21: note = TM domain
GWNPHLLLLLLLVIVFIPAFW

SEQ ID NO: 12 - protein, 21 aa, synthetic construct
  REGION 1..21: note = CD8a signal sequence
MALPVTALLLPLALLLHAARP

SEQ ID NO: 13 - DNA, 1554 bp, synthetic construct
  source 1..1554: mol_type = other DNA
  misc_feature 1..1554: note = nucleotide sequence encoding a CAR
atggccttaccagtgaccgccttgctcctgccgctggccttgctgctccacgccgccaggccggatgtcgtgatgacccagacccccctcagcctcccagtgtccctcggtgaccaggcttctattagttgcagatccagccagtccctcgtgcactctaacggtaatacctacctgagatggtatctccagaagcccggacagagccctaaggtgctgatctacaaagtctccaaccgggtgtctggagtccctgaccgcttctcagggagcggttccggcaccgacttcaccctgaagatcaaccgggtggaggccgaagacctcggcgtctatttctgctctcagagtacacatgtgccctggaccttcggcggagggaccaagctggagatcaaaagctccgcagacgatgccaagaaagatgccgctaagaaagacgatgctaagaaagacgatgcaaagaaagacggtggcgtgaagctggatgaaaccggaggaggtctcgtccagccaggaggagccatgaagctgagttgcgtgaccagcggattcacctttgggcactactggatgaactgggtgcgacagtccccagagaaggggctcgaatgggtcgctcagttcaggaacaaaccctacaattatgagacatactattcagacagcgtgaagggcaggtttactatcagtagagacgattccaaatctagcgtgtacctgcagatgaacaatctcagggtcgaagatacaggcatctactattgcacaggggcatcctatggtatggagtatctcggtcaggggacaagcgtcacagtcagtttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 14 - protein, 517 aa, synthetic construct
  REGION 1..517: note = CAR amino acid sequence
MALPVTALLLPLALLLHAARPDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 1560 bp, synthetic construct
  source 1..1560: mol_type = other DNA
  misc_feature 1..1560: note = nucleotide insert
gccaccatggccttaccagtgaccgccttgctcctgccgctggccttgctgctccacgccgccaggccggatgtcgtgatgacccagacccccctcagcctcccagtgtccctcggtgaccaggcttctattagttgcagatccagccagtccctcgtgcactctaacggtaatacctacctgagatggtatctccagaagcccggacagagccctaaggtgctgatctacaaagtctccaaccgggtgtctggagtccctgaccgcttctcagggagcggttccggcaccgacttcaccctgaagatcaaccgggtggaggccgaagacctcggcgtctatttctgctctcagagtacacatgtgccctggaccttcggcggagggaccaagctggagatcaaaagctccgcagacgatgccaagaaagatgccgctaagaaagacgatgctaagaaagacgatgcaaagaaagacggtggcgtgaagctggatgaaaccggaggaggtctcgtccagccaggaggagccatgaagctgagttgcgtgaccagcggattcacctttgggcactactggatgaactgggtgcgacagtccccagagaaggggctcgaatgggtcgctcagttcaggaacaaaccctacaattatgagacatactattcagacagcgtgaagggcaggtttactatcagtagagacgattccaaatctagcgtgtacctgcagatgaacaatctcagggtcgaagatacaggcatctactattgcacaggggcatcctatggtatggagtatctcggtcaggggacaagcgtcacagtcagtttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 16 - protein, 496 aa, synthetic construct
  REGION 1..496: note = CAR amino acid sequence without the signal peptide
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 21 aa, synthetic construct
  REGION 1..21: note = CAR amino acid sequence signal peptide
MALPVTALLLPLALLLHAARP

SEQ ID NO: 18 - RNA, 20 nt, synthetic construct
  source 1..20: mol_type = other RNA
  misc_feature 1..20: note = B2M gRNA
ggccgagatgtctcgctccg

SEQ ID NO: 19 - RNA, 20 nt, synthetic construct
  source 1..20: mol_type = other RNA
  misc_feature 1..20: note = FKBP12 gRNA
cacgcaggtctggccgcgct

SEQ ID NO: 20 - RNA, 20 nt, synthetic construct
  source 1..20: mol_type = other RNA
  misc_feature 1..20: note = FKBP12 gRNA
tgctgagccgatgcgcgcgg

SEQ ID NO: 21 - RNA, 20 nt, synthetic construct
  source 1..20: mol_type = other RNA
  misc_feature 1..20: note = FKBP12 gRNA
ctcaccgtctcctggggaga

SEQ ID NO: 22 - DNA, 500 bp, synthetic construct
  source 1..500: mol_type = other DNA
  misc_feature 1..500: note = B2M 5 prime homology arm
tacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggc

SEQ ID NO: 23 - DNA, 500 bp, synthetic construct
  source 1..500: mol_type = other DNA
  misc_RNA 1..500: note = B2M 3 prime homology arm
tggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctggggga

SEQ ID NO: 24 - DNA, 1189 bp, synthetic construct
  source 1..1189: mol_type = other DNA
  misc_feature 1..1189: note = EF1alpha promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaa

SEQ ID NO: 25 - DNA, 1188 bp, synthetic construct
  source 1..1188: mol_type = other DNA
  misc_feature 1..1188: note = EF1alpha promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 26 - DNA, 1205 bp, synthetic construct
  source 1..1205: mol_type = other DNA
  misc_feature 1..1205: note = EF1alpha promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcactagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaaa

SEQ ID NO: 27 - DNA, 347 bp, synthetic construct
  source 1..347: mol_type = other DNA
  misc_feature 1..347: note = MND promoter
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 28 - protein, 231 aa, synthetic construct
  REGION 1..231: note = RACR (g)
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 29 - protein, 20 aa, synthetic construct
  REGION 1..20: note = Signal peptide
MPLGLLWLGLALLGALHAQA

SEQ ID NO: 30 - protein, 108 aa, synthetic construct
  REGION 1..108: note = FKBP domain (FKBP12)
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGE

SEQ ID NO: 31 - protein, 37 aa, synthetic construct
  REGION 1..37: note = IL2RG transmembrane domain and ectodomain portion
GSNTSKENPFLFALEAVVISVGSMGLIISLLCVYFWL

SEQ ID NO: 32 - DNA, 3354 bp, synthetic construct
  source 1..3354: mol_type = other DNA
  misc_feature 1..3354: note = RACR
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgccactgggactgctgtggctgggactggccctgctgggcgccctgcacgcccaggccggcgtgcaggtggagacaatcagcccaggcgacggacgcacctttcctaagcggggccagacatgcgtggtgcactacaccggcatgctggaggatggcaagaagttcgactctagcagagataggaacaagccctttaagttcatgctgggcaagcaggaagtgatcagaggctgggaggagggcgtggcccagatgtctgtgggccagagggccaagctgacaatcagcccagactacgcatatggagcaaccggacacccaggaatcatcccacctcacgccacactggtgttcgatgtggagctgctgaagctgggcgagggctctaacaccagcaaggagaatccatttctgttcgcactggaggcagtggtcatctccgtgggctctatgggcctgatcatctccctgctgtgcgtgtacttttggctggagagaacaatgcccaggatccctaccctgaagaacctggaggacctggtgaccgagtatcacggcaatttcagcgcctggtccggcgtgtctaagggactggcagagtccctgcagccagattactctgagcggctgtgcctggtgagcgagatcccacccaagggaggcgccctgggagagggacctggagcaagcccatgcaaccagcactccccatattgggcccctccatgttacaccctgaagcctgagacaggaagcggagcgacgaattttagtctactgaaacaagcgggagacgtggaggaaaaccctggacctatggcactgccagtgaccgccctgctgctgccactggccctgctgctgcacgcagcacgccccatcctgtggcacgaaatgtggcatgaaggcctggaggaggcaagcagactgtattttggcgagagaaatgtgaaaggaatgtttgaggtgctggagccactgcacgcaatgatggagaggggacctcagaccctgaaggagacatcctttaaccaggcctatggcagagacctgatggaggcccaggagtggtgcaggaagtacatgaagagcggaaatgtgaaagacctgctgcaggcctgggatctgtattatcacgtgttcagaaggatcagcaagggcaaggataccatcccttggctgggacacctgctggtgggactgtccggagcctttggcttcatcatcctggtgtacctgctgatcaactgccgcaatacaggcccatggctgaagaaggtgctgaagtgtaacaccccagacccctccaagttcttttctcagctgtcctctgagcacggcggcgatgtgcagaagtggctgagctccccttttccatctagctccttcagccctggaggactggcaccagagatctcccctctggaggtgctggagcgggacaaggtgacccagctgctgctgcagcaggataaggtgcccgagcctgcctccctgtctagcaaccacagcctgacctcctgctttacaaatcagggctatttctttttccacctgccagacgcactggagatcgaggcatgtcaggtgtacttcacatatgatccatacagcgaggaggacccagatgagggagtggccggcgcccctaccggatcctctccacagccactgcagccactgtctggcgaggacgatgcctactgtacatttcctagcagagacgatctgctgctgttctccccttctctgctgggaggaccatctccacctagcaccgcacctggaggatccggggcaggggaggagaggatgccaccatctctgcaggagagggtgccaagggactgggatcctcagccactgggacctccaaccccaggagtgcccgacctggtggatttccagccccctccagagctggtgctgagagaggcaggagaggaggtgccagacgcaggaccaagagagggcgtgagctttccatggtccaggccacctggacagggagagttcagagccctgaacgccaggctgccactgaatacagacgcctacctgtctctgcaggagctgcagggccaggatcccacccacctggtgtaaacaataaaagatctttattttcattagatctgtgtgttggttttttgtgtg

SEQ ID NO: 33 - protein, 429 aa, synthetic construct
  REGION 1..429: note = RACR (b)
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 34 - protein, 21 aa, synthetic construct
  REGION 1..21: note = CD8a Signal peptide
MALPVTALLLPLALLLHAARP

SEQ ID NO: 35 - protein, 29 aa, synthetic construct
  REGION 1..29: note = IL2RB transmembrane domain and ectodomain portion
GKDTIPWLGHLLVGLSGAFGFIILVYLLI

SEQ ID NO: 36 - protein, 25 aa, synthetic construct
  REGION 1..25: note = IL2RB transmembrane domain
IPWLGHLLVGLSGAFGFIILVYLLI

SEQ ID NO: 37 - DNA, 753 bp, synthetic construct
  source 1..753: mol_type = other DNA
  misc_feature 1..753: note = RACR (g) Nucleotides
atgccactgggactgctgtggctgggactggccctgctgggcgccctgcacgcccaggccggcgtgcaggtggagacaatcagcccaggcgacggacgcacctttcctaagcggggccagacatgcgtggtgcactacaccggcatgctggaggatggcaagaagttcgactctagcagagataggaacaagccctttaagttcatgctgggcaagcaggaagtgatcagaggctgggaggagggcgtggcccagatgtctgtgggccagagggccaagctgacaatcagcccagactacgcatatggagcaaccggacacccaggaatcatcccacctcacgccacactggtgttcgatgtggagctgctgaagctgggcgagggctctaacaccagcaaggagaatccatttctgttcgcactggaggcagtggtcatctccgtgggctctatgggcctgatcatctccctgctgtgcgtgtacttttggctggagagaacaatgcccaggatccctaccctgaagaacctggaggacctggtgaccgagtatcacggcaatttcagcgcctggtccggcgtgtctaagggactggcagagtccctgcagccagattactctgagcggctgtgcctggtgagcgagatcccacccaagggaggcgccctgggagagggacctggagcaagcccatgcaaccagcactccccatattgggcccctccatgttacaccctgaagcctgagaca

SEQ ID NO: 38 - DNA, 1287 bp, synthetic construct
  source 1..1287: mol_type = other DNA
  misc_feature 1..1287: note = RACR (b) nucleotides
atggcactgccagtgaccgccctgctgctgccactggccctgctgctgcacgcagcacgccccatcctgtggcacgaaatgtggcatgaaggcctggaggaggcaagcagactgtattttggcgagagaaatgtgaaaggaatgtttgaggtgctggagccactgcacgcaatgatggagaggggacctcagaccctgaaggagacatcctttaaccaggcctatggcagagacctgatggaggcccaggagtggtgcaggaagtacatgaagagcggaaatgtgaaagacctgctgcaggcctgggatctgtattatcacgtgttcagaaggatcagcaagggcaaggataccatcccttggctgggacacctgctggtgggactgtccggagcctttggcttcatcatcctggtgtacctgctgatcaactgccgcaatacaggcccatggctgaagaaggtgctgaagtgtaacaccccagacccctccaagttcttttctcagctgtcctctgagcacggcggcgatgtgcagaagtggctgagctccccttttccatctagctccttcagccctggaggactggcaccagagatctcccctctggaggtgctggagcgggacaaggtgacccagctgctgctgcagcaggataaggtgcccgagcctgcctccctgtctagcaaccacagcctgacctcctgctttacaaatcagggctatttctttttccacctgccagacgcactggagatcgaggcatgtcaggtgtacttcacatatgatccatacagcgaggaggacccagatgagggagtggccggcgcccctaccggatcctctccacagccactgcagccactgtctggcgaggacgatgcctactgtacatttcctagcagagacgatctgctgctgttctccccttctctgctgggaggaccatctccacctagcaccgcacctggaggatccggggcaggggaggagaggatgccaccatctctgcaggagagggtgccaagggactgggatcctcagccactgggacctccaaccccaggagtgcccgacctggtggatttccagccccctccagagctggtgctgagagaggcaggagaggaggtgccagacgcaggaccaagagagggcgtgagctttccatggtccaggccacctggacagggagagttcagagccctgaacgccaggctgccactgaatacagacgcctacctgtctctgcaggagctgcagggccaggatcccacccacctggtg

SEQ ID NO: 39 - DNA, 49 bp, synthetic construct
  source 1..49: mol_type = other DNA
  misc_feature 1..49: note = Synthetic polyA
aataaaagatctttattttcattagatctgtgtgttggttttttgtgtg

SEQ ID NO: 40 - DNA, 4354 bp, synthetic construct
  source 1..4354: mol_type = other DNA
  misc_feature 1..4354: note = RACR_B2M ssODN
tacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgccactgggactgctgtggctgggactggccctgctgggcgccctgcacgcccaggccggcgtgcaggtggagacaatcagcccaggcgacggacgcacctttcctaagcggggccagacatgcgtggtgcactacaccggcatgctggaggatggcaagaagttcgactctagcagagataggaacaagccctttaagttcatgctgggcaagcaggaagtgatcagaggctgggaggagggcgtggcccagatgtctgtgggccagagggccaagctgacaatcagcccagactacgcatatggagcaaccggacacccaggaatcatcccacctcacgccacactggtgttcgatgtggagctgctgaagctgggcgagggctctaacaccagcaaggagaatccatttctgttcgcactggaggcagtggtcatctccgtgggctctatgggcctgatcatctccctgctgtgcgtgtacttttggctggagagaacaatgcccaggatccctaccctgaagaacctggaggacctggtgaccgagtatcacggcaatttcagcgcctggtccggcgtgtctaagggactggcagagtccctgcagccagattactctgagcggctgtgcctggtgagcgagatcccacccaagggaggcgccctgggagagggacctggagcaagcccatgcaaccagcactccccatattgggcccctccatgttacaccctgaagcctgagacaggaagcggagcgacgaattttagtctactgaaacaagcgggagacgtggaggaaaaccctggacctatggcactgccagtgaccgccctgctgctgccactggccctgctgctgcacgcagcacgccccatcctgtggcacgaaatgtggcatgaaggcctggaggaggcaagcagactgtattttggcgagagaaatgtgaaaggaatgtttgaggtgctggagccactgcacgcaatgatggagaggggacctcagaccctgaaggagacatcctttaaccaggcctatggcagagacctgatggaggcccaggagtggtgcaggaagtacatgaagagcggaaatgtgaaagacctgctgcaggcctgggatctgtattatcacgtgttcagaaggatcagcaagggcaaggataccatcccttggctgggacacctgctggtgggactgtccggagcctttggcttcatcatcctggtgtacctgctgatcaactgccgcaatacaggcccatggctgaagaaggtgctgaagtgtaacaccccagacccctccaagttcttttctcagctgtcctctgagcacggcggcgatgtgcagaagtggctgagctccccttttccatctagctccttcagccctggaggactggcaccagagatctcccctctggaggtgctggagcgggacaaggtgacccagctgctgctgcagcaggataaggtgcccgagcctgcctccctgtctagcaaccacagcctgacctcctgctttacaaatcagggctatttctttttccacctgccagacgcactggagatcgaggcatgtcaggtgtacttcacatatgatccatacagcgaggaggacccagatgagggagtggccggcgcccctaccggatcctctccacagccactgcagccactgtctggcgaggacgatgcctactgtacatttcctagcagagacgatctgctgctgttctccccttctctgctgggaggaccatctccacctagcaccgcacctggaggatccggggcaggggaggagaggatgccaccatctctgcaggagagggtgccaagggactgggatcctcagccactgggacctccaaccccaggagtgcccgacctggtggatttccagccccctccagagctggtgctgagagaggcaggagaggaggtgccagacgcaggaccaagagagggcgtgagctttccatggtccaggccacctggacagggagagttcagagccctgaacgccaggctgccactgaatacagacgcctacctgtctctgcaggagctgcagggccaggatcccacccacctggtgtaaacaataaaagatctttattttcattagatctgtgtgttggttttttgtgtgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctggggga

SEQ ID NO: 41 - DNA, 66 bp, synthetic construct
  source 1..66: mol_type = other DNA
  misc_feature 1..66: note = PQR linker nucleotide
ggaagcggagcgacgaattttagtctactgaaacaagcgggagacgtggaggaaaaccctggacct

SEQ ID NO: 42 - protein, 22 aa, synthetic construct
  REGION 1..22: note = PQR linker
GSGATNFSLLKQAGDVEENPGP

SEQ ID NO: 43 - protein, 22 aa, synthetic construct
  REGION 1..22: note = F2A
VKQTLNFDLLKLAGDVESNPGP

SEQ ID NO: 44 - protein, 20 aa, synthetic construct
  REGION 1..20: note = E2A
QCTNYALLKLAGDVESNPGP

SEQ ID NO: 45 - protein, 24 aa, synthetic construct
  REGION 1..24: note = T2A
LEGGGEGRGSLLTCGDVEENPGPR

SEQ ID NO: 46 - protein, 18 aa, synthetic construct
  REGION 1..18: note = T2A
EGRGSLLTCGDVEENPGP

SEQ ID NO: 47 - protein, 22 aa, synthetic construct
  REGION 1..22: note = P2A
GSGATNFSLLKQAGDVEENPGP

SEQ ID NO: 48 - protein, 19 aa, synthetic construct
  REGION 1..19: note = P2A
ATNFSLLKQAGDVEENPGP

SEQ ID NO: 49 - protein, 106 aa, synthetic construct
  REGION 1..106: note = FKBP domain (FKBP12)
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKL

SEQ ID NO: 50 - DNA, 1380 bp, synthetic construct
  source 1..1380: mol_type = other DNA
  misc_feature 1..1380: note = CD19 CAR nucleotide
ctgctgctggtgacctccctgctgctgtgcgagctgcctcacccagcctttctgctgatccccgacatccagatgacacagaccacaagctccctgtctgccagcctgggcgacagagtgaccatctcctgtagggcctctcaggatatcagcaagtacctgaactggtatcagcagaagccagatggcacagtgaagctgctgatctaccacacctccaggctgcactctggagtgccaagccggttctccggatctggaagcggcaccgactattccctgacaatctctaacctggagcaggaggatatcgccacatacttttgccagcagggcaataccctgccatatacattcggcggaggaaccaagctggagatcaccggatccacatctggaagcggcaagccaggaagcggagagggatccacaaagggagaggtgaagctgcaggagagcggaccaggactggtggcaccatcccagtctctgagcgtgacctgtacagtgtccggcgtgtctctgcctgactacggcgtgtcctggatcaggcagccacctaggaagggactggagtggctgggcgtgatctggggctctgagaccacatactataattctgccctgaagagccgcctgaccatcatcaaggacaactccaagtctcaggtgtttctgaagatgaatagcctgcagaccgacgatacagccatctactattgcgccaagcactactattacggcggctcctacgccatggattattggggccagggcacctccgtgacagtgtctagcggcgctgtgcacaccagaggactggatttcgcctgcgacttctgggtgctggtggtggtgggaggcgtgctggcctgttactccctgctggtgaccgtggcctttatcatcttctgggtgaagagaggcaggaagaagctgctgtatatctttaagcagcccttcatgcgccctgtgcagaccacacaggaggaggacggctgcagctgtcggtttccagaggaggaggagggaggatgcgagctgcgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacatatgatgccctgcacatgcaggccctgccacctagg

SEQ ID NO: 51 - protein, 460 aa, synthetic construct
  REGION 1..460: note = CD19 CAR polypeptide
LLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - RNA, 20 nt, synthetic construct
  source 1..20: mol_type = other RNA
  misc_feature 1..20: note = AAVS1 gRNA
ggggccactagggacaggat

SEQ ID NO: 53 - DNA, 500 bp, synthetic construct
  source 1..500: mol_type = other DNA
  misc_feature 1..500: note = 5` homology arm for AAVS1
tttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccacta

SEQ ID NO: 54 - DNA, 500 bp, synthetic construct
  source 1..500: mol_type = other DNA
  misc_feature 1..500: note = 3` homology arm for AAVSI
gggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtc

SEQ ID NO: 55 - protein, 408 aa, synthetic construct
  REGION 1..408: note = RACR (b) without signal peptide
ILWHEMWHEGLEEASRLYFGERNVKGMFEVLEPLHAMMERGPQTLKETSFNQAYGRDLMEAQEWCRKYMKSGNVKDLLQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 56 - protein, 216 aa, synthetic construct
  REGION 1..216: note = FRB:IL2RG polypeptide
ILWHEMWHEGLEEASRLYFGERNVKGMFEVLEPLHAMMERGPQTLKETSFNQAYGRDLMEAQEWCRKYMKSGNVKDLLQAWDLYYHVFRRISKGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 57 - protein, 423 aa, synthetic construct
  REGION 1..423: note = FKBP12:IL2RB polypeptide
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV